SEQUENCE LISTING (25 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, synthetic construct
  REGION 1..16: note = Synthetic
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, synthetic construct
  REGION 1..288: note = Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
  REGION 1..183: note = Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - DNA, 22 bp, synthetic construct
  misc_feature 1..22: note = Synthetic
  source 1..22: mol_type = other DNA
tcaaggattccttgacttaagt

SEQ ID NO: 21 - DNA, 21 bp, synthetic construct
  misc_feature 1..21: note = Synthetic
  source 1..21: mol_type = other DNA
tcaaggattccttgacttaag

SEQ ID NO: 22 - DNA, 14 bp, synthetic construct
  misc_feature 1..14: note = Synthetic
  source 1..14: mol_type = other DNA
gtggatggatggag

SEQ ID NO: 23 - DNA, 13 bp, synthetic construct
  misc_feature 1..13: note = Synthetic
  source 1..13: mol_type = other DNA
gtggatggatgga

SEQ ID NO: 24 - DNA, 10 bp, synthetic construct
  misc_feature 1..10: note = Synthetic
  source 1..10: mol_type = other DNA
ccacacacca

SEQ ID NO: 25 - DNA, 11 bp, synthetic construct
  misc_feature 1..11: note = Synthetic
  source 1..11: mol_type = other DNA
cacacatatat

(19) United States
(12) Patent Application Publication
(10) Pub. No.: US 2024/0084387 A1
(43) Pub. Date: Mar. 14, 2024

(54) GENETIC VARIANTS ASSOCIATED WITH LOCAL FAT DEPOSITION TRAITS FOR THE TREATMENT OF HERITABLE METABOLIC DISORDERS

(71) Applicants: THE BROAD INSTITUTE, INC., Cambridge, MA (US); THE GENERAL HOSPITAL CORPORATION, Boston, MA (US)

(72) Inventors: Amit Khera, Boston, MA (US); Saaket Agrawal, Boston, MA (US); Marcus Klarqvist, Cambridge, MA (US); Puneet Batra, Cambridge, MA (US)

(21) Appl. No.: 18/454,465

(22) Filed: Aug. 23, 2023

Related U.S. Application Data
(60) Provisional application No. 63/401,069, filed on Aug. 25, 2022.

(51) Int. Cl. C12Q 1/6883; A61B 5/00

(52) U.S. Cl. CPC C12Q 1/6883; A61B 5/4866; C12Q 2600/106; C12Q 2600/156

(57) ABSTRACT

The subject matter disclosed herein is generally directed to genetic variants associated with local adiposity traits and metabolic disease. Embodiments disclosed herein provide genetic variants associated with local adiposity traits obtained by adjusting adiposity traits for BMI and height. Embodiments disclosed herein also provide genes linked to variants and associated with the local adiposity traits. The local adiposity traits are associated with metabolic disorders. In example embodiments, variants indicate risk for a metabolic disorder and can be used to determine treatment. In example embodiments, genes associated with local adiposity traits and/or variants can be targeted therapeutically. In example embodiments, a risk for a metabolic disorder can be determined by detecting one or more risk variants associated with a local adiposity trait.

### CROSS-REFERENCE TO RELATED APPLICATIONS

This application claims the benefit of U.S. Provisional Application No. 63/401,069, filed Aug. 25, 2022. The entire contents of the above-identified application are hereby fully incorporated herein by reference.

### REFERENCE TO AN ELECTRONIC SEQUENCE LISTING

The contents of the electronic sequence listing (“BROD-5670US_ST26.xml”; Size is 26,559 bytes and it was created on Aug. 23, 2023) is herein incorporated by reference in its entirety.

### TECHNICAL FIELD

The subject matter disclosed herein is generally directed to genetic variants associated with local adiposity traits and metabolic disease.

### BACKGROUND

Overall fat mass and fat distribution represent two correlated but distinct axes of variation that determine the health impacts of adipose tissue. Individuals with high body mass index (BMI)—defining obesity—are at elevated risk of type 2 diabetes and cardiovascular events, but increased cardiometabolic risk has also been noted in individuals with the same BMI when fat is disproportionally depleted in more favorable gluteofemoral fat depots and deposited instead in visceral and ectopic fat depots¹⁻⁵. An extreme example of this paradigm occurs in Mendelian lipodystrophies, such as those caused by missense mutations in the LAMA and PPARG genes⁶⁻¹⁰. By contrast, the genetic architecture of more subtle variation in fat distribution across the general population warrants further attention.

In general, prior studies aiming to elucidate common genetic variation contributing to fat distribution can be categorized into three study types: (1) genome-wide association studies (GWAS) on anthropometric proxies of fat distribution, (2) studies combining GWAS summary statistics of metabolic and anthropometric traits, and (3) GWASs on imaging-based measures of fat distribution. The first type has been spearheaded by the Genetic Investigation of Anthropometric Traits (GIANT) consortium and others, leading to the discovery of over 300 loci associated with waist-to-hip ratio adjusted for BMI (WHRadjBMI) in an analysis of nearly 700,000 individuals^{11,12}. Another recent GWAS aimed to examine fat distribution using estimates of body composition based on stepping on a scale equipped with impedance technology, known to be reasonably accurate for total fat volume but less so for fat distribution¹³⁻¹⁵. Despite the considerable value of these studies, a central limitation is an unclear relationship between each anthropometric trait and each fat depot of biological interest—for example, an increase in WHRadjBMI could be capturing increased visceral adipose tissue (VAT; around the abdominal organs), increased abdominal subcutaneous adipose tissue (ASAT; abdominal fat under the skin), decreased gluteofemoral adipose tissue (GFAT; hip and thigh fat), or some combination of these perturbations^{16,17}. Variation in WHRadjBMI could also reflect variation in muscle and bone mass, rather than adipose tissue burden.

A second category of studies has aimed to gain further resolution into anthropometric loci by combining summary statistics of metabolic and anthropometric traits, generating clusters of metabolically favorable and unfavorable loci¹⁸⁻²³. These studies have succeeded in establishing a common variant basis for metabolically distinct fat depots, with seminal work demonstrating that an insulin resistance polygenic score is associated with lower hip circumference in the general population, and that individuals with familial partial lipodystrophy type 1 (FPLD1) have a higher burden of this polygenic score¹⁹. Along with their reliance on anthropometric proxies of fat distribution, these studies are limited by their inclusion requirement of nominal significance across multiple metabolic traits which is likely leading to only a fraction of the genetic architecture of fat distribution being described.

Finally, the third category of studies performed GWASs on measurements derived from body imaging²⁴⁻²⁹. These include GWASs of CT-quantified VAT and ASAT in nearly 20,000 individuals, GWASs on Mill-quantified VAT and ASAT, and a GWAS of a predicted VAT trait using several anthropometric traits trained on over 4000 DEXA-measured VAT values²⁶⁻²⁹. These studies have been important for translating insights from anthropometric and metabolic trait GWASs to image-derived measurements of the fat depots of interest, but have been limited by (1) the absence of GFAT, which appears to have a metabolically protective role in contrast to VAT and ASAT, and frequently (2) a reliance on raw, unadjusted fat depot metrics which are highly correlated with both each other and BMI.

Citation or identification of any document in this application is not an admission that such a document is available as prior art to the present invention.

### SUMMARY

In one aspect, the present invention provides for a method of treating a metabolic disorder comprising: detecting one or more indicators of metabolic disease in a subject having a variant that increases risk for the metabolic disorder or a variant that decreases risk for the metabolic disorder; and treating the subject with one or more agents capable of treating the metabolic disorder if the one or more indicators of metabolic disease are detected in the subject having a variant that increases risk for the metabolic disorder, wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657.

In another aspect, the present invention provides for a method of treating a metabolic disorder comprising: detecting one or more indicators of metabolic disease in a subject having a variant that increases risk for the metabolic disorder or a variant that decreases risk for the metabolic disorder; and treating the subject with one or more agents capable of treating the metabolic disorder if the one or more indicators of metabolic disease are detected in the subject having a variant that increases risk for the metabolic disorder; or treating the subject with a healthy lifestyle regimen if the one or more indicators of metabolic disease are detected in the subject having a variant that decreases risk for the metabolic disorder, wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657.

In certain embodiments, the one or more indicators of metabolic disease is selected from the group consisting of: increased visceral adipose tissue (VAT), increased abdominal subcutaneous adipose tissue (ASAT), decreased gluteofemoral adipose tissue (GFAT), increased serum triglycerides, decreased HDL-c (HDL-cholesterol), increased LDL-c (LDL-cholesterol), increased liver enzymes, and increased HbA1C (hemoglobin A1C). In certain embodiments, the increased liver enzymes comprise alanine aminotransferase (ALT). In certain embodiments, the one or more indicators of metabolic disease are detected by a blood test. In certain embodiments, the one or more indicators of metabolic disease are detected by CT-scan, DEXA-scan, or MRI. In certain embodiments, the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance.

In another aspect, the present invention provides for a method of treating a metabolic disorder comprising: detecting one or more indicators of metabolic disease in a subject having a polygenic risk score (PRS) for an adiposity trait adjusted for BMI and height selected from the group consisting of GFAT, VAT, and ASAT; and treating the subject with one or more agents capable of treating the metabolic disorder if the one or more indicators of metabolic disease are detected in the subject having a low PRS for BMI and height adjusted GFAT, a high PRS for BMI and height adjusted VAT, and/or a high PRS for BMI and height adjusted ASAT; or treating the subject with a healthy lifestyle regimen if the one or more indicators of metabolic disease are detected in the subject having a high PRS for BMI and height adjusted GFAT, a low PRS for BMI and height adjusted VAT, and/or a low PRS for BMI and height adjusted ASAT. In certain embodiments, the variant activity of the PRS is enriched in adipose tissue. In certain embodiments, the PRS includes up to 1,125,301 variants. In certain embodiments, the one or more indicators of metabolic disease is selected from the group consisting of: increased visceral adipose tissue (VAT), increased abdominal subcutaneous adipose tissue (ASAT), decreased gluteofemoral adipose tissue (GFAT), increased serum triglycerides, decreased HDL-c (HDL-cholesterol), increased LDL-c (LDL-cholesterol), increased liver enzymes, and increased HbA1C (hemoglobin A1C). In certain embodiments, the increased liver enzymes comprise alanine aminotransferase (ALT). In certain embodiments, the one or more indicators of metabolic disease are detected by a blood test. In certain embodiments, the one or more indicators of metabolic disease are detected by CT-scan, DEXA-scan, or MRI. In certain embodiments, the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance.

In certain embodiments, the one or more agents comprise a PPAR-alpha agonist. In certain embodiments, the one or more agents comprise a PPAR-gamma agonist. In certain embodiments, the PPAR-gamma agonist is a thiazolidinedione selected from the group consisting of Pioglitazone, Rosiglitazone, Lobeglitazone, Ciglitazone, Darglitazone, Englitazone, Netoglitazone, Rivoglitazone, Troglitazone, Balaglitazone, and AS-605240. In certain embodiments, the one or more agents comprise a PPAR-delta agonist. In certain embodiments, the one or more agents comprise a dual or pan PPAR agonist. In certain embodiments, the one or more agents comprise a growth hormone-releasing hormone (GHRH). In certain embodiments, the GHRH is selected from the group consisting of Tesamorelin, Somatocrinin, CJC-1295, Modified GRF (1-29), Dumorelin, Rismorelin, Sermorelin, and Somatorelin. In certain embodiments, the one or more agents comprise a sodium-glucose transporter 2 (SGLT2) inhibitor. In certain embodiments, the SGLT2 inhibitor is selected from the group consisting of Canagliflozin, Dapagliflozin, Empagliflozin, Ertugliflozin, Ipragliflozin, Luseogliflozin, Remogliflozin, Sotagliflozin, and Tofogliflozin. In certain embodiments, the one or more agents comprise metformin. In certain embodiments, the one or more agents comprise an alpha-glucosidase inhibitor. In certain embodiments, the one or more agents comprise an incretin-based therapy. In certain embodiments, the one or more agents comprise a sulfonylurea. In certain embodiments, the one or more agents comprise Metreleptin. In certain embodiments, the one or more agents is an antisense oligonucleotide (ASO). In certain embodiments, the one or more agents is a gene modifying agent. In certain embodiments, the gene modifying agent is a CRISPR-Cas gene editing agent.

In another aspect, the present invention provides for a method of treating a metabolic disorder in a subject in need thereof comprising administering one or more agents targeting a gene associated with a variant selected from Supplementary Data 3. In certain embodiments, the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657. In certain embodiments, the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance. In certain embodiments, the expression of the gene is regulated by the variant. In certain embodiments, the gene is in contact with a genomic loci comprising the variant.

In another aspect, the present invention provides for a method of treating a metabolic disorder in a subject in need thereof comprising administering one or more agents targeting one or more genes associated with an adiposity trait adjusted for BMI and height selected from the group consisting of GFAT, VAT and ASAT, wherein the one or more genes are selected from Supplementary Data 13. In certain embodiments, the one or more genes are selected from the group consisting of: CEBPA-AS1, CCDC92, FLOT1, CYP21A1P, HLA-DRB6, and HLA-S; or CENPW, TIPARP, and AC103965.1; or CCDC92, DNAH100S, RP11-380L11.4, IRS1, ZNF664, RIMKLBP2, DNAH10, RP11-392O17.1, VEGFB, FAM13A, PDGFC, MAFF, TMEM165, RP11-177J6.1, CLOCK, and SRD5A3-AS1; or CEBPA-AS1, CCDC92, ADCY3, FLOT1, TIPARP, CEBPA-AS1, and IRS1; or CCDC92, CEBPA-AS1, RP11-380L11.4, DNAH100S, HLA-S, DNAH10, CCDC92, DNAH100S, CEBPA-AS1, RP11-380L11.4, XXbac-BPG248L24.12, HLA-S, and VEGFB; or CCDC92, and TIPARP. In certain embodiments, the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance.

In certain embodiments, the one or more agents is an agonist of the gene. In certain embodiments, the one or more agents is an antagonist of the gene. In certain embodiments, the one or more agents increase expression of the gene. In certain embodiments, the one or more agents decrease expression of the gene. In certain embodiments, the one or more agents is a small molecule. In certain embodiments, the one or more agents is an antisense oligonucleotide (ASO). In certain embodiments, the one or more agents is a gene modifying agent. In certain embodiments, the gene modifying agent is a CRISPR-Cas gene editing agent. In certain embodiments, the method further comprises monitoring treatment efficacy by detecting one or more indicators of the metabolic disorder in the subject.

In another aspect, the present invention provides for a method of detecting a risk for a metabolic disorder comprising detecting in a subject one or more risk variants associated with an adiposity trait adjusted for BMI and height selected from the group consisting of GFAT, VAT and ASAT. In certain embodiments, the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657. In certain embodiments, the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), Nonalcoholic fatty liver disease (NAFLD), and impaired glucose tolerance. In certain embodiments, the one or more variants are polygenic risk variants.

In certain embodiments, the subject is female. In certain embodiments, the subject is male.

In another aspect, the present invention provides for a method of detecting one or more risk variants in a sample from a subject, wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657. In certain embodiments, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of the risk variants are detected in the sample from the subject. In certain embodiments, the one or more risk variants are detected by hybridization, nucleic acid amplification, or sequencing.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which (color drawings are available in Agrawal S, Wang M, Klarqvist M D R, et al. Inherited basis of visceral, abdominal subcutaneous and gluteofemoral fat depots. Nat Commun. 2022; 13(1):3771):

FIG. 1A-1E—Genome-wide association studies of VATadj, ASATadj, and GFATadj. (FIG. 1A) Three female participants from the UK Biobank with similar age (67-70 years) and similar overweight BMI (27.6-28.6 kg/m 2) with highly discordant fat distributions (FIG. 1B, C, D) Manhattan plots for sex-combined GWASs with VAT adjusted for BMI and height (VATadj), ASATadj, and GFATadj. Lead SNPs are described in Supplementary Data 3. (FIG. 1E) Overlap between VATadj, ASATadj, and GFATadj loci denoted by the nearest gene; lead SNPs of two traits in high LD (R²≥0.1) were plotted in the intersection. GWAS significance at a commonly used threshold of p<5×10⁻⁸ was required for inclusion in the Venn diagram.

FIG. 2—Observational and genetic correlations between MRI-derived adiposity traits, BMI, and WHRadjBMI. Observational correlations displayed are Pearson correlation coefficients. Genetic correlations were obtained from cross-trait LD-score regression using sex-combined summary statistics. Additional correlogram entries, including sex-stratified analyses, are available in FIGS. 13 and 14.

FIG. 3A-3C—Common variant sex heterogeneity for VATadj, ASATadj, and GFATadj local adiposity traits. For each adiposity trait, independent loci that were associated with the trait in either sex-combined or sex-stratified analyses are plotted (Supplementary Data 10). Thirty-four such loci are plotted for VATadj, 27 for ASATadj, and 65 for GFATadj. Loci colored black were genome-wide significant (p<5×10⁻⁸) in sex-combined analysis, blue loci were significant for males, but neither females nor sex-combined, and red loci were significant for females, but neither males nor sex-combined. P_{diff} corresponds to the “calcpdiff” function in EasyStrata comparing SNP effects in males and females (Methods). Across six adiposity traits (VATadj, ASATadj, GFATadj, VAT/ASAT, VAT/GFAT, and ASAT/GFAT), 220 unique loci-trait pairs were tested for sex heterogeneity (FIG. 22), so a Bonferroni-corrected significance threshold of p_{diff}<0.05/220=2.3×10⁻⁴ was set.

FIG. 4A-4C—Effects of previously identified WHRadjBMI loci on local adiposity traits. In total, 345 of the 346 index SNPs associated with WHRadjBMI in a recent meta-analysis from the GIANT consortium were available in the studied cohort¹². Effect sizes of VATadj, ASATadj, and GFATadj are plotted against the effect size for WHRadjBMI as reported in the cited study (Supplementary Data 11). Betas and pvalues for VATadj, ASATadj, and GFATadj correspond to the BOLT-LMM association p values computed in this study for the 345 index SNPs.

FIG. 5—Rare variants in PDE3B selectively associate with fat distribution in female participants. A mask combining predicted loss-of-function variants and missense variants predicted to be deleterious by 5 out of 5 in silico prediction algorithms in PDE3B associated with GFATadj in females with exome-wide significance (Supplementary Data 15). Effect sizes with 95% confidence intervals are plotted for carrier status. Linear regressions were adjusted for age, age squared, imaging center, genotyping array, and the first ten principal components of genetic ancestry (Supplementary Data 16). Note that the carrier counts are with respect to individuals who had “adj” traits available. For the other six traits, the carrier counts are 26 carriers/9616 participants for males and 25 carriers/9879 participants for females.

FIG. 6—Enrichment of VATadj, ASATadj, and GFATadj genome-wide polygenic scores in tails of the distribution. For each fat depot “adj” trait, a polygenic score was trained using LDpred2 on 70% of the studied cohort and a 10% validation cohort was used to determine the optimal set of hyperparameters. Results in this figure correspond to the 20% imaged and testing set (N=7795). FIG. 25 shows the full distribution of each polygenic score in each tail of VATadj, ASATadj, and GFATadj.

FIG. 7—Effects of VATadj, ASATadj, and GFATadj polygenic scores on metabolically relevant biomarkers and diseases. The central density plots indicate the distributions of VATadj, ASATadj, and GFATadj polygenic scores in genotyped individuals of the UK Biobank who were not imaged (N=447,486). The dotted lines and shaded regions correspond to individuals in the top 5% and bottom 5% of the polygenic score. Forest plots to the right correspond to effect sizes of an indicator variable for being in the top 5% of the polygenic score (with identical color-coding to the density plots), while forest plots to the left correspond to effect sizes of an indicator variable for being in the bottom 5% of the polygenic score. Each polygenic score was residualized against the first ten principal components of genetic ancestry prior to being discretized, and each regression was adjusted for age at imaging, sex, and the first ten principal components of genetic ancestry. HbA1C hemoglobin A1C, HDL-c HDL-cholesterol, Trig triglycerides, ALT alanine aminotransferase, T2D prevalent type 2 diabetes (at time of imaging), CAD prevalent coronary artery disease, HTN prevalent hypertension. Corresponding data are found in Supplementary Data 20.

FIG. 8—Convolutional neural networks to quantify adipose tissue depots from body MRI images. (top row) Sample input into convolutional neural network (CNN): two-dimensional projections of MRIs in the coronal and sagittal directions with fat and water phases are used as input for each individual. (bottom row) In a 20% holdout set among each pre-labeled fat depot, the CNN achieves near-perfect prediction of that fat depot.

FIG. 9—Testing for VATadj collider bias with BMI and Height. (top row) Four of 30 VATadj lead SNPs are at risk of collider bias with BMI. (bottom row) Six of 30 VATadj lead SNPs are at risk of collider bias with height. SNPs showing collider bias are defined as −2<=−log10(P_{VAT}/P_{BMI})<0, while extreme collider bias is defined as −log10(P_{VAT}/P_{BMI})<−2. See Supplementary Data 22 for all data needed to plot these figures. P-values correspond to BOLT-LMM association P-values for each of the left panels.

FIG. 10—Testing for ASATadj collider bias with BMI and Height. (top row) Three of 21 ASATadj lead SNPs are at risk of collider bias with BMI. (bottom row) Six of 21 ASATadj lead SNPs are at risk of collider bias with height. SNPs showing collider bias are defined as −2<=−log10(P_{ASAT}/P_{BMI})<0, while extreme collider bias is defined as −log10(P_{ASAT}/P_{BMI})<−2. See Supplementary Data 22 for all data needed to plot these figures. P-values correspond to BOLT-LMM association P-values for each of the left panels.

FIG. 11—Testing for GFATadj collider bias with BMI and Height. (top row) One of 54 GFATadj lead SNPs are at risk of collider bias with BMI. (bottom row) Two of 54 GFATadj lead SNPs are at risk of collider bias with height. SNPs showing collider bias are defined as −2<=−log10(P_{GFAT}/P_{BMI})<0, while extreme collider bias is defined as −log10(P_{GFAT}/P_{BMI})<−2. See Supplementary Data 22 for all data needed to plot these figures. P-values correspond to BOLT-LMM association P-values for each of the left panels.

FIG. 12—Histograms for nine adiposity phenotypes. Individuals who passed imaging quality control and have been genotyped (Supplementary Data 1, n=39,076) are plotted here in a sex-stratified fashion. Note that BMI was unavailable in 1,326 (3%) of individuals, so 37,750 individuals are plotted for VATadj, ASATadj, and GFATadj. Note that sex-specific residuals prior to any additional normalization are plotted for VATadj, ASATadj, and GFATadj.

FIG. 13A-13B—(FIG. 13A) Observational correlations between adiposity phenotypes and anthropometric measurements (sex-combined). Pearson correlation coefficients between 9 adiposity traits and 5 anthropometric measures are shown. Each phenotype was scaled to mean 0 and variance 1 in sex-stratified groups prior to computing the Pearson correlation. (FIG. 13B) Observational correlations between adiposity phenotypes and anthropometric measurements (sex-stratified). Sex-stratified Pearson correlation coefficients between 9 adiposity traits and 5 anthropometric measures are shown.

FIG. 14A-14B—(FIG. 14A) Genetic correlation between adiposity phenotypes and anthropometric measurements (sex-combined). Genetic correlations (r g) between 9 adiposity traits and 5 anthropometric measures were estimated from cross-trait LD-score regression using summary statistics from sex-combined GWAS of these traits in UK Biobank. 14 (FIG. 14B) Genetic correlations (r g) estimated with cross-trait LD-score regression using summary statistics from sex-stratified GWAS of these traits in UK Biobank.

FIG. 15—Manhattan plots of unadjusted VAT, ASAT, and GFAT volumes.

FIG. 16—Manhattan plots of VATadj (sex-combined and sex-stratified).

FIG. 17—Manhattan plots of ASATadj (sex-combined and sex-stratified).

FIG. 18—Manhattan plots of GFATadj (sex-combined and sex-stratified).

FIG. 19—Manhattan plots of VAT/ASAT ratio (sex-combined and sex-stratified).

FIG. 20—Manhattan plots of VAT/GFAT ratio (sex-combined and sex-stratified).

FIG. 21—Manhattan plots of ASAT/GFAT ratio (sex-combined and sex-stratified).

FIG. 22—Common variant sex heterogeneity for VAT/ASAT, VAT/GFAT, and ASAT/GFAT. For each adiposity trait, independent loci that were associated with the trait in either sex-combined or sex-stratified analyses are plotted (Supplementary Data 10). 38 such loci are plotted for VAT/ASAT, 36 for VAT/GFAT, and 20 for ASAT/GFAT. Black loci were genome-wide significant (P<5E-08) in sex-combined analysis, blue loci were significant for males, but neither females nor sex-combined, and red loci were significant for females, but neither males nor sex-combined. P_{diff} indicates the P-value for a hypothesis test comparing SNP effects in males and females, as implemented in EasyStrata software (Methods). Across six adiposity traits (VATadj, ASATadj, GFATadj, VAT/ASAT, VAT/GFAT, and ASAT/GFAT), 220 unique loci-trait pairs were tested for sex heterogeneity, so a significance threshold of P_{diff}<0.05/220=2.3×10⁻⁴ was set—large circles indicate that a given locus met this criterion.

FIG. 23—Cell-type enrichment for VAT, ASAT, GFAT, and BMI. Top left: VAT; Top right: ASAT, Bottom left: GFAT, Bottom right: BMI. Each circle represents a tissue or cell type from either the GTEx dataset or the Franke lab dataset. Large circles pass the cutoff of FDR <5% at −log10 (P)=2.75. 17 Complete data tables corresponding to these plots are found in Supplementary Data 14.

FIG. 24—Cell-type enrichment for local adiposity traits. Top left: VATadj; Top right: ASATadj, Middle left: GFATadj, Middle right: VAT/ASAT, Bottom left: VAT/GFAT, Bottom right: ASAT/GFAT. Each circle represents a tissue or cell type from either the GTEx dataset or the Franke lab dataset. Large circles pass the cutoff of FDR <5% at −log10 (P)=2.75. 17 Complete data tables corresponding to these plots are found in Supplementary Data 14.

FIG. 25A-25B—Visualizing the relationship between VATadj, ASATadj, and GFATadj and their polygenic scores at the tails of the distributions. For each fat depot “adj” trait, a polygenic score was trained using LDpred2 on 70% of the studied cohort and a 10% validation cohort was used to determine the optimal set of hyperparameters. Results in this figure correspond to the 20% testing set (N=7,795). (FIG. 25A) shows distribution of polygenic scores at the phenotypic tails of VATadj, ASATadj, and GFATadj. (FIG. 25B) shows distribution of VATadj, ASATadj, and GFATadj across deciles of the polygenic scores. Boxes contain median values and are bounded by the 1st and 3rd quartiles.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

Reference is made to an article posted to medRxiv on Aug. 26, 2021, entitled, “Inherited basis of visceral, abdominal subcutaneous and gluteofemoral fat depots,” and having the following authors: Saaket Agrawal, Minxian Wang, Marcus D. R. Klarqvist, Joseph Shin, Hesam Dashti, Nathaniel Diamant, Seung Hoan Choi, Sean J. Jurgens, Patrick T. Ellinor, Anthony Philippakis, Kenney Ng, Melina Claussnitzer, Puneet Batra, Amit V. Khera (medRxiv 2021.08.24.21262564). Reference is also made to an article posted to medRxiv on May 10, 2021 and Jul. 28, 2022, entitled, “Association of machine learning-derived measures of body fat distribution with cardiometabolic diseases in >40,000 individuals,” and having the following authors: Saaket Agrawal, Marcus D. R. Klarqvist, Nathaniel Diamant, Takara L. Stanley, Patrick T. Ellinor, Nehal N. Mehta, Anthony Philippakis, Kenney Ng, Melina Claussnitzer, Steven K. Grinspoon, Puneet Batra, Amit V. Khera (medRxiv 2021.05.07.21256854). Reference is also made to Klarqvist M D R, Agrawal S, Diamant N, et al. Silhouette images enable estimation of body fat distribution and associated cardiometabolic risk. NPJ Digit Med. 2022; 5(1):105. Published 2022 Jul. 27. Reference is also made to Agrawal S, Wang M, Klarqvist M D R, et al. Inherited basis of visceral, abdominal subcutaneous and gluteofemoral fat depots. Nat Commun. 2022; 13(1):3771.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide genetic variants associated with local adiposity traits obtained by adjusting adiposity traits for BMI and height. Embodiments disclosed herein also provide genes linked to variants and associated with the local adiposity traits. The local adiposity traits are associated with metabolic disorders. In example embodiments, variants indicate risk for a metabolic disorder and can be used to determine treatment. In example embodiments, genes associated with local adiposity traits and/or variants can be targeted therapeutically. In example embodiments, a risk for a metabolic disorder can be determined by detecting one or more risk variants associated with a local adiposity trait.

For any given level of overall adiposity, individuals vary considerably in fat distribution. The inherited basis of fat distribution in the general population is not fully understood. Here, Applicants studied about 38,965 UK Biobank participants with MRI-derived visceral (VAT), abdominal subcutaneous (ASAT), and gluteofemoral (GFAT) adipose tissue volumes. Because these fat depot volumes are highly correlated with BMI, Applicants additionally studied six local adiposity traits: VAT adjusted for BMI and height (VATadj), ASAT adjusted for BMI and height (ASATadj), GFAT adjusted for BMI and height (GFATadj), VAT/ASAT, VAT/GFAT, and ASAT/GFAT. Applicants identified 250 independent common variants (39 newly-identified) associated with at least one trait, with many associations more pronounced in female participants. Rare variant association studies extended prior evidence for PDE3B as an important modulator of fat distribution. Local adiposity traits (1) highlighted depot-specific genetic architecture and (2) enabled construction of depot-specific polygenic risk scores (PRS) that had divergent associations with type 2 diabetes and coronary artery disease. To prioritize genes, Applicants conducted a transcriptome-wide association study (TWAS) using gene expression data from visceral and subcutaneous adipose tissue from GTEx v7. These results—using MM-derived, BMI-independent measures of local adiposity—confirmed fat distribution as a highly heritable trait with important implications for cardiometabolic health outcomes.

In example embodiments, variants associated with local adiposity traits are selected from Supplementary Data 3. In example embodiments, variants associated with local adiposity traits are selected from Table 1 (rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657). In example embodiments, variants in Table 1 and Supplementary Data 3 associated with GFATadj are favorable variants indicating a low risk for metabolic disorders and variants associated with VATadj and ASATadj are variants indicating a risk for metabolic disorders. In example embodiments, genome-wide polygenic risk scores (PRS) scores for each local adipose trait are used. In example embodiments, variants identified indicate risk for metabolic disorders or a healthy metabolic state.

In example embodiments, genes linked to variants and associated with local adiposity traits are selected. Any methods of linking enhancers to genes expressed in tissues can be used. In example embodiments, an Activity-by-Contact (ABC) model is used to link variants to genes. This model is based on the simple biochemical notion that an element's quantitative effect on a gene should depend on its strength as an enhancer (“Activity”) weighted by how often it comes into 3D contact with the promoter of the gene (“Contact”), and that the relative contribution of an element on a gene's expression should depend on the element's effect divided by the total effect of all elements (see, e.g., Fulco et al. Activity-by-contact model of enhancer-promoter regulation from thousands of CRISPR perturbations. Nat Genet. 2019; 51(12):1664-1669. doi:10.1038/s41588-019-0538-0; and Moonen et al., 2020, KLF4 Recruits SWI/SNF to Increase Chromatin Accessibility and Reprogram the Endothelial Enhancer Landscape under Laminar Shear Stress. bioRxiv 2020.07.10.195768, doi.org/10.1101/2020.07.10.195768). In example embodiments, an epigenome model, such as Roadmap, is used to link variants to gene modules (see, e.g., Ernst, J., Kheradpour, P., Mikkelsen, T. et al. Mapping and analysis of chromatin state dynamics in nine human cell types. Nature 473, 43-49 (2011); Kundaje, A., Meuleman, W., Ernst, J. et al. Integrative analysis of 111 reference human epigenomes. Nature 518, 317-330 (2015); and egg2.wustl.edu/roadmap/web_portal/index.html). In example embodiments, an Enhancer-to-gene (E2G) strategy is a combined union of Activity-By-Contact and Roadmap Enhancer-to-gene (E2G) strategy (Roadmap-U-ABC E2G strategy) (see, e.g., US patent application publication US20210071255A1). In example embodiments, genes linked to variants and associated with local adiposity traits are selected from Supplementary Data 13 (e.g., CEBPA-AS1, CCDC92, FLOT1, CYP21A1P, HLA-DRB6, and HLA-S; or CENPW, TIPARP, and AC103965.1; or CCDC92, DNAH100S, RP11-380L11.4, IRS1, ZNF664, RIMKLBP2, DNAH10, RP11-392O17.1, VEGFB, FAM13A, PDGFC, MAFF, TMEM165, RP11-177J6.1, CLOCK, and SRD5A3-AS1; or CEBPA-AS1, CCDC92, ADCY3, FLOT1, TIPARP, CEBPA-AS1, and IRS1; or CCDC92, CEBPA-AS1, RP11-380L11.4, DNAH100S, HLA-S, DNAH10, CCDC92, DNAH100S, CEBPA-AS1, RP11-380L11.4, XXbac-BPG248L24.12, HLA-S, and VEGFB; or CCDC92, and TIPARP). In example embodiments, the genes associated with local adiposity traits are therapeutic targets for treating metabolic disorders. In example embodiments, genes are targeted to increase expression or activity. In example embodiments, genes are targeted to decrease expression or activity.

### Methods of Treatment

### Metabolic Disorders

In example embodiments, the present invention provides for methods of treating metabolic disorders. As used herein a metabolic disorder refers to any condition that diverges from a healthy metabolic state. A healthy metabolic state refers to ideal levels of blood sugar, triglycerides, high-density lipoprotein (HDL) cholesterol, blood pressure, and waist circumference, without using medications. “Metabolic disorder” refers to disorders, diseases and conditions caused or characterized by abnormal weight gain, energy use or consumption, altered responses to ingested or endogenous nutrients, energy sources, hormones or other signaling molecules within the body or altered metabolism of carbohydrates, lipids, proteins, nucleic acids, or a combination thereof. A metabolic disorder may be associated with either a deficiency or an excess in a metabolic pathway resulting in an imbalance in metabolism of carbohydrates, lipids, proteins and/or nucleic acids. Examples of metabolic disorders include, but are not limited to, coronary artery disease (CAD), hypertension, type 2 diabetes (T2D), lipodystrophy, familial partial lipodystrophy (FPLD), insulin deficiency or insulin-resistance related disorders, dyslipidemia, metabolic syndrome, non-alcoholic steatohepatitis (NASH), non-alcoholic fatty liver disease (NAFLD), impaired glucose tolerance, and hyperglycemia. Metabolic syndrome includes high blood pressure, high blood sugar, excess body fat around the waist, and abnormal cholesterol levels. The syndrome increases a person's risk for heart attack and stroke. Examples of overweight and/or obesity related metabolic disorders include, but are not limited to metabolic syndrome, insulin-deficiency or insulin-resistance related disorders, Type 2 Diabetes, glucose intolerance, abnormal lipid metabolism, atherosclerosis, hypertension, cardiac pathology, stroke, non-alcoholic fatty liver disease, hyperglycemia, hepatic steatosis, dyslipidemia, dysfunction of the immune system associated with overweight and obesity, cardiovascular diseases, high cholesterol, elevated triglycerides, asthma, sleep apnea, osteoarthritis, neuro-degeneration, gallbladder disease, syndrome X, inflammatory and immune disorders, atherogenic dyslipidemia and cancer.

In example embodiments, CAD is treated. Coronary artery disease (CAD), also called coronary heart disease (CHD), ischemic heart disease (IHD), myocardial ischemia, or simply heart disease, involves the reduction of blood flow to the heart muscle due to build-up of atherosclerotic plaque in the arteries of the heart. It is the most common of the cardiovascular diseases. Types include stable angina, unstable angina, myocardial infarction, and sudden cardiac death. The heritability of coronary artery disease has been estimated between 40% and 60%. Ways to reduce CAD risk include eating a healthy diet, regularly exercising, maintaining a healthy weight, and not smoking. Medications for diabetes, high cholesterol, or high blood pressure are sometimes used. There is limited evidence for screening people who are at low risk and do not have symptoms. Treatment involves the same measures as prevention. Additional medications such as antiplatelets (including aspirin), beta blockers, or nitroglycerin may be recommended. Procedures such as percutaneous coronary intervention (PCI) or coronary artery bypass surgery (CABG) may be used in severe disease. In those with stable CAD it is unclear if PCI or CABG in addition to the other treatments improves life expectancy or decreases heart attack risk.

In example embodiments, type 2 diabetes (T2D) is treated. Type 2 diabetes, formerly known as adult-onset diabetes, is a form of diabetes mellitus that is characterized by high blood sugar, insulin resistance, and relative lack of insulin. Type 2 diabetes primarily occurs as a result of obesity and lack of exercise. Common symptoms include increased thirst, frequent urination, and unexplained weight loss. Symptoms may also include increased hunger, feeling tired, and sores that do not heal. Often symptoms come on slowly. Long-term complications from high blood sugar include heart disease, strokes, diabetic retinopathy which can result in blindness, kidney failure, and poor blood flow in the limbs which may lead to amputations. The sudden onset of hyperosmolar hyperglycemic state may occur; however, ketoacidosis is uncommon. The heritability of diabetes is estimated at 72%. The World Health Organization definition of diabetes (both type 1 and type 2) is for a single raised glucose reading with symptoms, otherwise raised values on two occasions of either: fasting plasma glucose ≥7.0 mmol/1 (126 mg/dl) or with a glucose tolerance test, two hours after the oral dose a plasma glucose ≥11.1 mmol/1 (200 mg/dl). A random blood sugar of greater than 11.1 mmol/1 (200 mg/dl) in association with typical symptoms or a glycated hemoglobin (HbA1c) of ≥48 mmol/mol (≥6.5 DCCT %) is another method of diagnosing diabetes. Onset of type 2 diabetes can be delayed or prevented through proper nutrition and regular exercise. Intensive lifestyle measures may reduce the risk by over half. There are several classes of anti-diabetic medications available (e.g., metformin, sulfonylureas, thiazolidinediones, dipeptidyl peptidase-4 inhibitors, SGLT2 inhibitors, and glucagon-like peptide-1 analogs).

In example embodiments, lipodystrophy is treated. As used herein “lipodystrophy” refers to a group of genetic or acquired disorders in which the body is unable to produce and maintain healthy fat tissue. The medical condition is characterized by abnormal or degenerative conditions of the body's adipose tissue. (“Lipo” is Greek for “fat”, and “dystrophy” is Greek for “abnormal or degenerative condition”.) This condition is also characterized by a lack of circulating leptin which may lead to osteosclerosis. The absence of fat tissue is associated with insulin resistance, hypertriglyceridemia, non-alcoholic fatty liver disease (NAFLD) and metabolic syndrome. Due to an insufficient capacity of subcutaneous adipose tissue to store fat, fat is deposited in non-adipose tissue (lipotoxicity), leading to insulin resistance. Patients display hypertriglyceridemia, severe fatty liver disease and little or no adipose tissue. Average patient lifespan is approximately 30 years before death, with liver failure being the usual cause of death. In contrast to the high levels seen in non-alcoholic fatty liver disease associated with obesity, leptin levels are very low in lipodystropy. In certain embodiments, polygenic lipodystrophy includes insulin resistance with a “lipodystrophy-like” fat distribution, insulin sensitivity, BMI-adjusted T2D, increased BMI-adjusted waist-to-hip ratio (WHRadjBMI), and/or Type-2 Diabetes (T2D).

### Identifying Subjects for Treatment

In example embodiments, subjects treated have a genetic risk for the metabolic disorder (e.g., by determining the presence of a risk variant or PRS). The risk for the metabolic disorder may be the presence or absence of one or more variants or combination of genetic variants that increases the risk for the metabolic disorder. The risk for the metabolic disorder may be the presence or absence of one or more variants or combination of genetic variants that decreases the risk for the metabolic disorder. For example, a subject having one or more variants or combination of genetic variants that increases the risk for the metabolic disorder is at greater risk for the metabolic disorder. For example, a subject having one or more variants or combination of genetic variants that decreases the risk for the metabolic disorder is at lower risk for the metabolic disorder. In another example embodiment, a polygenic risk score that indicates an increased or decreased risk for a metabolic disorder can be used to determine risk for the metabolic disorder. For example, a subject with a high polygenic risk score (PRS) associated with risk for the metabolic disorder has an increased risk for the metabolic disorder and a subject with a low polygenic risk score associated with risk for the metabolic disorder has a decreased risk for the metabolic disorder (e.g., VATadj PRS). For example, a subject with a high polygenic risk score associated with a healthy metabolic phenotype has a decreased risk for the metabolic disorder and a subject with a low polygenic risk score associated with healthy metabolic phenotype has an increased risk for the metabolic disorder (e.g., GFATadj PRS). In example embodiments, the one or more variants are associated with local adiposity traits. As used herein local adiposity traits can refer to fat deposition traits. As used herein fat deposition traits refer to the localization of fat deposits. For example, fat deposited in VAT, ASAT and GFAT.

In example embodiments, genetic risk can be determined by genotyping a subject to identify variants. Identifying the presence of a risk loci can be performed using any DNA detection method known in the art. In example embodiments, genotyping is determined by sequencing, polymerase chain reaction, or hybridization.

In example embodiments, the methods include sequencing at least part of a genome of one or more cells from the subject. In certain example embodiments, detection of variants can be done by sequencing. Sequencing can be, for example, whole genome sequencing. In one example embodiment, the invention involves high-throughput and/or targeted nucleic acid profiling (for example, sequencing, quantitative reverse transcription polymerase chain reaction, and the like).

In example embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In example embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In example embodiments, targeted sequencing is used in the present invention (see, e.g., Mantere et al., PLoS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

Variants may also be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction—restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

### Polygenic Risk Scores

In example embodiments, determining risk for a metabolic disorder includes identifying genome variants that are associated with a distinct functional or pathobiological mechanism. In preferred embodiments, the genome variants can be used to generate a polygenic risk score (PRS). As used herein, “polygenic risk score” refers to an assessment of the risk of a specific condition based on the collective influence of many genetic variants or a score based on the number of variants related to the disease a subject has. Variants can include variants associated with genes of known function and variants not known to be associated with genes relevant to the condition. In example embodiments, the polygenic risk score is a partitioned polygenic risk score (pPS) and is enriched for variants that share a similar pattern of genome-wide associations across disease related traits for the disease (see, Udler M S, Kim J, von Grotthuss M, et al. Type 2 diabetes genetic loci informed by multi-trait associations point to disease mechanisms and subtypes: A soft clustering analysis. PLoS medicine 2018; 15(9): e1002654).

In example embodiments, the polygenic risk score comprises the most common variants associated with the disease related traits, optionally, including additional variants that are progressively less common for the disease. In example embodiments, the polygenic risk score comprises less than 100 variants. In example embodiments, the polygenic risk score comprises 100 or more variants. In example embodiments, the polygenic risk score comprises between 100 to 400 variants. In example embodiments, the polygenic risk score comprises 1000 or more variants. In example embodiments, the polygenic risk score is obtained by a pipeline applying Bayesian Non-negative Factorization (bNMF). In example embodiments, the polygenic risk comprises 100,000, 200,000, 300,000, 400,000, 500,000, 750,000, or more than a million variants. In example embodiments, the PRS is enriched for variants linked to DNA regulatory elements active (e.g., enhancers) in the tissue associated with the disease.

### Indicators of Metabolic Disease

In example embodiments, a subject at risk for a metabolic disorder is identified by detection of the one or more variants or combination of genetic variants. In example embodiments, the subject that is treated has increased risk for the metabolic disorder in combination with one or more indicators of metabolic disease. Metabolic disorders can be identified by detecting one or more indicators of metabolic disease. Indicators of metabolic disease include but are not limited to increased visceral adipose tissue (VAT), increased abdominal subcutaneous adipose tissue (ASAT), decreased gluteofemoral adipose tissue (GFAT), increased serum triglycerides, decreased HDL-c (HDL-cholesterol), increased LDL-c (LDL-cholesterol), increased liver enzymes, such as alanine aminotransferase (ALT), and increased HbA1C (hemoglobin A1C). Thus, a subject at high risk for the metabolic disorder can be treated at the first sign for the metabolic disorder. In example embodiments, subjects at high risk for a metabolic disorder are treated by increasing monitoring of the subject for the metabolic disorder. For example, the one or more variants or combination of genetic variants are detected in the subject and upon determining that the subject is at high risk for the metabolic disorder treating the subject with one or more diagnostic tests to determine the metabolic state of the subject, such as the fat distribution state. The one or more diagnostic tests can be blood-based analysis or imaging analysis, such as computed tomography (CT scan) (see, e.g., Ryo, Miwa et al. “Clinical significance of visceral adiposity assessed by computed tomography: A Japanese perspective.” World journal of radiology vol. 6,7 (2014): 409-16), dual-energy X-ray absorptiometry (DXA or DEXA) scan (see, e.g., Meral R, Ryan B J, Malandrino N, et al. “Fat Shadows” From DXA for the Qualitative Assessment of Lipodystrophy: When a Picture Is Worth a Thousand Numbers. Diabetes Care. 2018; 41(10):2255-2258), or magnetic resonance imaging (MM) (see, e.g., Hu H H, Nayak K S, Goran M I. Assessment of abdominal adipose tissue and organ fat content by magnetic resonance imaging. Obes Rev. 2011; 12(5):e504-e515). In one example embodiment, upon determining that a high-risk subject also has one or more indicators of metabolic disease the subject can be treated with the one or more therapeutic agents.

### Therapeutic Agents

In example embodiments, a subject in need thereof is treated with one or more therapeutic agents. The one or more therapeutic agents may be agents that treat a metabolic disorder. The therapeutic agents may also shift a metabolic trait associated with the one or more variants. For example, the therapeutic agent may shift an unhealthy fat distribution to a healthier fat distribution (e.g., shift VAT to GFAT, reduce VAT, and/or reduce ASAT). The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

In one example embodiment, a method of treating subjects that are at risk for or suffering from a metabolic disorder (e.g., has a risk variant or a PRS that indicates risk), comprises administering to a subject at risk for or suffering from a metabolic disorder, a therapeutically effective amount of one or more agents that treat the metabolic disorder.

### PPAR Agonists

In example embodiments, a subject in need thereof is treated with a PPAR agonist. PPAR agonists are drugs which act upon the peroxisome proliferator-activated receptor. They are used for the treatment of symptoms of the metabolic syndrome, mainly for lowering triglycerides and blood sugar.

### PPAR-Alpha Agonists

PPARα (alpha) is the main target of fibrate drugs, a class of amphipathic carboxylic acids (clofibrate, gemfibrozil, ciprofibrate, bezafibrate, and fenofibrate). They were originally indicated for cholesterol disorders and more recently for disorders that feature high triglycerides. Fenofibrate is a fibric acid derivative, a prodrug comprising fenofibric acid linked to an isopropyl ester. It lowers lipid levels by activating peroxisome proliferator-activated receptor alpha (PPARα). PPARα activates lipoprotein lipase and reduces apoprotein CIII, which increases lipolysis and elimination of triglyceride-rich particles from plasma (see, e.g., Mahmoudi A, Moallem S A, Johnston T P, Sahebkar A. Liver Protective Effect of Fenofibrate in NASH/NAFLD Animal Models. PPAR Res. 2022; 2022:5805398). PPARα also increases apoproteins AI and AII, reduces VLDL- and LDL-containing apoprotein B, and increases HDL-containing apoprotein AI and AII. Id.

### PPAR-Gamma Agonists

PPARγ (gamma) is the main target of the drug class of thiazolidinediones (TZDs), used in diabetes mellitus and other diseases that feature insulin resistance. It is also mildly activated by certain NSAIDs (such as ibuprofen) and indoles, as well as from a number of natural compounds. Known inhibitors include the experimental agent GW-9662. The thiazolidinediones abbreviated as TZD, also known as glitazones after the prototypical drug ciglitazone, are a class of heterocyclic compounds consisting of a five-membered C₃NS ring. In example embodiments, PPAR-gamma agonists can be used to decrease visceral fat. For example, a thiazolidinedione significantly decreased visceral fat in women with obesity (White U, Fitch M D, Beyl R A, Hellerstein M K, Ravussin E. Adipose depot-specific effects of 16 weeks of pioglitazone on in vivo adipogenesis in women with obesity: a randomised controlled trial. Diabetologia. 2021; 64(1):159-167) (see also, Katoh S, Hata S, Matsushima M, et al. Troglitazone prevents the rise in visceral adiposity and improves fatty liver associated with sulfonylurea therapy—a randomized controlled trial. Metabolism. 2001; 50(4):414-417). PPAR-gamma agonists include Pioglitazone, Rosiglitazone, Lobeglitazone, Ciglitazone, Darglitazone, Englitazone, Netoglitazone, Rivoglitazone, Troglitazone, Balaglitazone, and AS-605240.

### PPAR-Delta Agonists

PPAR (delta) is the main target of a research chemical named GW501516. It has been shown that agonism of PPAR changes the body's fuel preference from glucose to lipids.

### Dual or Pan PPAR Agonists

A fourth class of dual PPAR agonists, so-called glitazars, which bind to both the α and γ PPAR isoforms, are currently under active investigation for treatment of a larger subset of the symptoms of the metabolic syndrome. These include the compounds aleglitazar, muraglitazar and tesaglitazar. Saroglitazar was the first glitazar to be approved for clinical use. In addition, there is continuing research and development of new dual α/δ and γ/δ PPAR agonists for additional therapeutic indications, as well as “pan” agonists acting on all three isoforms.

### Growth Hormone-Releasing Hormone (GHRH)

Growth hormone secretagogues or GH secretagogues (GHSs) are a class of drugs which act as secretagogues (i.e., induce the secretion) of growth hormone (GH). They include agonists of the ghrelin/growth hormone secretagogue receptor (GHSR), such as ghrelin (lenomorelin), pralmorelin (GHRP-2), GHRP-6, examorelin (hexarelin), ipamorelin, and ibutamoren (MK-677), and agonists of the growth hormone-releasing hormone receptor (GHRHR), such as growth hormone-releasing hormone (GHRH, somatorelin), CJC-1295, sermorelin, and tesamorelin. Growth hormone releasing hormone analogs, such as tesamorelin, have previously been shown to lead to a selective reduction of VAT in patients with obesity or HIV-associated lipodystrophy (Makimura H, et al. Metabolic effects of a growth hormone-releasing factor in obese subjects with reduced growth hormone secretion: a randomized controlled trial. J. Clin. Endocrinol. Metab. 2012; 97:4769-4779; and Stanley T L, et al. Effect of tesamorelin on visceral fat and liver fat in HIV-infected patients with abdominal fat accumulation: a randomized clinical trial. JAMA. 2014; 312:380-389). Growth hormone-releasing hormone (GHRH), also known as somatocrinin or by several other names in its endogenous forms and as somatorelin (INN) in its pharmaceutical form, is a releasing hormone of growth hormone (GH). It is a 44-amino acid peptide hormone produced in the arcuate nucleus of the hypothalamus. GHRHs include Tesamorelin, Somatocrinin, CJC-1295, Modified GRF (1-29), Dumorelin, Rismorelin, Sermorelin, and Somatorelin.

### Sodium-Glucose Transporter 2 (SGLT2) Inhibitors

SGLT2 inhibitors, also called gliflozins or flozins, are a class of medications that modulate sodium-glucose transport proteins in the nephron (the functional units of the kidney), unlike SGLT1 inhibitors that perform a similar function in the intestinal mucosa. The foremost metabolic effect of this is to inhibit reabsorption of glucose in the kidney and therefore lower blood sugar. They act by inhibiting sodium-glucose transport protein 2 (SGLT2). SGLT2 inhibitors are used in the treatment of type II diabetes mellitus (T2DM). Apart from blood sugar control, gliflozins have been shown to provide significant cardiovascular benefit in patients with type II diabetes (T2DM). Several medications of this class have been approved or are currently under development. In studies on canagliflozin, a member of this class, the medication was found to enhance blood sugar control as well as reduce body weight and systolic and diastolic blood pressure. SGLT2 inhibitors include Canagliflozin, Dapagliflozin, Empagliflozin, Ertugliflozin, Ipragliflozin, Luseogliflozin, Remogliflozin, Sotagliflozin, and Tofogliflozin.

### Metformin

Metformin, sold under the brand name Glucophage, among others, is the main first-line medication for the treatment of type 2 diabetes, particularly in people who are overweight. Metformin is a biguanide antihyperglycemic agent. It works by decreasing glucose production in the liver, by increasing the insulin sensitivity of body tissues, and by increasing GDF15 secretion, which reduces appetite and caloric intake.

### Alpha-Glucosidase Inhibitors

Alpha-glucosidase inhibitors (AGIs) are oral anti-diabetic drugs used for diabetes mellitus type 2 that work by preventing the digestion of carbohydrates (such as starch and table sugar). Carbohydrates are normally converted into simple sugars (monosaccharides) by alpha-glucosidase enzymes present on cells lining the intestine, enabling monosaccharides to be absorbed through the intestine. Hence, alpha-glucosidase inhibitors reduce the impact of dietary carbohydrates on blood sugar. Examples of alpha-glucosidase inhibitors include: Acarbose, Miglitol, and Voglibose. Miglitol has been shown to have anti-obesity potential, which was achieved by reducing abdominal fat accumulation and/or enhanced insulin requirement, and then corrected both the metabolic and hemodynamic aberrations seen in patients with the metabolic syndrome (see, e.g., Shimabukuro M, Higa M, Yamakawa K, Masuzaki H, Sata M. Miglitol, α-glycosidase inhibitor, reduces visceral fat accumulation and cardiovascular risk factors in subjects with the metabolic syndrome: a randomized comparable study. Int J Cardiol. 2013; 167(5):2108-2113). There are a large number of natural products with alpha-glucosidase inhibitor action (Benalla W, Bellahcen S, Bnouham M. Antidiabetic medicinal plants as a source of alpha glucosidase inhibitors. Curr Diabetes Rev. 2010; 6(4):247-254).

### Incretin Based Therapy

Incretin hormones are released from the intestine after nutrient intake (see, e.g., Michalowska J, Miller-Kasprzak E, Bogdanski P. Incretin Hormones in Obesity and Related Cardiometabolic Disorders: The Clinical Perspective. Nutrients. 2021; 13(2):351. Published 2021 Jan. 25). Incretin-based glucose-lowering medications, in particular GLP-1 receptor agonists (GLP-1RAs), have proven to be effective and are currently used in T2D treatment. Id. Randomized controlled trials showed that treatment with GLP-1RA, liraglutide, is associated with a decrease in visceral fat in obese patients with T2DM or prediabetes. Id. Glucagon-like peptide-1 receptor agonists, also known as GLP-1 receptor agonists or incretin mimetics, are agonists of the GLP-1 receptor. GLP-1 receptor agonists include, but are not limited to exenatide, liraglutide, lixisenatide, albiglutide, dulaglutide, semaglutide, tirzepatide, taspoglutide, and efpeglenatide.

### Sulfonylurea

Sulfonylureas are a class of organic compounds used in medicine and agriculture, for example as antidiabetic drugs widely used in the management of diabetes mellitus type 2. They act by increasing insulin release from the beta cells in the pancreas. Third-generation drugs include glimepiride. Second-generation drugs include glibenclamide (glyburide), glibornuride, gliclazide, glipizide, gliquidone, glisoxepide and glyclopyramide. First-generation drugs include acetohexamide, carbutamide, chlorpropamide, glycyclamide (tolcyclamide), metahexamide, tolazamide and tolbutamide.

### Recombinant Leptin or Leptin Mimetics

Recombinant leptin formulations or leptin mimetics can be used to treat lipodystrophy, where people have a loss of fatty tissue under the skin and a build-up of fat elsewhere in the body such as in the liver and muscles. Recombinant leptin formulations or leptin mimetics can also be used to treat the complications of leptin deficiency in people with congenital or acquired generalized lipodystrophy. Metreleptin, sold under the brand name Myalept among others, is a synthetic analog of the hormone leptin used to treat various forms of dyslipidemia. Metreleptin is also referred to as recombinant leptin (r-metHuLeptin).

In another example embodiment, a subject at risk for a metabolic disorder or having a trait associated with a metabolic disorder is treated with one or more therapeutic agents targeting one or more genes associated with local adiposity traits and/or variants. For example, genes associated with any variant associated with local adiposity traits are targeted (e.g., CEBPA-AS1, CCDC92, FLOT1, CYP21A1P, HLA-DRB6, and HLA-S; or CENPW, TIPARP, and AC103965.1; or CCDC92, DNAH100S, RP11-380L11.4, IRS1, ZNF664, RIMKLBP2, DNAH10, RP11-392O17.1, VEGFB, FAM13A, PDGFC, MAFF, TMEM165, RP11-177J6.1, CLOCK, and SRD5A3-AS1; or CEBPA-AS1, CCDC92, ADCY3, FLOT1, TIPARP, CEBPA-AS1, and IRS1; or CCDC92, CEBPA-AS1, RP11-380L11.4, DNAH100S, HLA-S, DNAH10, CCDC92, DNAH100S, CEBPA-AS1, RP11-380L11.4, XXbac-BPG248L24.12, HLA-S, and VEGFB; or CCDC92, and TIPARP). In example embodiments, the genes associated with local adiposity traits are targeted. In example embodiments, the one or more therapeutic agents treat the metabolic disorder by increasing the expression or activity of a target gene. In example embodiments, the one or more therapeutic agents treat the metabolic disorder by decreasing the expression or activity of a target gene.

In example embodiments, the one or more agents comprises a small molecule inhibitor, small molecule degrader (e.g., ATTEC, AUTAC, LYTAC, or PROTAC), genetic modifying agent, antisense oligonucleotides (ASO), antibody, antibody fragment, antibody-like protein scaffold, aptamer, protein, or any combination thereof.

### Small Molecules

One type of small molecule applicable to the present invention is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins.

### Nucleic Acid Molecules

In some embodiments, the agents may be a nucleic acid molecule. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8).

### Genetic Modifying Agents

In example embodiments, a genetic modifying agent, such as a programmable nuclease, may be used to alter expression of a target gene. Gene editing using programmable nucleases may utilize two different cell repair pathways, non-homologous end joining (NHEJ), and homology directed repair. Example programmable nucleases for use in this manner include zinc finger nucleases (ZEN), TALE nucleases (TALENS), meganucleases, and CRISPR-Cas systems.

### CRISPR-Cas

In one example embodiment, the gene editing system is a CRISPR-Cas system. The CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

### NHEJ-Based Editing

In one example embodiment, the CRISPR-Cas system is used to introduce one or more insertions or deletions in a target gene. More than one guide sequence may be selected to insert multiple insertion, deletions, or combination thereof. Likewise, more than one Cas protein type may be used, for example, to maximize targets sites adjacent to different PAMs. In one example embodiment, a guide sequence is selected that directs the CRISPR-Cas system to make one or more insertions or deletions within an enhancer region in a target gene.

### HDR Template Based Editing

In one example embodiment, a donor template is provided to replace a genomic sequence in a target gene. A donor template may comprise an insertion sequence flanked by two homology regions. The insertion sequence comprises an edited sequence to be inserted in place of the target sequence (e.g., a portion of genomic DNA comprising the one or more variants). The homology regions comprise sequences that are homologous to the genomic DNA strands at the site of the CRISPR-Cas induced double-strand break. Cellular HDR mechanisms then facilitate insertion of the insertion sequence at the site of the DSB. The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Class 1 Systems

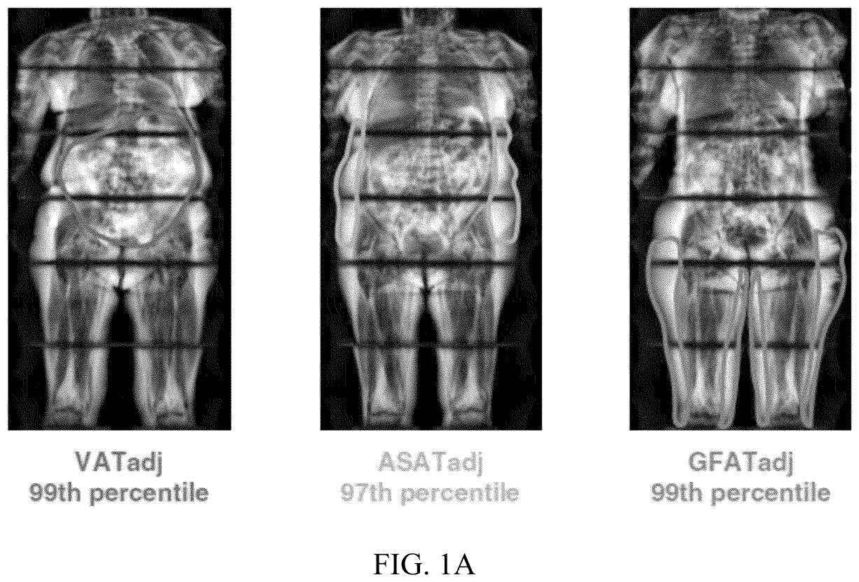

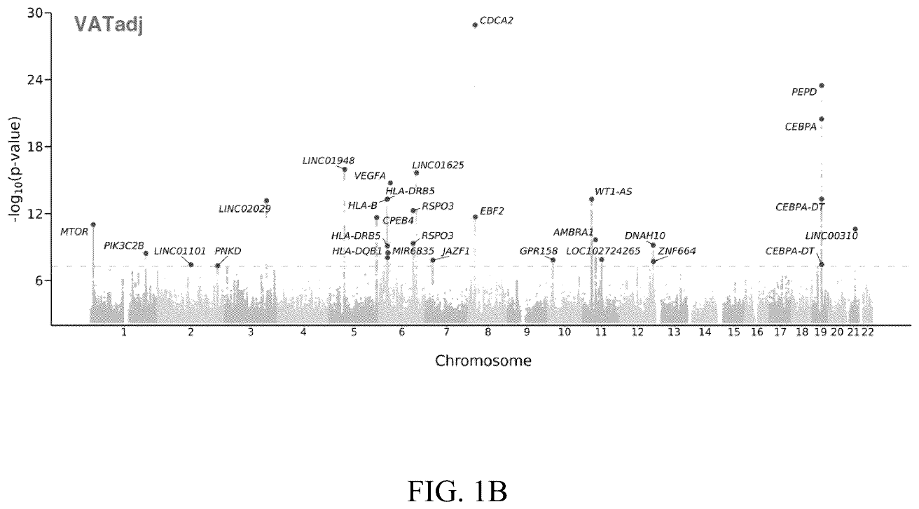

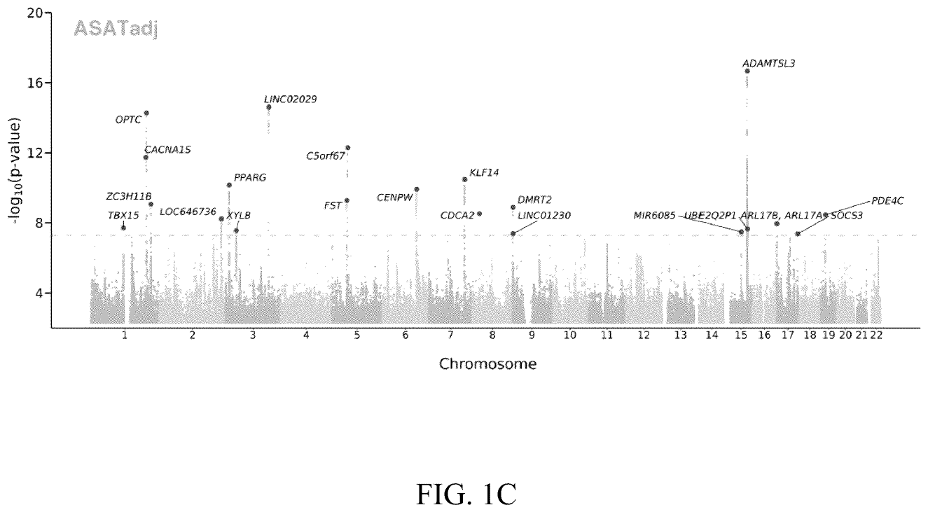

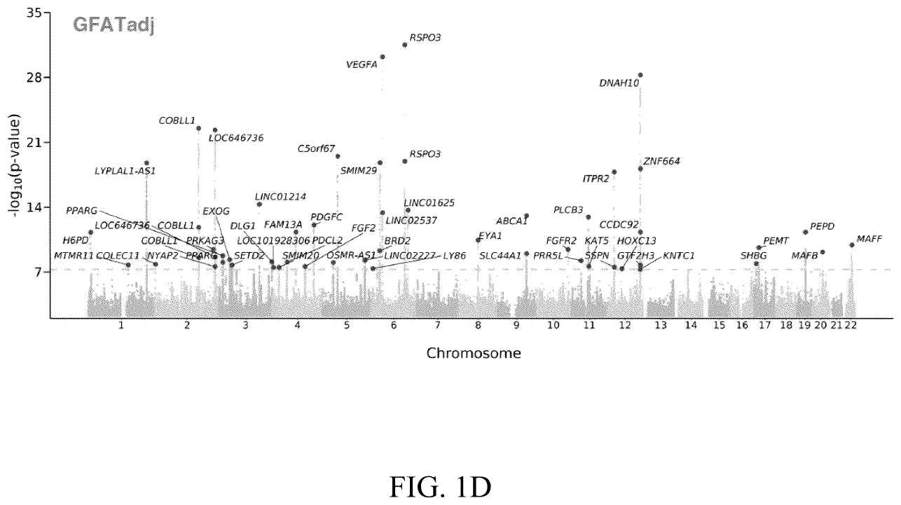

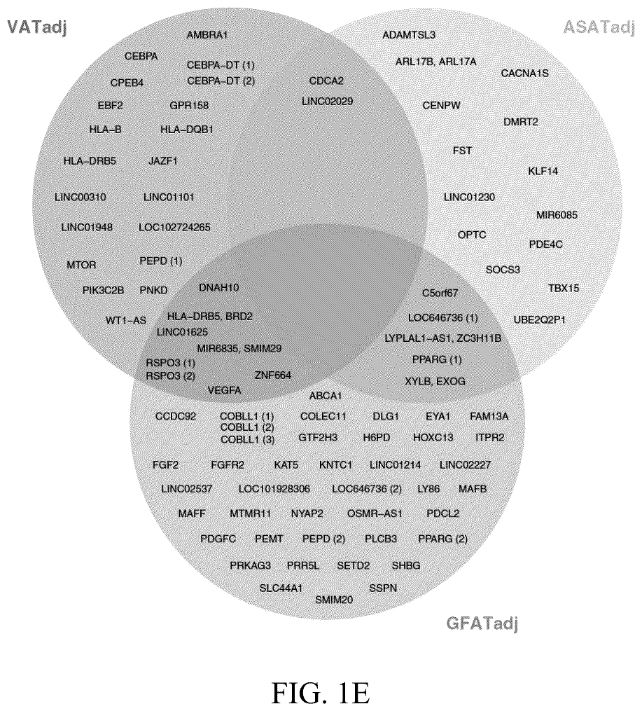

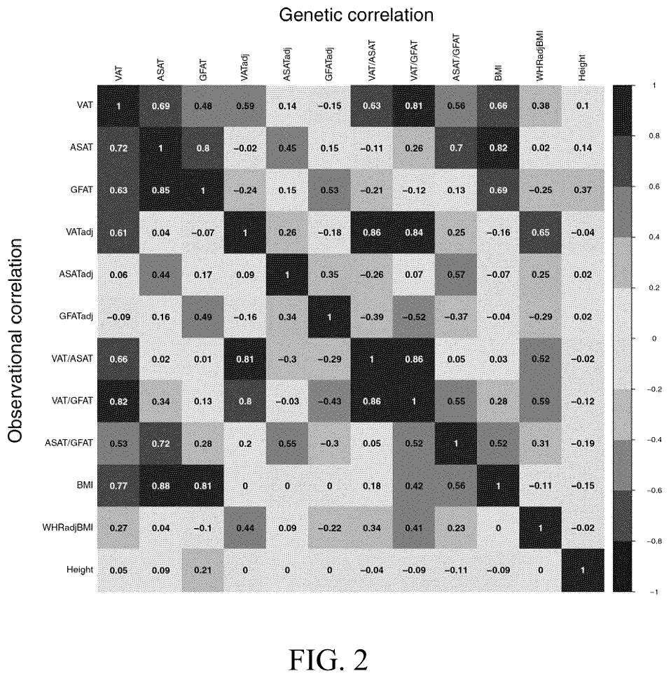

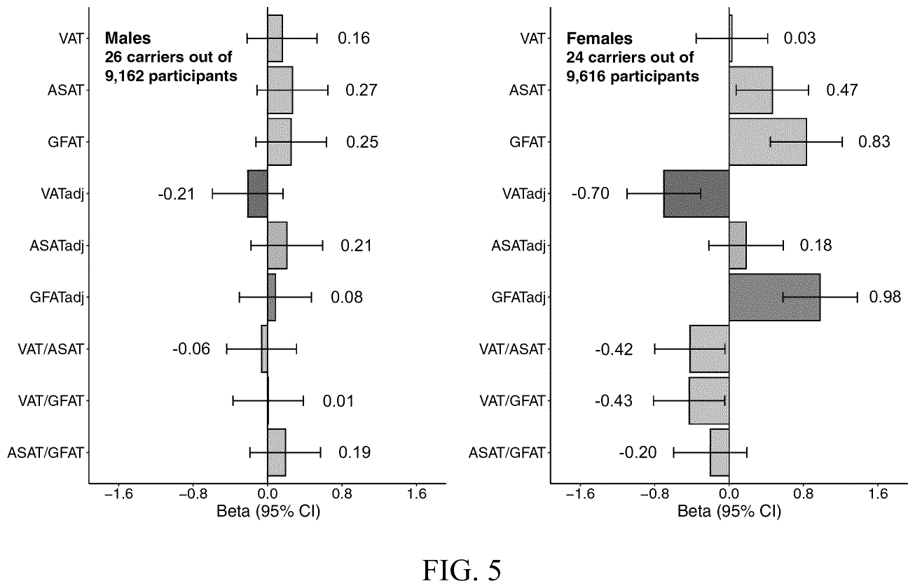

The CRISPR-Cas therapeutic methods disclosed herein may be designed for use with Class 1 CRISPR-Cas systems. In certain example embodiments, the Class 1 system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020), incorporated in its entirety herein by reference and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade, in particular Class1 proteins, can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The CRISPR-Cas therapeutic methods disclosed herein may be designed for use with. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a Ruv-C-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type II CRISPR-Cas system is a II-C1 CRISPR-Cas system. In one example embodiment, the Type II CRISPR-Cas system is a II-C2 CRISPR-Cas system. In sone example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2c1), Cas12c (C2c3), Cas12d (CasY), Cas12e (CasX), Cas14, and/or Cas(I).

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A (from Gleditzsch et al. 2019) below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, TAG, and |
| | CAC |
| Type I-C | NTTC |
| Type I-E | 5′-AAG-3′ |
| Type I-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al., Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

### Zinc Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain (e.g., activator domain). The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. TALENS

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011). each of which is incorporated herein by reference in its entirety.

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine, and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in one example embodiment, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L T G Q L L K I A K R G G V T A V E A V D
H A W R N A L T G A P L N

An exemplary amino acid sequence of a C-terminal capping region is:

(SEQ ID NO: 19)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in one example embodiment, fragments of the N-terminal and/or C-terminal capping regions are included in the TALE polypeptides described herein.

In one example embodiment, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In another example embodiment, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In one example embodiment, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In one example embodiment, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes, but is not limited to, a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include, but are not limited to, transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Engineered Transcriptional Activators (CRISPRa)

In one example embodiment, a programmable nuclease system is used to recruit an activator protein to a target gene in order to enhance expression. In one example embodiment, the activator protein is recruited to the enhancer region of the target gene. For example, a catalytically inactive Cas protein (“dCas”) fused to an activator can be used to recruit that activator protein to the target sequence. Accordingly, a guide sequence is designed to direct binding of the dCas-activator fusion such that the activator can interact with the target genomic region and induce target gene expression. The Cas protein used may be any of the Cas proteins disclosed above. In one example protein, the Cas protein is a dCas9.

In one embodiment, the programmable nuclease system is a CRISPRa system (see, e.g., US20180057810A1; and Konermann et al. “Genome-scale transcriptional activation by an engineered CRISPR-Cas9 complex” Nature. 2014 Dec. 10. doi: 10.1038/nature14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N-/C-terminus of the CRISPR enzyme.

In another example embodiment, a zinc finger system is used to recruit an activation domain to the target gene. In one example embodiment, the activation domain is linked to the zinc finger system. In general, the positioning of the one or more activator domains on the zinc finger system is one which allows for correct spatial orientation for the activator domain to affect the target with the attributed functional effect.

In another example embodiment, a TALE system is used to recruit an activation domain to the target gene. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target.

In another example embodiment, a meganuclease system is used to recruit an activation domain to the target gene. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target.

### Base Editing

In one example embodiment, a method of treating subjects comprises administering a base editing system that is directed to a target gene (e.g., a regulator). A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas12a or Cas12b polypeptide. The nucleobase deaminase may be cytosine base editor (CBE) or adenosine base editors (ABEs). CBEs convert CG base pairs into a TA base pair (Komor et al. 2016. Nature. 533:420-424; Nishida et al. 2016. Science. 353; and Li et al. Nat. Biotech. 36:324-327) and ABEs convert an AT base pair to a GC base pair. Collectively, CBEs and ABEs can mediate all four possible transition mutations (C to T, A to G, T to C, and G to A). Example base editing systems are disclosed in Rees and Liu. 2018. Nat. Rev. Genet. 19(12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

The editing window of a base editing system may range over a 5-8 nucleotide window, depending on the base editing system used. Id. Accordingly, given the base editing system used, a guide sequence may be selected to direct the base editing system to convert a base or base pair of one or more target genes.

### ARCUS Based Editing

In one example embodiment, a method of treating subjects comprises administering an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### Prime Editing

In one example embodiment, a method of treating subjects comprises administering a prime editing system directed to a target gene. In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g., genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposases (CAST)

In one example embodiment, a method of treating a subject comprises administering a CAST system that replaces a genomic region in a target gene. In one example embodiment, a CAST system is used to replace all or a portion of an enhancer controlling target gene expression.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g., genomic DNA). The guide molecule is capable of forming a CRISPR-Cas complex with the Cas polypeptide and can be programmed to direct the entire CAST complex such that the transposase is positioned to insert the donor sequence at the target site on the target polynucleotide. For multimeric transposase, only those transposases needed for recognition of the donor construct and transposition of the donor sequence into the target polypeptide may be required. The Cas may be naturally catalytically inactive or engineered to be catalytically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-1f Cas polypeptide. In one example embodiment, the Cas polypeptide may comprise a cas6, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type 1f-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-1b Cas polypeptide. In one example embodiment, the Cas polypeptide may comprise a cas6, a cas7, and a cas8b (e.g., a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO/2021/041922 which is incorporated herein by reference.

In one example embodiment, the CAST comprise a catalytically inactive Type II Cas polypeptide (e.g., dCas9) fused to one or more polypeptides of a Tn5 transposase. In another example embodiment, the CAST system comprises a catalytically inactive Type II Cas polypeptide (e.g., dCas9) fused to a piggyback transposase.

### Epigenetic Editing

In example embodiments, the one or more agents is an epigenetic modification polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides. As used herein, “linked to or otherwise capable of associating with” refers to a fusion protein or a recruitment domain or an adaptor protein, such as an aptamer (e.g., MS2) or an epitope tag. The recruitment domain or an adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example embodiments, the DNA binding domain is a programmable DNA binding protein linked to or otherwise capable of associating with an epigenetic modification domain. Programmable DNA binding proteins for modifying the epigenome include, but are not limited to CRISPR systems, transcription activator-like effectors (TALEs), Zn finger proteins and meganucleases (see, e.g., Thakore P I, Black J B, Hilton I B, Gersbach C A. Editing the epigenome: technologies for programmable transcription and epigenetic modulation. Nat Methods. 2016; 13(2):127-137; and described further herein). In example embodiments, the DNA binding domain is a nuclease-deficient RNA-guided DNA endonuclease enzyme or a nuclease-deficient endonuclease enzyme. In example embodiments, a CRISPR system having an inactivated nuclease activity (e.g., dCas) is used as the DNA binding domain.

In example embodiments, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150(1):12-27; Syding L A, Nickl P, Kasparek P, Sedlacek R. CRISPR/Cas9 Epigenome Editing Potential for Rare Imprinting Diseases: A Review. Cells. 2020; 9(4):993; and Zhang Y. Transcriptional regulation by histone ubiquitination and deubiquitination. Genes Dev.

2003; 17(22):2733-2740). Example epigenetic modification domains can be obtained from, but are not limited to chromatin modifying enzymes, such as, DNA methyltransferases (e.g., DNMT1, DNMT3a and DNMT3b), TET1, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SETS), MMSET, EZH2, and MLL family members), DOT1L, LSD1, Jumonji demethylases (e.g., KDM5A (JARID1A), KDM5C (JARID1C), and KDM6A (UTX)), kinases (e.g., Haspin, VRK1, PKCα, PKCβ, PIM1, IKKα, Rsk2, PKB/Akt, Aurora B, MSK1/2, JNK1, MLTKα, PRK1, Chk1, Dlk/ZIP, PKG5, MST1, AMPK, JAK2, Abl, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1). See, also, U.S. patent Ser. No. 11/001,829B2 for additional domains.

In example embodiments, histone acetylation is targeted to a target sequence using a CRISPR system (see, e.g., Hilton I B, et al. Epigenome editing by a CRISPR-Cas9-based acetyltransferase activates genes from promoters and enhancers. Nat Biotechnol. 2015). In example embodiments, histone deacetylation is targeted to a target sequence (see, e.g., Cong et al., 2012; and Konermann S, et al. Optical control of mammalian endogenous transcription and epigenetic states. Nature. 2013; 500:472-476). In example embodiments, histone methylation is targeted to a target sequence (see, e.g., Snowden A W, Gregory P D, Case C C, Pabo C O. Gene-specific targeting of H3K9 methylation is sufficient for initiating repression in vivo. Curr Biol. 2002; 12:2159-2166; and Cano-Rodriguez D, Gjaltema R A, Jilderda L J, et al. Writing of H3K4Me3 overcomes epigenetic silencing in a sustained but context-dependent manner. Nat Commun. 2016; 7:12284). In example embodiments, histone demethylation is targeted to a target sequence (see, e.g., Kearns N A, Pham H, Tabak B, et al. Functional annotation of native enhancers with a Cas9-histone demethylase fusion. Nat Methods. 2015; 12(5):401-403). In example embodiments, histone phosphorylation is targeted to a target sequence (see, e.g., Li J, Mahata B, Escobar M, et al. Programmable human histone phosphorylation and gene activation using a CRISPR/Cas9-based chromatin kinase. Nat Commun. 2021; 12(1):896). In example embodiments, DNA methylation is targeted to a target sequence (see, e.g., Rivenbark A G, et al. Epigenetic reprogramming of cancer cells via targeted DNA methylation. Epigenetics. 2012; 7:350-360; Siddique A N, et al. Targeted methylation and gene silencing of VEGF-A in human cells by using a designed Dnmt3a-Dnmt3L single-chain fusion protein with increased DNA methylation activity. J Mol Biol. 2013; 425:479-491; Bernstein D L, Le Lay J E, Ruano E G, Kaestner K H. TALE-mediated epigenetic suppression of CDKN2A increases replication in human fibroblasts. J Clin Invest. 2015; 125:1998-2006; Liu X S, Wu H, Ji X, et al. Editing DNA Methylation in the Mammalian Genome. Cell. 2016; 167(1):233-247.e17; Stepper P, Kungulovski G, Jurkowska R Z, et al. Efficient targeted DNA methylation with chimeric dCas9-Dnmt3a-Dnmt3L methyltransferase. Nucleic Acids Res. 2017; 45(4):1703-1713; and Pflueger C., Tan D., Swain T., Nguyen T., Pflueger J., Nefzger C., Polo J. M., Ford E., Lister R. A modular dCas9-SunTag DNMT3A epigenome editing system overcomes pervasive off-target activity of direct fusion dCas9-DNMT3A constructs. Genome Res. 2018; 28:1193-1206). In example embodiments, DNA demethylation is targeted to a target sequence using a CRISPR system (see, e.g., TET1, see Xu et al, Cell Discov. 2016 May 3; 2: 16009; Choudhury et al, Oncotarget. 2016 Jul. 19; 7(29):46545-46556; and Kang J G, Park J S, Ko J H, Kim Y S. Regulation of gene expression by altered promoter methylation using a CRISPR/Cas9-mediated epigenetic editing system. Sci Rep. 2019; 9(1):11960). In example embodiments, DNA demethylation is targeted to a target sequence (see, e.g., TDG, see, Gregory D J, Zhang Y, Kobzik L, Fedulov A V. Specific transcriptional enhancement of inducible nitric oxide synthase by targeted promoter demethylation. Epigenetics. 2013; 8:1205-1212).

Example epigenetic modification domains can be obtained from, but are not limited to transcription activators, such as, VP64 (see, e.g., Ji Q, et al. Engineered zinc-finger transcription factors activate OCT4 (POU5F1), SOX2, KLF4, c-MYC (MYC) and miR302/367. Nucleic Acids Res. 2014; 42:6158-6167; Perez-Pinera P, et al. Synergistic and tunable human gene activation by combinations of synthetic transcription factors. Nat Methods. 2013; 10:239-242; Farzadfard F, Perli S D, Lu T K. Tunable and multifunctional eukaryotic transcription factors based on CRISPR/Cas. ACS Synth Biol. 2013; 2:604-613; Black J B, Adler A F, Wang H G, et al. Targeted Epigenetic Remodeling of Endogenous Loci by CRISPR/Cas9-Based Transcriptional Activators Directly Converts Fibroblasts to Neuronal Cells. Cell Stem Cell. 2016; 19(3):406-414; and Maeder M L, Linder S J, Cascio V M, Fu Y, Ho Q H, Joung J K. CRISPR RNA-guided activation of endogenous human genes. Nat Methods. 2013; 10(10):977-979), p65 (see, e.g., Liu P Q, et al. Regulation of an endogenous locus using a panel of designed zinc finger proteins targeted to accessible chromatin regions. Activation of vascular endothelial growth factor A. J Biol Chem. 2001; 276:11323-11334; and Konermann S, et al. Genome-scale transcriptional activation by an engineered CRISPR-Cas9 complex. Nature. 2015; 517:583-588), HSF1, and RTA (see, e.g., Chavez A, et al. Highly efficient Cas9-mediated transcriptional programming. Nat Methods. 2015; 12:326-328). Example epigenetic modification domains can be obtained from, but are not limited to transcription repressors, such as, KRAB (see, e.g., Beerli R R, Segal D J, Dreier B, Barbas C F., 3rd Toward controlling gene expression at will: specific regulation of the erbB-2/HER-2 promoter by using polydactyl zinc finger proteins constructed from modular building blocks. Proc Natl Acad Sci USA. 1998; 95:14628-14633; Cong L, Zhou R, Kuo Y C, Cunniff M, Zhang F. Comprehensive interrogation of natural TALE DNA-binding modules and transcriptional repressor domains. Nat Commun. 2012; 3:968; Gilbert L A, et al. CRISPR-mediated modular RNA-guided regulation of transcription in eukaryotes. Cell. 2013; 154:442-451; and Yeo N C, Chavez A, Lance-Byrne A, et al. An enhanced CRISPR repressor for targeted mammalian gene regulation. Nat Methods. 2018; 15(8):611-616).

In example embodiments, the epigenetic modification domain linked to a DNA binding domain recruits an epigenetic modification protein to a target sequence. In example embodiments, a transcriptional activator recruits an epigenetic modification protein to a target sequence. For example, VP64 can recruit DNA demethylation, increased H3K27ac and H3K4me. In example embodiments, a transcriptional repressor protein recruits an epigenetic modification protein to a target sequence. For example, KRAB can recruit increased H3K9me3 (see, e.g., Thakore P I, D'Ippolito A M, Song L, et al. Highly specific epigenome editing by CRISPR-Cas9 repressors for silencing of distal regulatory elements. Nat Methods. 2015; 12(12):1143-1149). In an example embodiment, methyl-binding proteins linked to a DNA binding domain, such as MBD1, MBD2, MBD3, and MeCP2 recruits an epigenetic modification protein to a target sequence. In an example embodiment, Mi2/NuRD, Sin3A, or Co-REST recruit HDACs to a target sequence.

In example embodiments, the epigenetic modification domain can be a eukaryotic or prokaryotic (e.g., bacteria or Archaea) protein. In example embodiments, the eukaryotic protein can be a mammalian, insect, plant, or yeast protein and is not limited to human proteins (e.g., a yeast, insect, plant chromatin modifying protein, such as yeast HATs, HDACs, methyltransferases, etc.

In one aspect of the invention, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In embodiments, the epigenetic modification polypeptide comprises the nuclease-deficient RNA-guided DNA endonuclease enzyme. In embodiments, the fusion protein comprises the nuclease-deficient DNA endonuclease enzyme.

In some embodiments, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9 (see, e.g., U.S. patent Ser. No. 11/001,829B2).

In certain embodiments, the present invention provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present invention provides a method of activating a target nucleic acid sequence in a cell, the method comprising: (i) delivering a first polynucleotide encoding a epigenetic modification polypeptide described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length.

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

Delivery mechanisms for CAST systems includes those discussed above for CRISPR-Cas systems.

### Healthy Lifestyle Regimen

In example embodiments, a subject is treated with a customized lifestyle regimen. In example embodiments, a customized lifestyle regimen includes a customized diet and/or customized exercise regimen. For example, a customized diet can include increasing intake of fruits and vegetables, reducing saturated fat, dairy products, and sugar.

Further embodiments are illustrated in the following Examples which are given for illustrative purposes only and are not intended to limit the scope of the invention.

### EXAMPLES

### Example 1—Inherited Basis of Visceral, Abdominal Subcutaneous and Gluteofemoral Fat Depots

In this study, Applicants investigate the common and rare variant genetic architecture of three fat depots as quantified by MM in up to 38,965 UK Biobank participants. Beyond study of raw VAT, ASAT, and GFAT volumes, Applicants analyze six measures that better reflect local adiposity and fat distribution: VAT adjusted for BMI and height (VATadj), ASATadj, GFATadj, VAT/ASAT, VAT/GFAT, and ASAT/GFAT. Applicants show that these local adiposity traits (1) highlight depot-specific genetic architecture, (2) reflect sex-dimorphism previously appreciated with anthropometric traits, and (3) can be used to construct depot-specific polygenic scores that have divergent associations with type 2 diabetes and coronary artery disease. This study is to Applicants knowledge the largest imaging-based study to date to disentangle the genetic architecture of different fat depots—including GFAT, a fat depot that appears to confer protection from adverse cardiometabolic health^{5,30}.

### Results

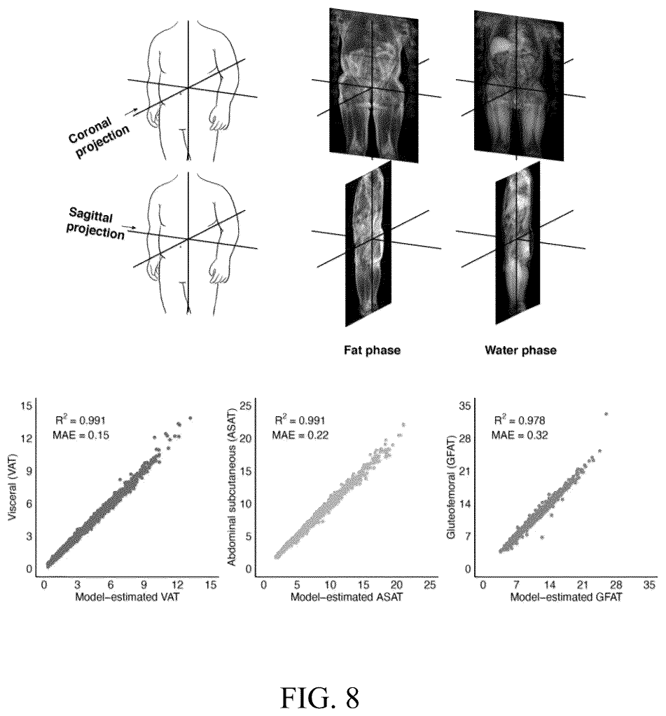

VAT, ASAT, and GFAT volumes were quantified in participants of the UK Biobank using a deep learning model trained on body MRI imaging, as previously described (FIG. 1, FIG. 8, and Supplementary Table 1)⁵. Among those with Mill-quantified fat depot volumes, 39,076 had genotyping array data available, enabling common variant association studies in up to 38,965 participants after quality control (“Methods”). Mean age in the genotyped cohort was 64.5 years, 51% were female, and 87% were of white British ancestry as previously defined in this study (Supplementary Data 1 and 2). As expected, significant sex differences in fat depot volumes were observed—male participants had higher mean VAT volume (5.0 vs. 2.6 L), while female participants had higher ASAT volume (7.9 vs. 5.9 L) and GFAT volume (11.3 vs. 9.3 L)^{31,32}.

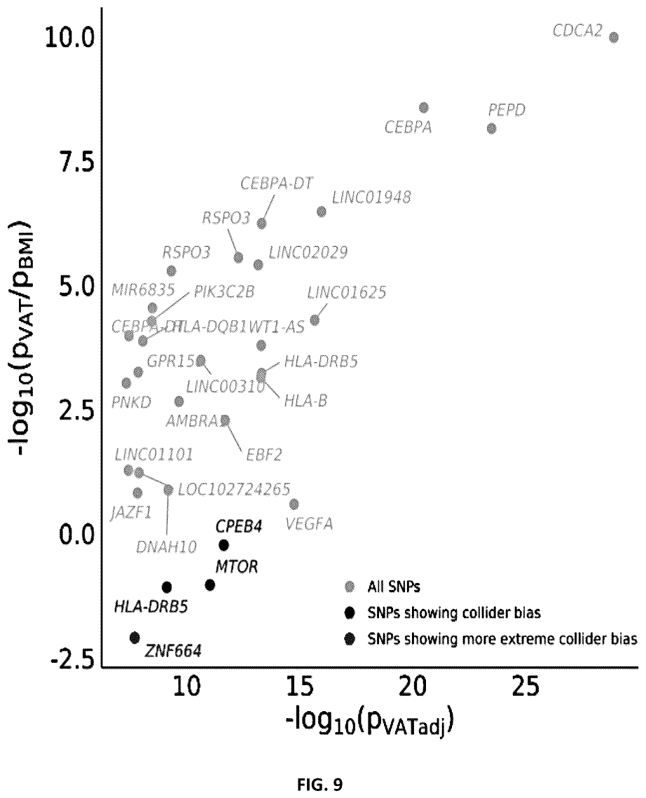

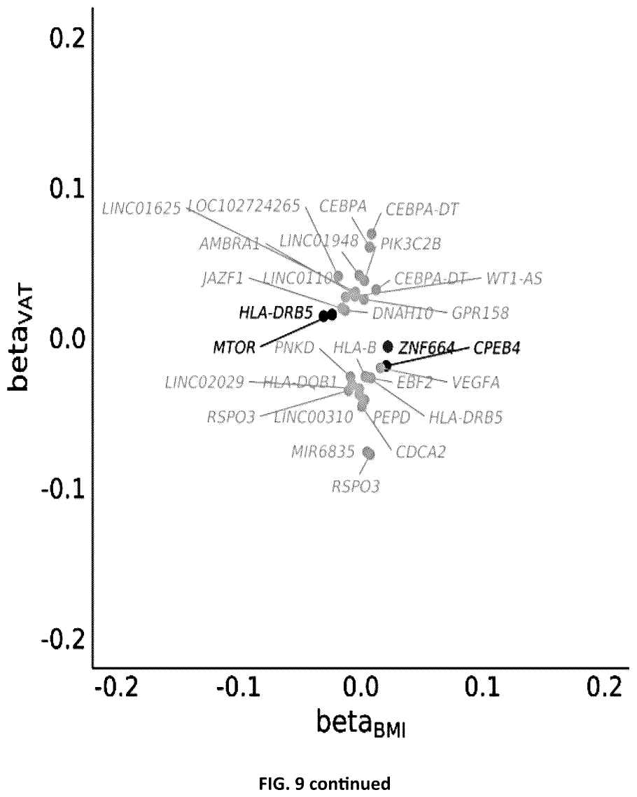

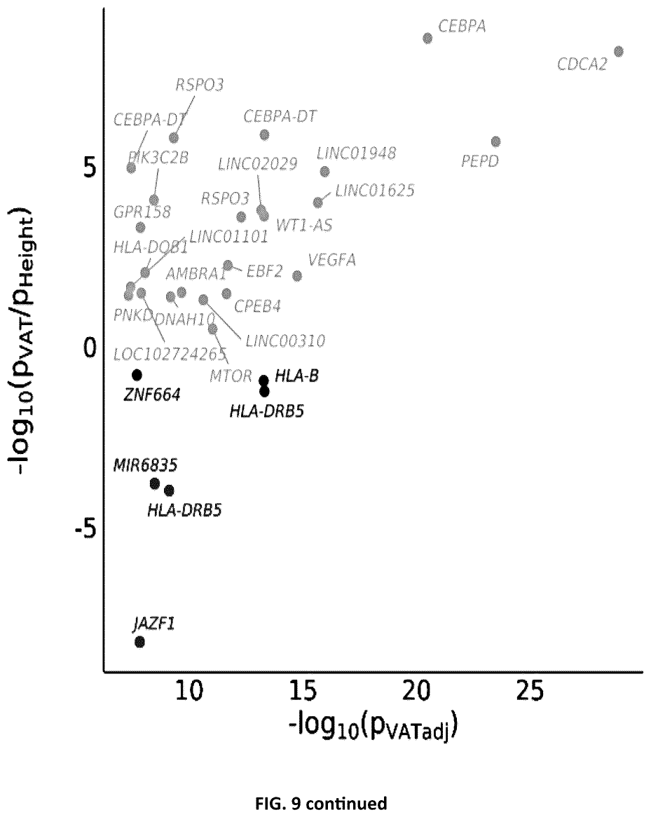

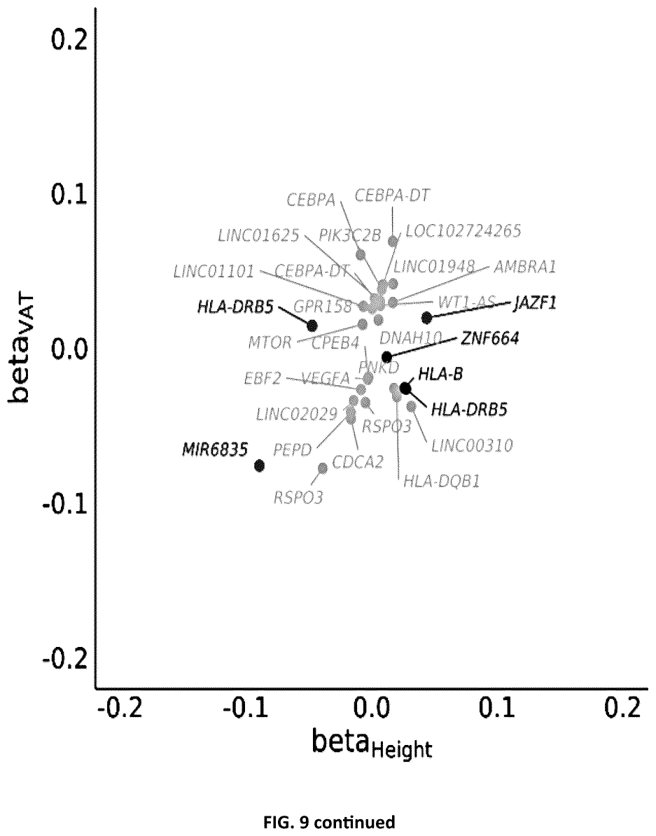

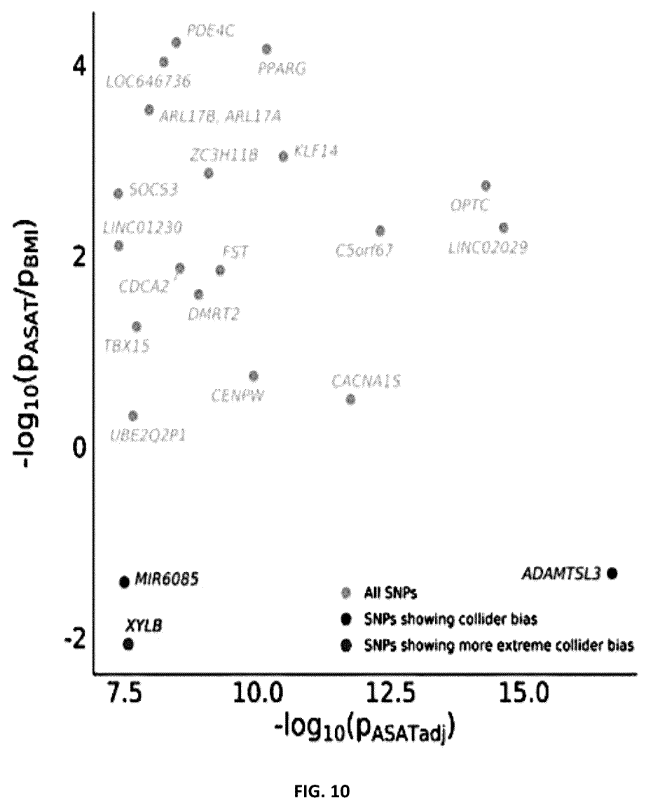

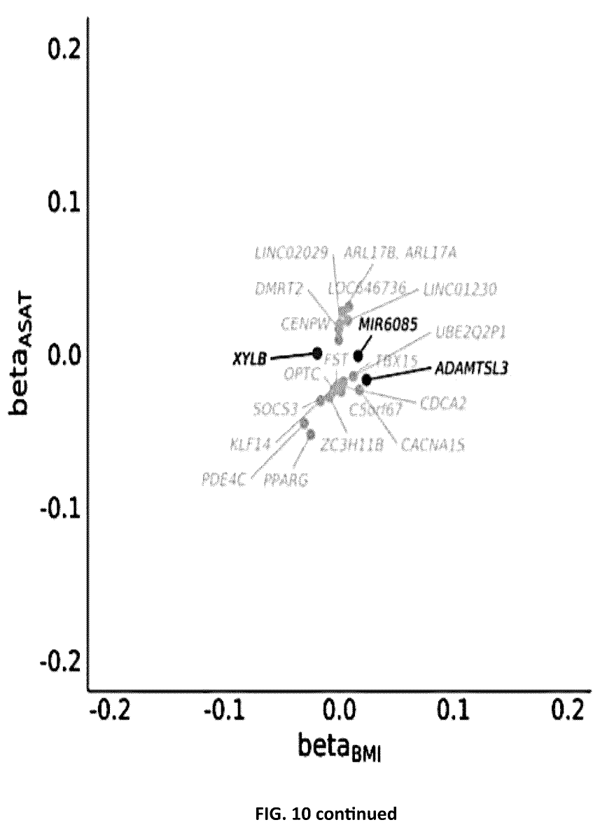

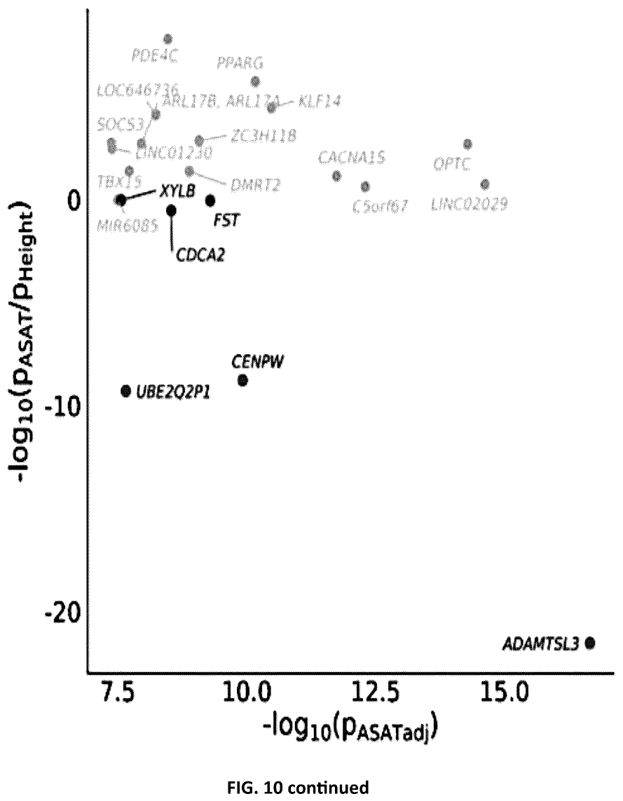

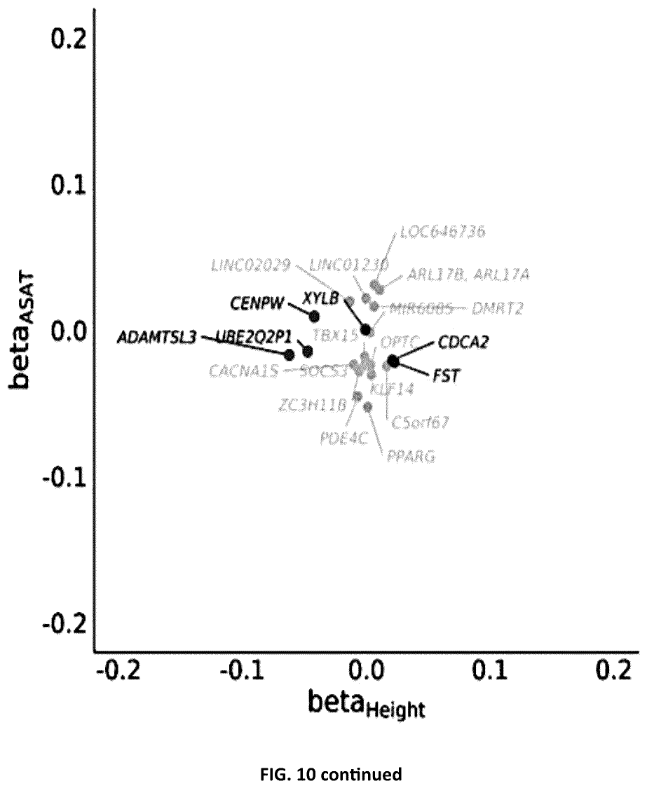

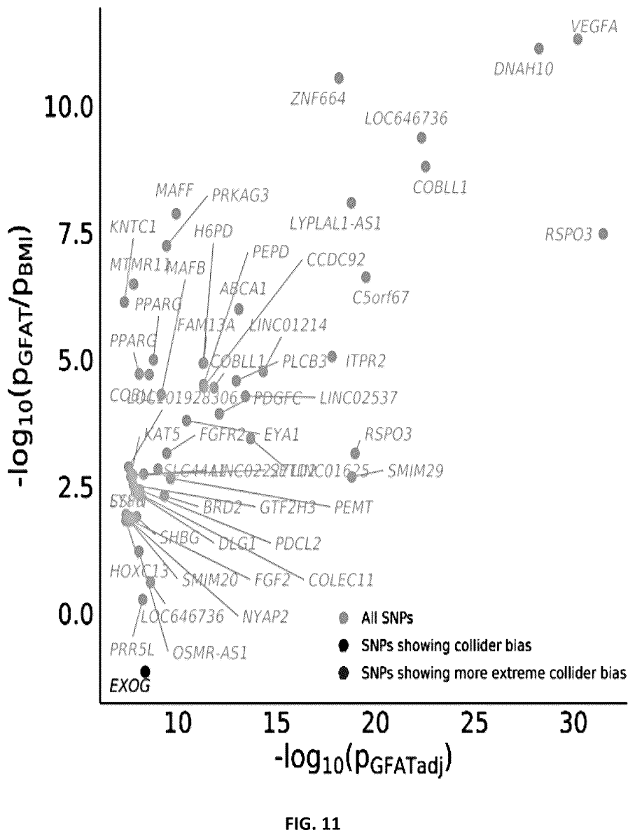

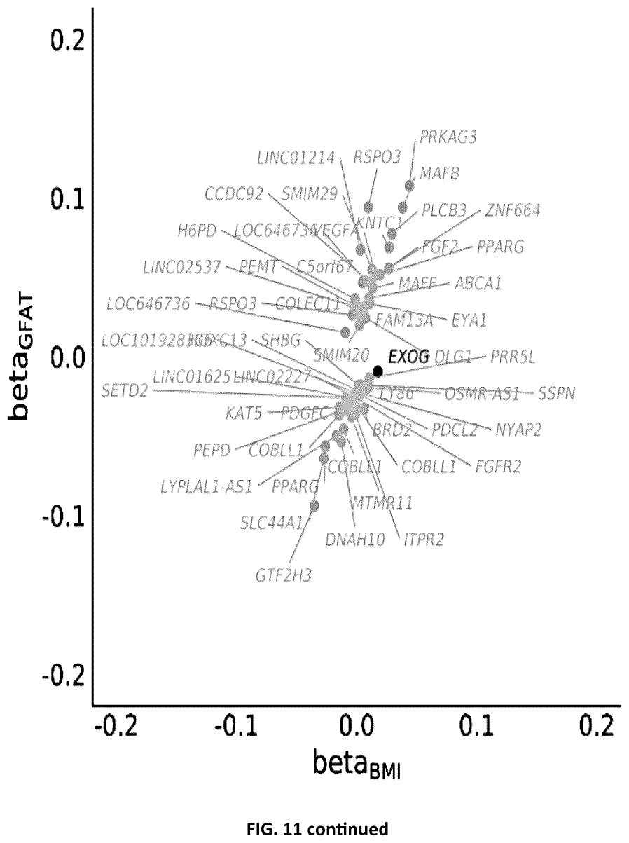

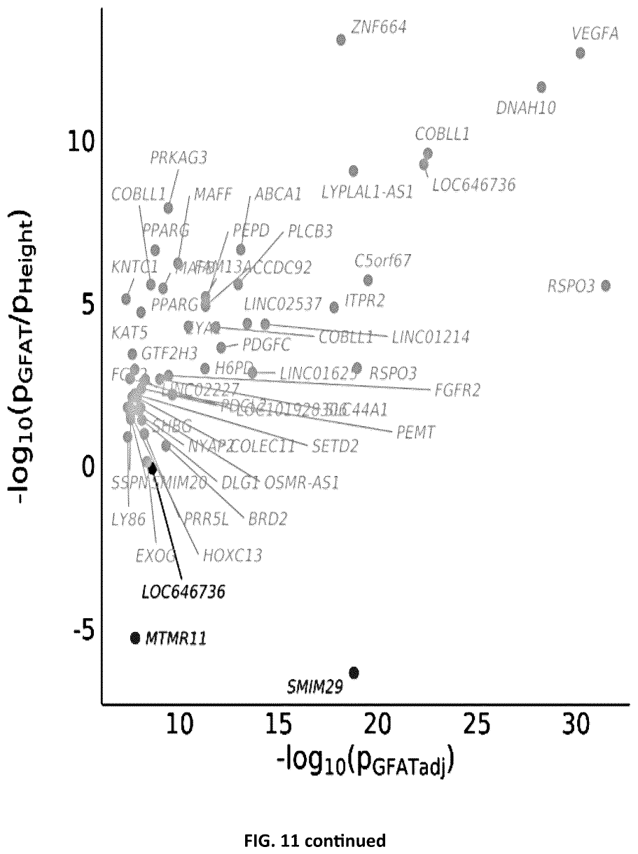

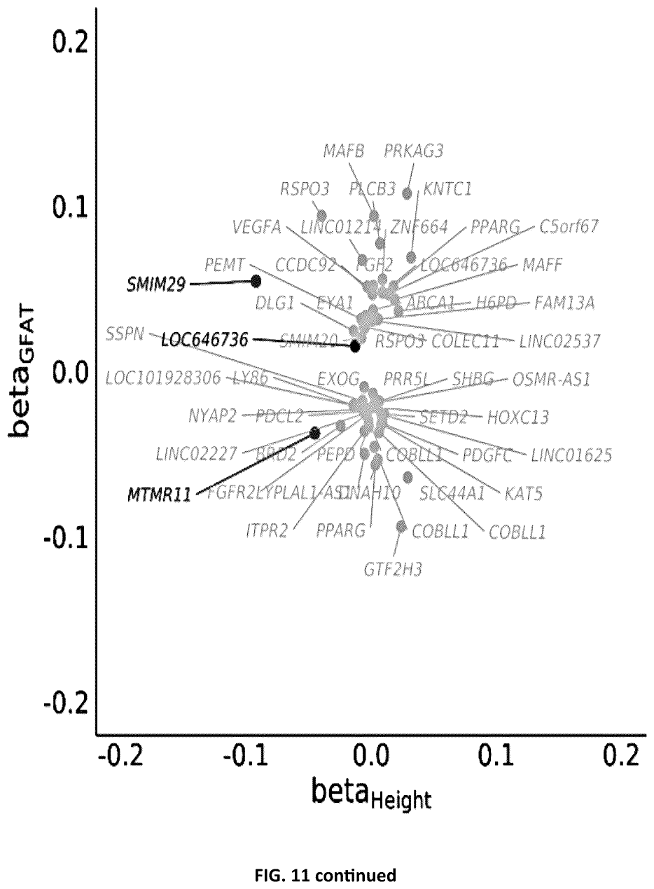

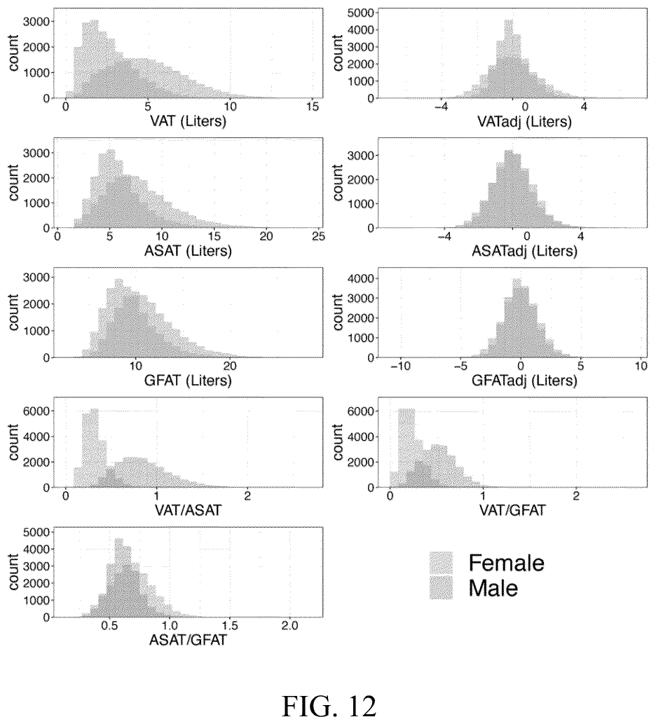

Six additional adiposity traits—designed to better capture local adiposity—were additionally computed for each individual: VATadj, ASATadj, GFATadj were computed by taking sex-specific residuals against age, age squared, BMI, and height, while VAT/ASAT, VAT/GFAT, and ASAT/GFAT were computed by taking ratios between each pair of fat depots without additional residualization (FIG. 12). Applicants tested VATadj, ASATadj, and GFATadj for possible collider bias with BMI or height and found minimal or no evidence of such bias for the majority of genome-wide significant loci (Methods, FIGS. 9-11, and Supplementary Tables 2-5). For example, 87% of VATadj, 86% of ASATadj, and 98% of GFATadj genome-wide significant loci had stronger effect size for the unadjusted fat depot volume compared to BMI, comparable to the 90% of WHRadjBMI loci that met analogous criteria in a recent meta-analysis' 2.

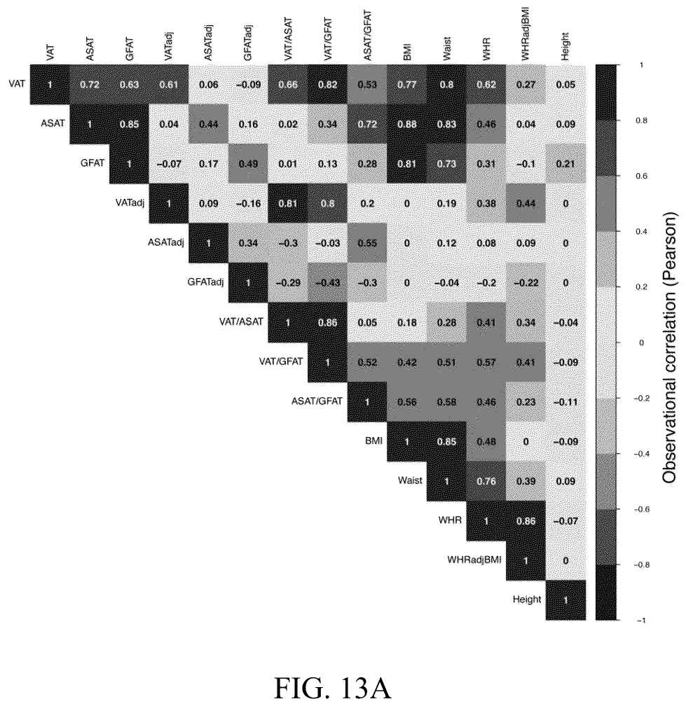

In contrast to VAT, ASAT, and GFAT volumes which were highly correlated with BMI (Pearson r ranging from 0.77-0.88), VATadj, ASATadj, GFATadj, and VAT/ASAT were nearly independent of BMI (Pearson r ranging from 0-0.18), while VAT/GFAT (Pearson r=0.42) and ASAT/GFAT (Pearson r=0.56) displayed attenuated correlations with BMI (FIG. 2 and FIG. 13A, B). These six derived adiposity traits provided useful, less BMI-dependent metrics for downstream analyses.

Local Adiposity Traits are Highly Heritable and Genetically Distinct from Each Other

To quantify the inherited component to each of these nine adiposity traits, Applicants used the BOLT-REML algorithm to estimate SNP-heritability. Heritability estimates for VAT, ASAT, and GFAT ranged from 0.31-0.36 (standard error (SE)=0.01), comparable to that observed for BMI in the same individuals (h_{g}²: 0.31, SE=0.02)) (Supplementary Table 6). BMI-adjusted fat depots and fat depot ratios tended to have higher heritability compared to unadjusted fat depots and BMI (h_{g}² ranging from 0.34-0.41, SE=0.01-0.02). In contrast, WHRadjBMI, an anthropometric proxy for local adiposity, was less heritable than these traits (h_{g}²: 0.21, SE=0.01). In sex-stratified analyses, most adiposity traits were more heritable in females as compared to males, with the greatest heritability across all analyses for GFATadj in females (h_{g}²: 0.52, SE=0.03).

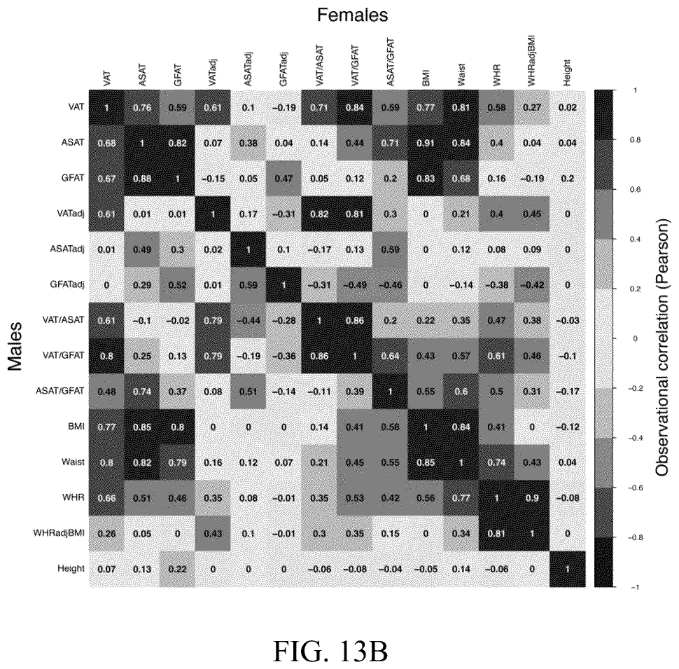

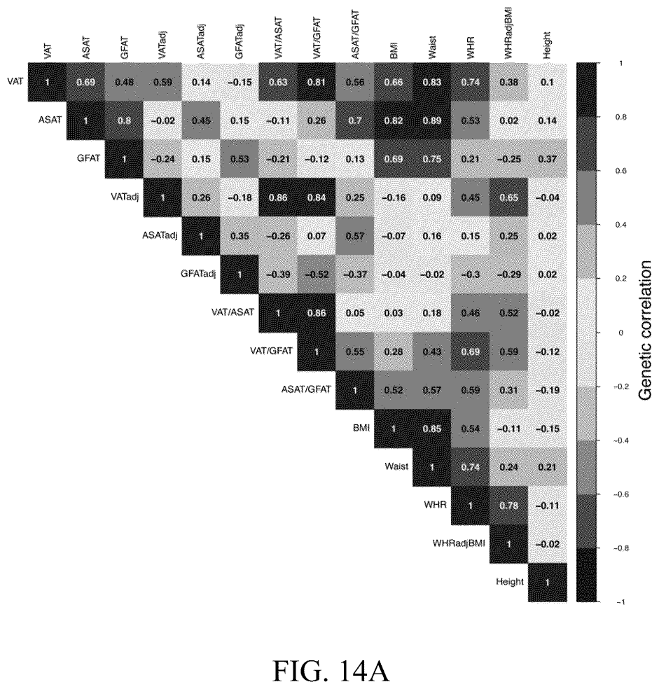

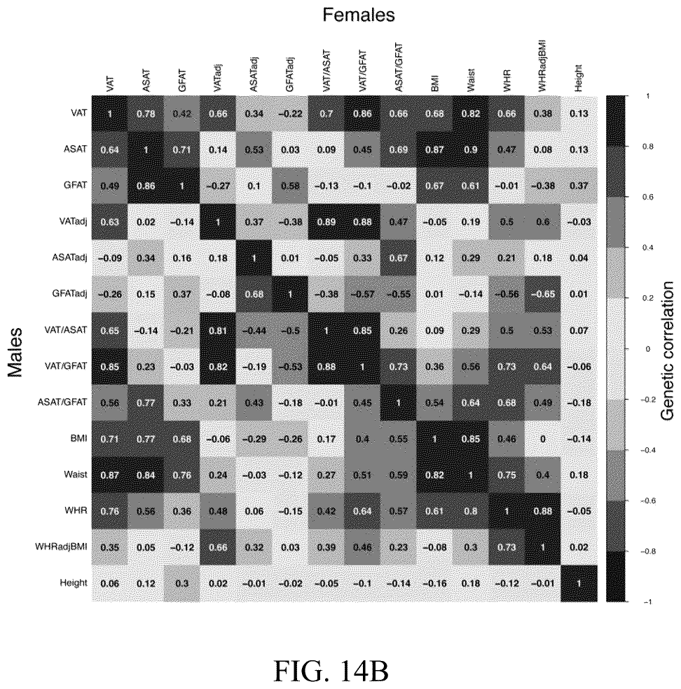

To study the genetic correlations (r_{g}) between the adiposity and related anthropometric traits, Applicants used LD-score regression^{33,34}. Results were generally consistent with observational correlations—raw VAT, ASAT, and GFAT volumes were highly genetically correlated with BMI (r_{g} ranging from 0.66-0.82), while the three adjusted fat depots, VAT/ASAT, and VAT/GFAT exhibited low genetic correlation with BMI (r_{g} ranging from −0.16-0.28) (FIG. 2 and FIG. 14A, B). In sex-combined analyses, VATadj, ASATadj, and GFATadj were genetically correlated with their unadjusted counterparts (r_{g} ranging from 0.45-0.59), but nearly independent of the other two fat depots (r_{g} ranging from −0.24-0.15), suggesting that adjusted-for-BMI traits can enable fat depot-specific genetic analyses. Finally, WHRadjBMI exhibited positive genetic correlations with VATadj (r_{g}: 0.65) and ASATadj (r_{g}: 0.25), and a negative genetic correlation with GFATadj (r_{g}: −0.29), consistent with the perturbations needed in each fat depot to drive a change in WHRadjBMI.

### Common Variant Architecture of Adiposity Traits

Applicants next conducted GWAS for each of the nine adiposity traits—VAT, ASAT, GFAT, VATadj, ASATadj, GFATadj, VAT/ASAT, VAT/GFAT, and ASAT/GFAT—in sex-combined and sex-stratified groups using BOLT-LMM. After genotyping quality control, Applicants tested associations with 11.5 million imputed SNPs with minor allele frequency (MAF)>0.005. Across all 27 association studies, 250 loci were associated with at least one adiposity trait at a p value threshold of 5×10⁻⁸ (Supplementary Data 3). If a more stringent genome-wide significance threshold of 5×10⁻⁹ had been used, Applicants would have identified 136 loci, or 85 loci at the most conservative Bonferroni-corrected threshold of 5×10⁹/27=1.9×10⁻¹⁰. Of the 250 loci across all adiposity traits, 39 were newly-identified (defined as R²<0.1 with all genome-wide significant associations with prior adiposity and relevant anthropometric traits in the GWAS catalog) (Table 1; Methods; and Supplementary Data 4)³⁵. Of these 39 loci, 35 have been previously associated with at least one cardiometabolic trait with nominal significance (p<0.05) (Supplementary Table 7). Consistent with heritability estimates, the greatest number of loci were identified in association with GFATadj (54 lead SNPs), while the fewest were identified in association with ASAT (6 lead SNPs). The greatest genomic inflation parameter (λ_{GC}) was observed with GFATadj (λ_{GC}: 1.14)—the LD-score regression intercept was 1.05, consistent with polygenicity rather than significant population structure (Supplementary Table 8)³³.

TABLE 1
Forty-two newly-identified locus-trait associations in this study.

| | | | | Effect | Other | | | | | |
|---|---|---|---|---|---|---|---|---|---|---|
| Trait | CHR | BP | SNP | allele | allele | EAF | BETA | SE | p value | Nearest gene |
| GFAT | 11 | 95840436 | rs1074742 | A | G | 0.401 | 0.041 | 0.007 | 1.40E−08 | MAML2 |
| GFAT | 12 | 124344710 | rs138756410 | T | C | 0.986 | −0.172 | 0.031 | 3.00E−08 | DNAH10 |
| GFAT | 12 | 125092343 | rs4765159 | A | G | 0.018 | 0.146 | 0.027 | 3.50E−08 | NCOR2 |
| VATadj | 2 | 121310704 | rs35932591 | C | T | 0.879 | 0.061 | 0.011 | 3.80E−08 | LINC01101 |
| VATadj | 10 | 25767521 | rs1329254 | C | T | 0.37 | 0.042 | 0.007 | 1.40E−08 | GPR158 |
| VATadj | 11 | 69195097 | rs7933253 | T | C | 0.048 | 0.098 | 0.017 | 1.30E−08 | LOC102724265 |
| VATadj | 2 | 121310704 | rs35932591 | C | T | 0.88 | 0.086 | 0.016 | 3.90E−08 | LINC01101 |
| (Male) | | | | | | | | | | |
| VATadj | 3 | 56901687 | rs1500714 | C | G | 0.854 | 0.081 | 0.015 | 1.80E−08 | ARHGEF3 |
| (Female) | | | | | | | | | | |
| ASATadj | 1 | 201016296 | rs3850625 | G | A | 0.882 | −0.079 | 0.011 | 1.80E−12 | CACNA1S |
| ASATadj | 9 | 1044400 | rs2048235 | C | T | 0.384 | 0.041 | 0.007 | 4.10E−08 | LINC01230 |
| ASATadj | 9 | 1052722 | rs6474550 | G | T | 0.66 | 0.045 | 0.008 | 1.30E−09 | DMRT2 |
| ASATadj | 15 | 62757857 | rs17205757 | A | G | 0.674 | −0.042 | 0.008 | 3.20E−08 | MIR6085 |
| ASATadj | 17 | 76324751 | rs4444401 | A | G | 0.473 | −0.04 | 0.007 | 4.20E−08 | SOCS3 |
| ASATadj | 1 | 116916645 | rs749166380 | CT | C | 0.102 | 0.102 | 0.018 | 2.20E−08 | ATP1A1 |
| (Female) | | | | | | | | | | |
| ASATadj | 8 | 58352327 | rs776481989 | ATAAT | A | 0.998 | 0.795 | 0.134 | 8.60E−09 | LOC101929488 |
| (Female) | | | | | | | | | | |
| GFATadj | 2 | 3648186 | rs7588285 | C | G | 0.188 | 0.053 | 0.009 | 1.40E−08 | COLEC11 |
| GFATadj | 2 | 226768344 | 2:226768344_CA_C | CA | C | 0.193 | −0.051 | 0.009 | 2.60E−08 | NYAP2 |
| GFATadj | 3 | 196818853 | rs13099700 | A | G | 0.722 | 0.047 | 0.008 | 7.90E−09 | DLG1 |
| GFATadj | 5 | 38810354 | rs142369482 | G | GT | 0.656 | −0.044 | 0.008 | 9.10E−09 | OSMR-AS1 |
| GFATadj | 10 | 122970216 | rs1907218 | T | C | 0.314 | −0.049 | 0.008 | 3.60E−10 | FGFR2 |
| GFATadj | 4 | 104780790 | rs528845403 | A | AATGTGT | 0.991 | −0.325 | 0.061 | 2.40E−08 | TACR3 |
| (Male) | | | | | | | | | | |
| GFATadj | 1 | 181161153 | rs7550430 | A | G | 0.998 | 0.892 | 0.144 | 1.80E−09 | LINC01732 |
| (Female) | | | | | | | | | | |
| GFATadj | 2 | 165533198 | rs386652275 | T | TC | 0.974 | −0.19 | 0.034 | 3.20E−08 | COBLL1 |
| (Female) | | | | | | | | | | |
| VAT/ASAT | 2 | 178121005 | rs13028464 | C | T | 0.631 | −0.039 | 0.007 | 4.80E−08 | NFE2L2 |
| VAT/ASAT | 6 | 19947871 | rs70987287 | T | TTTTTA | 0.728 | 0.064 | 0.008 | 1.70E−17 | ID4 |
| VAT/ASAT | 8 | 25459001 | rs3890765 | C | A | 0.941 | −0.084 | 0.015 | 6.80E−09 | CDCA2 |
| VAT/ASAT | 9 | 1054362 | rs6474552 | G | C | 0.432 | −0.04 | 0.007 | 1.20E−08 | DMRT2 |
| VAT/ASAT | 10 | 63702572 | rs55767272 | A | C | 0.937 | 0.085 | 0.014 | 6.80E−09 | ARID5B |
| VAT/ASAT | 10 | 122992475 | rs11199845 | C | T | 0.46 | 0.055 | 0.007 | 1.50E−14 | FGFR2 |
| VAT/ASAT | 2 | 61760756 | rs13390751 | A | C | 0.838 | 0.076 | 0.013 | 1.30E−08 | XPO1 |
| (Male) | | | | | | | | | | |
| VAT/ASAT | 6 | 19949170 | 6:19949170_GT_G | GT | G | 0.746 | 0.068 | 0.012 | 3.70E−09 | ID4 |
| (Male) | | | | | | | | | | |
| VAT/ASAT | 10 | 122992442 | rs11199844 | C | T | 0.463 | 0.059 | 0.01 | 5.90E−09 | FGFR2 |
| (Male) | | | | | | | | | | |
| VAT/ASAT | 6 | 19947871 | rs70987287 | T | TTTTTA | 0.729 | 0.064 | 0.011 | 8.50E−10 | ID4 |
| (Female) | | | | | | | | | | |
| VAT/ASAT | 12 | 121319417 | rs59757908 | T | C | 0.995 | −0.425 | 0.076 | 4.20E−08 | SPPL3 |
| (Female) | | | | | | | | | | |
| VAT/GFAT | 14 | 94844947 | rs28929474 | C | T | 0.982 | 0.16 | 0.026 | 4.80E−10 | SERPINA1 |
| VAT/GFAT | 1 | 162430821 | rs9660318 | G | C | 0.203 | 0.068 | 0.012 | 1.80E−08 | UHMK1 |
| (Female) | | | | | | | | | | |
| VAT/GFAT | 2 | 116072770 | rs11399916 | T | TA | 0.256 | 0.06 | 0.011 | 3.70E−08 | DPP10 |
| (Female) | | | | | | | | | | |
| VAT/GFAT | 6 | 32975699 | rs9276981 | G | C | 0.809 | −0.064 | 0.012 | 4.60E−08 | HLA-DOA |
| (Female) | | | | | | | | | | |
| ASAT/GFAT | 5 | 55830865 | rs39837 | C | T | 0.667 | 0.043 | 0.007 | 2.60E−08 | LINC01948 |
| ASAT/GFAT | 14 | 95219657 | rs8006225 | G | T | 0.817 | 0.055 | 0.009 | 2.60E−09 | GSC |
| ASAT/GFAT | 16 | 86424697 | rs1552657 | G | A | 0.549 | −0.037 | 0.007 | 4.90E−08 | LINC00917 |
| ASAT/GFAT | 5 | 55830865 | rs39837 | C | T | 0.666 | 0.061 | 0.01 | 9.10E−09 | LINC01948 |
| (Female) | | | | | | | | | | |

Newly-identified loci were defined as loci that associated with an adiposity trait with p<5×10⁻⁸ and that were not in LD (R 2<0.10) with any of the loci in the GWAS catalog for adiposity or related anthropometric traits (see “Methods”)³⁵. “adj” traits are adjusted for BMI and height (see “Methods”). Note that rs35932591 (VATadj and VATadj (Male)), rs70987287 (VAT/ASAT and VAT/ASAT (Female)), and rs39837 (ASAT/GFAT and ASAT/GFAT (Female)) are duplicated, so 39 unique lead SNPs are presented in this table. Loci were additionally cross-referenced with prior studies using the Type 2 Diabetes Knowledge Portal (Supplementary Table 7). BP GRCh37 position, EAF effect allele frequency, BETA effect size per effect allele, p value BOLT-LA/1M association p value.

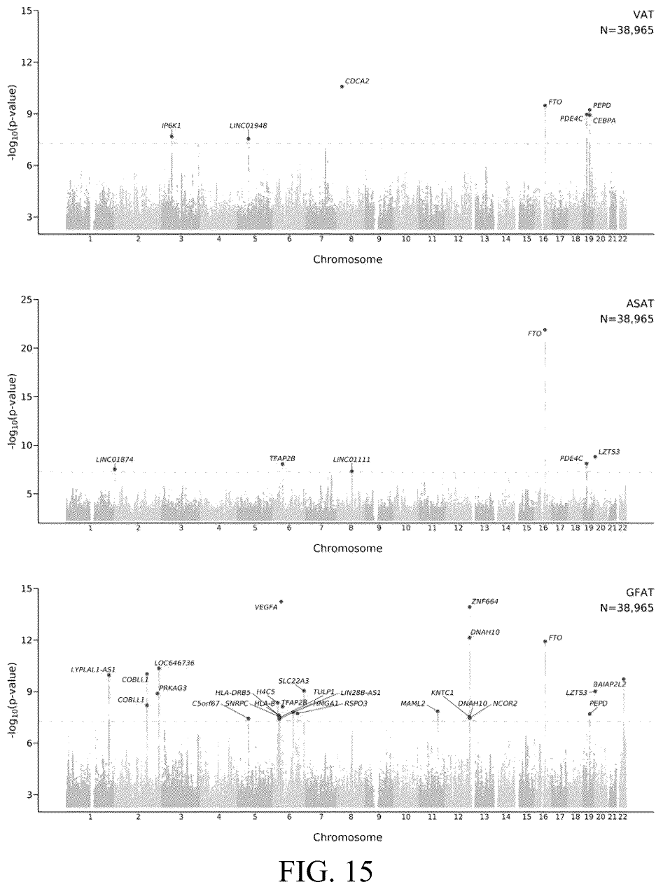

Applicants began by investigating the genetic architecture of VAT, ASAT, and GFAT volumes (FIG. 15). All three traits shared a genome-wide significant association with an intronic FTO variant (r556094641) previously associated with childhood and adult obesity³⁶⁻³⁸. ASAT harbored the most significant association with this locus (p=1.3×10⁻²²), followed by GFAT (p=1.2×10⁻¹²), and finally VAT (p=3.3×10⁻¹⁹), reflecting the strength of observational and genetic correlation of each fat depot with BMI. Given observational and genetic evidence that a large component of each fat depot volume trait is accounted for by BMI—or “overall adiposity”—Applicants focused further common variant analyses to the three adjusted-for-BMI-and-height measures and three fat depot ratios, aiming to study the genetic architecture of “local adiposity.”

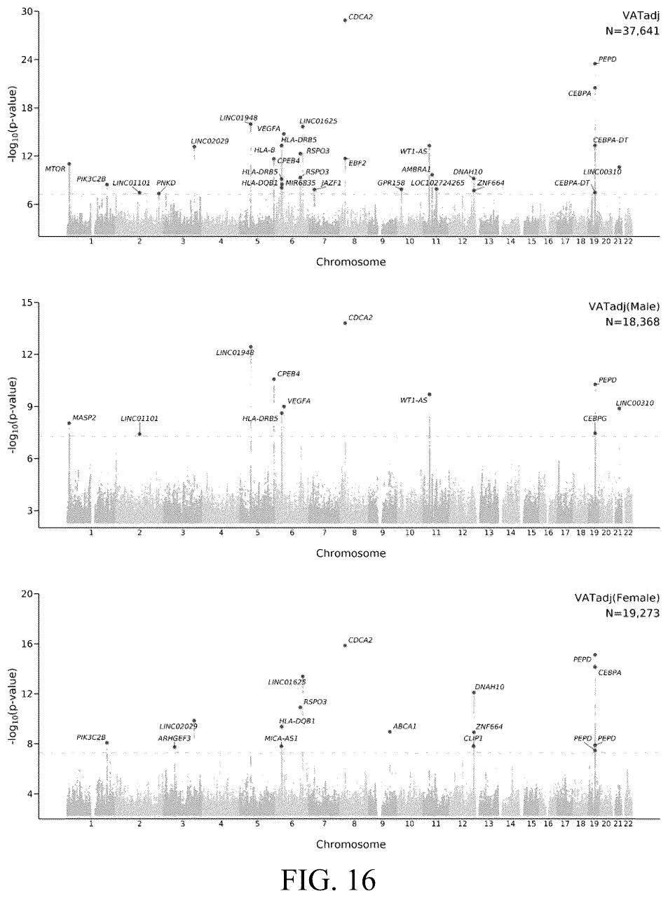

For VATadj, 30 genome-wide significant associations were identified (p<5×10⁻⁸) (FIG. 1 and FIG. 16). The two most significantly associated variants were an intronic CDCA2variant (r511992444; p=1.3×10⁻²⁹) previously associated with WHRadjBMI and serum triglycerides, and an intronic PEPD variant (r510406327; p=3.3×10⁻²⁴) previously associated with waist circumference adjusted for BMI (WCadjBMI) and type 2 diabetes^{12,39-41}. Newly-identified loci in association with VATadj included an intronic GPR158 variant (rs1329254; p=1.4×10⁻⁸), and an intronic ARHGEF3 variant exclusively in females (r51500714; p=1.8×10⁻⁸). Prior work has similarly noted female-specific effects of variation in this gene including an association with postmenopausal osteoporosis in humans and Arhgef3-KO mice being found to have improved muscle regeneration following injury, with an enhanced rate in females, although the role of this gene on fat distribution is uncertain^{42,43}.

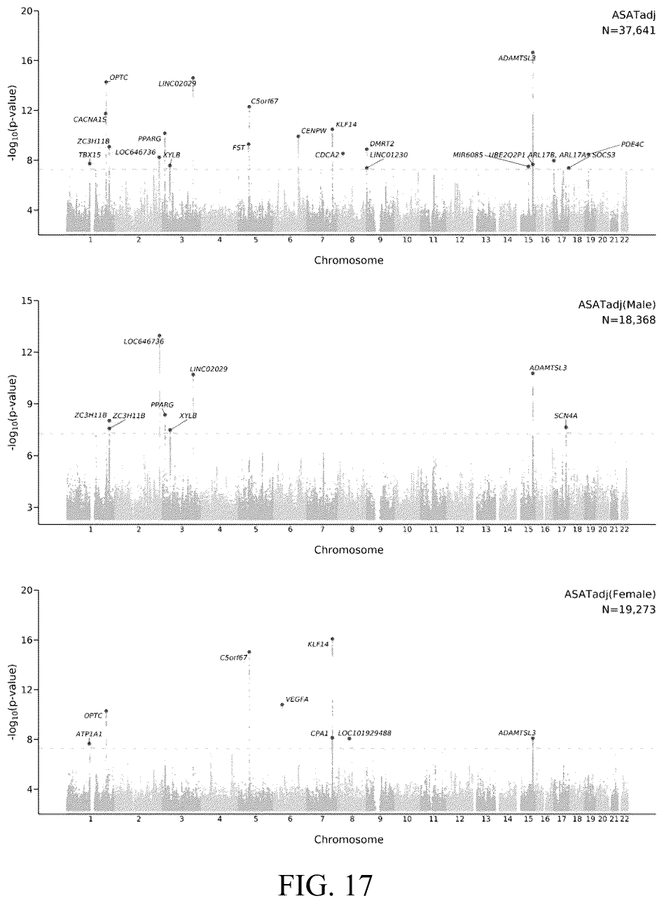

The most statistically significant association with ASATadj was an intronic ADAMTSL3 variant (rs768397327; p=2.2×10⁻¹⁷), which was in near-perfect linkage disequilibrium (R²=0.97) with another intronic ADAMTSL3 variant (r511856122) previously associated with bioelectrical impedance-derived arm fat ratio, leg fat ratio, and trunk fat ratio (FIG. 1 and FIG. 17)¹³. Another genome-wide significant signal was observed with an intronic PPARG variant (r5527620413). Rare variants in PPARG have previously been associated with familial partial lipodystrophy^{6,7}. The minor alleles at this locus (MAF=0.12), which additionally consisted of rs17036328 and rs71304101 (R²>0.90), were associated with increased ASATadj (r5527620413; beta=0.071; p=6.8×10⁻¹¹), increased GFATadj (r571304101; beta=0.062; p=1.7×10⁻⁹), decreased VAT/ASAT ratio (r517036328; beta=−0.080; p=5.8×10⁻¹⁵), and decreased VAT/GFAT ratio (rs17036328; beta=−0.058; p=2.4×10⁻⁸). These three SNPs are also in high LD (R²≥0.94) with rs1801282, a missense variant in PPARG previously associated with reduced risk of type 2 diabetes⁴⁴⁻⁴⁶. These data suggest that common variation at PPARG can lead to adiposity variation along the lipodystrophy axis—for this locus, the minor alleles associated with a pattern of favorable adiposity. FST is another gene that promotes adipogenesis and may have a causal role in insulin resistance—an intronic variant in FST (rs557 44247) associated with ASATadj (p=5.1×10⁻¹⁰), but not VATadj (p=0.80) or GFATadj (p=0.25)⁴⁷. Finally, a newly-identified intronic DMRT2 variant (r56474550; p=1.3×10⁻⁹) associated with ASATadj. In a study investigating fat depot-specific transcriptome signatures before and after exercise, DMRT2 was one of three genes with higher expression in ASAT vs. GFAT both before and after exercise⁴⁸.

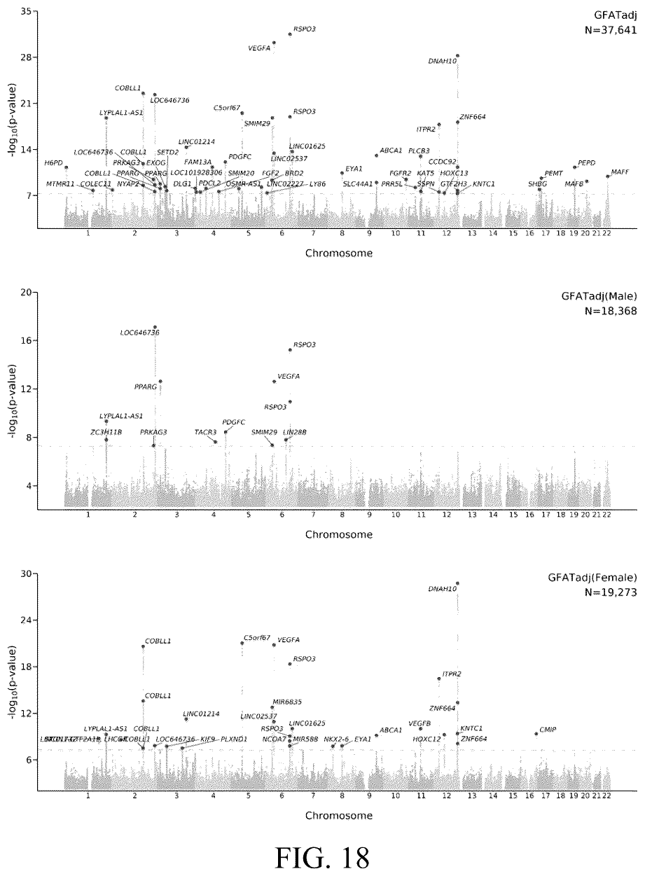

The top GFATadj signal was an intronic RSPO3 variant (r572959041; p=3.2×10⁻³²) that has previously been shown to be a top signal for WHRadjBMI (FIG. 1 and FIG. 18)¹². Recent work clarified this SNP as the causal variant at the locus and suggested that the minor allele concurrently reduces leg fat mass and increases android fat mass⁴⁹. The results confirm and further clarify these findings—the minor allele (MAF=0.05) associated with marked reduction of GFATadj (beta=−0.195; p=3.2×10⁻³²) and increased of VATadj (beta=0.118; p=7.8×10⁻¹³), but a nonsignificant effect on ASATadj (beta=−0.029; p=0.09). Three independent intronic COBLL1 variants (R 2<0.1) were associated with GFATadj (r513389219; p=3.0×10⁻²³, rs3820981; p=1.5×10⁻¹², rs34224594; p=2.8×10⁻⁹), but not VATadj (pₘᵢₙ=0.009) or ASATadj (pₘᵢₙ=2.7×10⁻³). One of these variants (rs13389219) is in LD with another intronic COBLL1 variant (rs6738627) which has previously been implicated in a metabolically healthy obesity phenotype characterized by increased HDL cholesterol and reduced triglycerides despite increased body fat percentage⁵⁰. In this study, aligning rs13389219 to the BMI-increasing direction (beta=0.011, p=7.3×10⁻³) revealed a concurrent increase in GFATadj (beta=0.073), consistent with a metabolically healthy fat depot shift. Finally, a GFATadj association was observed at an intronic PDGFC variant (rs6822892; p=8.0×10⁻¹³)—PDGFC was recently prioritized as a candidate causal gene for insulin resistance in human preadipocytes and adipocytes⁴⁷.

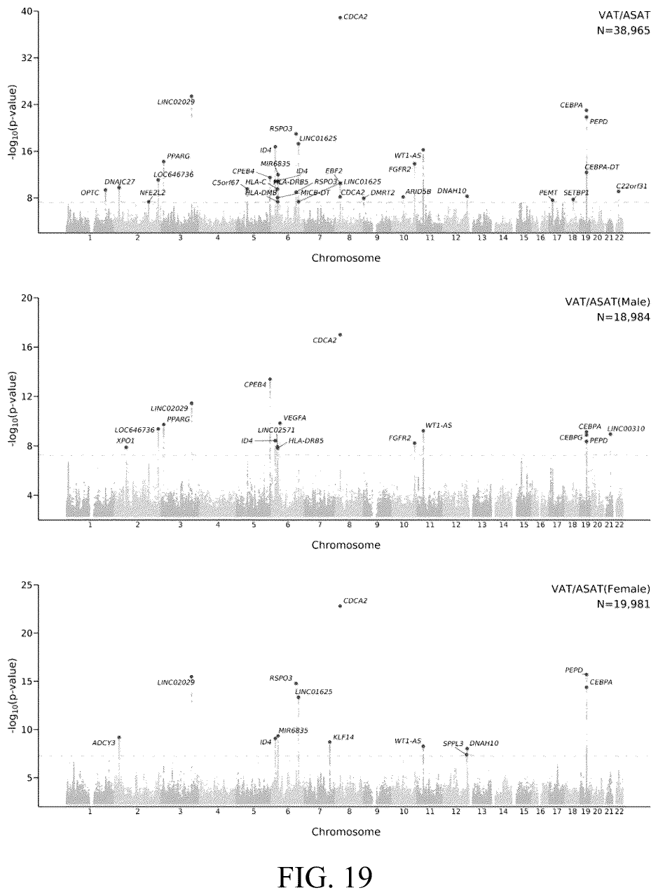

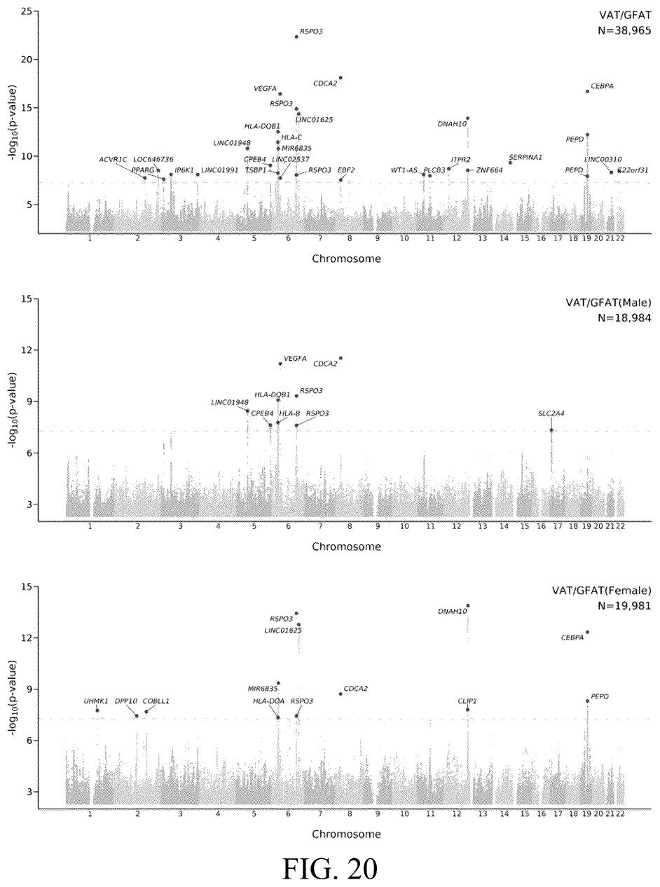

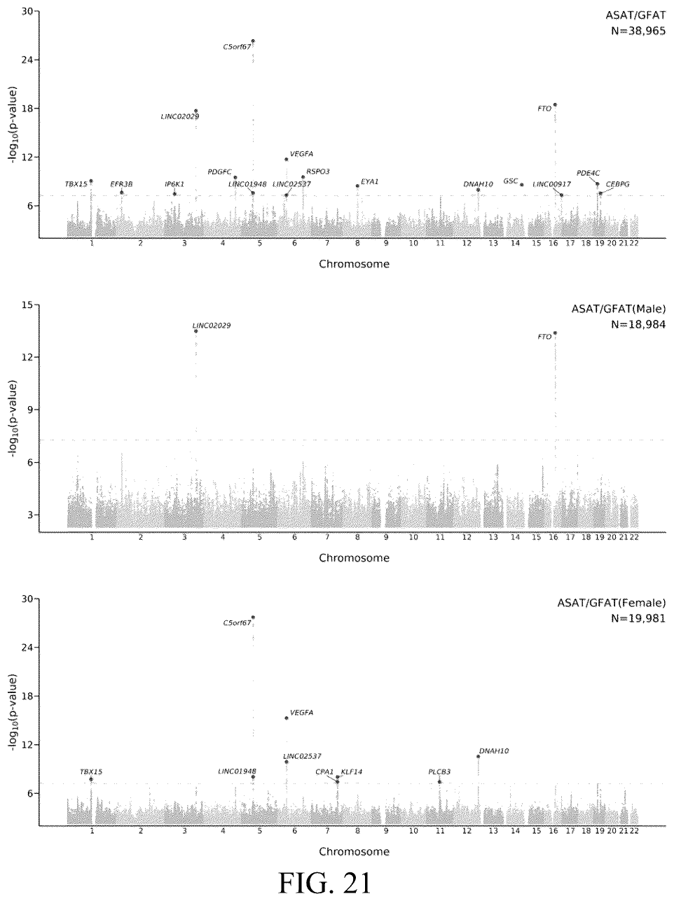

Several associations were exclusive to GWASs of fat depot ratios (FIGS. 19-21). A missense variant in ACVR1C significantly reduced VAT/GFAT ratio (r555920843; MAF=0.01; beta=−0.18; p=1.9×10⁻⁸). Prior work demonstrated that sequence variation in ACVR1C—including this variant—reduces WHRadjBMI and risk of type 2 diabetes 51. Another missense variant in ACVR1C was nominally associated with reduced VAT/GFAT ratio, strengthening the importance of this gene (r556188432 (p.Ile195Thr); beta=−0.21, p=0.006) (Supplementary Data 5). Finally, a newly-identified association was present between VAT/GFAT ratio and a missense variant in SERPINA1 (rs28929474; MAF=0.02; beta=−0.16; p=4.8×10⁻¹⁰). Homozygous carriers of this variant are known to harbor alpha-1-antitrypsin deficiency, and heterozygous carriers have higher serum ALT and increased risk of cirrhosis^{51,52}. Interestingly, this missense variant has also been associated with reduced risk of type 2 diabetes (odds ratio: 0.90, p=5.9×10⁻⁶) and coronary artery disease (odds ratio: 0.88, p=9.4×10⁻⁹)^{41,53}. The present association with reduced VAT/GFAT ratio suggests that a shift toward a metabolically healthy fat distribution could partially explain a reduced risk of cardiometabolic disease. In a large meta-analysis, this SERPINA1 variant had only a nominally significant association with waist-to-hip ratio (beta=−0.03, p=3.4×10⁻⁴)—the closest anthropometric correlate of VAT/GFAT ratio—highlighting the utility of image-derived phenotypes for this discovery¹².

### Gluteofemoral Adiposity Signal Classification

Applicants aimed to categorize genetic loci associated with gluteofemoral adiposity postulated to be metabolically protective—into distinct clusters. Starting with the 250 lead SNPs that were associated (p<5×10⁻⁸) with any of the nine adiposity traits in this study, Applicants selected 101 LD-pruned (r²=0.1) SNPs that were nominally associated (p<0.05) with GFATadj. Each SNP was aligned to the GFATadj increasing direction. Applicants used Bayesian non-negative matrix factorization (bNMF)—a soft clustering approach—with 32 cardiometabolic traits including anthropometric traits (e.g., BMI, body fat percentage), lipid traits (e.g., triglycerides, HDL-cholesterol, and total cholesterol), and diabetes-related traits (e.g., glucose, hemoglobin A1C) to identify clusters (Supplementary Data 6).

In all 100 iterations, the data converged to three clusters (Supplementary Data 7). The most strongly weighted traits for the first cluster included increased HDL-cholesterol, decreased serum triglycerides, decreased hemoglobin A1C, and decreased alanine aminotransferase, consistent with a metabolically healthier fat distribution. Top loci in this cluster included several well-known associations with WHRadjBMI and insulin resistance including COBLL1, RSPO3, PPARG, and DNAH10^{12,47,54,55}. A second cluster appeared to be related to inflammatory pathways, with top loci including HLA-DRB5, HLA-B, and MAFB—MAFB has previously been implicated as a regulator of adipose tissue inflammation⁵⁶. Strongly weighted traits in this cluster included decreased aspartate aminotransferase, decreased total cholesterol, and decreased C-reactive protein. The third and final cluster appeared to reflect the interplay between hepatocyte biology and fat distribution with top loci including a missense variant in SERPINA1 and SHBG—the former is known to cause alpha-1-antitrypsin deficiency and has been previously associated with increased ALT and cirrhosis, and sex-hormone binding globulin is synthesized by hepatocytes and is reduced in patients with non-alcoholic fatty liver disease^{57,58}. Strongly weighted traits in this cluster included increased albumin, increased sex-hormone binding globulin, and increased total protein.

To test the robustness of these results, Applicants performed two sensitivity analyses. First, Applicants performed clustering using 85 LD-pruned SNPs nominally associated (p<0.05) with unadjusted GFAT. The three aforementioned clusters were reproduced along with a fourth cluster representing overall adiposity—the top locus in this cluster was FTO and the most strongly weighted trait was increased BMI (Supplementary Data 8). Finally, Applicants performed one additional clustering analysis of the same 101 LD-pruned SNPs for GFATadj, this time including VATadj and ASATadj as clustering traits alongside the 32 previously used cardiometabolic traits, resulting in a nearly identical set of three clusters (Supplementary Data 9).

Sex Heterogeneity in Genetic Associations with Local Adiposity Traits

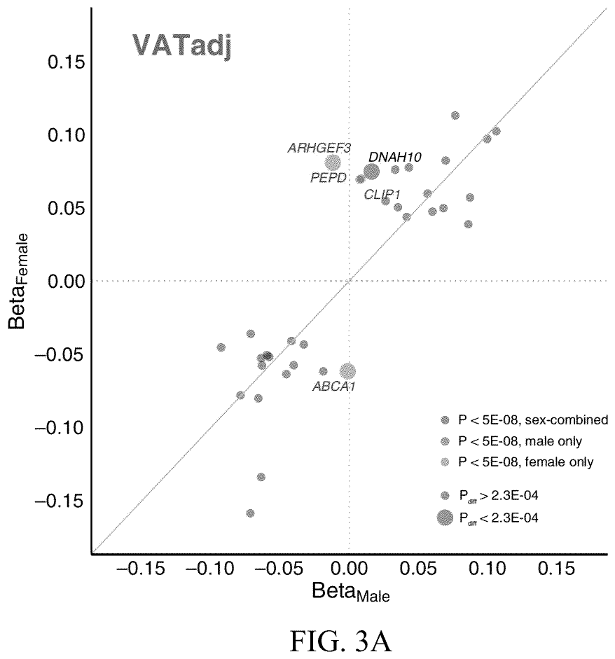

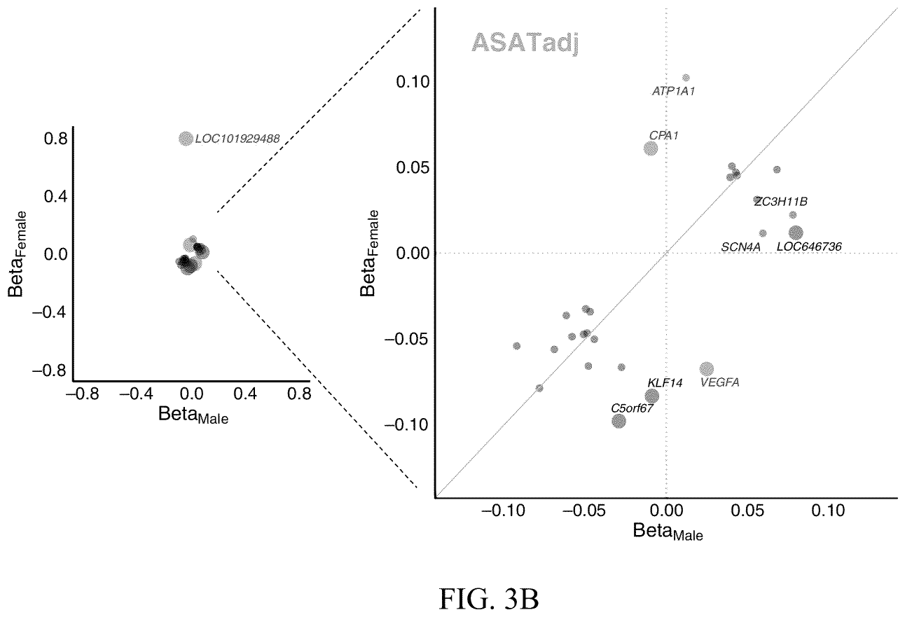

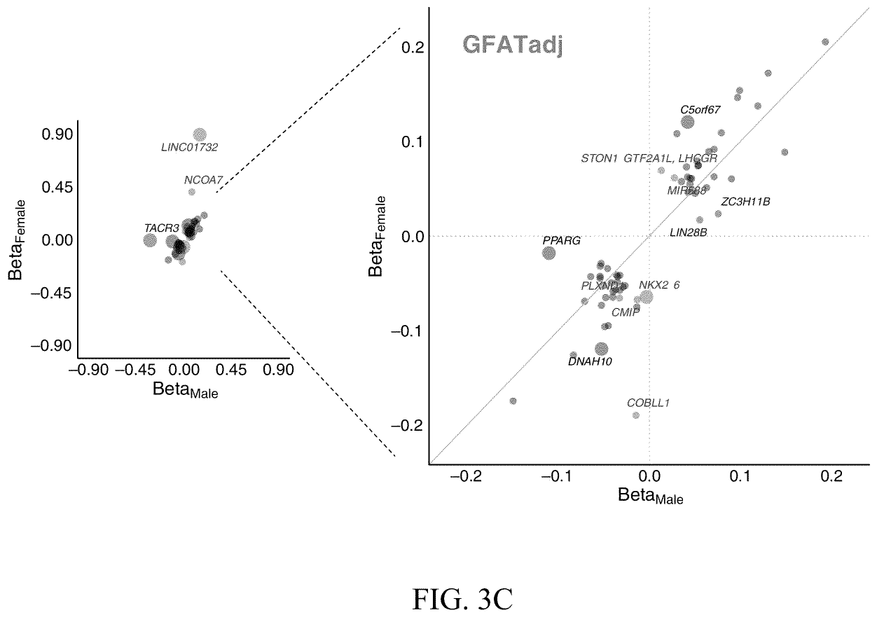

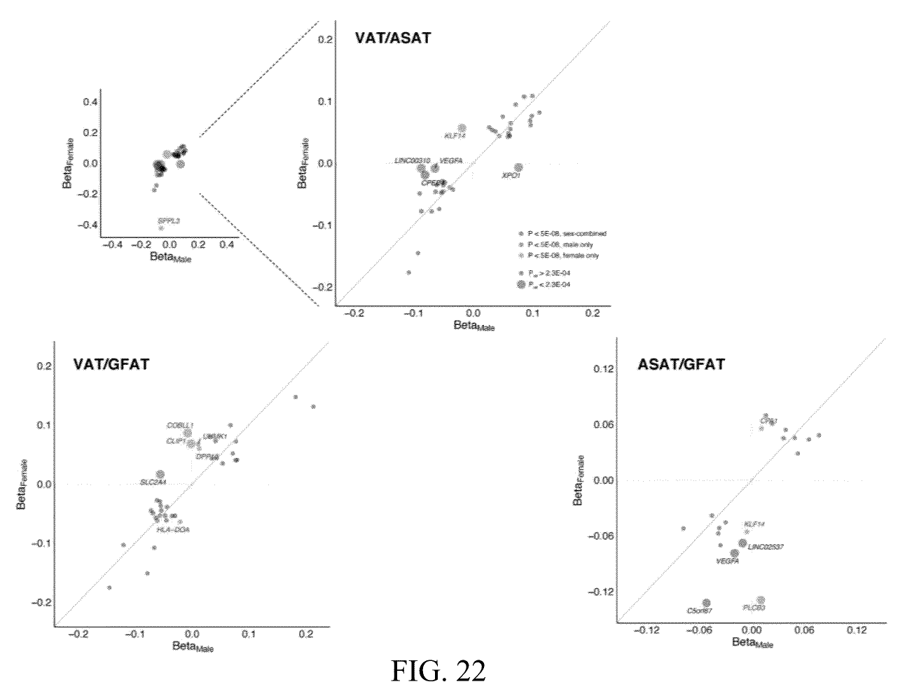

Given prior work has noted significant sex heterogeneity in the genetic basis of anthropometric traits, Applicants next tested for such heterogeneity for each of the six local adiposity traits (VATadj, ASATadj, GFATadj, VAT/ASAT, VAT/GFAT, and ASAT/GFAT)^{11,12,55,59}. Genetic correlations between sex-stratified summary statistics indicated overall high correlation between traits, with r g somewhat higher for VATadj (r_{g}=0.87) as compared to ASATadj or GFATadj (r_{g}=0.80 and 0.79 respectively) (Supplementary Table 9). Applicants next tested for sex-dimorphism across loci that were genome-wide significant for either sex-combined or sex-stratified analyses for each local adiposity trait (FIG. 3A-C, FIG. 22, and Supplementary Data 10). Three of 34 VATadj loci (9%), six of 27 ASATadj loci (22%), and six of 65 GFATadj (9%) showed significant sex dimorphism (p_{diff}<0.05/220 independent loci-trait pairs tested=2.3×10⁻⁴). The majority of these signals were driven by a greater magnitude of effect in female participants, which is consistent with prior investigations of WHRadjBMI 12,55. Across all six local adiposity traits, 26 trait-loci associations were only genome-wide significant in females, while 9 loci were only genome-wide significant in males.

Overlap of Local Adiposity Traits with WHRadjBMI Findings

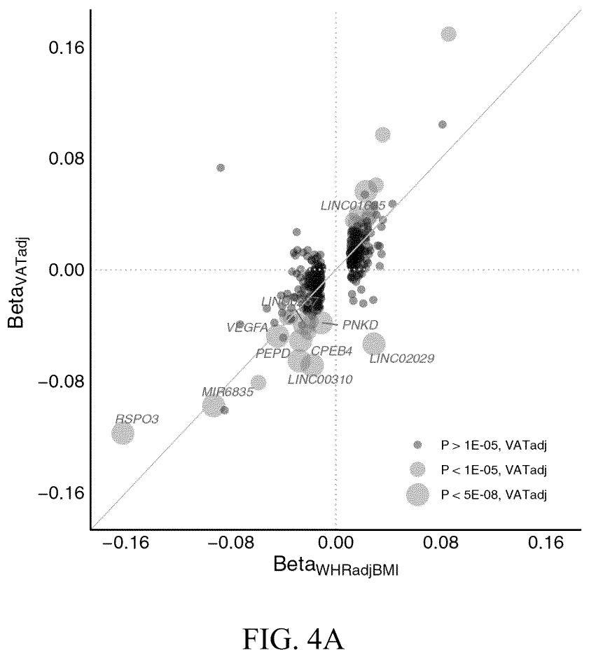

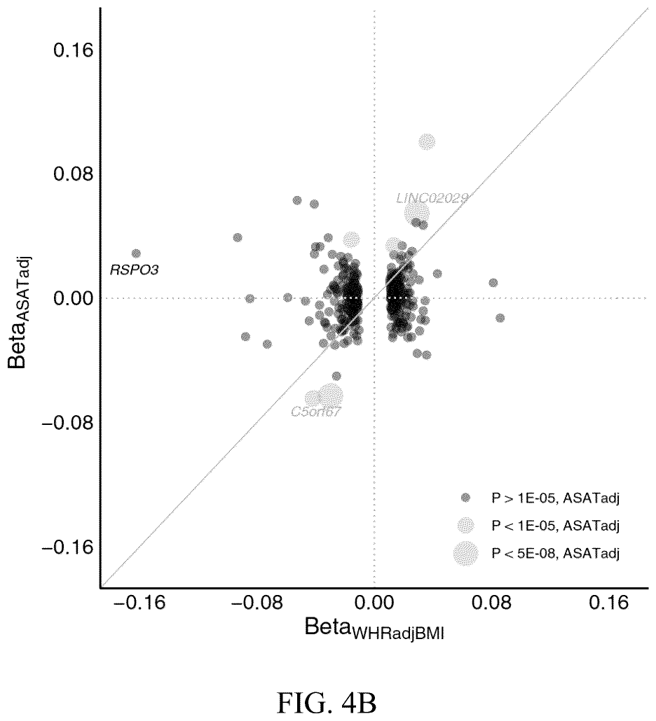

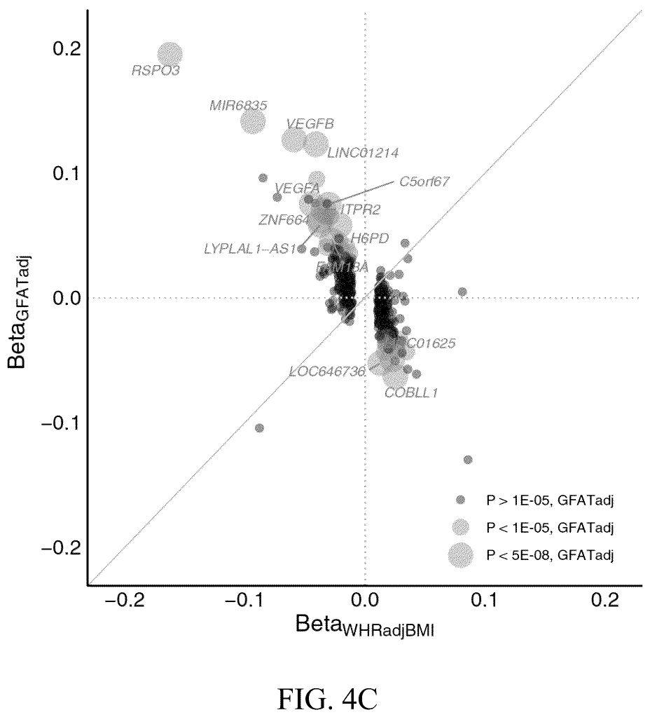

To investigate the added value of precisely quantifying fat depots with MRI in a smaller number of individuals as compared to WHRadjBMI in a larger cohort, Applicants studied the effects of 345 loci identified in the most recent WHRadjBMI meta-analysis of up to 694,649 individuals on VATadj, ASATadj, and GFATadj (FIG. 4A-C and Supplementary Data 11)¹². Of the 345 loci, 10 (3%) achieved genome-wide significance in association with VATadj (p<5×10⁻⁸), 2 with ASATadj (0.6%), and 14 (4%) with GFATadj. A unit increase in WHRadjBMI might be expected to be reflecting a unit increase in VATadj or ASATadj, or a unit decrease in GFATadj. Applicants quantified how often a locus was discordant from this pattern (e.g., a unit increase in WHRadjBMI corresponding to a unit decrease in VATadj), excluding loci where the fat depot effect size was smaller in magnitude than the SE. Fifteen of 242 loci (6%) were VATadj-discordant, 71 of 166 loci (43%) were ASATadj-discordant, and 22 of 231 loci (10%) were GFATadj-discordant (Supplementary Data 11).

Two illustrative examples indicate how follow-up of WHRadjBMI associations from a very large study in a smaller study with specific fat depots quantified may prove useful. The top WHRadjBMI signal is located at an intronic RSPO3 locus (rs72959041; beta=−0.162; p=2.1×10⁻²⁹³)—the work further clarifies that this signal is driven by an effect on VATadj (beta=−0.118; p=7.8×10⁻¹³) and GFATadj (beta=0.195; p=3.2×10⁻³²), but not ASATadj (beta=0.029; p=0.09). In contrast, a WHRadjBMI signal near LINC02029 (r510049088; beta=0.029; p=1.5×10⁻⁵⁹) is driven by ASATadj (beta=0.054; p=7.3×10⁻¹⁴) and GFATadj (beta=−0.034, p=6.0×10⁻⁶), but has a VATadj-discordant signal (beta=−0.053, p=8.7×10⁻¹³).

### External Validation

Applicants pursued replication of the genome-wide significant loci with a prior meta-analysis of CT and MRI-derived VAT, ASAT, VAT adjusted for BMI (VATadjBMI), and VAT/ASAT ratio in up to 18,332 individuals²⁷. Of the 76 SNP-trait associations across the traits of VAT, ASAT, VATadj, and VAT/ASAT ratio in this study, association results for 17 were available for comparison in published summary statistics 27. Of these, 16 (94%) had directionally consistent effects (binomial test p=2.7×10⁻⁴, Supplementary Data 12).

### Transcriptome-Wide Association Study

To prioritize genes, Applicants conducted a transcriptome-wide association study (TWAS) using gene expression data from visceral and subcutaneous adipose tissue from GTEx v7⁶⁰. Across all traits, the most significant association was observed between GFATadj and CCDC92 (TWAS Z-score=12.0; TWAS p=2.7×10⁻³³) in subcutaneous adipose tissue (Supplementary Data 13). The most significant eQTL for this association was shared with DNAH10OS (TWAS Z-score=10.5; p=8.2×10⁻²⁶) and DNAH10 (TWAS Z-score=7.9; p=3.5×10⁻¹⁵). Prior work demonstrated that knockdown of CCDC92 or DNAH10 led to significant reduction of lipid accumulation in an adipocyte model¹⁹. Of note, predicted VATadj associations with CCDC92 and DNAH10 in visceral adipose tissue samples demonstrated the opposite direction of effect (CCDC92 Z-score=−6.7; p=2.7×10⁻¹¹; DNAH10 Z-score=−5.3; p=1.1×10⁻⁷), suggesting fat depot discordant effects.

Another top TWAS signal was observed with GFATadj and IRS1 (Z-score=9.1; p=6.2×10⁻²⁰) with the corresponding association with ASATadj having the same direction of effect (Z-score=5.5; p=4.6×10⁻⁸). Prior work has demonstrated that decreased IRS1 expression, the gene encoding the insulin receptor substrate, causes insulin resistance—the work further suggests that impaired expansion of the gluteofemoral and abdominal subcutaneous fat depots may be involved in this physiological insult^{47,61}. Finally, a significant association was observed between VEGFB and GFATadj (Z-score=7.0; p=2.0×10⁻¹²), but not ASATadj (Z-score=0.44, p=0.66). Endothelial VEGFB is known to facilitate endothelial targeting of fatty acids to peripheral tissues and induce adipocyte thermogenesis, and transduction of VEGFB into mice improved metabolic health without changes in body weight^{62,63}. These results suggest that maintenance of the gluteofemoral fat depot may partially explain the metabolic effects of VEGFB.

### Tissue-Specific Enrichment Analyses

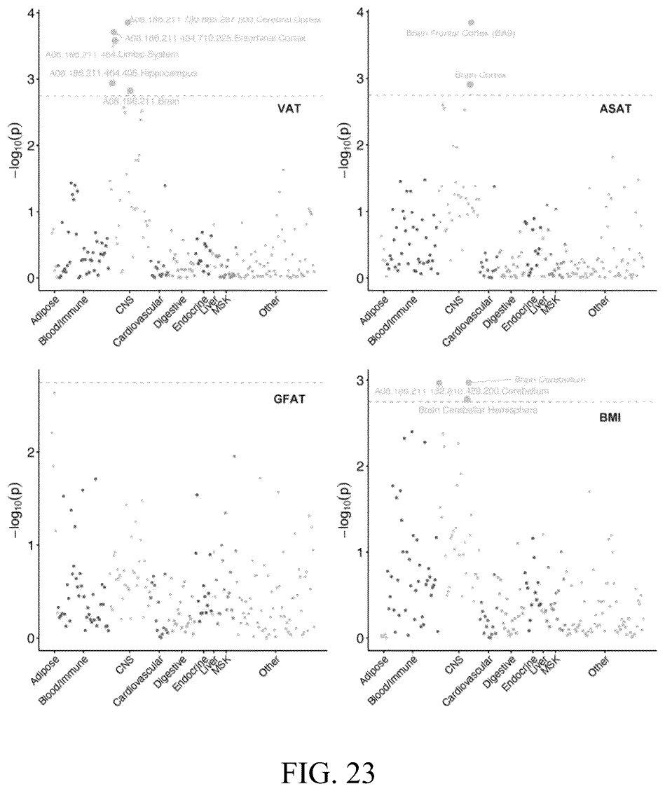

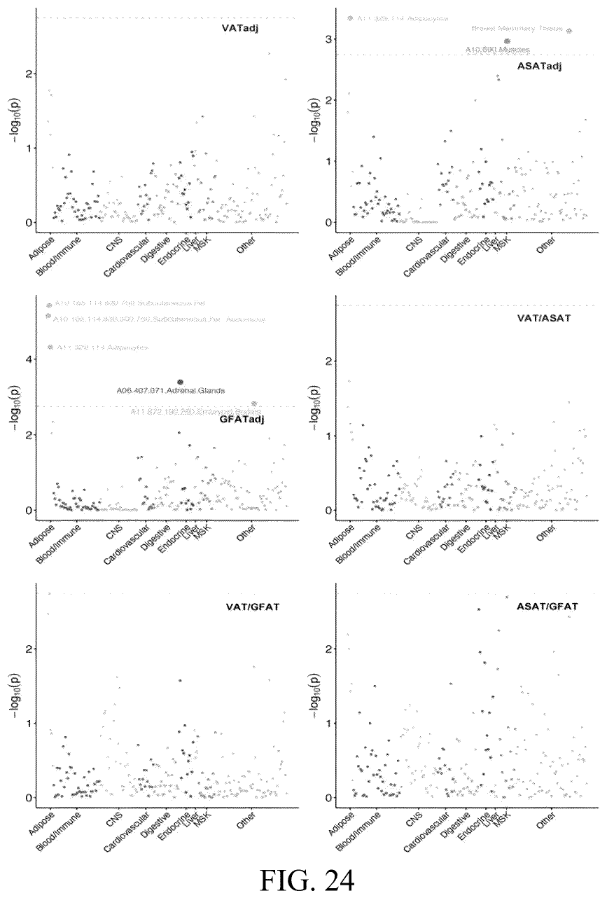

Applicants used stratified LD-score regression to probe for tissue-specific enrichment for each adiposity trait (Supplementary Data 14)⁶⁴. A marked dichotomy was observed between the three raw fat depot volumes (VAT, ASAT, GFAT)—each highly genetically correlated with BMI- and the six derived local adiposity traits (VATadj, ASATadj, GFATadj, VAT/ASAT, VAT/GFAT, ASAT/GFAT). While VAT, ASAT, and GFAT showed a pattern of central nervous system (CNS) tissue enrichment—consistent with the enrichment pattern for BMI-local adiposity traits were characterized by adipose tissue signals with reduced CNS signals (FIGS. 23 and 24). These results further emphasize that the genetic basis of overall adiposity is driven largely by CNS processes—such as those governing appetite and satiety—whereas fat distribution is regulated at the level of the adipocyte and other peripheral tissues.

### Rare Variant Association Study

Up to 19,255 individuals with fat depots quantified and exome sequencing data available were included in rare variant association studies. Applicants utilized two masks: one containing only predicted loss-of-function variants (pLoF) and a second combining pLoF with missense variants predicted to be deleterious by 5 out of 5 in silico prediction algorithms (pLoF+missense). Applicants tested the association between the aggregated rare variant score with each mask and each inverse normal transformed phenotype using multivariable regression. Analyses were restricted to genes with at least ten variant carriers in the analyzed cohort, yielding up to 12,020 tested genes. Exome-wide significance was considered to be p<0.05/12,020=4.2×10⁻⁶, while a Bonferroni-corrected study-wide significance threshold was set to p<4.2×10⁻⁶/27=1.5×10⁻⁷. One exome-wide significant association was identified: pLoF+missense variants in PDE3B associated with increased GFATadj in females (24 carriers; beta=0.98; p=1.7×10⁻⁶) (Supplementary Data 15). Individuals who carry loss-of-function variants in PDE3B have previously been demonstrated to have reduced WHRadjBMI⁶⁵. This study confirms and extends this result by demonstrating that females who carry pLoF+missense variants in PDE3B harbor increased GFATadj and reduced VATadj (beta=−0.70; p=5.1×10⁻⁴)—consistent with a metabolically favorable fat distribution—and that these effects are attenuated in males (GFATadj beta=0.08; p=0.67; VATadj beta=−0.21; p=0.27) (FIG. 5 and Supplementary Data 16).

Rare variant signals in two additional genes, while they did not reach the threshold for exome-wide significance, warrant discussion. pLoF+missense variants in PCSK1 associated with GFAT in sex-combined analysis (101 carriers; beta=1.11; p=7.5×10⁻⁶) and pLoF+missense variants in ACAT1 associated with VAT in females (23 carriers; beta=2.66; p=6.4×10⁻⁶). Both of these genes have previously been implicated in altering adiposity. Rare mutations in PCSK1 are known to cause monogenic obesity—here, a relatively symmetric pattern of increased GFAT, VAT (beta=0.87; p=4.1×10⁻⁴), and ASAT (beta=1.04; p=3.1×10⁻⁵) were observed in sex-combined analyses (Supplementary Data 16)^{66,67}. In a study comparing obese women with or without type 2 diabetes, gene expression of ACAT1 was downregulated in the VAT and ASAT of obese women with type 2 diabetes and expression was restored after bariatric surgery and weight loss, suggesting a role in obesity-associated insulin resistance⁶⁸.

Finally, Applicants investigated if rare variants in known familial partial lipodystrophy genes PPARG and LAMA were associated with the adiposity traits defined in this study (Supplementary Data 17)^{8,10,69}. The 17 carriers of a pLoF+missense variant in PPARG tended to have reduced GFATadj in sex-combined analysis (beta −0.99, p=0.05), consistent with a lipodystrophic-pattern of reduced peripheral adipose tissue deposition. Applicants were unable to detect a significant association among the 51 carriers of rare LANA variants, potentially related to inadequate statistical power or variant annotation.

### Polygenic Contribution to Extremes of VATadj, ASATadj, and GFATadj

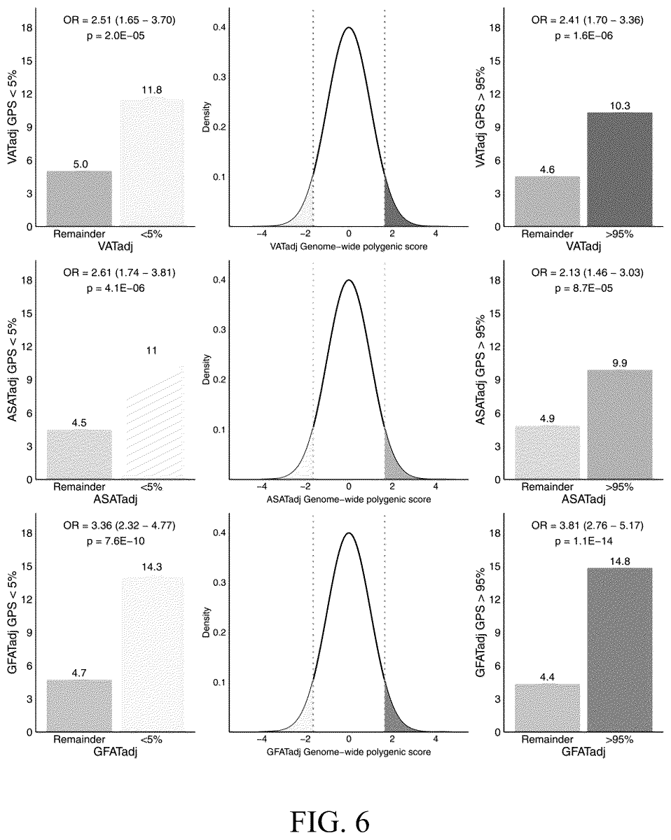

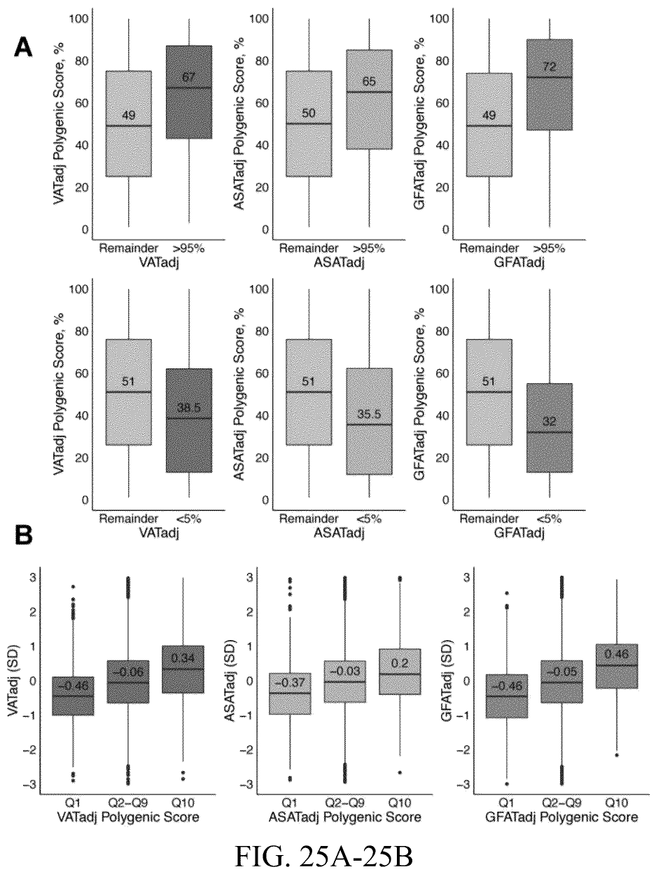

Because many individuals with lipodystrophy-like phenotypes—especially in its more subtle forms—do not harbor a known pathogenic rare variant, prior studies have begun to explore a potential “polygenic lipodystrophy,” in which an inherited component is instead driven by the cumulative impact of many common DNA variants^{10,19,20,70}. In the context of the traits defined in this study, a lipodystrophy-like phenotype might be characterized by increased VATadj, decreased ASATadj, and/or decreased GFATadj. Applicants set out to quantify the potential for genetic prediction of these traits by generating polygenic scores consisting of up to 1,125,301 variants for VATadj, ASATadj, and GFATadj traits using the LDpred2 algorithm⁷¹. To ensure no overlap between summary statistics and tested individuals, GWAS was conducted using a randomly selected 70% of participants. An additional 10% of participants was used as training data to select optimal LDpred2 hyperparameters and the remaining 20% of participants were held out for testing. In the test set, VATadj, ASATadj, and GFATadj polygenic scores explained 5.8%, 3.6%, and 7.0% of the corresponding trait variance, respectively (Supplementary Data 18 and 19). Participants at the tails of the distribution for any of the three local adiposity traits were enriched in extreme polygenic scores—for example, participants in the top 5% of the GFATadj distribution were nearly four times as likely to have a GFATadj polygenic score in the top 5% of the distribution (14.8% vs. 4.4%; OR=3.81; 95% CI: 2.76-5.17) (FIG. 6 and FIG. 25). Conversely, individuals with less than the 5th percentile of GFATadj were over three times as likely to have a GFATadj polygenic score less than the 5th percentile (14.3% vs. 4.7%; OR=3.36; 95% CI: 2.32-4.77). These findings suggest that polygenic inheritance plays an important role in fat distribution, and that polygenic scores could feasibly be used to enrich cohorts for individuals with extreme imaging phenotypes.

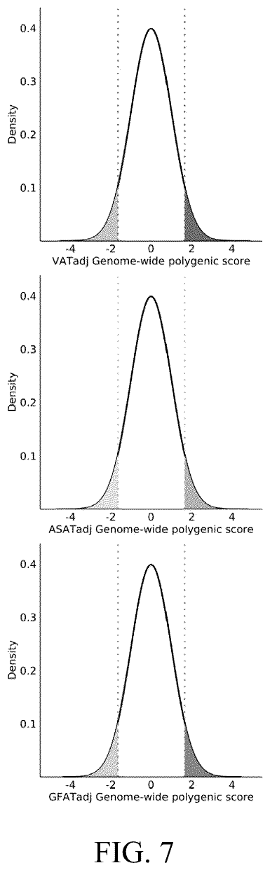

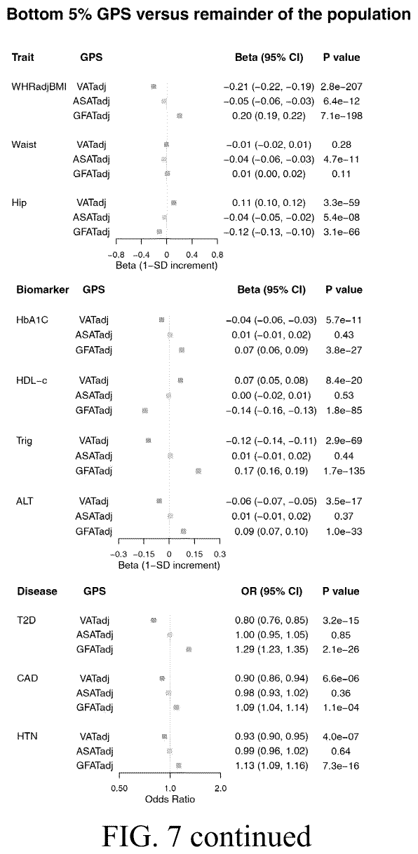

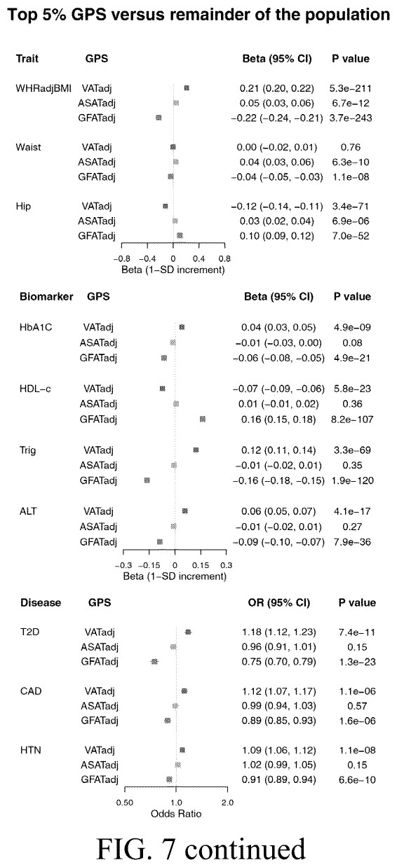

Applicants next tested the relationship between VATadj, ASATadj, and GFATadj polygenic scores and biomarkers of metabolic health (hemoglobin A1C, HDL cholesterol, serum triglycerides, and alanine aminotransferase (ALT)) and disease outcomes (type 2 diabetes, hypertension, and coronary artery disease) (FIG. 7 and Supplementary Data 20).

Within an independent dataset of 447,486 individuals of the UK Biobank who were genotyped, but not imaged, individuals in the top 5% of the GFATadj polygenic score had higher HDL-cholesterol (beta: 0.16 SD; 95% CI: 0.15-0.18; p=8.2×10⁻¹⁰⁷), lower serum triglycerides (beta: −0.16 SD; 95% CI: −0.18-−0.15; p=1.9×10⁻¹²⁰), lower serum ALT (beta: −0.09; 95% CI: −0.10-−0.07; p=7.9×10⁻³⁶), lower risk of type 2 diabetes (OR: 0.75; 95% CI: 0.70-0.79; p=1.3×10⁻²³), and lower risk of coronary artery disease (OR: 0.89; 95% CI: 0.85-0.93; p=1.6×10⁻⁶). By contrast, those in the top 5% of the VATadj polygenic score tended to have increased risk of these disease outcomes with odds ratios for type 2 diabetes, coronary artery disease, and hypertension of 1.18, 1.12, and 1.09, respectively.

Applicants aimed to externally validate associations with VATadj, ASATadj, and GFATadj polygenic scores in 7888 White participants of the Atherosclerosis Risk in Communities (AMC) study⁷². Each polygenic score was associated with HDL-cholesterol, triglycerides, and type 2 diabetes in ARIC. Results were broadly consistent with the UK Biobank with the strongest associations observed with the GFATadj polygenic score—individuals in the top 10% of the GFATadj polygenic score had higher HDL-cholesterol (beta: 0.14 SD, 95% CI: 0.07-0.22, p=1.5×10⁻⁴), lower serum triglycerides (beta: −0.16 SD; 95% CI: −0.23-−0.08, p=3.2×10⁻⁵), and lower risk of prevalent type 2 diabetes (OR: 0.57; 95% CI: 0.41-0.78, p=5.5×10⁻⁴) (Supplementary Data 21).

### DISCUSSION

In this study, Applicants investigated the inherited basis of body fat distribution using VAT, ASAT, and GFAT volumes quantified from body MM in up to 38,965 individuals. Local adiposity traits derived from these fat depots had a significant inherited component, enabling identification of 250 unique loci across all traits. The increased precision afforded by image-derived quantification confirmed and extended prior work indicating significant sex-dimorphism, refined depot-specific associations for loci previously identified for WHRadjBMI and led to the discovery of newly-associated loci, including a missense variant in SERPINA1 that predisposes to a metabolically healthier fat distribution. Polygenic scores for local adiposity traits were highly enriched among those with “lipodystrophy-like” fat distributions and were associated with cardiometabolic traits in a depot-specific fashion. These results have at least four implications.

First, traits aiming to quantify variation in body habitus—even when they are image-derived measurements of specific fat depot volumes as in this study—tend to be highly observationally and genetically correlated with one another and with BMI. GWAS of raw VAT, ASAT, and GFAT volumes each identified a well-known intronic FTO variant—characteristic of BMI—as a top signal, and cell-enrichment analyses of each unadjusted fat depot displayed a pattern of CNS cell-enrichment, consistent with the signal for BMI⁶⁴. By contrast, fat depot volumes adjusted-for-BMI-and-height and fat depot ratios—traits that capture local adiposity were more heritable than measures of overall adiposity, revealed depot-specific genetic architecture, and displayed a pattern of adipose tissue cell-enrichment. As large cohorts with body imaging become more prominent, careful consideration of this correlation structure is warranted to enable interpretation of genetic association results. For example, a measurement of VAT predicted from a model using primarily anthropometric traits was very highly genetically correlated with BMI (r_{g}=0.93), suggesting that the resultant genetic associations may predominantly reflect a component of VAT that is complementary to VATadj (r_{g} with BMI=−0.16) in this study²⁹. Additional investigation of how best to utilize composite phenotypes that jointly represent several correlated adiposity traits may prove useful^{73,74}.

Second, GFAT is highly heritable (GFATadj h²=0.41)—particularly in females (GFATadj h²=0.52)—with a genetic architecture that is distinct from VAT and ASAT when adjusted for overall adiposity. Most prior genetic studies of imaging-derived adiposity traits to date have been limited to VAT and ASAT—in this study, only 13 of 54 genome-wide significant loci for GFATadj overlapped with either VATadj or ASATadj²⁶⁻²⁸. Individuals with a GFATadj polygenic score in the bottom 5% were enriched for adverse cardiometabolic biomarker profiles and increased risk of type 2 diabetes and coronary artery disease. These observations lend further support to the hypothesis that a primary insult in a metabolically unhealthy fat distribution is the inability of the gluteofemoral fat depot to adequately expand^{4,75}. Additional study of GFAT depots—or related measures such as gynoid fat from DEXA scans—in future biobank-scale studies is warranted to determine the consistency of these genetic associations across diverse age and ancestry groups.

Third, this study extends prior work suggesting that common genetic variation—as captured by a polygenic score—contributes to extreme fat distribution phenotypes^{10,19,20,70}. While several of the familial partial lipodystrophies (FPLD) are known to be caused by monogenic variation in genes like LMNA and PPARG, FPLD type 1 has not been linked to a single mutation, leading some to suggest that this disease may be polygenic in nature¹⁰. Lotta et al. provided evidence for this by demonstrating that individuals with FPLD1 had a higher burden of a 53-SNP insulin resistance polygenic score compared to the general population¹⁹. In this study, individuals who harbor lower than average GFATadj or ASATadj and/or higher than average VATadj tended to manifest a mild lipodystrophy-like phenotype. Applicants demonstrate that individuals at the extremes of these local adiposity traits are enriched in extreme polygenic scores suggesting that polygenic scores may be helpful in identifying this subgroup of individuals for future focused investigations. For example, growth hormone releasing hormone analogs—such as tesamorelin—have previously been shown to lead to a selective reduction of VAT in patients with obesity or HIV-associated lipodystrophy^{76,77}. Whether a local adiposity polygenic score—perhaps in combination with emerging imaging tools for identifying lipodystrophies—could identify a subset of individuals with obesity and polygenic lipodystrophy who may benefit from these fat redistribution agents in addition to traditional obesity therapy is an area for future investigation⁷⁸.

Fourth, these results lay the scientific foundation for variant-to-function studies to link fat distribution-associated genetic risk loci to effector genes and mechanisms of action in depot-specific adipocyte model systems⁷⁹. Such targeted perturbation studies in subcutaneous and visceral adipocyte cell lines may reveal key biological pathways driving fat distribution and may generate therapeutic hypotheses for adverse fat distribution-related traits^{19,80}.

In conclusion, Applicants carried out genetic association studies of local adiposity traits in a large cohort of individuals with MM imaging. The work characterizes the depot-specific genetic architecture of visceral, abdominal subcutaneous, and gluteofemoral adipose tissue, and extends efforts to define and identify individuals with polygenic lipodystrophy.

### Example 2—Methods

### Study Population

The UK Biobank is an observational study that enrolled over 500,000 individuals between the ages of 40 and 69 years between 2006 and 2010, of whom 43,521 underwent MM imaging between 2014 and 2020^{81,82}. Applicants previously estimated VAT, ASAT, and GFAT volumes in 40,032 individuals of the imaged cohort after excluding 3489 (8.0%) scans based on technical problems or artifacts 5. A subset of 39,076 individuals with genotype array data available was studied here. Compared to non-imaged individuals of the UK Biobank at enrollment, imaged individuals were younger (mean age 56 years vs. 57 years), less likely to be female (51% vs. 55%), and more likely to be of white British ancestry (87% vs. 84%) (Supplementary Data 2). Individuals were not excluded on the basis of ancestry. This analysis of data from the UK Biobank was approved by the Mass General Brigham institutional review board and was performed under UK Biobank application #7089.

### Deriving Local Adiposity Traits

The focus of this study was to investigate the genetic architecture of fat distribution independent of the overall size of an individual. Two sets of traits were derived for this purpose: “adj” traits and fat depot ratios. “adj” traits represent residuals of the fat depot in question in sex-specific linear regressions against age, age squared, BMI, and height. Applicants provide justification in the Supplementary Methods for adjusting for both BMI and height as opposed to only BMI. In brief, adjusting only for BMI introduces a significant genetic correlation of each adj trait with height (most pronounced with ASAT and GFAT). Several prior studies have suggested that adjusting for heritable covariates can lead to spurious genetic associations due to collider bias^{83,84}. Applicants investigated the extent to which VATadj, ASATadj, and GFATadj loci may be driven by collider bias with BMI or height and found little evidence for collider bias making a significant contribution to these results (Supplementary Methods and Supplementary Data 22).

### Genotyping, Imputation, and OC

Genotyping in the UK Biobank was done with two custom genotyping arrays: UK BiLEVE and Axiom⁸⁵. Imputation was done using the UK10K and 1000 Genomes Phase 3 reference panels^{86,87}. Prior to analysis, genotyped SNPs were filtered based on the following criteria, only including variants if: (1) MAF≥1%, (2) Hardy-Weinberg equilibrium (HWE) p>1×10⁻¹⁵, (3) genotyping rate≥99%, and (4) LD pruning using R² threshold of 0.9 with window size of 1000 markers and step size of 100 marker^{88,89}. This process resulted in 433,616 SNPs available for genetic relationship matrix (GRM) construction. Imputed SNPs with MAF<0.005 or imputation quality (INFO) score <0.3 were excluded. Note that the MAF filter was applied to the UK Biobank imputed file prior to subsetting to the imaged substudy. These criteria resulted in a total of 11,485,690 imputed variants available for analysis.

Participant were excluded from analysis if they met any of the following criteria: (1) mismatch between self-reported sex and sex chromosome count, (2) sex chromosome aneuploidy, (3) genotyping call rate <0.95, or (4) were outliers for heterozygosity. Up to 38,965 participants were available for analysis (37,641 for adj traits because these individuals also had to have BMI and height available).

### Common Variant Association Studies

Nine traits were analyzed (VAT, ASAT, GFAT, VATadj, ASATadj, GFATadj, VAT/ASAT, VAT/GFAT, and ASAT/GFAT) in three contexts (sex-combined, male only, female only), leading to 27 analyses in total. SNP-heritability was estimated using BOLT-REML v2.3.4^{90,91}. Genetic correlations between traits were estimated using cross-trait LD-score regression (ldsc v1.0.1) using default settings^{33,34}.

Prior to conducting GWAS, each trait was inverse-normal transformed. Each analysis was adjusted for age at the time of MRI, age squared, sex (except in sex-stratified analyses), the first ten principal components of genetic ancestry, genotyping array, and MM imaging center. BOLT-LMM v2.3.4 was used to carry out GWAS accounting for cryptic population structure and sample relatedness^{90,91}. After the QC protocol detailed above, 433,616 SNPs were available for GRM construction. A threshold of p<5×10⁻⁸ was used to denote genome-wide significance, while a threshold of p<5×10⁻⁸/27=1.9×10⁻⁹ was used to denote study-wide significance.

Lead SNPs were prioritized with LD clumping. LD clumping was done with the -clump function in PLINK to isolate independent signals for each GWAS. The parameters were as follows: -clump-p1 5E-08, -clump-p2 5E-06, -clump-r2 0.1, -clump-kb 1000, which can be interpreted as follows: variants with p<5E-08 are chosen starting with the lowest p value, and for each variant chosen, all other variants with p<5E-06 within a 1000 kb region and r²>0.1 with the index variant are assigned to that index variant. This process is repeated until all variants with p<5E-08 are assigned an LD clump. An LD reference panel for this task was constructed using a random sample of 3000 individuals from the studied.

The extent of genomic inflation vs. polygenicity was assessed by computing the LD-score regression intercept (ldsc v1.0.1) using default settings³³.

A lead SNP was defined as newly-identified if it was not in LD (R 2<0.1) with any SNP in the GWAS catalog (downloaded Jun. 8, 2021) with genome-wide significant association (p<5×10⁻⁸) with any “DISEASE/TRAIT” containing the following characters: (1) “body mass”, (2) “BMI”, (3) “adipos”, (4) “fat”, (5) “waist”, (6) “hip circ”, or (7) “whr”. These characters captured key anthropometric traits of interest (e.g., BMI, waist circumference, hip circumference, waist-to-hip ratio) as well as other related traits of interest (e.g., VAT, predicted VAT, fat impedance measures).

### Clustering to Classify Gluteofemoral Adiposity Signals

Clustering analysis was performed for GFATadj and GFAT association signals.

Applicants started with all 250 lead SNPs significantly associated with any of the nine adiposity traits and extracted those associated with the primary trait (e.g., GFATadj) with nominal significance (p<0.05) for each analysis. To ensure that only independent signals were used for the clustering, variants were LD-pruned using a LD threshold of r²=0.1. When two SNPs were found to be in LD above this threshold, the variant with the lower p value was retained.

Summary statistics were gathered from GWAS performed in the UK Biobank for 32 cardiometabolic traits (Supplementary Data 6). For each trait GWAS, the regression coefficient betas was divided by the SE to obtain standardized effect sizes. These standardized effects were further scaled by dividing by the square root of the variant's sample size for the given trait GWAS and then multiplying by the square root of the median sample size of all GWAS. Since all summary statistics were sourced from UK Biobank, this additional scaling had a negligible effect.

The clustering traits were then filtered to retain those relevant to the analysis by removing any that were not associated with at least one variant at a Bonferroni p value threshold (0.05/number of SNPs). When two traits had highly correlated Z-scores (|r|>0.85), the trait with the lower minimum p value was kept and the other removed. The remaining standardized effect sizes made up the variant-trait association matrix, Z (N variants by M traits).

In order to satisfy the non-negative requirement of Bayesian non-negative matrix factorization (bNMF), each column was split into two arrays: one with the positive Z-scores and the other with the absolute value of the negative Z-scores. This means that the final association matrix, X, contained N variants by 2M traits.

The bNMF clustering was performed as previously described²⁰. The procedure attempts to approximate the association matrix by factorizing X into two matrices, W (2M by K) and HT (N by K), with an optimal rank K. bNMF is designed to suggest an optimal K best explaining X at the balance between an error measure, ||X−WH|², and a penalty for model complexity derived from a non-negative half-normal prior for W and H. In addition, bNMF exploits an automatic relevance determination technique to iteratively regress out irrelevant components in explaining the observed data X. The exact objective function optimized by bNMF is a posterior, which has two opposing contributions from the likelihood (Frobenius norm) and the regularization penalty (L2-norm of W and H coupled by the relevance weights). For all analyses, bNMF was run with 100 iterations for each. All analyses converged in ≥92% of iterations to their given K solution. Code used in the bNMF clustering is available on GitHub: github.com/kwesterman/bnmf-clustering.

### Identification of Sex-Dimorphic Signals

Genetic correlations between sexes for each of the adiposity traits were computed using cross-trait LD-score regression as described above.

Using sex-specific GWAS summary statistics for each of the six local adiposity traits (VATadj, ASATadj, GFATadj, VAT/ASAT, VAT/GFAT, ASAT/GFAT), Applicants tested each of the 220 genetic loci that were genome-wide significant for any of the six local adiposity traits in either sex-combined or sex-stratified analyses for sex dimorphism by computing the t-statistic:

$$t = \frac{beta ( males ) - beta ( females )}{\sqrt{{se ( males )}^{2} + {se ( females )}^{2} - 2 * r * se ( males ) * se ( females )}} \qquad Equation 1$$

where beta is the effect size for an adiposity trait in sex-stratified GWAS, se is the standard error, and r is the genome-wide Spearman rank correlation coefficient between males and females. The t-statistic and associated p value (p_{diff}) were computed using the EasyStrata software⁹². Given that 220 independent loci were tested, a significance threshold of p_{diff}<0.05/220=2.3×10⁻⁴ was used.

### WHRadjBMI Loci Lookups

A recent meta-analysis for the WHRadjBMI trait across 694,649 individuals revealed 346 unique associated loci¹². Of these 346 loci, the primary signals for 345 loci were among the imputed variants available for analysis in this study. Applicants plotted the effect sizes for VATadj, ASATadj, and GFATadj for each of these 345 loci and further quantified the frequency of “WHRadjBMI-discordance” defined as either (1) WHRadjBMI and VATadj effects going in opposite directions, (2) WHRadjBMI and ASATadj effects going in opposite directions, or (3) WHRadjBMI and GFATadj effects going in the same direction. For each adiposity trait in the “WHRadjBMI-discordance” analysis, Applicants excluded loci for which the effect size beta was smaller than the SE to avoid inflating the fraction of “WHRadjBMI-discordant” loci.

External Validation with Prior Meta-Analysis

External validation for 76 genome-wide significant SNP-trait associations with VAT, ASAT, VATadj, and VAT/ASAT ratio was pursued using summary statistics downloaded from the GWAS catalog of a multiethnic genome-wide meta-analysis of ectopic fat depots in up to 2.6 million SNPs in up to 18,332 individuals^{27,35}. Alleles were aligned and the z-score for each SNP from the previous study were compared with the effect sizes in the current study to determine concordance.

### Transcriptome-Wide Association Study

For each of the six local adiposity traits (VATadj, ASATadj, GFATadj, VAT/ASAT, VAT/GFAT, ASAT/GFAT), Applicants performed a TWAS to prioritize genes on the basis of imputed cis-regulated gene expression using FUSION with default settings^{60,93,94}. Pre-computed gene expression weights from GTEx v7 were used as downloaded from the FUSION website (gusevlab.org/projects/fusion/)⁶⁰. Reference weights for visceral adipose tissue were used for VATadj, while those for subcutaneous adipose tissue were used for ASATadj, GFATadj, and ASAT/GFAT ratio. Weights from both visceral and subcutaneous adipose tissue were used for VAT/ASAT and VAT/GFAT ratios.

### Cell- and Tissue-Specific Enrichment

Applicants used stratified LD-score regression to identify cell types that are most relevant for each of the nine adiposity traits (VAT, ASAT, GFAT, VATadj, ASATadj, GFATadj, VAT/ASAT, VAT/GFAT, and ASAT/GFAT) and BMI⁶⁴. Applicants carried out this analysis using ldsc v1.0.1 with default settings and using two gene expression datasets that are described in the manuscript outlining stratified LD-score regression⁶⁴: GTEx⁹⁵ and the “Franke lab” 9697 dataset.

### Sequencing and Sample Quality Control for Rare-Variant Association Study

Applicants conducted rare-variant association studies using data from the 200,643 exomes released by the UK Biobank⁹⁸. Whole-exome sequencing was performed by the Regeneron Genetics Center using an updated Functional Equivalence protocol that retains original quality scores in the CRAM files (referred to as the OQFE protocol) as previously described⁹⁸. The DTxGen Exome Research Panel v1.0 including supplemental probes was used for exome capture for this dataset (biobank.ctsu.ox.ac.uk/showcase/label.cgi?id=170). In total, 19,396 genes in the targets of 38 Mbp were covered. In total, 75×75 bp paired-end reads were sequenced on the Illumina NovaSeq 6000 platform. For each sample in the targeted region, more than 95.2% of sites were covered by more than 20 reads. Applicants downloaded the pVCF file provided by the UK Biobank, and then applied additional genotype call, variant, and sample quality control⁹⁹.

The individual genotype call was set as missing if reads depth (DP)≤10 or DP≥200, if homozygous reference allele with genotype quality (GQ)≤20 or the ratio of alt allele reads over all of the covered reads >0.1, if heterozygous with the ratio of alt allele reads over all of the covered reads <0.2 or Phred-scaled likelihood (PL) of the reference allele <20, or if homozygous alternate with the ratio of alt allele reads over all of the covered reads <0.9 or PL of reference allele <20. The variant quality control was performed using the following exclusion criteria:
Variants in low-complexity regions of the genome that preclude accurate read alignment as previously definer¹⁰⁰.
Variants in segmental duplication region of the genome^{100,101}.
Hardy-Weinberg disequilibrium (HWE)p value <1×10⁻¹⁵.
Variant call rate <90%.
Monomorphic sites after the above genotype call quality control.

After the above genotype call and variant QC, Applicants selected a subset of high-quality variants for inferring the genetic kinship matrix and genetic sex used for sample QC. Applicants selected independent autosome variants by MAF >0.1%, missingness <1%, and HWE p>10⁻⁶. Applicants further pruned the variants using PLINK2 software¹⁰² with a window size of 200, step size 100, and R²=0.1 and removed indels and strand ambiguous SNPs. Based on these variants, Applicants used KING (version 2.2.5)¹⁰³ to infer the genetic kinship matrix. Applicants further selected X-chromosomal variants, not within the pseudo-autosomal regions, based on the sample variant QC criteria as for the autosome variants and did the same variant pruning procedure. Applicants then inferred the genetic sex based on the F statistics by PLINK2 software, F>0.8 was set to male, while samples with F<0.5 were set to female. Eighty samples were removed because of the discordance of genetic sex with self-reported sex. Applicants further removed samples if:
The ratio of heterozygote/homozygote beyond 8 standard deviations (N=100 samples removed).
The ratio of the number of SNVs/indels beyond 8 standard deviations (N=1 samples removed).
The number of singletons was beyond 8 standard deviations (N=111 samples removed).
Genotype call rate <90% (N=1 sample removed).
Withdrawal of informed consent (N=13 samples removed).

Applicants further randomly removed one sample if a pair of samples had second-degree relative or closer kinship, defined as kinship coefficient >0.088474 (N=1563 samples removed). Of all the above QC passed samples, 19,255 samples out of the 40,032 having image-derived traits were used in the downstream rare variant burden test. Applicants converted the genetic coordinates from GRCh38 to GRCh37 using CrossMap software (version: v0.3.3)¹⁰⁴.

### Approach to Variant Annotation and Weighting

To identify rare (MAF <0.1%) high-confidence predicted inactivating variants, Applicants applied the previously validated Loss-Of-Function Transcript Effect Estimator (LOFTEE) algorithm implemented within the Ensembl Variant Effect Predictor (VEP) software program as a plugin, VEP version 96.0^{105,106}. The LOFTEE algorithm identifies stop-gain, splice-site disrupting, and frameshift variants. The algorithm includes a series of flags for each variant class that collectively represent “low-confidence” inactivating variants. In this study, Applicants studied only variants that were “high-confidence” inactivating variants without any flag values. This aggregation strategy will be referred to hereafter as putative loss-of-function (“pLoF”).

To identify rare (MAF <0.1%) predicted damaging missense variants, Applicants included variants predicted to be damaging by all of five computational prediction algorithms¹⁰⁷⁻¹⁰⁹. In brief, predictions were retrieved from the dbNSFP database¹¹⁰, version 2.9.3, with the most severe prediction across multiple transcripts used. Applicants focused on five prediction algorithms: SIFT¹¹¹ (including variants annotated as damaging), PolyPhen2-HDIV and PolyPhen2-HVAR¹¹² (including variants annotated as possibly or probably damaging), LRT¹¹³ (including variants annotated as deleterious), and MutationTaster¹¹⁴ (including variants annotated as disease-causing-automatic or disease-causing). Within the association testing framework, this class of variants was given a gene-specific weight based on the relative cumulative frequency of these predicted damaging missense variants as compared to the cumulative frequency of high-confidence predicted inactivating variants identified by LOFTEE algorithm using a previously recommended approach:^{115,116} given the cumulative allele frequency of all of the LOFTEE high-confidence rare variants of a gene (G) as f_{L}, the cumulative allele frequency of all of the predicted damaging missense variants as f_{M}, the weight for the missense variants was estimated as the quantity in Eq. (2) and capped at 1.0:

$${( \frac{{f}_{L} \times ( 1 - {f}_{L} )}{{f}_{M} \times ( 1 - {f}_{M} )} )}^{0.5} \qquad Equation 2$$

For genes without LOFTEE high-confidence rare variants, the weight for missense variants is 1.0. This aggregation strategy will be referred to hereafter as putative loss-of-function plus missense (“pLoF+missense”).

### Statistical Analysis

Applicants tested the association between the aggregated rare variant score (the weighted sum of the qualified variant of each gene) and each inverse normal transformed phenotype using a multivariable regression model in sex-combined and sex-stratified models. Analyses were restricted to genes that had at least ten variant carriers in the analyzed cohort. An individual's gene-specific score was computed according to the weighting strategy described above and capped at one. The covariates were the same as the common variant association test. Given the filter of ten variant carriers, sex-combined analyses tested 12,020 genes and so a gene was recognized as exome-wide significant if the gene's p value was smaller than the Bonferroni-corrected p value threshold of 0.05/12,020=4.2×10⁻⁶.

### Polygenic Score

Applicants used the LDpred2 algorithm⁷¹ to derive genome-wide polygenic scores for each trait. Applicants randomly selected 350,000 White British ancestry individuals from the UK Biobank to use as the LD reference panel⁸⁵, and used HapMap3 variants with MAF >0.5% in the LD reference panel to compute the LD correlation matrix. For each trait, Applicants partitioned the samples into three independent portions: 70% to run the GWAS for making the summary statistics, 10% to select the optimal hyperparameters, and 20% to test performance. Applicants randomly removed one sample in a pair if the pair had a genetic relationship closer than a second-degree genetic relationship in the last two partitions of samples and checked the pairwise relationship across the whole dataset. For the hyperparameters of the LDpred2 algorithm, Applicants grid searched three parameters: (1) 0.7, 1, and 1.4 times of genome-wide heritability estimation, (2) whether or not to use a sparse LD correlation matrix, and (3) 17 different estimates of the proportion of causal variants selecting from [0.18,0.32,0.56,1]×10^{[0,-1,-2,-3]} and 0.0001. In total, Applicants tested 3×2×17=102 grid points.

For all downstream analyses, each polygenic score was residualized against the first ten principal components of genetic ancestry prior to regression with the dependent variable of interest, and each regression was adjusted for age at the time of imaging, sex, and the first ten principal components of genetic ancestry.

### Polygenic Score External Validation in ARIC

The ARIC study is a prospective cohort study that—beginning in 1987—enrolled white and black participants between the ages of 45 and 64 years⁷². Genotype and clinical data were retrieved from the National Center for Biotechnology Information dbGAP server (accession number phg000035.v1). VATadj, ASATadj, and GFATadj polygenic scores were computed using identical LDpred2 weights and the optimal hyperparameter set for UK Biobank analyses. Circulating biomarkers and clinical risk factor ascertainment was performed at time of enrollment as previously described⁷².

### REFERENCES

1. González-Muniesa P, et al. Obesity. Nat. Rev. Dis. Prim. 2017; 3:1-18.
2. Kivimäki M, et al. Overweight, obesity, and risk of cardiometabolic multimorbidity: pooled analysis of individual-level data for 120 813 adults from 16 cohort studies from the USA and Europe. Lancet Public Health. 2017; 2:e277-e285. doi: 10.1016/S2468-2667(17)30074-9.
3. Stefan N, Schick F, Häring H-U. Causes, characteristics, and consequences of metabolically unhealthy normal weight in humans. Cell Metab. 2017; 26:292-300. doi: 10.1016/j.cmet.2017.07.008.
4. Stefan N. Causes, consequences, and treatment of metabolically unhealthy fat distribution. Lancet Diabetes Endocrinol. 2020; 8:616-627. doi: 10.1016/S2213-8587(20)30110-8.
5. Agrawal, S. et al. Association of machine learning-derived measures of body fat distribution in >40,000 individuals with cardiometabolic diseases. medRxiv. 10.1101/2021.05.07.21256854 (2021).
6. Agarwal A K, Garg A. A novel heterozygous mutation in peroxisome proliferator-activated receptor-γ gene in a patient with familial partial lipodystrophy. J. Clin. Endocrinol. Metab. 2002; 87:408-408.
7. Agostini M, et al. Non-DNA binding, dominant-negative, human PPARgamma mutations cause lipodystrophic insulin resistance. Cell Metab. 2006; 4:303-311. doi: 10.1016/j.cmet.2006.09.003.
8. Shackleton S, et al. LMNA, encoding lamin A/C, is mutated in partial lipodystrophy. Nat. Genet. 2000; 24:153-156. doi: 10.1038/72807.
9. Ajluni N, et al. Spectrum of disease associated with partial lipodystrophy: lessons from a trial cohort. Clin. Endocrinol. 2017; 86:698-707. doi: 10.1111/cen.13311.
10. Lim K, Haider A, Adams C, Sleigh A, Savage D B. Lipodistrophy: a paradigm for understanding the consequences of ‘overloading’ adipose tissue. Physiol. Rev. 2021; 101:907-993.
11. Shungin D, et al. New genetic loci link adipose and insulin biology to body fat distribution. Nature. 2015; 518:187-196. doi: 10.1038/nature14132.
12. Pulit S L, et al. Meta-analysis of genome-wide association studies for body fat distribution in 694 649 individuals of European ancestry. Hum. Mol. Genet. 2019; 28:166-174. doi: 10.1093/hmg/ddy327.
13. Rask-Andersen M, Karlsson T, Ek APPLICANTS, Johansson Å. Genome-wide association study of body fat distribution identifies adiposity loci and sex-specific genetic effects. Nat. Commun. 2019; 10:339. doi: 10.1038/s41467-018-08000-4.
14. Pietiläinen K H, et al. Agreement of bioelectrical impedance with dual-energy X-ray absorptiometry and MM to estimate changes in body fat, skeletal muscle and visceral fat during a 12-month weight loss intervention. Br. J. Nutr. 2013; 109:1910-1916. doi: 10.1017/S0007114512003698.
15. Ling C H Y, et al. Accuracy of direct segmental multi-frequency bioimpedance analysis in the assessment of total body and segmental body composition in middle-aged adult population. Clin. Nutr. Edinb. Scott. 2011; 30:610-615. doi: 10.1016/j.clnu.2011.04.001.
16. Emdin C A, et al. Genetic association of waist-to-hip ratio with cardiometabolic traits, type 2 diabetes, and coronary heart disease. JAMA. 2017; 317:626-634. doi: 10.1001/jama.2016.21042.
17. Lotta L A, et al. Association of genetic variants related to gluteofemoral vs abdominal fat distribution with type 2 diabetes, coronary disease, and cardiovascular risk factors. JAMA. 2018; 320:2553-2563. doi: 10.1001/jama.2018.19329.
18. Yaghootkar H, et al. Genetic evidence for a link between favorable adiposity and lower risk of type 2 diabetes, hypertension, and heart disease. Diabetes. 2016; 65:2448-2460. doi: 10.2337/db15-1671.
19. Lotta L A, et al. Integrative genomic analysis implicates limited peripheral adipose storage capacity in the pathogenesis of human insulin resistance. Nat. Genet. 2017; 49:17-26. doi: 10.1038/ng.3714.
20. Udler M S, et al. Type 2 diabetes genetic loci informed by multi-trait associations point to disease mechanisms and subtypes: a soft clustering analysis. PLoS Med. 2018; 15:e1002654. doi: 10.1371/journal.pmed.1002654.
21. Ji Y, et al. Genome-wide and abdominal MRI data provide evidence that a genetically determined favorable adiposity phenotype is characterized by lower ectopic liver fat and lower risk of type 2 diabetes, heart disease, and hypertension. Diabetes. 2019; 68:207-219. doi: 10.2337/db18-0708.
22. Martin, S. et al. Genetic evidence for different adiposity phenotypes and their opposing influence on ectopic fat and risk of cardiometabolic disease. Diabetes. 10.2337/db21-0129 (2021).
23. Heald A H, et al. Genetically defined favourable adiposity is not associated with a clinically meaningful difference in clinical course in people with type 2 diabetes but does associate with a favourable metabolic profile. Diabet. Med. J. Br. Diabet. Assoc. 2021; 38:e14531. doi: 10.1111/dme.14531.
24. Wilman H R, et al. Genetic studies of abdominal MRI data identify genes regulating hepcidin as major determinants of liver iron concentration. J Hepatol. 2019; 71:594-602. doi: 10.1016/j.jhep.2019.05.032.
25. Haas, M. E. et al. Machine learning enables new insights into clinical significance of and genetic contributions to liver fat accumulation. medRxiv10.1101/2020.09.03.20187195 (2020).
26. Fox C S, et al. Genome-wide association for abdominal subcutaneous and visceral adipose reveals a novel locus for visceral fat in women. PLoS Genet. 2012; 8:e1002695. doi: 10.1371/journal.pgen.1002695.
27. Chu A Y, et al. Multiethnic genome-wide meta-analysis of ectopic fat depots identifies loci associated with adipocyte development and differentiation. Nat. Genet. 2017; 49:125-130. doi: 10.1038/ng.3738.
28. Liu Y, et al. Genetic architecture of 11 organ traits derived from abdominal MRI using deep learning. eLife. 2021; 10:e65554. doi: 10.7554/eLife.65554.
29. Karlsson T, et al. Contribution of genetics to visceral adiposity and its relation to cardiovascular and metabolic disease. Nat. Med. 2019; 25:1390-1395. doi: 10.1038/s41591-019-0563-7.
30. Chen G-C, et al. Association between regional body fat and cardiovascular disease risk among postmenopausal women with normal body mass index. Eur. Heart J. 2019; 40:2849-2855. doi: 10.1093/eurheartj/ehz391.
31. Pou K M, et al. Patterns of abdominal fat distribution: the Framingham Heart Study. Diabetes Care. 2009; 32:481-485. doi: 10.2337/dc08-1359.
32. Hiuge-Shimizu A, et al. Absolute value of visceral fat area measured on computed tomography scans and obesity-related cardiovascular risk factors in large-scale Japanese general population (the VACATION-J study) Ann. Med. 2012; 44:82-92. doi: 10.3109/07853890.2010.526138.
33. Bulik-Sullivan B K, et al. LD score regression distinguishes confounding from polygenicity in genome-wide association studies. Nat. Genet. 2015; 47:291-295. doi: 10.1038/ng.3211.
34. Bulik-Sullivan B, et al. An atlas of genetic correlations across human diseases and traits. Nat. Genet. 2015; 47:1236-1241. doi: 10.1038/ng.3406.
35. Buniello A, et al. The NHGRI-EBI GWAS Catalog of published genome-wide association studies, targeted arrays and summary statistics 2019. Nucleic Acids Res. 2019; 47:D1005-D1012. doi: 10.1093/nar/gkyl120.
36. Bradfield J P, et al. A trans-ancestral meta-analysis of genome-wide association studies reveals loci associated with childhood obesity. Hum. Mol. Genet. 2019; 28:3327-3338. doi: 10.1093/hmg/ddz161.
37. Frayling T M, et al. A common variant in the FTO gene is associated with body mass index and predisposes to childhood and adult obesity. Science. 2007; 316:889-894. doi: 10.1126/science.1141634.
38. Locke A E, et al. Genetic studies of body mass index yield new insights for obesity biology. Nature. 2015; 518:197-206. doi: 10.1038/nature14177.
39. Sinnott-Armstrong N, et al. Genetics of 35 blood and urine biomarkers in the UK Biobank. Nat. Genet. 2021; 53:185-194. doi: 10.1038/s41588-020-00757-z.
40. Zhu Z, et al. Shared genetic and experimental links between obesity-related traits and asthma subtypes in UK Biobank. J. Allergy Clin. Immunol. 2020; 145:537-549. doi: 10.1016/j.jaci.2019.09.035.
41. Mahajan A, et al. Fine-mapping type 2 diabetes loci to single-variant resolution using high-density imputation and islet-specific epigenome maps. Nat. Genet. 2018; 50:1505-1513. doi: 10.1038/s41588-018-0241-6.
42. Mullin B H, et al. Identification of a role for the ARHGEF3 gene in postmenopausal osteoporosis. Am. J. Hum. Genet. 2008; 82:1262-1269. doi: 10.1016/j.ajhg.2008.04.016.
43. You J-S, et al. ARHGEF3 regulates skeletal muscle regeneration and strength through autophagy. Cell Rep. 2021; 34:108594. doi: 10.1016/j.celrep.2020.108594.
44. Diabetes Genetics Initiative of Broad Institute of Harvard and MIT, Lund University, and Novartis Institutes of BioMedical Research. et al. Genome-wide association analysis identifies loci for type 2 diabetes and triglyceride levels. Science. 2007; 316:1331-1336. doi: 10.1126/science.1142358.
45. Zeggini E, et al. Replication of genome-wide association signals in UK samples reveals risk loci for type 2 diabetes. Science. 2007; 316:1336-1341. doi: 10.1126/science.1142364.
46. Scott L J, et al. A genome-wide association study of type 2 diabetes in Finns detects multiple susceptibility variants. Science. 2007; 316:1341-1345. doi: 10.1126/science.1142382.
47. Chen Z, et al. Functional screening of candidate causal genes for insulin resistance in human preadipocytes and adipocytes. Circ. Res. 2020; 126:330-346. doi: 10.1161/CIRCRESAHA.119.315246.
48. Nono Nankam P A, et al. Distinct abdominal and gluteal adipose tissue transcriptome signatures are altered by exercise training in African women with obesity. Sci. Rep. 2020; 10:10240. doi: 10.1038/s41598-020-66868-z.
49. Loh N Y, et al. RSPO3 impacts body fat distribution and regulates adipose cell biology in vitro. Nat. Commun. 2020; 11:2797. doi: 10.1038/s41467-020-16592-z.
50. Loos R J F, Kilpelainen T O. Genes that make you fat, but keep you healthy. J. Intern. Med. 2018; 284:450-463. doi: 10.1111/joim.12827.
51. Emdin C A, et al. DNA sequence variation in ACVR1C encoding the activin receptor-like kinase 7 influences body fat distribution and protects against type 2 diabetes. Diabetes. 2019; 68:226-234. doi: 10.2337/db18-0857.
52. Zorzetto M, et al. SERPINA1 gene variants in individuals from the general population with reduced al-antitrypsin concentrations. Clin. Chem. 2008; 54:1331-1338. doi: 10.1373/clinchem.2007.102798.
53. van der Harst P, Verweij N. Identification of 64 novel genetic loci provides an expanded view on the genetic architecture of coronary artery disease. Circ. Res. 2018; 122:433-443. doi: 10.1161/CIRCRESAHA.117.312086.
54. Justice A E, et al. Protein-coding variants implicate novel genes related to lipid homeostasis contributing to body-fat distribution. Nat. Genet. 2019; 51:452-469. doi: 10.1038/s41588-018-0334-2.
55. Lumish H S, O'Reilly M, Reilly M P. Sex differences in genomic drivers of adipose distribution and related cardiometabolic disorders: opportunities for precision medicine. Arterioscler. Thromb. Vasc. Biol. 2020; 40:45-60. doi: 10.1161/ATVBAHA.119.313154.
56. Pettersson A M L, et al. MAFB as a novel regulator of human adipose tissue inflammation. Diabetologia. 2015; 58:2115-2123. doi: 10.1007/s00125-015-3673-x.
57. Emdin C A, et al. Association of genetic variation with cirrhosis: a multi-trait genome-wide association and gene-environment interaction study. Gastroenterology. 2021; 160:1620-1633.e13. doi: 10.1053/j.gastro.2020.12.011.
58. Hua X, et al. Non-alcoholic fatty liver disease is an influencing factor for the association of SHBG with metabolic syndrome in diabetes patients. Sci. Rep. 2017; 7:14532. doi: 10.1038/s41598-017-15232-9.
59. Randall J C, et al. Sex-stratified genome-wide association studies including 270,000 individuals show sexual dimorphism in genetic loci for anthropometric traits. PLoS Genet. 2013; 9:e1003500. doi: 10.1371/journal.pgen.1003500.
60. Gusev A, et al. Integrative approaches for large-scale transcriptome-wide association studies. Nat. Genet. 2016; 48:245-252. doi: 10.1038/ng.3506.
61. Kilpelainen T O, et al. Genetic variation near IRS1 associates with reduced adiposity and an impaired metabolic profile. Nat. Genet. 2011; 43:753-760. doi: 10.1038/ng.866.
62. Hagberg C E, et al. Vascular endothelial growth factor B controls endothelial fatty acid uptake. Nature. 2010; 464:917-921. doi: 10.1038/nature08945.
63. Robciuc M R, et al. VEGFB/VEGFR1-induced expansion of adipose vasculature counteracts obesity and related metabolic complications. Cell Metab. 2016; 23:712-724. doi: 10.1016/j.cmet.2016.03.004.
64. Finucane H K, et al. Heritability enrichment of specifically expressed genes identifies disease-relevant tissues and cell types. Nat. Genet. 2018; 50:621-629. doi: 10.1038/s41588-018-0081-4.
65. Emdin C A, et al. Analysis of predicted loss-of-function variants in UK Biobank identifies variants protective for disease. Nat. Commun. 2018; 9:1613. doi: 10.1038/s41467-018-03911-8.
66. Jackson R S, et al. Obesity and impaired prohormone processing associated with mutations in the human prohormone convertase 1 gene. Nat. Genet. 1997; 16:303-306. doi: 10.1038/ng0797-303.
67. Akbari, P. et al. Sequencing of 640,000 exomes identifies GPR75 variants associated with protection from obesity. Science 373, eabf8683 (2021).
68. Dharuri H, et al. Downregulation of the acetyl-CoA metabolic network in adipose tissue of obese diabetic individuals and recovery after weight loss. Diabetologia. 2014; 57:2384-2392. doi: 10.1007/s00125-014-3347-0.
69. Hegele R A, Cao H, Frankowski C, Mathews S T, Leff T. PPARG F388L, a transactivation-deficient mutant, in familial partial lipodystrophy. Diabetes. 2002; 51:3586-3590. doi: 10.2337/diabetes.51.12.3586.
70. Srinivasan S, et al. A polygenic lipodystrophy genetic risk score characterizes risk independent of BMI in the diabetes prevention program. J. Endocr. Soc. 2019; 3:1663-1677. doi: 10.1210/js.2019-00069.
71. Prive F, Arbel J, Vilhjalmsson B J. LDpred2: better, faster, stronger. Bioinformatics. 2020; 36:5424-5431. doi: 10.1093/bioinformatics/btaa1029.
72. The ARIC investigators. The Atherosclerosis Risk in Communities (ARIC) study: design and objectives. Am. J Epidemiol. 129, 687-702 (1989).
73. Ried J S, et al. A principal component meta-analysis on multiple anthropometric traits identifies novel loci for body shape. Nat. Commun. 2016; 7:13357. doi: 10.1038/ncomms13357.
74. Sulc J, et al. Composite trait Mendelian randomization reveals distinct metabolic and lifestyle consequences of differences in body shape. Commun. Biol. 2021; 4:1-13. doi: 10.1038/s42003-021-02550-y.
75. Despres J-P, Lemieux I. Abdominal obesity and metabolic syndrome. Nature. 2006; 444:881-887. doi: 10.1038/nature05488.
76. Makimura H, et al. Metabolic effects of a growth hormone-releasing factor in obese subjects with reduced growth hormone secretion: a randomized controlled trial. J. Clin. Endocrinol. Metab. 2012; 97:4769-4779. doi: 10.1210/jc.2012-2794.
77. Stanley T L, et al. Effect of tesamorelin on visceral fat and liver fat in HIV-infected patients with abdominal fat accumulation: a randomized clinical trial. JAMA. 2014; 312:380-389. doi: 10.1001/jama.2014.8334.
78. Meral R, et al. ‘Fat Shadows’ from DXA for the qualitative assessment of lipodystrophy: when a picture is worth a thousand numbers. Diabetes Care. 2018; 41:2255-2258. doi: 10.2337/dc18-0978.
79. Laber, S. et al. Discovering cellular programs of intrinsic and extrinsic drivers of metabolic traits using LipocyteProfiler. 10.1101/2021.07.17.452050 (2021).
80. Sinnott-Armstrong N, et al. A regulatory variant at 3q21.1 confers an increased pleiotropic risk for hyperglycemia and altered bone mineral density. Cell Metab. 2021; 33:615-628.e13. doi: 10.1016/j.cmet.2021.01.001.
81. Sudlow C, et al. UK biobank: an open access resource for identifying the causes of a wide range of complex diseases of middle and old age. PLoS Med. 2015; 12:e1001779. doi: 10.1371/journal.pmed.1001779.
82. Littlejohns T J, et al. The UK Biobank imaging enhancement of 100,000 participants: rationale, data collection, management and future directions. Nat. Commun. 2020; 11:2624. doi: 10.1038/s41467-020-15948-9.
83. Aschard H, Vilhjalmsson B J, Joshi A D, Price A L, Kraft P. Adjusting for heritable covariates can bias effect estimates in genome-wide association studies. Am. J. Hum. Genet. 2015; 96:329-339. doi: 10.1016/j.ajhg.2014.12.021.
84. Day F R, Loh P-R, Scott R A, Ong K K, Perry J R B. A robust example of collider bias in a genetic association study. Am. J. Hum. Genet. 2016; 98:392-393. doi: 10.1016/j.ajhg.2015.12.019.
85. Bycroft C, et al. The UK Biobank resource with deep phenotyping and genomic data. Nature. 2018; 562:203-209. doi: 10.1038/s41586-018-0579-z.
86. UK10K Consortium. et al. The UK10K project identifies rare variants in health and disease. Nature. 2015; 526:82-90. doi: 10.1038/nature14962.
87. 1000 Genomes Project Consortium. et al. A global reference for human genetic variation. Nature. 2015; 526:68-74. doi: 10.1038/nature15393.
88. Mbatchou J, et al. Computationally efficient whole-genome regression for quantitative and binary traits. Nat. Genet. 2021; 53:1097-1103. doi: 10.1038/s41588-021-00870-7.
89. Zhou W, et al. Efficiently controlling for case-control imbalance and sample relatedness in large-scale genetic association studies. Nat. Genet. 2018; 50:1335-1341. doi: 10.1038/s41588-018-0184-y.
90. Loh P-R, et al. Efficient Bayesian mixed-model analysis increases association power in large cohorts. Nat. Genet. 2015; 47:284-290. doi: 10.1038/ng.3190.
91. Loh P-R, Kichaev G, Gazal S, Schoech A P, Price A L. Mixed-model association for biobank-scale datasets. Nat. Genet. 2018; 50:906-908. doi: 10.1038/s41588-018-0144-6.
92. Winkler T W, et al. EasyStrata: evaluation and visualization of stratified genome-wide association meta-analysis data. Bioinformatics. 2015; 31:259-261. doi: 10.1093/bioinformatics/btu621.
93. Gamazon E R, et al. A gene-based association method for mapping traits using reference transcriptome data. Nat. Genet. 2015; 47:1091-1098. doi: 10.1038/ng.3367.
94. Zhu Z, et al. Integration of summary data from GWAS and eQTL studies predicts complex trait gene targets. Nat. Genet. 2016; 48:481-487. doi: 10.1038/ng.3538.
95. GTEx Consortium. Human genomics. The Genotype-Tissue Expression (GTEx) pilot analysis: multitissue gene regulation in humans. Science. 2015; 348:648-660. doi: 10.1126/science.1262110.
96. Pers T H, et al. Biological interpretation of genome-wide association studies using predicted gene functions. Nat. Commun. 2015; 6:5890. doi: 10.1038/ncomms6890.
97. Fehrmann R S N, et al. Gene expression analysis identifies global gene dosage sensitivity in cancer. Nat. Genet. 2015; 47:115-125. doi: 10.1038/ng.3173.
98. Szustakowski J D, et al. Advancing human genetics research and drug discovery through exome sequencing of the UK Biobank. Nat. Genet. 2021; 53:942-948. doi: 10.1038/s41588-021-00885-0.
99. Jurgens S J, et al. Analysis of rare genetic variation underlying cardiometabolic diseases and traits among 200,000 individuals in the UK Biobank. Nat. Genet. 2022; 54:240-250. doi: 10.1038/s41588-021-01011-w.
100. Li H. Toward better understanding of artifacts in variant calling from high-coverage samples. Bioinformatics. 2014; 30:2843-2851. doi: 10.1093/bioinformatics/btu356.
101. Bailey J A. Segmental duplications: organization and impact within the current human genome project assembly. Genome Res. 2001; 11:1005-1017. doi: 10.1101/gr.187101.
102. Chang C C, et al. Second-generation PLINK: rising to the challenge of larger and richer datasets. GigaScience. 2015; 4:7. doi: 10.1186/s13742-015-0047-8.
103. Manichaikul A, et al. Robust relationship inference in genome-wide association studies. Bioinformatics. 2010; 26:2867-2873. doi: 10.1093/bioinformatics/btq559.
104. Zhao H, et al. CrossMap: a versatile tool for coordinate conversion between genome assemblies. Bioinformatics. 2014; 30:1006-1007. doi: 10.1093/bioinformatics/btt73 O.
105. Karczewski K J, et al. The mutational constraint spectrum quantified from variation in 141,456 humans. Nature. 2020; 581:434-443. doi: 10.1038/s41586-020-2308-7.
106. Aken B L, et al. The Ensembl gene annotation system. Database. 2016; 2016:baw093. doi: 10.1093/database/baw093.
107. Do R, et al. Exome sequencing identifies rare LDLR and APOAS alleles conferring risk for myocardial infarction. Nature. 2015; 518:102-106. doi: 10.1038/nature13917.
108. Khera A V, et al. Diagnostic yield and clinical utility of sequencing familial hypercholesterolemia genes in patients with severe hypercholesterolemia. J. Am. Coll. Cardiol. 2016; 67:2578-2589. doi: 10.1016/j.jacc.2016.03.520.
109. Khera A V, et al. Association of rare and common variation in the lipoprotein lipase gene with coronary artery disease. JAMA. 2017; 317:937-946. doi: 10.1001/jama.2017.0972.
110. Liu X, Wu C, Li C, Boerwinkle E. dbNSFP v3.0: a one-stop database of functional predictions and annotations for human nonsynonymous and splice-site SNVs. Hum. Mutat. 2016; 37:235-241. doi: 10.1002/humu.22932.
111. Ng, P. C. & Henikoff, S. SIFT: predicting amino acid changes that affect protein function. Nucleic Acids Res. 31, 3812-3814 (2003).
112. Adzhubei I A, et al. A method and server for predicting damaging missense mutations. Nat. Methods. 2010; 7:248-249. doi: 10.1038/nmeth0410-248.
113. Chun S, Fay J C. Identification of deleterious mutations within three human genomes. Genome Res. 2009; 19:1553-1561. doi: 10.1101/gr.092619.109.
114. Schwarz J M, Cooper D N, Schuelke M, Seelow D. MutationTaster2: mutation prediction for the deep-sequencing age. Nat. Methods. 2014; 11:361-362. doi: 10.1038/nmeth.2890.
115. Lee S, Abecasis G R, Boehnke M, Lin X. Rare-variant association analysis: study designs and statistical tests. Am. J. Hum. Genet. 2014; 95:5-23. doi: 10.1016/j.ajhg.2014.06.009.
116. Park J-H, et al. Distribution of allele frequencies and effect sizes and their interrelationships for common genetic susceptibility variants. Proc. Natl Acad. Sci. USA. 2011; 108:18026-18031. doi: 10.1073/pnas.1114759108.

### Example 3—Additional Methods and Data

### Convolutional Neural Networks to Compute VAT, ASAT, and GFAT Volumes

A full description of the machine learning methods used to predict VAT, ASAT, and GFAT volumes including performance metrics and associations with type 2 diabetes and coronary artery disease is available in a prior manuscript.¹

Among UK Biobank participants who underwent MM imaging study, a subset had visceral adipose tissue (VAT) volume, abdominal subcutaneous adipose tissue (ASAT) volume, and total adipose tissue between the bottom of the thigh muscles to the top of vertebrae T9 (TAT) volume quantified and made available via the UK Biobank portal to the broader research community.²⁻⁷ VAT (field 22407, “volume of the adipose tissue within the abdominal cavity, excluding adipose tissue outside the abdominal skeletal muscles and adipose tissue and lipids within and posterior of the spine and posterior of the back muscles”) was available in 9,978 participants, ASAT (field 22408, “volume of the subcutaneous adipose tissue in the abdomen from the top of the femoral head to the top of the thoracic vertebrae T9”) was available in 9,979, and TAT (field 22415, “total volume of adipose tissue, measured by MM, between the bottom of the thigh muscles to the top of vertebrae T9”) was available in 8,524. Based on these definitions, Applicants additionally computed gluteofemoral adipose tissue (GFAT) volume:

GFAT=TAT (between top of T9 and bottom of thigh muscles)−VAT−ASAT

Given that the vast majority of adipose tissue between the top of vertebrae T9 and the top of the femoral head is accounted for by VAT or ASAT, GFAT was defined as total adipose tissue between the top of the femoral head and the bottom of the thigh muscles.

To train convolutional neural network models to measure VAT, ASAT, and GFAT, Applicants first simplified the three-dimensional MRI images into composite two-dimensional projections of coronal and sagittal views, leading to an 830-fold reduction in data input size (Supplementary FIG. 1). These machine learning models—trained on 80% of the participants with fat depots previously quantified—demonstrated near-perfect estimation association of each fat depot in the 20% of remaining individuals for each depot (r2=0.991, 0.991, and 0.978 for VAT, ASAT, and GFAT, respectively).

Finally, given that the gold standard for GFAT was derived from three other UK Biobank fields (VAT, ASAT, and TAT), Applicants sought additional validation using DEXA-derived gynoid fat—corresponding to fat between the greater femoral trochanter and the mid-thigh—in UK Biobank. Among the 40,032 individuals with GFAT quantified from the above pipeline, 33,989 had gynoid fat mass available from DEXA imaging (multiplying gynoid total mass field 23265 and gynoid fat percent field 23264). Correlation between MM-derived GFAT volume and DEXA-derived gynoid fat mass was very good (Pearson r=0.96), supporting the validity of GFAT

(Supplementary Table 1).
Supplementary Table 1 Observational correlation between MRI-
derived GFAT volume and DEXA-derived gynoid fat mass

| Subgroup | Pearson correlation (r) |
|---|---|
| Males | 0.956 |
| Females | 0.962 |

### Justification for BMI and Height Adjustment for Fat Depot Volumes

Initially motivated by seminal work on waist-hip ratio adjusted for BMI led by the GIANT consortium, Applicants started by examining the properties of VAT, ASAT, and GFAT adjusted for BMI (but not height). 8 While genetic correlation with BMI was markedly reduced as desired, Applicants noted that this adjustment introduced a significant genetic correlation with height (r_{g} ranging from 0.29-0.67) (Supplementary Table 2). As an example, GFAT adjusted for BMI (but not height) associated with rs67807996 (P=4.1×10⁻¹⁴) and rs59985551 (P=2.1×10⁻¹³) which have previously been identified as height-associated variants. 9,1°

A similar phenomenon has previously been noted with waist circumference adjusted for BMI (WCadjBMI) and hip circumference (HIPadjBMI) adjusted for BMI in work led by the GIANT consortium:
“In contrast to WHRadjBMI, which has almost no genetic correlation with height (r_{g}<0.04), WCadjBMI (r_{g}=0.42) and HIPadjBMI (r_{g}=0.82) have moderate genetic correlations with height. These data suggest that some, but not all, WCadjBMI and HIPadjBMI loci would be associated with height.”⁸
   Accordingly, one of the height-associated variants noted above—rs59985551—has also been associated with WCadjBMI and HIPadjBMI.¹¹

By additionally adjusting for height, VAT adjusted for BMI and height (VATadj), ASATadj, and GFATadj achieved near height-independence (r_{g} ranging from −0.04-0.02) as desired. This strategy is consistent with the goal of this study to nominate genetic variants associated with “local adiposity”—i.e., genetic variants that influence adipose tissue volume in specific fat depots independent of the “overall size” of an individual. Of note, adjustment of each fat depot for BMI and height led to values that were nearly identical—both in terms of observational and genetic correlation—to adjusting each fat depot for weight and height. This latter strategy has previously been used to adjust CT-derived pericardial fat prior to genetic association.^{12,13}

Hence, the “adj” traits in this study are adjusted for BMI and height. More precisely, each adj trait represents residuals of sex-specific regressions of the fat depot of interest against age, age squared, BMI, and height.

Supplementary Table 2 Genetic correlations between VAT, ASAT, and GFAT
with various adjustment strategies and BMI and height

| | Genetic Correlation | Genetic Correlation |
|---|---|---|
| | (r_{g}) with BMI | (r_{g}) with Height |
| VAT | 0.663 (0.04) | 0.104 (0.04) |
| ASAT | 0.823 (0.02) | 0.145 (0.04) |
| GFAT | 0.692 (0.03) | 0.367 (0.03) |
| VAT adjusted for BMI | −0.199 (0.06) | 0.290 (0.04) |
| ASAT adjusted for BMI | −0.111 (0.05) | 0.502 (0.03) |
| GFAT adjusted for BMI | −0.101 (0.05) | 0.666 (0.03) |
| VAT adjusted for BMI and Height | −0.165 (0.05) | −0.040 (0.05) |
| ASAT adjusted for BMI and Height | −0.068 (0.06) | 0.018 (0.05) |
| GFAT adjusted for BMI and Height | −0.045 (0.05) | 0.020 (0.04) |
| VAT adjusted for Weight and Height | −0.176 (0.05) | −0.033 (0.04) |
| ASAT adjusted for Weight and Height | −0.077 (0.06) | 0.027 (0.05) |
| GFAT adjusted for Weight and Height | −0.055 (0.05) | 0.026 (0.04) |

All genetic correlations are computed using LD-score regression as described in the Methods section of the manuscript.^{14,15}

Quantifying Extent of Collider Bias with BMI or Height

Applicants determined that collider bias with BMI or height is minimally contributing to these results by conducting sensitivity analyses outlined in a recent large meta-analysis of WHRadjBMI¹⁶:

First, Applicants determined the genome-wide genetic correlation between each of VATadj, ASATadj, and GFATadj with BMI and height, and compared to genetic correlations between WHRadjBMI and BMI and height (Supplementary Table 3). The greatest magnitude of genetic correlation was observed between VATadj and BMI (r_{g}=−0.165, SE=0.05) and this was comparable to the genetic correlation between WHRadjBMI and BMI (r_{g}=−0.109, SE=0.07). Hence, from a genome-wide standpoint, the extent of collider bias with BMI and height was no more than that of WHRadjBMI.

Supplementary Table 3 Genetic correlations between VATadj, ASATadj, and GFATadj
with BMI and height are comparable to those corresponding to WHRadjBMI

| | Genetic Correlation | Genetic Correlation |
|---|---|---|
| | (r_{g}) with BMI | (r_{g}) with Height |
| VAT adjusted for BMI and Height | −0.165 (0.05) | −0.040 (0.05) |
| (VATadj) | | |
| ASAT adjusted for BMI and Height | −0.068 (0.06) | 0.018 (0.05) |
| (ASATadj) | | |
| GFAT adjusted for BMI and Height | −0.045 (0.05) | 0.020 (0.04) |
| (GFATadj) | | |
| WHRadjBMI | −0.109 (0.07) | −0.017 (0.05) |

Genetic correlations between WHRadjBMI, BMI, and height are obtained using summary statistics from GWAS carried out in the same imaging cohort where analyses of VATadj, ASATadj, and GFATadj were done.

Next, Applicants evaluated the fraction of lead SNPs (P<5×10⁻⁸) for VATadj, ASATadj, and GFATadj that had stronger effect sizes for the unadjusted fat depot compared to effect sizes for BMI or height. Applicants found that the majority of SNPs associated with adjusted fat depots were more strongly associated with the unadjusted fat depot than either of BMI or height (71-98%; Supplementary Table 4). For reference, 311/346 (90%) of the WHRadjBMI lead SNPs from a recent meta-analysis had a greater effect size magnitude for WHR than BMI. 16 This observation indicates that most genetic associations are unlikely to be secondary to collider bias with BMI or height.

Supplementary Table 4 The majority of lead SNPs identified with VATadj, ASATadj, and
GFATadj are more strongly associated with the unadjusted fat depot than BMI or height

| | | Lead SNPs where effect size for | Lead SNPs where effect size for |
|---|---|---|---|
| | Lead | unadjusted fat depot is greater | unadjusted fat depot is greater |
| | SNPs | than BMI effect size | than height effect size |
| VAT adjusted for BMI and Height | 30 | 26 (87%) | 24 (80%) |
| (VATadj) | | | |
| ASAT adjusted for BMI and | 21 | 18 (86%) | 15 (71%) |
| Height (ASATadj) | | | |
| GFAT adjusted for BMI and | 54 | 53 (98%) | 52 (96%) |
| Height (GFATadj) | | | |

Applicants additionally plotted each adjusted fat depot lead SNP on four plots to visualize data summarized in Supplementary Table 4 (FIG. 9-11):
Plot 1 (top left):
   y-axis: −log10 (P(unadjusted fat depot)/P(BMI))
   x-axis: −log10(P(adjusted fat depot)
Plot 2 (top right):
   y-axis: beta(unadjusted fat depot)
   x-axis: beta(BMI)
Plot 3 (bottom left):
   y-axis: −log10 (P (unadjusted fat depot)/P(height))
   x-axis: −log10(P(adjusted fat depot)
Plot 4 (bottom right):
   y-axis: beta(unadjusted fat depot)
   x-axis: beta(height)

Finally, Applicants aimed to determine the effect of the VATadj, ASATadj, and GFATadj polygenic scores derived in this study on the corresponding metric, the corresponding unadjusted fat depot volume, BMI, and height. Applicants found in each case that the polygenic score was significantly associated with the adjusted fat depot and the corresponding unadjusted fat depot, but not BMI or height (Supplementary Table 5). Taking GFATadj as an example, a 1-standard deviation increase in the polygenic score associated with increased GFATadj (beta=0.27, P=5.9e-122) and increased GFAT (beta =0.15, P=2.5e-38), but a null effect with BMI (beta=0.02, P=0.15) and height (beta=0.02, P=0.10).

Supplementary Table 5 Association of VATadj, ASATadj, and GFATadj polygenic
scores with VATadj, ASATadj, GFATadj, unadjusted metrics, BMI, and height

| PRS | Trait | Beta (95% CI) | P-value | Adjusted R² |
|---|---|---|---|---|
| VATadj | VATadj | 0.24 | 4.8e−101 | 0.0577 |
| | | (0.22-0.26) | | |
| | VAT | 0.13 | 4.8e−33 | 0.0179 |
| | | (0.11-0.16) | | |
| | BMI | −0.02 | 0.13 | 0.0001 |
| | | (−0.04-0.01) | | |
| | Height | −0.01 | 0.54 | 0.0000 |
| | | (−0.03-0.01) | | |
| ASATadj | ASATadj | 0.19 | 3.9e−62 | 0.0355 |
| | | (0.17-0.21) | | |
| | ASAT | 0.08 | 6.0e−14 | 0.0070 |
| | | (0.06-0.11) | | |
| | BMI | 0.00 | 0.91 | −0.0002 |
| | | (−0.02-0.02) | | |
| | Height | 0.00 | 0.78 | −0.0001 |
| | | (−0.02-0.02) | | |
| GFATadj | GFATadj | 0.27 | 5.9e−122 | 0.0703 |
| | | (0.24-0.29) | | |
| | GFAT | 0.15 | 2.5e−38 | 0.0210 |
| | | (0.12-0.17) | | |
| | BMI | 0.02 | 0.15 | 0.0001 |
| | | (−0.01-0.04) | | |
| | Height | 0.02 | 0.1 | 0.0003 |
| | | (0.00-0.04) | | |

Results reported here are from the 20% holdout set that was used to determine performance of polygenic scores. For all of VATadj, ASATadj, and GFATadj, the optimal set of LDpred2 hyperparameters in the validation set were p = 0.0056, h2 = 0.7, sparse = FALSE (Supplementary Table S22). To report performance metrics, each polygenic score was first adjusted for the first 10 PCs of genetic ancestry. Each PC-residualized polygenic score was then used to predict the trait of interest in a model that was adjusted for age at the time of imaging, sex, and the first 10 PCs of genetic ancestry. Betas correspond to sex-specific standard deviations per 1-standard deviation of the polygenic score. P-values correspond to the polygenic score term in each linear regression. The adjusted R2 corresponds to R2 of the full model minus R2 of a model containing only covariates.

In summary, the goal with the adjusted fat depot analyses was to understand the genetic architecture of “local adiposity”—i.e., adipose tissue volume in a given fat depot out of proportion to an individual's body size as captured by BMI and height. Sensitivity analyses above suggest:
Adjusting for BMI+height avoids undesired genetic correlations with height that were previously noted for WCadjBMI and HIPadjBM⁸; of note, adjustment for BMI+height is nearly identical to adjustment for weight+height, which was employed previously to adjust CT-derived pericardial fat prior to genetic association.^{12,13}
Carrying out sensitivity analyses to determine the extent of collider bias as outlined by Pulit et al. for WHRadjBMI¹⁶, Applicants determine that collider bias with BMI or height is unlikely to be driving the majority of the discovered associations for VATadj, ASATadj, and GFATadj.

Supplementary Table 6 Heritability of adiposity phenotypes

| | | | | baselineLD |
|---|---|---|---|---|
| | h_{g}² (BOLT-REML) | | | model |
| Phenotype | Combined | Males | Females | Combined |
| VAT | 0.310 (0.014) | 0.296 (0.028) | 0.401 (0.027) | 0.194 (0.021) |
| ASAT | 0.313 (0.014) | 0.295 (0.028) | 0.382 (0.027) | 0.174 (0.023) |
| GFAT | 0.360 (0.014) | 0.332 (0.028) | 0.422 (0.026) | 0.207 (0.024) |
| VATadj | 0.407 (0.015) | 0.435 (0.029) | 0.455 (0.027) | 0.291 (0.027) |
| ASATadj | 0.339 (0.015) | 0.400 (0.029) | 0.411 (0.027) | 0.238 (0.024) |
| GFATadj | 0.411 (0.015) | 0.418 (0.029) | 0.518 (0.027) | 0.271 (0.028) |
| VAT/ASAT | 0.407 (0.014) | 0.453 (0.028) | 0.430 (0.026) | 0.288 (0.025) |
| VAT/GFAT | 0.395 (0.014) | 0.402 (0.028) | 0.473 (0.026) | 0.278 (0.022) |
| ASAT/GFAT | 0.367 (0.014) | 0.359 (0.028) | 0.497 (0.026) | 0.228 (0.023) |
| BMI | 0.307 (0.015) | 0.318 (0.029) | 0.330 (0.028) | 0.201 (0.024) |
| Waist circ. | 0.248 (0.015) | 0.229 (0.029) | 0.297 (0.028) | 0.140 (0.023) |
| WHR | 0.216 (0.015) | 0.223 (0.029) | 0.275 (0.027) | 0.128 (0.021) |
| WHRadjBMI | 0.206 (0.014) | 0.226 (0.028) | 0.240 (0.027) | 0.146 (0.021) |

The first three columns are SNP-heritability estimates (hg2) obtained from BOLT-REML18-20, while the fourth column contains heritability parameter estimates from LD-score regression with the baseline LD model.21 On average, the heritability parameter estimate for the baselineLD model is 67% of the SNP-heritability estimates from BOLT-LMM, which is consistent with prior comparisons.20 General trends include: (1) measures of local adiposity (adjusted-for-BMI and fat depot ratios) being more heritable than measures strongly correlated with global adiposity (BMI, VAT, ASAT, GFAT) and (2) most traits being more heritable in female participants (VAT/ASAT is the exception).

SUPPLEMENTARY TABLE 7
Nominally significant associations between the newly-identified adiposity loci in this study and cardiometabolic traits

| | | | | | Nearest | Nominally significant associations with cardiometabolic |
|---|---|---|---|---|---|---|
| Trait | CHR | BP | SNP | P-value | Gene | in the Type 2 Diabetes Knowledge Portal (P < 0.05) |
| GFAT | 11 | 95840436 | rs1074742 | 1.40E−08 | MAML2 | Assorted MAGIC insulin secretion during OGTT traits22 |
| | | | | | | (incremental insulin at 30 min OGTT, insulin at 30 min |
| | | | | | | OGTT adjBMI, AUCins over AUCgluc), assorted IVGTT- |
| | | | | | | based insulin secretion traits23 (peak insulin response, |
| | | | | | | acute insulin response), HbA1c adjBMI24 |
| GFAT | 12 | 124344710 | rs138756410 | 3.00E−08 | DNAH10 | Obese vs. control OR Obese vs. thin25, coronary artery |
| | | | | | | disease26, acute insulin response23 |
| GFAT | 12 | 125092343 | rs4765159 | 3.50E−08 | NCOR2 | Waist circumference (+/−adj BMI-smoking status)27, 28, |
| | | | | | | ratio total to HDL cholesterol, two-hour insulin |
| VATadj | 2 | 121310704 | rs35932591 | 3.80E−08 | LINC01101 | Triglcyerides29, 30, LDL-cholesterol29, 30, eGFR and |
| | | | | | | BUN31, Fasting insulin adjBMI24, Systolic blood pressure32, |
| | | | | | | BMI30, coronary artery disease26, AST/ALT ratio33, type 2 |
| | | | | | | diabetes34, WHRadjBMI16, HDL-cholesterol |
| VATadj | 10 | 25767521 | rs1329254 | 1.40E−08 | GPR158 | Diastolic blood pressure and systolic blood pressure32, |
| | | | | | | random blood glucose29, BMI16 |
| VATadj | 11 | 69195097 | rs7933253 | 1.30E−08 | LOC102724265 | WHRadjBMI16, BMI35, Hip circumference8 |
| VATadj | 2 | 121310704 | rs35932591 | 3.90E−08 | LINC01101 | See entry for VATadj |
| (Male) | | | | | | |
| VATadj | 3 | 56901687 | rs1500714 | 1.80E−08 | ARHGEF3 | Assorted MAGIC insulin secretion during OGTT traits22 |
| (Female) | | | | | | (AUC for insulin, insulin at 30 min OGTT, AUCins over |
| | | | | | | AUCgluc, incremental insulin at 30 min OGTT, Matsuda |
| | | | | | | insulin sensitivity index, corrected insulin response, |
| | | | | | | insulin at 30 min OGTT adj BMI), WHRadjBMIsmoking |
| | | | | | | and WaistadjBMIsmoking28, TOAST small artery |
| | | | | | | occlusion36, ALT |
| ASATadj | 1 | 201016296 | rs3850625 | 1.80E−12 | CACNA1S | eGFR31, Diastolic blood pressure and systolic blood |
| | | | | | | pressure32, Fasting insulin adjBMI24, Body fat percentage, |
| | | | | | | AST/ALT ratio33, WaistadjBMIsmoking28, WaistadjBMI8, |
| | | | | | | Hip adjBMI8, Leptin, BMI, coronary artery disease26, |
| | | | | | | HDL3 cholesterol37, two-hour glucose adjBMI24, Waist |
| | | | | | | circumference, Controls vs. thin25 |
| ASATadj | 9 | 1044400 | rs2048235 | 4.10E−08 | LINC01230 | Fasting insulin adjBMI24, type 2 diabetes (or adjBMI)38, |
| | | | | | | AST/ALT ratio33, ALT33, coronary artery disease26, body |
| | | | | | | fat percentage, random blood glucose29, eGFR-cys39, |
| | | | | | | obesity, |
| ASATadj | 9 | 1052722 | rs6474550 | 1.30E−09 | DMRT2 | AST/ALT ratio33, Waist circumference (+/−adjBMI or |
| | | | | | | adjBMIsmoking)8, 28, Triglycerides, Hip circumference |
| | | | | | | (+/−adjBMI)8, type 2 diabetes (+/−adjBMI)38, |
| | | | | | | BMIadjsmoking28, WHR (+/−adjBMI)8, Assorted MAGIC |
| | | | | | | insulin secretion during OGTT traits22 (AUC for insulin), |
| | | | | | | ALT, BUN, eGFR-cys |
| ASATadj | 15 | 62757857 | rs17205757 | 3.20E−08 | MIR6085 | Pulse, systolic, and diastolic blood pressure32, eGFR31, |
| | | | | | | LDL-cholesterol, BMI, Triglycerides, HbA1c, ALT, insulin |
| | | | | | | sensitivity adjBMI, Obese vs. control25, TOAST other |
| | | | | | | determined, WHRadjBMI16 |
| ASATadj | 17 | 76324751 | rs4444401 | 4.20E−08 | SOCS3 | Type 2 diabetes, AST33, Assorted MAGIC insulin secretion |
| | | | | | | during OGTT traits22 (corrected insulin response), systolic |
| | | | | | | and pulse blood pressure32, HbA1cadjBMI24, HDL- |
| | | | | | | cholesterol, two-hour glucoseadjBMI24, HipadjBMI8 |
| ASATadj | 1 | 116916645 | rs749166380 | 2.20E−08 | ATP1A1 | Obese vs. control²⁵, trunk fat ratio⁴⁰ |
| (Female) | | | | | | |
| ASATadj | 8 | 58352327 | rs776481989 | 8.60E−09 | LOC101929488 | |
| (Female) | | | | | | |
| GFATadj | 2 | 3648186 | rs7588285 | 1.40E−08 | COLEC11 | LDL-cholesterol, triglycerides, total cholesterol, diastolic |
| | | | | | | and systolic blood pressure32, HDL-cholesterol, eGFR31, |
| | | | | | | obesity, coronary artery disease26, AST/ALT ratio33, Weight, |
| | | | | | | Assorted MAGIC insulin secretion during OGTT traits22 |
| | | | | | | (Matsuda insulin sensitivity), Fasting insulin adjBMI24 |
| GFATadj | 2 | 226768344 | 2:226768344_CA_C | 2.60E−08 | NYAP2 | |
| GFATadj | 3 | 196818853 | rs13099700 | 7.90E−09 | DLG1 | eGFR31, WHRadjBMI (or WHR)16, systolic and diastolic |
| | | | | | | blood pressure32, BMI, NAFLD in type 2 diabetes, Rankin |
| | | | | | | stroke severity |
| GFATadj | 5 | 38810354 | rs142369482 | 9.10E−09 | OSMR-AS1 | Hypertension, waist circumference, weight |
| GFATadj | 10 | 122970216 | rs1907218 | 3.60E−10 | FGFR2 | Systolic, pulse, and diastolic blood pressure32, type 2 |
| | | | | | | diabetes (or adjBMI)38, WHRadjBMI (or WHR or |
| | | | | | | adjBMIsmoking)16, 28, AST/ALT ratio33, Triglycerides, |
| | | | | | | HDL-cholesterol, BMI, HipadjBMI8, random glucose, |
| | | | | | | Fasting insulin adjBMI24, ALT |
| GFATadj | 4 | 104780790 | rs528845403 | 2.40E−08 | TACR3 | Arm fat ratio40, Trunk fat ratio40, Hypertension41 |
| (Male) | | | | | | |
| GFATadj | 1 | 181161153 | rs7550430 | 1.80E−09 | LINC01732 | Weight42, hip circumference42 |
| (Female) | | | | | | |
| GFATadj | 2 | 165533198 | rs386652275 | 3.20E−08 | COBLL1 | |
| (Female) | | | | | | |
| VAT/ | 2 | 178121005 | rs13028464 | 4.80E−08 | NFE2L2 | eGFR or BUN31, C-reactive protein, triglycerides, systolic, |
| ASAT | | | | | | pulse, or diastolic blood pressure32, LDL-cholesterol, |
| | | | | | | WHRadjBMI16, type 1 diabetes, TOAST other |
| | | | | | | undetermined, stroke in type 2 diabetes, Arm fat ratio40, |
| | | | | | | Adiponectin, assorted IVGTT-based insulin secretion |
| | | | | | | traits23 (acute insulin response adj SI or adj BMI-SI), |
| | | | | | | HDL-cholesterol, TOAST large artery atherosclerosis36 |
| VAT/ | 6 | 19947871 | rs70987287 | 1.70E−17 | ID4 | Ischemic stroke |
| ASAT | | | | | | |
| VAT/ | 8 | 25459001 | rs3890765 | 6.80E−09 | CDCA2 | WHRadjBMI (or WHR)16, BUN, TOAST other |
| ASAT | | | | | | undetermined, AST/ALT ratio33, fasting plasma |
| | | | | | | glucose43 |
| VAT/ | 9 | 1054362 | rs6474552 | 1.20E−08 | DMRT2 | AST/ALT ratio33, Waist circumference (or adjBMI or |
| ASAT | | | | | | adjBMIsmoking)8, 28, Triglycerides, Fasting insulin |
| | | | | | | adjBMI24, LDL-cholesterol, Assorted MAGIC insulin |
| | | | | | | secretion during OGTT traits22 (AUC for insulin, |
| | | | | | | Matsuda insulin sensitivity), type 2 diabetes |
| | | | | | | adjBMI38, BUN, eGFR, Hip circumference8, Obese |
| | | | | | | vs. thin25 |
| VAT/ | 10 | 63702572 | rs55767272 | 6.80E−09 | ARID5B | Triglycerides29, WHR (or adjBMI)16, BMI |
| ASAT | | | | | | |
| VAT/ | 10 | 122992475 | rs11199845 | 1.50E−14 | FGFR2 | Systolic, pulse, and diastolic blood pressure32, type 2 |
| ASAT | | | | | | diabetes (or adjBMI)38, triglycerides29, Fasting insulin |
| | | | | | | adjBMI24, BMI, AST/ALT ratio33, coronary artery |
| | | | | | | disease26, random glucose, HDL-cholesterol30 |
| VAT/ | 2 | 61760756 | rs13390751 | 1.30E−08 | XPO1 | AST/ALT ratio33, pulse and systolic blood pressure32, |
| ASAT | | | | | | BMI, LDL-cholesterol, triglycerides, coronary artery |
| (Male) | | | | | | disease26, ALT, total cholesterol, type 2 diabetes38 |
| VAT/ | 6 | 19949170 | 6:19949170_GT_G | 3.70E−09 | ID4 | |
| ASAT | | | | | | |
| (Male) | | | | | | |
| VAT/ | 10 | 122992442 | rs11199844 | 5.90E−09 | FGFR2 | Systolic, pulse, and diastolic blood pressure32, type 2 |
| ASAT | | | | | | diabetes (or adjBMI)38, Triglycerides29, Fasting insulin |
| (Male) | | | | | | adjBMI24, BMI, AST/ALT ratio33, coronary artery |
| | | | | | | disease26, HDL-cholesterol, random glucose, ALT |
| VAT/ | 6 | 19947871 | rs70987287 | 8.50E−10 | ID4 | See entry for VAT/ASAT |
| ASAT | | | | | | |
| (Female) | | | | | | |
| VAT/ | 12 | 121319417 | rs59757908 | 4.20E−08 | SPPL3 | HbA1c, pulse pressure |
| ASAT | | | | | | |
| (Female) | | | | | | |
| VAT/ | 14 | 94844947 | rs28929474 | 4.80E−10 | SERPINA1 | AST, AST/ALT ratio, ALT, coronary artery disease, C- |
| GFAT | | | | | | reactive protein, systolic, diastolic, and pulse blood |
| | | | | | | pressure, type 2 diabetes (or adjBMI), trunk fat ratio |
| | | | | | | and leg fat ratio, fasting insulin adjBMI, BMI, BUN, |
| | | | | | | WHR (or adjBMI), triglycerides, total cholesterol, |
| | | | | | | TOAST small artery occlusion, hip circumference, |
| | | | | | | random glucose, serum ApoB, HbA1c adjBMI |
| VAT/ | 1 | 162430821 | rs9660318 | 1.80E−08 | UHMK1 | ratio total to HDL cholesterol, HbA1c, TOAST other |
| GFAT | | | | | | determined |
| (Female) | | | | | | |
| VAT/ | 2 | 116072770 | rs11399916 | 3.70E−08 | DPP10 | any cardiovascular disease41 |
| GFAT | | | | | | |
| (Female) | | | | | | |
| VAT/ | 6 | 32975699 | rs9276981 | 4.60E−08 | HLA-DOA | type 1 diabetes44, WHR (or adjBMI)16, BMI, AST/ALT |
| GFAT | | | | | | ratio33 |
| (Female) | | | | | | |
| ASAT/ | 5 | 55830865 | rs39837 | 2.60E−08 | LINC01948 | AST/ALT ratio33, WHR (or adjBMI)16, type 2 diabetes |
| GFAT | | | | | | adjBMI38, LDL cholesterol, systolic and diastolic blood |
| | | | | | | pressure32, Fasting insulin adjBMI24, HOMA-IR45, coronary |
| | | | | | | artery disease26, eGFR, triglycerides, Stumvoll insulin |
| | | | | | | sensitivity index46, HDL3 cholesterol37 |
| ASAT/ | 14 | 95219657 | rs8006225 | 2.60E−09 | GSC | WHRadjBMI (or WHR)16, HbA1c adjBMI24, systolic blood |
| GFAT | | | | | | pressure32, eGFR31, TOAST small artery occlusion36, |
| | | | | | | HbA1c47, two-hour glucose (or adjBMI)48, coronary artery |
| | | | | | | disease in type 2 diabetes34, total cholesterol, hip |
| | | | | | | circumference8 |
| ASAT/ | 16 | 86424697 | rs1552657 | 4.90E−08 | LINC00917 | Systolic, pulse, and diastolic blood pressure32, |
| GFAT | | | | | | triglycerides, LDL-cholesterol, Stumvoll insulin sensitivity |
| | | | | | | index46, eGFR31, type 2 diabetes (or adjBMI)38, arm fat |
| | | | | | | ratio40, Fasting insulin adjBMI24, coronary artery disease26 |
| ASAT/ | 5 | 55830865 | rs39837 | 9.10E−09 | LINC01948 | See entry for ASAT/GFAT |
| GFAT | | | | | | |
| (Female) | | | | | | |

All nominally significant associations with cardiometabolic traits (P < 0.05) were determined with the Type 2 Diabetes Knowledge Portal. In select cases where a large study made up most of the N for a given association, the individual study citation was included. Note that rs35932591 (VATadj and VATadj (Male)), rs70987287 (VAT/ASAT and VAT/ASAT (Female)), and rs39837 (ASAT/GFAT and ASAT/GFAT (Female)) are duplicated, so 39 unique lead SNPs are presented in this table. BP, GRCh37 position. P-value, BOLT-LMM association P-value.

Supplementary Table 8 Genomic inflation and LD-score intercepts

| | λ_{GC} (Genomic | LD-score |
|---|---|---|
| | inflation) | regression intercept |
| Phenotype (Combined) | | |
| VAT | 1.115 | 1.029 (0.007) |
| ASAT | 1.110 | 1.025 (0.007) |
| GFAT | 1.124 | 1.032 (0.008) |
| VATadj | 1.136 | 1.031 (0.008) |
| ASATadj | 1.125 | 1.026 (0.009) |
| GFATadj | 1.137 | 1.050 (0.009) |
| VAT/ASAT | 1.129 | 1.037 (0.008) |
| VAT/GFAT | 1.135 | 1.032 (0.008) |
| ASAT/GFAT | 1.138 | 1.028 (0.008) |
| Phenotype (Males) | | |
| VAT | 1.055 | 1.006 (0.007) |
| ASAT | 1.059 | 1.019 (0.007) |
| GFAT | 1.067 | 1.028 (0.007) |
| VATadj | 1.077 | 1.010 (0.008) |
| ASATadj | 1.079 | 1.021 (0.007) |
| GFATadj | 1.077 | 1.031 (0.008) |
| VAT/ASAT | 1.081 | 1.019 (0.007) |
| VAT/GFAT | 1.072 | 1.005 (0.007) |
| ASAT/GFAT | 1.061 | 1.017 (0.006) |
| Phenotype (Females) | | |
| VAT | 1.084 | 1.023 (0.006) |
| ASAT | 1.082 | 1.019 (0.007) |
| GFAT | 1.072 | 1.017 (0.008) |
| VATadj | 1.069 | 1.024 (0.007) |
| ASATadj | 1.090 | 1.023 (0.008) |
| GFATadj | 1.104 | 1.031 (0.007) |
| VAT/ASAT | 1.075 | 1.026 (0.007) |
| VAT/GFAT | 1.090 | 1.026 (0.007) |
| ASAT/GFAT | 1.109 | 1.030 (0.008) |

Genomic inflation parameters (λ_{GC}) were computed from GWAS summary statistics including all directly genotyped and imputed SNPs. LD-score regression intercepts were computed using the original LD model with HapMap3 SNPs and default settings.¹⁴

Supplementary Table 9 Genetic correlations between adiposity traits in males and females

| Phenotype | Genetic correlation (r_{g}) between male and female summary statistics |
|---|---|
| VAT | 0.73 (0.09) |
| ASAT | 0.90 (0.10) |
| GFAT | 1.04 (0.11) |
| VATadj | 0.87 (0.08) |
| ASATadj | 0.80 (0.09) |
| GFATadj | 0.79 (0.08) |
| VAT/ASAT | 0.83 (0.08) |
| VAT/GFAT | 0.70 (0.08) |
| ASAT/GFAT | 0.80 (0.08) |

### Example 3 References

1. Agrawal S, Klarqvist M D R, Diamant N, et al. Association of machine learning-derived measures of body fat distribution in >40,000 individuals with cardiometabolic diseases. medRxiv 2021; 2021.05.07.21256854.
2. Leinhard O D, Johansson A, Rydell J, et al. Quantitative abdominal fat estimation using MRI. In: 2008 19th International Conference on Pattern Recognition. 2008. p. 1-4.
3. Borga M, Thomas E L, Romu T, et al. Validation of a fast method for quantification of intra-abdominal and subcutaneous adipose tissue for large-scale human studies. NMR Biomed 2015; 28(12): 1747-53.
4. West J, Leinhard O D, Romu T, et al. Feasibility of MR-Based Body Composition Analysis in Large Scale Population Studies. PLOS ONE 2016; 11(9):e0163332.
5. Borga M, West J, Bell J D, et al. Advanced body composition assessment: from body mass index to body composition profiling. J Investig Med Off Publ Am Fed Clin Res 2018; 66(5):1-9.
6. Linge J, Borga M, West J, et al. Body Composition Profiling in the UK Biobank Imaging Study. Obes Silver Spring Md 2018; 26(11):1785-95.
7. Linge J, Whitcher B, Borga M, Dahlqvist Leinhard O. Sub-phenotyping Metabolic Disorders Using Body Composition: An Individualized, Nonparametric Approach Utilizing Large Data Sets. Obes Silver Spring Md 2019; 27(7):1190-9.
8. Shungin D, Winkler T W, Croteau-Chonka D C, et al. New genetic loci link adipose and insulin biology to body fat distribution. Nature 2015; 518(7538):187-96.
9. Rüeger S, McDaid A, Kutalik Z. Evaluation and application of summary statistic imputation to discover new height-associated loci. PLoS Genet 2018; 14(5):e1007371.
10. Kichaev G, Bhatia G, Loh P-R, et al. Leveraging Polygenic Functional Enrichment to Improve GWAS Power. Am J Hum Genet 2019; 104(1):65-75.
11. Christakoudi S, Evangelou E, Riboli E, Tsilidis K K. GWAS of allometric body-shape indices in U K Biobank identifies loci suggesting associations with morphogenesis, organogenesis, adrenal cell renewal and cancer. Sci Rep 2021; 11(1):10688.
12. Chu A Y, Deng X, Fisher V A, et al. Multiethnic genome-wide meta-analysis of ectopic fat depots identifies loci associated with adipocyte development and differentiation. Nat Genet 2017; 49(1):125-30.
13. Fox C S, White C C, Lohman K, et al. Genome-wide association of pericardial fat identifies a unique locus for ectopic fat. PLoS Genet 2012; 8(5):e1002705.
14. Bulik-Sullivan B K, Loh P-R, Finucane H K, et al. L D Score regression distinguishes confounding from polygenicity in genome-wide association studies. Nat Genet 2015; 47(3):291-5.
15. Bulik-Sullivan B, Finucane H K, Anttila V, et al. An atlas of genetic correlations across human diseases and traits. Nat Genet 2015; 47(11):1236-41.
16. Pulit S L, Stoneman C, Morris A P, et al. Meta-analysis of genome-wide association studies for body fat distribution in 694 649 individuals of European ancestry. Hum Mol Genet 2019; 28(1): 166-74.
17. Finucane H K, Reshef Y A, Anttila V, et al. Heritability enrichment of specifically expressed genes identifies disease-relevant tissues and cell types. Nat Genet 2018; 50(4):621-9.
18. Loh P-R, Bhatia G, Gusev A, et al. Contrasting genetic architectures of schizophrenia and other complex diseases using fast variance-components analysis. Nat Genet 2015; 47(12):1385-92.
19. Loh P-R, Tucker G, Bulik-Sullivan B K, et al. Efficient Bayesian mixed-model analysis increases association power in large cohorts. Nat Genet 2015; 47(3):284-90.
20. Loh P-R, Kichaev G, Gazal S, Schoech A P, Price A L. Mixed-model association for biobank-scale datasets. Nat Genet 2018; 50(7):906-8.
21. Gazal S, Finucane H K, Furlotte N A, et al. Linkage disequilibrium-dependent architecture of human complex traits shows action of negative selection. Nat Genet 2017; 49(10):1421-7.
22. Prokopenko I, Poon W, Magi R, et al. A central role for GRB10 in regulation of islet function in man. PLoS Genet 2014; 10(4):e1004235.
23. Wood A R, Jonsson A, Jackson A U, et al. A Genome-Wide Association Study of IVGTT-Based Measures of First-Phase Insulin Secretion Refines the Underlying Physiology of Type 2 Diabetes Variants. Diabetes 2017; 66(8):2296-309.
24. Chen J, Spracklen C N, Marenne G, et al. The trans-ancestral genomic architecture of glycemic traits. Nat Genet 2021; 53(6):840-60.
25. Riveros-McKay F, Mistry V, Bounds R, et al. Genetic architecture of human thinness compared to severe obesity. PLoS Genet 2019; 15(1):e1007603.
26. van der Harst P, Verweij N. Identification of 64 Novel Genetic Loci Provides an Expanded View on the Genetic Architecture of Coronary Artery Disease. Circ Res 2018; 122(3):433-43.
27. Graff M, Scott R A, Justice A E, et al. Genome-wide physical activity interactions in adiposity—A meta-analysis of 200,452 adults. PLoS Genet 2017; 13(4):e1006528.
28. Justice A E, Winkler T W, Feitosa M F, et al. Genome-wide meta-analysis of 241,258 adults accounting for smoking behaviour identifies novel loci for obesity traits. Nat Commun 2017; 8:14977.
29. Forgetta V, Jiang L, Vulpescu N A, et al. An Effector Index to Predict Causal Genes at GWAS Loci [Internet]. 2021 [cited 2021 Nov. 7]. Available from: https://www.biorxiv.org/content/10.1101/2020.06.28.171561v2
30. Kanai M, Akiyama M, Takahashi A, et al. Genetic analysis of quantitative traits in the Japanese population links cell types to complex human diseases. Nat Genet 2018; 50(3):390-400.
31. Wuttke M, Li Y, Li M, et al. A catalog of genetic loci associated with kidney function from analyses of a million individuals. Nat Genet 2019; 51(6):957-72.
32. Evangelou E, Warren H R, Mosen-Ansorena D, et al. Genetic analysis of over 1 million people identifies 535 new loci associated with blood pressure traits. Nat Genet 2018; 50(10):1412-25.
33. Sinnott-Armstrong N, Tanigawa Y, Amar D, et al. Genetics of 35 blood and urine biomarkers in the UK Biobank. Nat Genet 2021; 53(2):185-94.
34. Zhao W, Rasheed A, Tikkanen E, et al. Identification of new susceptibility loci for type 2 diabetes and shared etiological pathways with coronary heart disease. Nat Genet 2017; 49(10): 1450-7.
35. Yengo L, Sidorenko J, Kemper K E, et al. Meta-analysis of genome-wide association studies for height and body mass index in −700000 individuals of European ancestry. Hum Mol Genet 2018; 27(20):3641-9.
36. Malik R, Chauhan G, Traylor M, et al. Multiancestry genome-wide association study of 520,000 subjects identifies 32 loci associated with stroke and stroke subtypes. Nat Genet 2018; 50(4):524-37.
37. Locke A E, Steinberg K M, Chiang C W K, et al. Exome sequencing of Finnish isolates enhances rare-variant association power. Nature 2019; 572(7769):323-8.
38. Mahajan A, Taliun D, Thurner M, et al. Fine-mapping type 2 diabetes loci to single-variant resolution using high-density imputation and islet-specific epigenome maps. Nat Genet 2018; 50(11):1505-13.
39. Gorski M, van der Most P J, Teumer A, et al. 1000 Genomes-based meta-analysis identifies 10 novel loci for kidney function. Sci Rep 2017; 7:45040.
40. Rask-Andersen M, Karlsson T, Ek APPLICANTS, Johansson A. Genome-wide association study of body fat distribution identifies adiposity loci and sex-specific genetic effects. Nat Commun 2019; 10(1):339.
41. Guindo-Martinez M, Amela R, Bonds-Guarch S, et al. The impact of non-additive genetic associations on age-related complex diseases. Nat Commun 2021; 12(1):2436.
42. Gurdasani D, Carstensen T, Fatumo S, et al. Uganda Genome Resource Enables Insights into Population History and Genomic Discovery in Africa. Cell 2019; 179(4):984-1002.e36.
43. Nagy R, Boutin T S, Marten J, et al. Exploration of haplotype research consortium imputation for genome-wide association studies in 20,032 Generation Scotland participants. Genome Med 2017; 9(1):23.
44. Robertson C C, Inshaw J R J, Onengut-Gumuscu S, et al. Fine-mapping, trans-ancestral and genomic analyses identify causal variants, cells, genes and drug targets for type 1 diabetes. Nat Genet 2021; 53 (7): 962-71.

45. Dupuis J, Langenberg C, Prokopenko I, et al. New genetic loci implicated in fasting glucose homeostasis and their impact on type 2 diabetes risk. Nat Genet 2010; 42(2):105-16.
46. Walford G A, Gustafsson S, Rybin D, et al. Genome-Wide Association Study of the Modified Stumvoll Insulin Sensitivity Index Identifies BCL2 and FAM19A2 as Novel Insulin Sensitivity Loci. Diabetes 2016; 65(10):3200-11.
47. Wheeler E, Leong A, Liu C-T, et al. Impact of common genetic determinants of Hemoglobin Alc on type 2 diabetes risk and diagnosis in ancestrally diverse populations: A transethnic genome-wide meta-analysis. PLoS Med 2017; 14(9):e1002383.
48. Saxena R, Hivert M-F, Langenberg C, et al. Genetic variation in GIPR influences the glucose and insulin responses to an oral glucose challenge. Nat Genet 2010; 42(2):142-8.

### Supplementary Data

Full Supplementary Data is available at Agrawal S, Wang M, Klarqvist M D R, et al. Inherited basis of visceral, abdominal subcutaneous and gluteofemoral fat depots. Nat Commun. 2022; 13(1):3771.

### Supplementary Data 3. Lead SNPs

VAT—visceral adipose tissue, ASAT—abdominal subcutaneous adipose tissue, GFAT−gluteofemoral adipose tissue volumes.

CHR—chromosome, BP—GRCh37 position, EAF—effect allele frequency, BETA—effect size, SE standard error of effect size.

For VATadj, ASATadj, and GFATadj results, effect sizes for unadjusted fat depots, BMI, and height are included in Supplementary Data 22.

Full table available at Agrawal S, Wang M, Klarqvist M D R, et al. Inherited basis of visceral, abdominal subcutaneous and gluteofemoral fat depots. Nat Commun. 2022; 13(1):3771.

| | | | | Effect | Other | | | | | Nearest |
|---|---|---|---|---|---|---|---|---|---|---|
| Trait | CHR | BP | SNP | Allele | Allele | EAF | BETA | SE | P-value | Gene |
| VAT | 3 | 49799046 | 3:49799046_CA_C | CA | C | 0.547 | −0.042 | 0.007 | 2.10E−08 | IP6K1 |
| VAT | 5 | 55802127 | 5:55802127_TCAAGGATTCCTTGACTTAAG_T | TCAAGGATTCCTTGACTTAAG | T | 0.201 | 0.049 | 0.009 | 2.90E−08 | LINC01948 |
| | | | (SEQ ID NO: 20) | (SEQ ID NO: 21) | | | | | | |
| VAT | 8 | 25464670 | rs73221948 | G | T | 0.709 | 0.054 | 0.008 | 2.60E−11 | CDCA2 |
| VAT | 16 | 53806453 | rs56094641 | A | G | 0.602 | −0.046 | 0.007 | 3.30E−10 | FTO |
| VAT | 19 | 18338709 | rs62120394 | G | A | 0.716 | −0.048 | 0.008 | 1.10E−09 | PDE4C |
| VAT | 19 | 33785832 | 19:33785832_CA_C | CA | C | 0.824 | 0.06 | 0.01 | 1.20E−09 | CEBPA |
| VAT | 19 | 33893008 | rs3786897 | A | G | 0.577 | −0.044 | 0.007 | 5.90E−10 | PEPD |
| VAT(Male) | 17 | 7103861 | rs34670319 | C | CT | 0.443 | −0.057 | 0.01 | 4.40E−08 | DLG4 |
| VAT(Female) | 2 | 60036763 | rs147603433 | G | A | 0.968 | −0.157 | 0.028 | 3.80E−08 | LINC01793 |
| VAT(Female) | 19 | 49279612 | rs4801774 | C | T | 0.274 | −0.064 | 0.012 | 4.50E−08 | FGF21 |
| ASAT | 2 | 417167 | rs62106258 | T | C | 0.951 | 0.09 | 0.016 | 2.90E−08 | LINC01874 |
| ASAT | 6 | 50968152 | rs1325033 | T | C | 0.465 | −0.041 | 0.007 | 8.50E−09 | TFAP2B |
| ASAT | 8 | 77222269 | rs7461961 | G | A | 0.463 | −0.039 | 0.007 | 4.90E−08 | LINC01111 |
| ASAT | 16 | 53806453 | rs56094641 | A | G | 0.602 | −0.071 | 0.007 | 1.30E−22 | FTO |
| ASAT | 19 | 18338709 | rs62120394 | G | A | 0.716 | −0.045 | 0.008 | 7.70E−09 | PDE4C |
| ASAT | 20 | 3139717 | rs79818747 | A | G | 0.997 | −0.431 | 0.07 | 1.50E−09 | LZTS3 |
| ASAT(Male) | 16 | 53806453 | rs56094641 | A | G | 0.596 | −0.081 | 0.01 | 8.30E−15 | FTO |
| ASAT(Female) | 16 | 53802494 | rs11642015 | C | T | 0.608 | −0.059 | 0.01 | 7.20E−09 | FTO |
| GFAT | 1 | 219673705 | rs2820468 | A | G | 0.341 | 0.048 | 0.007 | 1.10E−10 | LYPLAL1-AS1 |
| GFAT | 2 | 165544573 | rs200472737 | GAA | G | 0.597 | −0.046 | 0.007 | 9.40E−11 | COBLL1 |
| GFAT | 2 | 165642448 | rs355906 | G | A | 0.557 | −0.041 | 0.007 | 6.30E−09 | COBLL1 |
| GFAT | 2 | 219699999 | rs78058190 | G | A | 0.95 | 0.108 | 0.018 | 1.30E−09 | PRKAG3 |
| GFAT | 2 | 227099854 | rs2972147 | T | C | 0.35 | 0.048 | 0.007 | 4.50E−11 | LOC646736 |
| GFAT | 5 | 55841824 | rs16885714 | A | G | 0.902 | 0.066 | 0.012 | 3.70E−08 | C5orf67 |
| GFAT | 6 | 26207175 | rs9379833 | C | A | 0.728 | 0.045 | 0.008 | 4.50E−09 | H4C5 |
| GFAT | 6 | 31311376 | rs9265830 | A | G | 0.321 | 0.043 | 0.008 | 2.40E−08 | HLA-B |
| GFAT | 6 | 32509842 | rs115250958 | C | A | 0.886 | −0.068 | 0.012 | 2.50E−08 | HLA-DRB5 |
| GFAT | 6 | 34211341 | rs35381162 | GT | G | 0.033 | 0.11 | 0.02 | 3.90E−08 | HMGA1 |
| GFAT | 6 | 34746957 | rs529311472 | G | GT | 0.733 | −0.044 | 0.008 | 2.90E−08 | SNRPC |
| GFAT | 6 | 35504030 | rs141958096 | C | T | 0.982 | −0.145 | 0.027 | 3.30E−08 | TULP1 |
| GFAT | 6 | 43757082 | rs4711750 | T | A | 0.5 | 0.054 | 0.007 | 5.80E−15 | VEGFA |
| GFAT | 6 | 50968152 | rs1325033 | T | C | 0.465 | −0.041 | 0.007 | 7.50E−09 | TFAP2B |
| GFAT | 6 | 105373111 | 6:105373111_CT_C | CT | C | 0.683 | −0.042 | 0.008 | 1.60E−08 | LIN28B-AS1 |
| GFAT | 6 | 127454893 | rs72959041 | G | A | 0.953 | 0.094 | 0.017 | 1.90E−08 | RSPO3 |
| GFAT | 6 | 160774459 | rs487060 | C | T | 0.53 | −0.042 | 0.007 | 9.10E−10 | SLC22A3 |
| GFAT | 11 | 95840436 | rs1074742 | A | G | 0.401 | 0.041 | 0.007 | 1.40E−08 | MAML2 |
| GFAT | 12 | 123024476 | rs147730268 | G | T | 0.913 | 0.069 | 0.013 | 2.90E−08 | KNTC1 |
| GFAT | 12 | 124344710 | rs138756410 | T | C | 0.986 | −0.172 | 0.031 | 3.00E−08 | DNAH10 |
| GFAT | 12 | 124409502 | rs7133378 | G | A | 0.68 | −0.053 | 0.008 | 7.30E−13 | DNAH10 |
| GFAT | 12 | 124508758 | rs825453 | A | T | 0.394 | 0.056 | 0.007 | 1.20E−14 | ZNF664 |
| GFAT | 12 | 125092343 | rs4765159 | A | G | 0.018 | 0.146 | 0.027 | 3.50E−08 | NCOR2 |
| GFAT | 16 | 53806453 | rs56094641 | A | G | 0.602 | −0.052 | 0.007 | 1.20E−12 | FTO |
| GFAT | 19 | 34019403 | 19:34019403_GAC_G | GAC | G | 0.621 | 0.042 | 0.007 | 2.00E−08 | PEPD |
| GFAT | 20 | 3139717 | rs79818747 | A | G | 0.997 | −0.434 | 0.07 | 9.70E−10 | LZTS3 |
| GFAT | 22 | 38505347 | rs6001008 | G | A | 0.569 | −0.046 | 0.007 | 1.90E−10 | BAIAP2L2 |
| GFAT(Male) | 2 | 227047771 | rs2943653 | C | T | 0.325 | 0.065 | 0.011 | 2.90E−09 | LOC646736 |
| GFAT(Male) | 16 | 53806453 | rs56094641 | A | G | 0.596 | −0.06 | 0.01 | 6.30E−09 | FTO |
| GFAT(Female) | 2 | 165528876 | rs13389219 | C | T | 0.608 | −0.064 | 0.01 | 2.50E−10 | COBLL1 |
| GFAT(Female) | 4 | 819323 | rs146623665 | C | T | 0.953 | 0.135 | 0.023 | 9.60E−09 | CPLX1 |
| GFAT(Female) | 6 | 43757082 | rs4711750 | T | A | 0.5 | 0.06 | 0.01 | 1.00E−09 | VEGFA |
| GFAT(Female) | 6 | 105373111 | 6:105373111_CT_C | CT | C | 0.685 | −0.061 | 0.011 | 1.20E−08 | LIN28B-AS1 |
| GFAT(Female) | 12 | 124409502 | rs7133378 | G | A | 0.68 | −0.074 | 0.01 | 8.70E−13 | DNAH10 |
| GFAT(Female) | 12 | 124508758 | rs825453 | A | T | 0.393 | 0.065 | 0.01 | 1.00E−10 | ZNF664 |
| VATadj | 1 | 11220187 | rs12089366 | C | T | 0.777 | 0.058 | 0.009 | 9.40E−12 | MTOR |
| VATadj | 1 | 204430834 | rs56006999 | C | T | 0.821 | 0.054 | 0.009 | 3.60E−09 | PIK3C2B |
| VATadj | 2 | 121310704 | rs35932591 | C | T | 0.879 | 0.061 | 0.011 | 3.80E−08 | LINC01101 |
| VATadj | 2 | 219191256 | rs3731861 | T | C | 0.622 | −0.038 | 0.007 | 4.70E−08 | PNKD |
| VATadj | 3 | 156797225 | rs56082403 | T | C | 0.593 | −0.056 | 0.007 | 6.90E−14 | LINC02029 |
| VATadj | 5 | 55794632 | rs30351 | G | A | 0.264 | 0.071 | 0.008 | 1.10E−16 | LINC01948 |
| VATadj | 5 | 173307328 | rs72810972 | G | T | 0.716 | −0.054 | 0.008 | 2.30E−12 | CPEB4 |
| VATadj | 6 | 31325115 | rs9266218 | A | G | 0.385 | −0.057 | 0.007 | 5.30E−14 | HLA-B |
| VATadj | 6 | 32479878 | rs76072243 | T | C | 0.562 | −0.055 | 0.007 | 4.90E−14 | HLA-DRB5 |
| VATadj | 6 | 32509842 | rs115250958 | C | A | 0.886 | 0.074 | 0.012 | 7.60E−10 | HLA-DRB5 |
| VATadj | 6 | 32625967 | rs2858856 | C | A | 0.721 | −0.047 | 0.008 | 8.80E−09 | HLA-DQB1 |
| VATadj | 6 | 34177853 | rs185139895 | G | A | 0.958 | −0.1 | 0.018 | 3.30E−09 | MIR6835 |
| VATadj | 6 | 43757896 | rs998584 | C | A | 0.517 | −0.057 | 0.007 | 1.80E−15 | VEGFA |
| VATadj | 6 | 127419811 | rs2800736 | G | A | 0.465 | −0.043 | 0.007 | 4.80E−10 | RSPO3 |
| VATadj | 6 | 127440047 | rs577721086 | T | C | 0.952 | −0.118 | 0.017 | 5.20E−13 | RSPO3 |
| VATadj | 6 | 139829695 | rs5880430 | T | TTGAA | 0.37 | 0.06 | 0.007 | 2.20E−16 | LINC01625 |
| VATadj | 7 | 28197805 | rs149643430 | C | CACACAG | 0.424 | 0.043 | 0.007 | 1.50E−08 | JAZF1 |
| VATadj | 8 | 25464690 | rs11992444 | G | T | 0.492 | −0.078 | 0.007 | 1.30E−29 | CDCA2 |
| VATadj | 8 | 25917711 | rs4872393 | G | A | 0.773 | −0.06 | 0.008 | 2.00E−12 | EBF2 |
| VATadj | 10 | 25767521 | rs1329254 | C | T | 0.37 | 0.042 | 0.007 | 1.40E−08 | GPR158 |
| VATadj | 11 | 32479807 | rs11031796 | G | A | 0.612 | 0.052 | 0.007 | 5.10E−14 | WT1-AS |
| VATadj | 11 | 46610325 | 11:46610325_CA_C | CA | C | 0.793 | 0.057 | 0.009 | 2.20E−10 | AMBRA1 |
| VATadj | 11 | 69195097 | rs7933253 | T | C | 0.048 | 0.098 | 0.017 | 1.30E−08 | LOC102724265 |
| VATadj | 12 | 124409502 | rs7133378 | G | A | 0.68 | 0.046 | 0.008 | 6.60E−10 | DNAH10 |
| VATadj | 12 | 124503803 | 12:124503803_CAA_C | CAA | C | 0.438 | −0.039 | 0.007 | 2.00E−08 | ZNF664 |
| VATadj | 19 | 33785832 | 19:33785832_CA_C | CA | C | 0.824 | 0.094 | 0.01 | 3.30E−21 | CEBPA |
| VATadj | 19 | 33805720 | rs7250362 | C | G | 0.41 | 0.038 | 0.007 | 3.60E−08 | CEBPA-DT |
| VATadj | 19 | 33832399 | rs55865721 | G | A | 0.927 | 0.102 | 0.014 | 4.90E−14 | CEBPA-DT |
| VATadj | 19 | 33890838 | rs10406327 | C | G | 0.524 | −0.071 | 0.007 | 3.30E−24 | PEPD |
| VATadj | 21 | 35593827 | rs28451064 | G | A | 0.868 | −0.069 | 0.011 | 2.40E−11 | LINC00310 |
| VATadj(Male) | 1 | 11099387 | 1:11099387_GTGGATGGATGGA_G | GTGGATGGATGGA | G | 0.475 | −0.07 | 0.012 | 9.10E−09 | MASP2 |
| | | | (SEQ ID NO: 22) | (SEQ ID NO: 23) | | | | | | |
| VATadj(Male) | 2 | 121310704 | rs35932591 | C | T | 0.88 | 0.086 | 0.016 | 3.90E−08 | LINC01101 |
| VATadj(Male) | 5 | 55794632 | rs30351 | G | A | 0.265 | 0.088 | 0.012 | 3.70E−13 | LINC01948 |
| VATadj(Male) | 5 | 173392398 | rs10054063 | A | T | 0.692 | −0.075 | 0.011 | 2.70E−11 | CPEB4 |
| VATadj(Male) | 6 | 32468804 | rs113602321 | T | A | 0.656 | −0.071 | 0.012 | 2.40E−09 | HLA-DRB5 |
| VATadj(Male) | 6 | 43757896 | rs998584 | C | A | 0.517 | −0.064 | 0.01 | 9.80E−10 | VEGFA |
| VATadj(Male) | 8 | 25464690 | rs11992444 | G | T | 0.492 | −0.079 | 0.01 | 1.60E−14 | CDCA2 |
| VATadj(Male) | 11 | 32470775 | rs35641603 | C | T | 0.833 | 0.087 | 0.014 | 2.00E−10 | WT1-AS |
| VATadj(Male) | 19 | 33834096 | rs73026242 | A | G | 0.93 | 0.109 | 0.02 | 3.50E−08 | CEBPG |
| VATadj(Male) | 19 | 33890838 | rs10406327 | C | G | 0.526 | −0.066 | 0.01 | 5.50E−11 | PEPD |
| VATadj(Male) | 21 | 35593827 | rs28451064 | G | A | 0.868 | −0.093 | 0.015 | 1.30E−09 | LINC00310 |
| VATadj(Female) | 1 | 204430834 | rs56006999 | C | T | 0.821 | 0.076 | 0.013 | 8.40E−09 | PIK3C2B |
| VATadj(Female) | 3 | 56901687 | rs1500714 | C | G | 0.854 | 0.081 | 0.015 | 1.80E−08 | ARHGEF3 |
| VATadj(Female) | 3 | 156795468 | rs13322435 | A | G | 0.589 | −0.064 | 0.01 | 1.40E−10 | LINC02029 |
| VATadj(Female) | 6 | 31346805 | rs9266627 | A | G | 0.661 | −0.059 | 0.011 | 1.60E−08 | MICA-AS1 |
| VATadj(Female) | 6 | 32621590 | 6:32621590_T_C | T | C | 0.65 | −0.075 | 0.011 | 4.30E−10 | HLA-DQB1 |
| VATadj(Female) | 6 | 127440047 | rs577721086 | T | C | 0.952 | −0.159 | 0.023 | 1.20E−11 | RSPO3 |
| VATadj(Female) | 6 | 139842576 | rs4052908 | A | AATT | 0.364 | 0.079 | 0.01 | 4.10E−14 | LINC01625 |
| VATadj(Female) | 8 | 25464670 | rs73221948 | G | T | 0.708 | 0.094 | 0.011 | 1.40E−16 | CDCA2 |
| VATadj(Female) | 9 | 107722705 | rs1962883 | C | T | 0.528 | −0.062 | 0.01 | 1.10E−09 | ABCA1 |
| VATadj(Female) | 12 | 122820960 | 12:122820960_TAA_T | TAA | T | 0.214 | 0.07 | 0.012 | 1.60E−08 | CLIP1 |
| VATadj(Female) | 12 | 124409502 | rs7133378 | G | A | 0.68 | 0.075 | 0.011 | 8.00E−13 | DNAH10 |
| VATadj(Female) | 12 | 124503803 | 12:124503803_CAA_C | CAA | C | 0.436 | −0.062 | 0.01 | 1.20E−09 | ZNF664 |
| VATadj(Female) | 19 | 33785832 | 19:33785832_CA_C | CA | C | 0.825 | 0.113 | 0.014 | 7.40E−15 | CEBPA |
| VATadj(Female) | 19 | 33890838 | rs10406327 | C | G | 0.522 | −0.08 | 0.01 | 7.70E−16 | PEPD |
| VATadj(Female) | 19 | 34001331 | rs73041147 | A | C | 0.929 | 0.103 | 0.019 | 3.40E−08 | PEPD |
| VATadj(Female) | 19 | 34014316 | rs33845 | A | G | 0.222 | 0.069 | 0.012 | 1.30E−08 | PEPD |
| ASATadj | 1 | 119508412 | rs1779445 | T | C | 0.194 | −0.049 | 0.009 | 1.90E−08 | TBX15 |
| ASATadj | 1 | 201016296 | rs3850625 | G | A | 0.882 | −0.079 | 0.011 | 1.80E−12 | CACNA1S |
| ASATadj | 1 | 203516075 | rs6685593 | T | A | 0.506 | −0.057 | 0.007 | 5.20E−15 | OPTC |
| ASATadj | 1 | 219788530 | rs7538503 | A | G | 0.71 | −0.047 | 0.008 | 8.40E−10 | ZC3H11B |
| ASATadj | 2 | 227099975 | rs2943647 | T | C | 0.348 | 0.043 | 0.007 | 5.80E−09 | LOC646736 |
| ASATadj | 3 | 12360357 | rs527620413 | G | GT | 0.875 | −0.071 | 0.011 | 6.80E−11 | PPARG |
| ASATadj | 3 | 38467753 | rs7649153 | T | A | 0.329 | 0.042 | 0.008 | 2.70E−08 | XYLB |
| ASATadj | 3 | 156795468 | rs13322435 | A | G | 0.591 | 0.057 | 0.007 | 2.40E−15 | LINC02029 |
| ASATadj | 5 | 52777864 | rs55744247 | G | A | 0.796 | −0.053 | 0.009 | 5.10E−10 | FST |
| ASATadj | 5 | 55860866 | rs3936510 | G | T | 0.798 | −0.063 | 0.009 | 5.00E−13 | C5orf67 |
| ASATadj | 6 | 126801144 | rs1159619 | C | A | 0.545 | 0.046 | 0.007 | 1.20E−10 | CENPW |
| ASATadj | 7 | 130432913 | rs553015785 | A | AT | 0.517 | −0.048 | 0.007 | 3.30E−11 | KLF14 |
| ASATadj | 8 | 25464670 | rs73221948 | G | T | 0.709 | −0.05 | 0.008 | 2.90E−09 | CDCA2 |
| ASATadj | 9 | 1044400 | rs2048235 | C | T | 0.384 | 0.041 | 0.007 | 4.10E−08 | LINC01230 |
| ASATadj | 9 | 1052722 | rs6474550 | G | T | 0.66 | 0.045 | 0.008 | 1.30E−09 | DMRT2 |
| ASATadj | 15 | 62757857 | rs17205757 | A | G | 0.674 | −0.042 | 0.008 | 3.20E−08 | MIR6085 |
| ASATadj | 15 | 84575367 | rs768397327 | CCACACACCA | C | 0.484 | −0.06 | 0.007 | 2.20E−17 | ADAMTSL3 |
| | | | | (SEQ ID NO: 24) | | | | | | |
| ASATadj | 15 | 85091836 | 15:85091836_CA_C | CA | C | 0.75 | −0.047 | 0.008 | 2.20E−17 | UBE2Q2P1 |
| ASATadj | 17 | 404300 | rs8077609 | A | C | 0.674 | 0.042 | 0.008 | 1.10E−08 | ARL17B, ARL17A |
| ASATadj | 17 | 76324751 | rs4444401 | A | G | 0.473 | −0.04 | 0.007 | 4.20E−08 | SOCS3 |
| ASATadj | 19 | 18324329 | rs2302209 | C | T | 0.719 | −0.046 | 0.008 | 3.40E−09 | PDE4C |
| ASATadj(Male) | 1 | 219769374 | rs6704389 | A | C | 0.828 | 0.078 | 0.014 | 9.50E−09 | ZC3H11B |
| ASATadj(Male) | 1 | 219788530 | rs7538503 | A | G | 0.713 | −0.062 | 0.011 | 2.70E−08 | ZC3H11B |
| ASATadj(Male) | 2 | 227099534 | rs2943646 | A | G | 0.349 | 0.081 | 0.011 | 1.10E−13 | LOC646736 |
| ASATadj(Male) | 3 | 12360357 | rs527620413 | G | GT | 0.873 | −0.093 | 0.016 | 4.40E−09 | PPARG |
| ASATadj(Male) | 3 | 38460062 | rs6807940 | C | G | 0.398 | 0.057 | 0.01 | 3.30E−08 | XYLB |
| ASATadj(Male) | 3 | 156795525 | rs9854955 | A | G | 0.596 | 0.069 | 0.011 | 2.00E−11 | LINC02029 |
| ASATadj(Male) | 15 | 84575367 | rs768397327 | CCACACACCA | C | 0.483 | −0.069 | 0.01 | 1.70E−11 | ADAMTSL3 |
| | | | | (SEQ ID NO: 24) | | | | | | |
| ASATadj(Male) | 17 | 62016727 | rs112489358 | C | CACACATATAT | 0.464 | 0.06 | 0.011 | 2.30E−08 | SCN4A |
| | | | | | (SEQ ID NO: 25) | | | | | |
| ASATadj(Female) | 1 | 116916645 | rs749166380 | CT | C | 0.102 | 0.102 | 0.018 | 2.20E−08 | ATP1A1 |
| ASATadj(Female) | 1 | 203510048 | rs6691427 | G | C | 0.509 | −0.068 | 0.01 | 5.10E−11 | OPTC |
| ASATadj(Female) | 5 | 55860907 | 5:55860907_GC_G | GC | G | 0.817 | −0.104 | 0.013 | 9.30E−16 | C5orf67 |
| ASATadj(Female) | 6 | 43757896 | rs998584 | C | A | 0.517 | −0.068 | 0.01 | 1.60E−11 | VEGFA |
| ASATadj(Female) | 7 | 130029508 | rs1558919 | A | T | 0.657 | 0.061 | 0.011 | 7.50E−09 | CPA1 |
| ASATadj(Female) | 7 | 130432913 | rs553015785 | A | AT | 0.519 | −0.084 | 0.01 | 8.40E−17 | KLF14 |
| ASATadj(Female) | 8 | 58352327 | rs776481989 | ATAAT | A | 0.998 | 0.795 | 0.134 | 8.60E−09 | LOC101929488 |
| ASATadj(Female) | 15 | 84570588 | 15:84570588_TGA_T | TGA | T | 0.476 | −0.058 | 0.01 | 8.20E−09 | ADAMTSL3 |
| GFATadj | 1 | 9336116 | rs72641832 | C | A | 0.751 | 0.058 | 0.008 | 5.20E−12 | H6PD |
| GFATadj | 1 | 149906413 | rs11205303 | T | C | 0.596 | −0.039 | 0.007 | 1.70E−08 | MTMR11 |
| GFATadj | 1 | 219754012 | rs559230165 | C | CT | 0.713 | −0.071 | 0.008 | 1.70E−19 | LYPLAL1-AS1 |
| GFATadj | 2 | 3648186 | rs7588285 | C | G | 0.188 | 0.053 | 0.009 | 1.40E−08 | COLEC11 |
| GFATadj | 2 | 165528876 | rs13389219 | C | T | 0.607 | −0.073 | 0.007 | 3.00E−23 | COBLL1 |
| GFATadj | 2 | 165566877 | rs3820981 | A | G | 0.56 | −0.053 | 0.007 | 1.50E−12 | COBLL1 |
| GFATadj | 2 | 165645349 | rs34224594 | C | CA | 0.614 | −0.046 | 0.008 | 2.80E−09 | COBLL1 |
| GFATadj | 2 | 219699999 | rs78058190 | G | A | 0.951 | 0.115 | 0.019 | 3.70E−10 | PRKAG3 |
| GFATadj | 2 | 226768344 | 2:226768344_CA_C | CA | C | 0.193 | −0.051 | 0.009 | 2.60E−08 | NYAP2 |
| GFATadj | 2 | 227068080 | rs2943634 | A | C | 0.327 | 0.075 | 0.008 | 4.80E−23 | LOC646736 |
| GFATadj | 2 | 227205783 | rs35414396 | A | G | 0.739 | 0.05 | 0.008 | 2.40E−09 | LOC646736 |
| GFATadj | 3 | 12396913 | rs71304101 | G | A | 0.879 | −0.062 | 0.011 | 1.70E−09 | PPARG |
| GFATadj | 3 | 12493347 | rs9855622 | C | T | 0.878 | 0.063 | 0.011 | 8.80E−09 | PPARG |
| GFATadj | 3 | 38541318 | rs2300669 | C | A | 0.615 | −0.042 | 0.007 | 4.40E−09 | EXOG |
| GFATadj | 3 | 47069275 | rs199874557 | T | TG | 0.587 | −0.039 | 0.007 | 1.80E−08 | SETD2 |
| GFATadj | 3 | 150066540 | rs62271373 | T | A | 0.942 | 0.123 | 0.015 | 4.80E−15 | LINC01214 |
| GFATadj | 3 | 196818853 | rs13099700 | A | G | 0.722 | 0.047 | 0.008 | 7.90E−09 | DLG1 |
| GFATadj | 4 | 4990298 | rs4450871 | A | G | 0.555 | −0.038 | 0.007 | 3.10E−08 | LOC101928306 |
| GFATadj | 4 | 26108197 | rs874040 | G | C | 0.702 | 0.045 | 0.008 | 3.00E−08 | SMIM20 |
| GFATadj | 4 | 56432458 | rs13142096 | A | G | 0.727 | −0.047 | 0.008 | 8.40E−09 | PDCL2 |
| GFATadj | 4 | 89741269 | rs3822072 | G | A | 0.546 | 0.048 | 0.007 | 4.90E−12 | FAM13A |
| GFATadj | 4 | 123812187 | rs546560809 | T | G | 0.961 | 0.098 | 0.018 | 2.50E−08 | FGF2 |
| GFATadj | 4 | 157734675 | rs6822892 | A | G | 0.662 | −0.054 | 0.008 | 8.00E−13 | PDGFC |
| GFATadj | 5 | 38810354 | rs142369482 | G | GT | 0.656 | −0.044 | 0.008 | 9.10E−09 | OSMR-AS1 |
| GFATadj | 5 | 55857025 | rs11429307 | G | GT | 0.809 | 0.082 | 0.009 | 3.10E−20 | C5orf67 |
| GFATadj | 5 | 157931500 | rs10044492 | C | T | 0.732 | −0.048 | 0.008 | 5.30E−09 | LINC02227 |
| GFATadj | 6 | 6749789 | rs1294437 | C | T | 0.641 | −0.04 | 0.008 | 4.10E−08 | LY86 |
| GFATadj | 6 | 32936748 | 6:32936748_TG_T | TG | T | 0.866 | −0.064 | 0.01 | 4.80E−10 | BRD2 |
| GFATadj | 6 | 34234953 | rs199679345 | C | CA | 0.953 | 0.15 | 0.017 | 1.60E−19 | SMIM29 |
| GFATadj | 6 | 43757896 | rs998584 | C | A | 0.517 | 0.08 | 0.007 | 6.10E−31 | VEGFA |
| GFATadj | 6 | 43806315 | rs5875852 | C | CTAAG | 0.306 | 0.058 | 0.008 | 3.80E−14 | LINC02537 |
| GFATadj | 6 | 127454893 | rs72959041 | G | A | 0.953 | 0.195 | 0.017 | 3.20E−32 | RSPO3 |
| GFATadj | 6 | 127457071 | 6:127457071_CA_C | CA | C | 0.464 | 0.066 | 0.007 | 1.10E−19 | RSPO3 |
| GFATadj | 6 | 139835329 | rs2982521 | A | T | 0.372 | −0.055 | 0.007 | 2.10E−14 | LINC01625 |
| GFATadj | 8 | 72469241 | rs11390479 | A | AG | 0.741 | 0.053 | 0.008 | 3.60E−11 | EYA1 |
| GFATadj | 9 | 107722705 | rs1962883 | C | T | 0.529 | 0.055 | 0.007 | 8.20E−14 | ABCA1 |
| GFATadj | 9 | 107901019 | rs111874795 | T | C | 0.955 | −0.103 | 0.017 | 1.00E−09 | SLC44A1 |
| GFATadj | 10 | 122970216 | rs1907218 | T | C | 0.314 | −0.049 | 0.008 | 3.60E−10 | FGFR2 |
| GFATadj | 11 | 36386755 | rs10501153 | C | T | 0.677 | −0.044 | 0.008 | 5.90E−09 | PRR5L |
| GFATadj | 11 | 64018104 | rs71468663 | A | AC | 0.953 | 0.127 | 0.017 | 1.10E−13 | PLCB3 |
| GFATadj | 11 | 65457567 | rs71455776 | G | T | 0.741 | −0.047 | 0.009 | 2.40E−08 | KAT5 |
| GFATadj | 12 | 26366830 | rs748889 | T | C | 0.538 | −0.037 | 0.007 | 2.90E−08 | SSPN |
| GFATadj | 12 | 26440698 | rs12814794 | G | A | 0.248 | −0.072 | 0.008 | 1.60E−18 | ITPR2 |
| GFATadj | 12 | 54342786 | rs4759309 | G | A | 0.221 | −0.044 | 0.009 | 4.20E−08 | HOXC13 |
| GFATadj | 12 | 123024476 | rs147730268 | G | T | 0.913 | 0.069 | 0.013 | 5.00E−08 | KNTC1 |
| GFATadj | 12 | 124150118 | rs150792771 | G | A | 0.982 | −0.157 | 0.028 | 1.80E−08 | GTF2H3 |
| GFATadj | 12 | 124409502 | rs7133378 | G | A | 0.68 | −0.088 | 0.008 | 5.60E−29 | DNAH10 |
| GFATadj | 12 | 124430767 | rs11057402 | T | A | 0.887 | 0.077 | 0.011 | 4.90E−12 | CCDC92 |
| GFATadj | 12 | 124508758 | rs825453 | A | T | 0.394 | 0.062 | 0.007 | 7.20E−19 | ZNF664 |
| GFATadj | 17 | 7538785 | rs2955617 | C | A | 0.348 | −0.042 | 0.007 | 1.20E−08 | SHBG |
| GFATadj | 17 | 17455192 | rs8075019 | G | A | 0.872 | 0.063 | 0.011 | 2.30E−10 | PEMT |
| GFATadj | 19 | 33994417 | rs3786920 | T | C | 0.581 | −0.051 | 0.007 | 5.00E−12 | PEPD |
| GFATadj | 20 | 39179822 | rs1883711 | G | C | 0.969 | 0.127 | 0.021 | 6.80E−10 | MAFB |
| GFATadj | 22 | 38601430 | rs55951234 | C | CCT | 0.419 | 0.046 | 0.007 | 1.20E−10 | MAFF |
| GFATadj(Male) | 1 | 219730799 | rs4846303 | G | T | 0.688 | −0.069 | 0.011 | 4.60E−10 | LYPLAL1-AS1 |
| GFATadj(Male) | 1 | 219769374 | rs6704389 | A | C | 0.828 | 0.076 | 0.014 | 1.60E−08 | ZC3H11B |
| GFATadj(Male) | 2 | 219699999 | rs78058190 | G | A | 0.951 | 0.149 | 0.027 | 4.80E−08 | PRKAG3 |
| GFATadj(Male) | 2 | 227100490 | rs2943648 | A | G | 0.349 | 0.093 | 0.011 | 7.80E−18 | LOC646736 |
| GFATadj(Male) | 3 | 12396913 | rs71304101 | G | A | 0.877 | −0.11 | 0.016 | 2.40E−13 | PPARG |
| GFATadj(Male) | 4 | 104780790 | rs528845403 | A | AATGTGT | 0.991 | −0.325 | 0.061 | 2.40E−08 | TACR3 |
| GFATadj(Male) | 4 | 157734675 | rs6822892 | A | G | 0.662 | −0.065 | 0.011 | 3.60E−09 | PDGFC |
| GFATadj(Male) | 6 | 34234953 | rs199679345 | C | CA | 0.953 | 0.13 | 0.024 | 4.50E−08 | SMIM29 |
| GFATadj(Male) | 6 | 43760327 | rs11967262 | C | G | 0.511 | 0.073 | 0.01 | 2.50E−13 | VEGFA |
| GFATadj(Male) | 6 | 105443189 | rs364663 | T | A | 0.446 | 0.055 | 0.01 | 1.60E−08 | LIN28B |
| GFATadj(Male) | 6 | 127454893 | rs72959041 | G | A | 0.953 | 0.193 | 0.025 | 6.00E−16 | RSPO3 |
| GFATadj(Male) | 6 | 127457071 | 6:127457071_CA_C | CA | C | 0.465 | 0.071 | 0.011 | 1.10E−11 | RSPO3 |
| GFATadj(Female) | 1 | 181161153 | rs7550430 | A | G | 0.998 | 0.892 | 0.144 | 1.80E−09 | LINC01732 |
| GFATadj(Female) | 1 | 219754012 | rs559230165 | C | CT | 0.71 | −0.069 | 0.011 | 5.40E−10 | LYPLAL1-AS1 |
| GFATadj(Female) | 2 | 48962291 | rs17326656 | G | T | 0.761 | 0.069 | 0.012 | 2.60E−09 | STON1-GTF2A1L, |
| | | | | | | | | | | LHCGR |
| GFATadj(Female) | 2 | 165528876 | rs13389219 | C | T | 0.608 | −0.096 | 0.01 | 2.40E−21 | COBLL1 |
| GFATadj(Female) | 2 | 165533198 | rs386652275 | T | TC | 0.974 | −0.19 | 0.034 | 3.20E−08 | COBLL1 |
| GFATadj(Female) | 2 | 165580775 | rs13410987 | C | T | 0.886 | −0.119 | 0.016 | 2.60E−14 | COBLL1 |
| GFATadj(Female) | 2 | 165645349 | rs34224594 | C | CA | 0.616 | −0.057 | 0.011 | 3.10E−08 | COBLL1 |
| GFATadj(Female) | 2 | 227068080 | rs2943634 | A | C | 0.328 | 0.06 | 0.011 | 1.50E−08 | LOC646736 |
| GFATadj(Female) | 3 | 47265877 | rs55664914 | A | AG | 0.635 | −0.058 | 0.01 | 1.80E−08 | KIF9 |
| GFATadj(Female) | 3 | 129322824 | rs1872113 | G | A | 0.778 | −0.066 | 0.012 | 3.10E−08 | PLXND1 |
| GFATadj(Female) | 3 | 150066540 | rs62271373 | T | A | 0.941 | 0.147 | 0.021 | 5.60E−12 | LINC01214 |
| GFATadj(Female) | 5 | 55857025 | rs11429307 | G | GT | 0.812 | 0.121 | 0.013 | 9.00E−22 | C5orf67 |
| GFATadj(Female) | 6 | 34203893 | rs115177000 | G | A | 0.956 | 0.182 | 0.024 | 1.70E−13 | MIR6835 |
| GFATadj(Female) | 6 | 43757896 | rs998584 | C | A | 0.517 | 0.092 | 0.01 | 1.60E−21 | VEGFA |
| GFATadj(Female) | 6 | 43804103 | rs140626545 | A | AGTCGGT | 0.3 | 0.075 | 0.011 | 1.20E−11 | LINC02537 |
| GFATadj(Female) | 6 | 126207917 | rs191578827 | A | G | 0.994 | 0.403 | 0.07 | 3.70E−09 | NCOA7 |
| GFATadj(Female) | 6 | 126964510 | rs4273712 | A | G | 0.731 | 0.061 | 0.011 | 1.60E−08 | MIR588 |
| GFATadj(Female) | 6 | 127454893 | rs72959041 | G | A | 0.952 | 0.205 | 0.024 | 4.50E−19 | RSPO3 |
| GFATadj(Female) | 6 | 127457071 | 6:127457071_CA_C | CA | C | 0.463 | 0.063 | 0.01 | 8.90E−10 | RSPO3 |
| GFATadj(Female) | 6 | 139842576 | rs4052908 | A | AATT | 0.364 | −0.067 | 0.01 | 9.50E−11 | LINC01625 |
| GFATadj(Female) | 8 | 23610799 | rs1561105 | T | G | 0.764 | −0.065 | 0.012 | 1.80E−08 | NKX2-6 |
| GFATadj(Female) | 8 | 72493185 | rs6994124 | T | C | 0.731 | 0.062 | 0.011 | 1.60E−08 | EYA1 |
| GFATadj(Female) | 9 | 107722705 | rs1962883 | C | T | 0.528 | 0.061 | 0.01 | 7.00E−10 | ABCA1 |
| GFATadj(Female) | 11 | 64004723 | rs56271783 | G | C | 0.954 | 0.158 | 0.024 | 1.00E−10 | VEGFB |
| GFATadj(Female) | 12 | 26440698 | rs12814794 | G | A | 0.249 | −0.095 | 0.011 | 3.40E−17 | ITPR2 |
| GFATadj(Female) | 12 | 54346869 | rs894739 | T | C | 0.221 | −0.076 | 0.012 | 5.70E−10 | HOXC12 |
| GFATadj(Female) | 12 | 123024476 | rs147730268 | G | T | 0.913 | 0.108 | 0.018 | 4.10E−10 | KNTC1 |
| GFATadj(Female) | 12 | 124409502 | rs7133378 | G | A | 0.68 | −0.12 | 0.011 | 1.80E−29 | DNAH10 |
| GFATadj(Female) | 12 | 124508758 | rs825453 | A | T | 0.393 | 0.075 | 0.01 | 4.30E−14 | ZNF664 |
| GFATadj(Female) | 12 | 124524638 | rs139254114 | A | T | 0.91 | 0.101 | 0.018 | 7.80E−09 | ZNF664 |
| GFATadj(Female) | 16 | 81534790 | rs2925979 | T | C | 0.297 | −0.067 | 0.011 | 4.40E−10 | CMIP |
| VAT/ASAT | 1 | 203518873 | rs13303359 | A | C | 0.471 | 0.043 | 0.007 | 4.40E−10 | OPTC |
| VAT/ASAT | 2 | 25156773 | rs2384054 | T | C | 0.511 | 0.043 | 0.007 | 1.80E−10 | DNAJC27 |
| VAT/ASAT | 2 | 178121005 | rs13028464 | C | T | 0.631 | −0.039 | 0.007 | 4.80E−08 | NFE2L2 |
| VAT/ASAT | 2 | 227133527 | rs2396316 | A | T | 0.36 | −0.048 | 0.007 | 8.50E−12 | LOC646736 |
| VAT/ASAT | 3 | 12390484 | rs17036328 | T | C | 0.877 | 0.08 | 0.01 | 5.80E−15 | PPARG |
| VAT/ASAT | 3 | 156797225 | rs56082403 | T | C | 0.593 | −0.073 | 0.007 | 3.80E−26 | LINC02029 |
| VAT/ASAT | 5 | 55860907 | 5:55860907_GC_G | GC | G | 0.816 | 0.055 | 0.009 | 3.10E−10 | C5orf67 |
| VAT/ASAT | 5 | 173339531 | rs112299234 | T | C | 0.7 | −0.05 | 0.007 | 3.20E−12 | CPEB4 |
| VAT/ASAT | 6 | 19868603 | rs6903044 | G | C | 0.783 | −0.056 | 0.008 | 1.50E−11 | ID4 |
| VAT/ASAT | 6 | 19947871 | rs70987287 | T | TTTTTA | 0.728 | 0.064 | 0.008 | 1.70E−17 | ID4 |
| VAT/ASAT | 6 | 31236115 | rs2853951 | C | T | 0.407 | −0.044 | 0.007 | 3.20E−10 | HLA-C |
| VAT/ASAT | 6 | 31454887 | rs17193640 | T | A | 0.881 | 0.076 | 0.013 | 9.40E−09 | MICB-DT |
| VAT/ASAT | 6 | 32479878 | rs76072243 | T | C | 0.562 | −0.048 | 0.007 | 1.50E−11 | HLA-DRB5 |
| VAT/ASAT | 6 | 32900378 | 6:32900378_CCT_C | CCT | C | 0.936 | 0.085 | 0.016 | 4.70E−08 | HLA-DMB |
| VAT/ASAT | 6 | 34177853 | rs185139895 | G | A | 0.958 | −0.121 | 0.017 | 1.10E−12 | MIR6835 |
| VAT/ASAT | 6 | 127419737 | rs1936789 | G | A | 0.465 | −0.04 | 0.007 | 1.10E−09 | RSPO3 |
| VAT/ASAT | 6 | 127440047 | rs577721086 | T | C | 0.952 | −0.143 | 0.016 | 1.10E−19 | RSPO3 |
| VAT/ASAT | 6 | 139835329 | rs2982521 | A | T | 0.372 | 0.061 | 0.007 | 5.60E−18 | LINC01625 |
| VAT/ASAT | 6 | 139963500 | rs9484299 | C | T | 0.629 | −0.039 | 0.007 | 4.50E−08 | LINC01625 |
| VAT/ASAT | 8 | 25459001 | rs3890765 | C | A | 0.941 | −0.084 | 0.015 | 6.80E−09 | CDCA2 |
| VAT/ASAT | 8 | 25464670 | rs73221948 | G | T | 0.709 | 0.103 | 0.008 | 1.30E−39 | CDCA2 |
| VAT/ASAT | 8 | 25891653 | rs6997996 | A | G | 0.742 | −0.051 | 0.008 | 3.30E−11 | EBF2 |
| VAT/ASAT | 9 | 1054362 | rs6474552 | G | C | 0.432 | −0.04 | 0.007 | 1.20E−08 | DMRT2 |
| VAT/ASAT | 10 | 63702572 | rs55767272 | A | C | 0.937 | 0.085 | 0.014 | 6.80E−09 | ARID5B |
| VAT/ASAT | 10 | 122992475 | rs11199845 | C | T | 0.46 | 0.055 | 0.007 | 1.50E−14 | FGFR2 |
| VAT/ASAT | 11 | 32479807 | rs11031796 | G | A | 0.612 | 0.058 | 0.007 | 5.80E−17 | WT1-AS |
| VAT/ASAT | 12 | 124409502 | rs7133378 | G | A | 0.68 | 0.043 | 0.007 | 5.40E−09 | DNAH10 |
| VAT/ASAT | 17 | 17533991 | rs4925049 | G | A | 0.917 | −0.069 | 0.013 | 2.60E−08 | PEMT |
| VAT/ASAT | 18 | 42776435 | rs269967 | A | T | 0.825 | 0.048 | 0.009 | 1.90E−08 | SETBP1 |
| VAT/ASAT | 19 | 33785832 | 19:33785832_CA_C | CA | C | 0.824 | 0.095 | 0.01 | 1.00E−23 | CEBPA |
| VAT/ASAT | 19 | 33832399 | rs55865721 | G | A | 0.927 | 0.095 | 0.013 | 4.50E−13 | CEBPA-DT |
| VAT/ASAT | 19 | 33890838 | rs10406327 | C | G | 0.523 | −0.065 | 0.007 | 1.50E−22 | PEPD |
| VAT/ASAT | 22 | 29453193 | rs12321 | G | C | 0.561 | 0.041 | 0.007 | 8.20E−10 | C22orf31 |
| VAT/ASAT(Male) | 2 | 61760756 | rs13390751 | A | C | 0.838 | 0.076 | 0.013 | 1.30E−08 | XPO1 |
| VAT/ASAT(Male) | 2 | 227100579 | 2:227100579_TC_T | TC | T | 0.343 | −0.064 | 0.01 | 4.10E−10 | LOC646736 |
| VAT/ASAT(Male) | 3 | 12360357 | rs527620413 | G | GT | 0.873 | 0.098 | 0.015 | 1.80E−10 | PPARG |
| VAT/ASAT(Male) | 3 | 156797225 | rs56082403 | T | C | 0.595 | −0.07 | 0.01 | 3.50E−12 | LINC02029 |
| VAT/ASAT(Male) | 5 | 173392398 | rs10054063 | A | T | 0.692 | −0.082 | 0.011 | 4.00E−14 | CPEB4 |
| VAT/ASAT(Male) | 6 | 19949170 | 6:19949170_GT_G | GT | G | 0.746 | 0.068 | 0.012 | 3.70E−09 | ID4 |
| VAT/ASAT(Male) | 6 | 31264582 | rs2524137 | C | T | 0.306 | −0.062 | 0.011 | 1.20E−08 | LINCO2571 |
| VAT/ASAT(Male) | 6 | 32485679 | rs375009120 | C | CCTTTT | 0.463 | −0.063 | 0.011 | 1.50E−08 | HLA-DRB5 |
| VAT/ASAT(Male) | 6 | 43760327 | rs11967262 | C | G | 0.511 | −0.064 | 0.01 | 1.40E−10 | VEGFA |
| VAT/ASAT(Male) | 8 | 25464670 | rs73221948 | G | T | 0.709 | 0.099 | 0.011 | 9.80E−18 | CDCA2 |
| VAT/ASAT(Male) | 10 | 122992442 | rs11199844 | C | T | 0.463 | 0.059 | 0.01 | 5.90E−09 | FGFR2 |
| VAT/ASAT(Male) | 11 | 32479807 | rs11031796 | G | A | 0.61 | 0.062 | 0.01 | 5.80E−10 | WT1-AS |
| VAT/ASAT(Male) | 19 | 33785832 | 19:33785832_CA_C | CA | C | 0.823 | 0.085 | 0.014 | 7.20E−10 | CEBPA |
| VAT/ASAT(Male) | 19 | 33834096 | rs73026242 | A | G | 0.93 | 0.117 | 0.02 | 1.30E−09 | CEBPG |
| VAT/ASAT(Male) | 19 | 33890838 | rs10406327 | C | G | 0.525 | −0.057 | 0.01 | 4.30E−09 | PEPD |
| VAT/ASAT(Male) | 21 | 35593827 | rs28451064 | G | A | 0.867 | −0.088 | 0.015 | 1.10E−09 | LINC00310 |
| VAT/ASAT(Female) | 2 | 25082273 | rs916485 | T | C | 0.554 | 0.059 | 0.01 | 6.50E−10 | ADCY3 |
| VAT/ASAT(Female) | 3 | 156795468 | rs13322435 | A | G | 0.589 | −0.079 | 0.01 | 3.30E−16 | LINC02029 |
| VAT/ASAT(Female) | 6 | 19947871 | rs70987287 | T | TTTTTA | 0.729 | 0.064 | 0.011 | 8.50E−10 | ID4 |
| VAT/ASAT(Female) | 6 | 34177853 | rs185139895 | G | A | 0.957 | −0.145 | 0.024 | 4.70E−10 | MIR6835 |
| VAT/ASAT(Female) | 6 | 127440047 | rs577721086 | T | C | 0.952 | −0.177 | 0.023 | 1.70E−15 | RSPO3 |
| VAT/ASAT(Female) | 6 | 139835329 | rs2982521 | A | T | 0.371 | 0.075 | 0.01 | 4.60E−14 | LINC01625 |
| VAT/ASAT(Female) | 7 | 130451984 | 7:130451984_CTTTA_C | CTTTA | C | 0.519 | 0.057 | 0.01 | 2.00E−09 | KLF14 |
| VAT/ASAT(Female) | 8 | 25464670 | rs73221948 | G | T | 0.708 | 0.109 | 0.011 | 1.60E−23 | CDCA2 |
| VAT/ASAT(Female) | 11 | 32458807 | rs3809060 | G | T | 0.619 | 0.057 | 0.01 | 5.60E−09 | WT1-AS |
| VAT/ASAT(Female) | 12 | 121319417 | rs59757908 | T | C | 0.995 | −0.425 | 0.076 | 4.20E−08 | SPPL3 |
| VAT/ASAT(Female) | 12 | 124409502 | rs7133378 | G | A | 0.68 | 0.058 | 0.01 | 9.70E−09 | DNAH10 |
| VAT/ASAT(Female) | 19 | 33785832 | 19:33785832_CA_C | CA | C | 0.824 | 0.107 | 0.014 | 4.30E−15 | CEBPA |
| VAT/ASAT(Female) | 19 | 33892409 | rs889138 | C | T | 0.547 | −0.077 | 0.01 | 2.00E−16 | PEPD |
| VAT/GFAT | 2 | 158412701 | rs55920843 | T | G | 0.989 | 0.18 | 0.033 | 1.90E−08 | ACVR1C |
| VAT/GFAT | 2 | 227133527 | rs2396316 | A | T | 0.36 | −0.042 | 0.007 | 3.10E−09 | LOC646736 |
| VAT/GFAT | 3 | 12390484 | rs17036328 | T | C | 0.877 | 0.058 | 0.011 | 2.40E−08 | PPARG |
| VAT/GFAT | 3 | 49799046 | 3:49799046_CA_C | CA | C | 0.547 | −0.042 | 0.007 | 8.00E−09 | IP6K1 |
| VAT/GFAT | 3 | 187678619 | rs490701 | A | C | 0.795 | −0.052 | 0.009 | 8.00E−09 | LINC01991 |
| VAT/GFAT | 5 | 55816888 | rs455660 | T | C | 0.191 | 0.058 | 0.009 | 1.60E−11 | LINC01948 |
| VAT/GFAT | 5 | 173356752 | rs72812818 | G | C | 0.702 | −0.044 | 0.008 | 8.90E−10 | CPEB4 |
| VAT/GFAT | 6 | 31236115 | rs2853951 | C | T | 0.407 | −0.05 | 0.007 | 3.70E−12 | HLA-C |
| VAT/GFAT | 6 | 32340871 | rs3117109 | C | T | 0.877 | 0.061 | 0.011 | 5.80E−09 | TSBP1 |
| VAT/GFAT | 6 | 32621590 | 6:32621590_T_C | T | C | 0.651 | −0.058 | 0.008 | 3.00E−13 | HLA-DQB1 |
| VAT/GFAT | 6 | 34177853 | rs185139895 | G | A | 0.958 | −0.116 | 0.017 | 1.70E−11 | MIR6835 |
| VAT/GFAT | 6 | 43757896 | rs998584 | C | A | 0.517 | −0.058 | 0.007 | 3.70E−17 | VEGFA |
| VAT/GFAT | 6 | 43810021 | rs9472136 | C | T | 0.604 | 0.041 | 0.007 | 1.90E−08 | LINC02537 |
| VAT/GFAT | 6 | 127333964 | 6:127333964_AG_A | AG | A | 0.966 | −0.112 | 0.02 | 8.90E−09 | RSPO3 |
| VAT/GFAT | 6 | 127419737 | rs1936789 | G | A | 0.465 | −0.055 | 0.007 | 1.30E−15 | RSPO3 |
| VAT/GFAT | 6 | 127440047 | rs577721086 | T | C | 0.952 | −0.16 | 0.016 | 4.60E−23 | RSPO3 |
| VAT/GFAT | 6 | 139835329 | rs2982521 | A | T | 0.372 | 0.056 | 0.007 | 4.40E−15 | LINC01625 |
| VAT/GFAT | 8 | 25464690 | rs11992444 | G | T | 0.492 | −0.06 | 0.007 | 7.80E−19 | CDCA2 |
| VAT/GFAT | 8 | 25888110 | rs10086575 | G | A | 0.744 | −0.045 | 0.008 | 2.90E−08 | EBF2 |
| VAT/GFAT | 11 | 32479992 | rs568011588 | A | AT | 0.703 | 0.042 | 0.008 | 7.90E−09 | WT1-AS |
| VAT/GFAT | 11 | 64031241 | rs35169799 | C | T | 0.936 | −0.084 | 0.014 | 1.10E−08 | PLCB3 |
| VAT/GFAT | 12 | 26453283 | rs718314 | A | G | 0.756 | −0.047 | 0.008 | 2.00E−09 | ITPR2 |
| VAT/GFAT | 12 | 124409502 | rs7133378 | G | A | 0.68 | 0.057 | 0.007 | 1.20E−14 | DNAH10 |
| VAT/GFAT | 12 | 124503803 | 12:124503803_CAA_C | CAA | C | 0.438 | −0.04 | 0.007 | 3.00E−09 | ZNF664 |
| VAT/GFAT | 14 | 94844947 | rs28929474 | C | T | 0.982 | 0.16 | 0.026 | 4.80E−10 | SERPINA1 |
| VAT/GFAT | 19 | 33785832 | 19:33785832_CA_C | CA | C | 0.824 | 0.082 | 0.01 | 2.00E−17 | CEBPA |
| VAT/GFAT | 19 | 33890838 | rs10406327 | C | G | 0.523 | −0.049 | 0.007 | 6.00E−13 | PEPD |
| VAT/GFAT | 19 | 34001331 | rs73041147 | A | C | 0.929 | 0.076 | 0.013 | 1.20E−08 | PEPD |
| VAT/GFAT | 21 | 35593827 | rs28451064 | G | A | 0.868 | −0.059 | 0.01 | 4.90E−09 | LINC00310 |
| VAT/GFAT | 22 | 29453193 | rs12321 | G | C | 0.561 | 0.041 | 0.007 | 3.70E−09 | C22orf31 |
| VAT/GFAT(Male) | 5 | 55794632 | rs30351 | G | A | 0.266 | 0.069 | 0.012 | 3.50E−09 | LINC01948 |
| VAT/GFAT(Male) | 5 | 173324971 | rs55646464 | G | T | 0.703 | −0.062 | 0.011 | 2.40E−08 | CPEB4 |
| VAT/GFAT(Male) | 6 | 31325756 | rs9266247 | G | A | 0.477 | −0.059 | 0.01 | 1.70E−08 | HLA-B |
| VAT/GFAT(Male) | 6 | 32660582 | rs2647006 | A | C | 0.417 | −0.063 | 0.01 | 8.50E−10 | HLA-DQB1 |
| VAT/GFAT(Male) | 6 | 43760327 | rs11967262 | C | G | 0.511 | −0.069 | 0.01 | 6.40E−12 | VEGFA |
| VAT/GFAT(Male) | 6 | 127435106 | rs6916318 | A | T | 0.469 | −0.057 | 0.01 | 2.50E−08 | RSPO3 |
| VAT/GFAT(Male) | 6 | 127454893 | rs72959041 | G | A | 0.953 | −0.147 | 0.024 | 4.90E−10 | RSPO3 |
| VAT/GFAT(Male) | 8 | 25464670 | rs73221948 | G | T | 0.709 | 0.08 | 0.012 | 3.00E−12 | CDCA2 |
| VAT/GFAT(Male) | 17 | 7185092 | rs5418 | G | A | 0.431 | −0.056 | 0.01 | 4.60E−08 | SLC2A4 |
| VAT/GFAT(Female) | 1 | 162430821 | rs9660318 | G | C | 0.203 | 0.068 | 0.012 | 1.80E−08 | UHMK1 |
| VAT/GFAT(Female) | 2 | 116072770 | rs11399916 | T | TA | 0.256 | 0.06 | 0.011 | 3.70E−08 | DPP10 |
| VAT/GFAT(Female) | 2 | 165577164 | rs10221833 | G | C | 0.887 | 0.086 | 0.015 | 2.10E−08 | COBLL1 |
| VAT/GFAT(Female) | 6 | 32975699 | rs9276981 | G | C | 0.809 | −0.064 | 0.012 | 4.60E−08 | HLA-DOA |
| VAT/GFAT(Female) | 6 | 34177853 | rs185139895 | G | A | 0.957 | −0.151 | 0.024 | 4.40E−10 | MIR6835 |
| VAT/GFAT(Female) | 6 | 127419737 | rs1936789 | G | A | 0.464 | −0.053 | 0.01 | 3.70E−08 | RSPO3 |
| VAT/GFAT(Female) | 6 | 127440047 | rs577721086 | T | C | 0.952 | −0.175 | 0.023 | 3.70E−14 | RSPO3 |
| VAT/GFAT(Female) | 6 | 139839768 | rs151288714 | A | AAAAC | 0.483 | 0.072 | 0.01 | 1.70E−13 | LINC01625 |
| VAT/GFAT(Female) | 8 | 25464690 | rs11992444 | G | T | 0.491 | −0.057 | 0.01 | 1.90E−09 | CDCA2 |
| VAT/GFAT(Female) | 12 | 122820960 | 12:122820960_TAA_T | TAA | T | 0.214 | 0.068 | 0.012 | 1.60E−08 | CLIP1 |
| VAT/GFAT(Female) | 12 | 124409502 | rs7133378 | G | A | 0.68 | 0.08 | 0.01 | 1.30E−14 | DNAH10 |
| VAT/GFAT(Female) | 19 | 33785832 | 19:33785832_CA_C | CA | C | 0.824 | 0.099 | 0.014 | 4.60E−13 | CEBPA |
| VAT/GFAT(Female) | 19 | 33897478 | rs3786901 | A | C | 0.575 | −0.057 | 0.01 | 4.90E−09 | PEPD |
| ASAT/GFAT | 1 | 119508412 | rs1779445 | T | C | 0.194 | −0.054 | 0.009 | 8.50E−10 | TBX15 |
| ASAT/GFAT | 2 | 25310860 | rs564667 | A | T | 0.566 | 0.04 | 0.007 | 2.40E−08 | EFR3B |
| ASAT/GFAT | 3 | 49803078 | 3:49803078_TA_T | TA | T | 0.595 | −0.043 | 0.008 | 3.60E−08 | IP6K1 |
| ASAT/GFAT | 3 | 156795525 | rs9854955 | A | G | 0.593 | 0.063 | 0.007 | 1.90E−18 | LINC02029 |
| ASAT/GFAT | 4 | 157681274 | rs28730491 | G | C | 0.668 | 0.047 | 0.007 | 3.20E−10 | PDGFC |
| ASAT/GFAT | 5 | 55830865 | rs39837 | C | T | 0.667 | 0.043 | 0.007 | 2.60E−08 | LINC01948 |
| ASAT/GFAT | 5 | 55856375 | rs3843467 | G | T | 0.793 | −0.091 | 0.009 | 4.80E−27 | C5orf67 |
| ASAT/GFAT | 6 | 43757896 | rs998584 | C | A | 0.517 | −0.049 | 0.007 | 1.90E−12 | VEGFA |
| ASAT/GFAT | 6 | 43805362 | rs744103 | T | A | 0.315 | −0.041 | 0.008 | 5.00E−08 | LINCO2537 |
| ASAT/GFAT | 6 | 127397240 | rs9375487 | T | C | 0.624 | 0.045 | 0.007 | 2.80E−10 | RSPO3 |
| ASAT/GFAT | 8 | 72475748 | rs7843475 | C | G | 0.737 | −0.045 | 0.008 | 3.60E−09 | EYA1 |
| ASAT/GFAT | 12 | 124409502 | rs7133378 | G | A | 0.68 | 0.043 | 0.007 | 1.10E−08 | DNAH10 |
| ASAT/GFAT | 14 | 95219657 | rs8006225 | G | T | 0.817 | 0.055 | 0.009 | 2.60E−09 | GSC |
| ASAT/GFAT | 16 | 53800954 | rs1421085 | T | C | 0.603 | −0.064 | 0.007 | 3.40E−19 | FTO |
| ASAT/GFAT | 16 | 86424697 | rs1552657 | G | A | 0.549 | −0.037 | 0.007 | 4.90E−08 | LINC00917 |
| ASAT/GFAT | 19 | 18324329 | rs2302209 | C | T | 0.719 | −0.047 | 0.008 | 2.00E−09 | PDE4C |
| ASAT/GFAT | 19 | 33846522 | rs1423062 | A | G | 0.567 | 0.039 | 0.007 | 2.90E−08 | CEBPG |
| ASAT/GFAT (Male) | 3 | 156794425 | rs4680338 | C | G | 0.591 | 0.077 | 0.01 | 3.30E−14 | LINC02029 |
| ASAT/GFAT (Male) | 16 | 53806453 | rs56094641 | A | G | 0.596 | −0.078 | 0.01 | 4.10E−14 | FTO |
| ASAT/GFAT (Female) | 1 | 119471908 | rs2645290 | A | G | 0.213 | −0.068 | 0.012 | 1.80E−08 | TBX15 |
| ASAT/GFAT (Female) | 5 | 55830865 | rs39837 | C | T | 0.666 | 0.061 | 0.01 | 9.10E−09 | LINC01948 |
| ASAT/GFAT (Female) | 5 | 55860866 | rs3936510 | G | T | 0.801 | −0.137 | 0.012 | 1.90E−28 | C5orf67 |
| ASAT/GFAT (Female) | 6 | 43757896 | rs998584 | C | A | 0.517 | −0.079 | 0.01 | 5.10E−16 | VEGFA |
| ASAT/GFAT (Female) | 6 | 43805362 | rs744103 | T | A | 0.314 | −0.068 | 0.011 | 1.30E−10 | LINC02537 |
| ASAT/GFAT (Female) | 7 | 130029811 | rs10246191 | G | A | 0.672 | 0.056 | 0.01 | 3.80E−08 | CPA1 |
| ASAT/GFAT (Female) | 7 | 130432913 | rs553015785 | A | AT | 0.519 | −0.056 | 0.01 | 9.40E−09 | KLF14 |
| ASAT/GFAT (Female) | 11 | 64018104 | rs71468663 | A | AC | 0.952 | −0.129 | 0.023 | 3.90E−08 | PLCB3 |
| ASAT/GFAT (Female) | 12 | 124409502 | rs7133378 | G | A | 0.68 | 0.07 | 0.01 | 2.80E−11 | DNAH10 |

### Supplementary Data 13. Transcriptome-Wide Association Study Results

Implementation was done in FUSION with default settings using GTEx v7 tissue library.

Phenotype-tissue pairs are as follows: VATadj—visceral adipose (VAT); ASATadj—subcutaneous adipose (SAT); GFATadj—SAT; VAT/ASAT—VAT and SAT; VAT/GFAT—VAT and SAT; ASAT/GFAT—SAT.

Table shows data for p value less than or equal to 9.82E-05. Full table available at Agrawal S, Wang M, Klarqvist M D R, et al. Inherited basis of visceral, abdominal subcutaneous and gluteofemoral fat depots. Nat Commun. 2022; 13(1):3771.

| pheno | ID | CHR | P0 | P1 | HSQ | BEST.GWAS.ID | BEST.GWAS.Z | EQTL.ID |
|---|---|---|---|---|---|---|---|---|
| VATadj | CEBPA-AS1 | 19 | 33793763 | 33795941 | 0.1559 | rs3786897 | 9.26 | rs17529595 |
| VATadj | CCDC92 | 12 | 124403207 | 124457378 | 0.3169 | rs7133378 | −6.17 | rs4930721 |
| VATadj | FLOT1 | 6 | 30695486 | 30710510 | 0.0716 | rs1265093 | 5.42 | rs3130557 |
| VATadj | CYP21A1P | 6 | 31973466 | 31976176 | 0.3074 | rs389883 | −6.07 | rs2269426 |
| VATadj | HLA-DRB6 | 6 | 32520490 | 32527799 | 0.8525 | rs28366298 | 5.97 | rs28366298 |
| VATadj | HLA-S | 6 | 31349851 | 31350065 | 0.5473 | rs2523578 | −6.71 | rs2523578 |
| VATadj | ATG13 | 11 | 46638826 | 46696368 | 0.0726 | rs1489192 | −5.74 | rs12272795 |
| VATadj | APOM | 6 | 31623248 | 31625987 | 0.0567 | rs2523578 | −6.71 | rs2855812 |
| VATadj | EXOSC10 | 1 | 11126675 | 11158213 | 0.1137 | rs1057079 | −6.71 | rs2791655 |
| VATadj | PRRT1 | 6 | 32116136 | 32121621 | 0.1097 | rs389883 | −6.07 | rs521977 |
| VATadj | MAST3 | 19 | 18208603 | 18262502 | 0.0917 | rs8112975 | 5.39 | rs740691 |
| VATadj | HCG23 | 6 | 32358287 | 32361463 | 0.0794 | rs389883 | −6.07 | rs9271055 |
| VATadj | DNAH10 | 12 | 124247042 | 124420168 | 0.4157 | rs7133378 | −6.17 | rs12309481 |
| VATadj | HLA-DQA2 | 6 | 32709119 | 32714992 | 0.8413 | rs28366298 | 5.97 | rs28366298 |
| VATadj | HLA-DRB1 | 6 | 32546546 | 32557625 | 0.3931 | rs28366298 | 5.97 | rs532098 |
| VATadj | PNKD | 2 | 219135115 | 219211516 | 0.164 | rs3731861 | 5.46 | rs4672884 |
| VATadj | RP11-380L11.4 | 12 | 124410008 | 124410630 | 0.0798 | rs7133378 | −6.17 | rs4930726 |
| VATadj | RP11-378A13.1 | 2 | 219120042 | 219122087 | 0.4016 | rs3731861 | 5.46 | rs736731 |
| VATadj | XXbac-BPG248L24.12 | 6 | 31324424 | 31325414 | 0.2052 | rs2523578 | −6.71 | rs2844623 |
| VATadj | HCG27 | 6 | 31165915 | 31171745 | 0.3102 | rs2523578 | −6.71 | rs1265100 |
| VATadj | HLA-C | 6 | 31236526 | 31239882 | 0.5466 | rs2523578 | −6.71 | rs1265087 |
| VATadj | TBX15 | 1 | 119425669 | 119532179 | 0.0951 | rs10923724 | −4.94 | rs2645294 |
| VATadj | NAA25 | 12 | 112464500 | 112546826 | 0.0709 | rs11065987 | 4.63 | rs4767293 |
| VATadj | C4B | 6 | 31982539 | 32003195 | 0.1199 | rs389883 | −6.07 | rs652888 |
| VATadj | NCKIPSD | 3 | 48701364 | 48723797 | 0.2129 | rs4513485 | −4.68 | rs12493578 |
| VATadj | TMBIM1 | 2 | 219138915 | 219157309 | 0.0981 | rs3731861 | 5.46 | rs10932766 |
| VATadj | DALRD3 | 3 | 49053387 | 49059726 | 0.052 | rs4513485 | −4.68 | rs7626445 |
| VATadj | DNAH10OS | 12 | 124410971 | 124419531 | 0.1162 | rs7133378 | −6.17 | rs4765127 |
| VATadj | JAZF1 | 7 | 27870192 | 28220362 | 0.1375 | rs1635853 | 5.39 | rs1635852 |
| VATadj | PSORS1C1 | 6 | 31082527 | 31107869 | 0.5408 | rs2523578 | −6.71 | rs1042147 |
| VATadj | HLA-DQB1-AS1 | 6 | 32628132 | 32628506 | 0.5356 | rs28366298 | 5.97 | rs1063355 |
| VATadj | WDR6 | 3 | 49044588 | 49053236 | 0.2343 | rs4513485 | −4.68 | rs9311433 |
| VATadj | DSTYK | 1 | 205111632 | 205180727 | 0.0742 | rs11240358 | 4.47 | rs1572993 |
| VATadj | P4HTM | 3 | 49027422 | 49044494 | 0.0588 | rs4513485 | −4.68 | rs7431857 |
| VATadj | IFT80 | 3 | 159974774 | 160117061 | 0.0657 | rs1159747 | −4.31 | rs4679903 |
| VATadj | CCDC36 | 3 | 49235861 | 49295537 | 0.1368 | rs4513485 | −4.68 | rs4955418 |
| VATadj | RP11-3B7.1 | 3 | 49297518 | 49298744 | 0.1103 | rs4513485 | −4.68 | rs4955418 |
| VATadj | C3orf62 | 3 | 49306219 | 49315263 | 0.05 | rs4513485 | −4.68 | rs9874474 |
| VATadj | CYP21A2 | 6 | 32006042 | 32009447 | 0.1939 | rs389883 | −6.07 | rs3131382 |
| VATadj | RP5-935K16.1 | 2 | 128601127 | 128603261 | 0.2899 | rs17600636 | 4.03 | rs17600636 |
| VATadj | CD79B | 17 | 62006100 | 62009714 | 0.1142 | rs1051684 | 4.01 | rs1051684 |
| VATadj | LMBR1L | 12 | 49490919 | 49504681 | 0.1049 | rs2293445 | −4.29 | rs12580349 |
| VATadj | ALKBH5 | 17 | 18086392 | 18113268 | 0.2119 | rs3818717 | 4.46 | rs860568 |
| VATadj | ADCY3 | 2 | 25042038 | 25142708 | 0.1236 | rs713586 | −4.4 | rs1541984 |
| ASATadj | CENPW | 6 | 126661320 | 126670021 | 0.0447 | rs9388496 | −6.33 | rs9375435 |
| ASATadj | TIPARP | 3 | 156391024 | 156424559 | 0.1228 | rs10049090 | −7.79 | rs10049090 |
| ASATadj | AC103965.1 | 15 | 84867600 | 84898888 | 0.1881 | rs7183263 | −8.34 | rs12912934 |
| ASATadj | CSPG4P11 | 15 | 84855504 | 84866136 | 0.3219 | rs7183263 | −8.34 | rs12912934 |
| ASATadj | IRS1 | 2 | 227596033 | 227664475 | 0.1263 | rs1515116 | 5.466 | rs1515116 |
| ASATadj | RP11-671M22.4 | 15 | 84949210 | 84950212 | 0.0835 | rs7183263 | −8.34 | rs4842939 |
| ASATadj | RIMKLBP2 | 1 | 219373256 | 219373909 | 0.0694 | rs2494196 | 5.5 | rs3001032 |
| ASATadj | PAN2 | 12 | 56710121 | 56727837 | 0.0699 | rs17118439 | −4.95 | rs17118439 |
| ASATadj | XYLB | 3 | 38388251 | 38462839 | 0.1079 | rs7372545 | 5.45 | rs1002675 |
| ASATadj | EXOG | 3 | 38537618 | 38583437 | 0.0974 | rs7372545 | 5.45 | rs4371464 |
| ASATadj | CTD-2007L18.5 | 11 | 68380367 | 68384179 | 0.0536 | rs901823 | 5.24 | rs599083 |
| ASATadj | RP11-977G19.11 | 12 | 56693926 | 56708592 | 0.2602 | rs17118439 | −4.95 | rs11171806 |
| ASATadj | STAT2 | 12 | 56735381 | 56753910 | 0.1739 | rs17118439 | −4.95 | rs11575229 |
| ASATadj | RP4-712E4.1 | 1 | 119542967 | 119543516 | 0.2441 | rs6428790 | −4.81 | rs1409159 |
| ASATadj | ACO2 | 22 | 41865129 | 41921352 | 0.0662 | rs3927 | 5.14 | rs8135804 |
| ASATadj | THBS3 | 1 | 155165379 | 155177708 | 0.0666 | rs12040970 | 4.46 | rs4971079 |
| ASATadj | RP11-392O17.1 | 1 | 219583023 | 219585283 | 0.1575 | rs2494196 | 5.5 | rs2605097 |
| ASATadj | RFTN2 | 2 | 198432948 | 198540769 | 0.0771 | rs17731449 | 5.123 | rs4850808 |
| ASATadj | RP11-43F13.3 | 5 | 987295 | 997423 | 0.2311 | rs6882848 | 4.36 | rs13160308 |
| ASATadj | EYA1 | 8 | 72109668 | 72274467 | 0.1586 | rs10093418 | 4.71 | rs35510588 |
| ASATadj | CD79B | 17 | 62006100 | 62009714 | 0.4361 | rs2070776 | 4.57 | rs1051684 |
| ASATadj | KLF14 | 7 | 130417401 | 130418888 | 0.1596 | rs4731702 | 6.48 | rs13233731 |
| ASATadj | RN7SL417P | 15 | 84948770 | 84949050 | 0.1619 | rs7183263 | −8.34 | rs11635505 |
| ASATadj | TBX15 | 1 | 119425669 | 119532179 | 0.0973 | rs6428790 | −4.81 | rs984225 |
| ASATadj | NKD2 | 5 | 1008944 | 1039058 | 0.3 | rs6882848 | 4.36 | rs13160308 |
| ASATadj | MEST | 7 | 130126025 | 130146088 | 0.1716 | rs4731702 | 6.48 | rs17164872 |
| ASATadj | SCAND2P | 15 | 85174682 | 85185695 | 0.1083 | rs765524 | 6.92 | rs7179643 |
| ASATadj | ARNT | 1 | 150782181 | 150849244 | 0.1432 | rs9659073 | 5.28 | rs7412746 |
| ASATadj | RPS18P9 | 6 | 149915220 | 149915679 | 0.047 | rs7769115 | 4.22 | rs9498368 |
| ASATadj | NMT1 | 17 | 43129030 | 43186334 | 0.2442 | rs4986172 | 4.93 | rs6503422 |
| ASATadj | LINC00933 | 15 | 85114155 | 85123406 | 0.2501 | rs11638600 | 6.92 | rs12912934 |
| ASATadj | RP11-347119.8 | 12 | 122235417 | 122235778 | 0.3143 | rs7962930 | 4.34 | rs895951 |
| ASATadj | RAF1 | 3 | 12625213 | 12705725 | 0.1119 | rs11709077 | 6.39 | rs4234512 |
| ASATadj | RP11-419C23.1 | 8 | 36924959 | 36926936 | 0.0983 | rs16885494 | −4.08 | rs10110651 |
| ASATadj | RHOF | 12 | 122231057 | 122240536 | 0.1349 | rs7962930 | 4.34 | rs11043203 |
| ASATadj | AC084018.1 | 12 | 122233173 | 122241812 | 0.3344 | rs7962930 | 4.34 | rs11043203 |
| ASATadj | MEI1 | 22 | 42095503 | 42195460 | 0.1384 | rs3927 | 5.14 | rs5758405 |
| ASATadj | RP11-182J1.13 | 15 | 84977316 | 84980581 | 0.0814 | rs7183263 | −8.34 | rs11638788 |
| ASATadj | EP300 | 22 | 41487790 | 41576081 | 0.0531 | rs3927 | 5.14 | rs2273085 |
| ASATadj | GOLGA6L5 | 15 | 85051116 | 85060045 | 0.6077 | rs7183263 | −8.34 | rs150968 |
| ASATadj | GBAP1 | 1 | 155183616 | 155197214 | 0.3574 | rs12040970 | 4.46 | rs2990245 |
| ASATadj | RP11-328C8.2 | 12 | 42825467 | 42827159 | 0.0996 | rs1234032 | −4.89 | rs1796357 |
| ASATadj | RP11-182J1.5 | 15 | 85154920 | 85158200 | 0.052 | rs11638600 | 6.92 | rs11631921 |
| GFATadj | CCDC92 | 12 | 124403207 | 124457378 | 0.3102 | rs7133378 | 11.17 | rs7307053 |
| GFATadj | DNAH10OS | 12 | 124410971 | 124419531 | 0.131 | rs7133378 | 11.17 | rs4930726 |
| GFATadj | RP11-380L11.4 | 12 | 124410008 | 124410630 | 0.1109 | rs7133378 | 11.17 | rs4930726 |
| GFATadj | IRS1 | 2 | 227596033 | 227664475 | 0.1263 | rs2713552 | 9.3 | rs1515116 |
| GFATadj | ZNF664 | 12 | 124457670 | 124499986 | 0.1843 | rs7133378 | 11.17 | rs863750 |
| GFATadj | RIMKLBP2 | 1 | 219373256 | 219373909 | 0.0694 | rs4846567 | 8.84 | rs3001032 |
| GFATadj | DNAH10 | 12 | 124247042 | 124420168 | 0.2465 | rs7133378 | 11.17 | rs12309481 |
| GFATadj | RP11-392O17.1 | 1 | 219583023 | 219585283 | 0.1575 | rs4846567 | 8.84 | rs2605097 |
| GFATadj | VEGFB | 11 | 64002010 | 64006259 | 0.1728 | rs35169799 | −7.03 | rs35169799 |
| GFATadj | FAM13A | 4 | 89647106 | 90032549 | 0.155 | rs9991328 | −6.6 | rs9991328 |
| GFATadj | PDGFC | 4 | 157681606 | 157892546 | 0.0706 | rs1425486 | 7.03 | rs2113992 |
| GFATadj | MAFF | 22 | 38597889 | 38612518 | 0.1332 | rs2267373 | 6.42 | rs133024 |
| GFATadj | TMEM165 | 4 | 56262124 | 56319564 | 0.1347 | rs13120134 | 5.73 | rs819269 |
| GFATadj | RP11-177J6.1 | 4 | 56254116 | 56254438 | 0.1128 | rs13120134 | 5.73 | rs476184 |
| GFATadj | CLOCK | 4 | 56294070 | 56413278 | 0.2082 | rs13120134 | 5.73 | rs11133377 |
| GFATadj | SRD5A3-AS1 | 4 | 56230138 | 56262009 | 0.1374 | rs13120134 | 5.73 | rs12641881 |
| GFATadj | PEPD | 19 | 33877856 | 34012700 | 0.3693 | rs3786920 | 6.91 | rs10404460 |
| GFATadj | EXOG | 3 | 38537618 | 38583437 | 0.0974 | rs2300669 | 5.87 | rs4371464 |
| GFATadj | ATP6V0A2 | 12 | 124196865 | 124246302 | 0.1793 | rs7133378 | 11.17 | rs7975233 |
| GFATadj | BAIAP2L2 | 22 | 38480896 | 38506677 | 0.2142 | rs2267373 | 6.42 | rs133029 |
| GFATadj | RP11-32D16.1 | 5 | 157912198 | 157961446 | 0.148 | rs10044492 | 5.84 | rs6872907 |
| GFATadj | RP11-211G23.2 | 11 | 69186231 | 69187279 | 0.3191 | rs7102705 | −5.18 | rs12808959 |
| GFATadj | GRB14 | 2 | 165349326 | 165478358 | 0.1738 | rs6717858 | 9.836 | rs3942459 |
| GFATadj | XXbac-BPG248L24.12 | 6 | 31324424 | 31325414 | 0.2306 | rs2523578 | 4.81 | rs2844623 |
| GFATadj | CTC-228N24.3 | 5 | 127276118 | 127418864 | 0.418 | rs17764730 | 5.19 | rs3749748 |
| GFATadj | RP11-708J19.1 | 3 | 47420579 | 47422489 | 0.0347 | rs11130126 | 5.48 | rs11710322 |
| GFATadj | SUMO2 | 17 | 73163408 | 73179078 | 0.0743 | rs9907177 | −4.31 | rs35271045 |
| GFATadj | KREMEN1 | 22 | 29469066 | 29564321 | 0.2595 | rs134657 | 4.95 | rs134609 |
| GFATadj | PTPN23 | 3 | 47422501 | 47454931 | 0.0271 | rs11130126 | 5.48 | rs11705957 |
| GFATadj | ROM1 | 11 | 62379884 | 62382592 | 0.2782 | rs7124057 | −4.83 | rs11231161 |
| GFATadj | XYLB | 3 | 38388251 | 38462839 | 0.1079 | rs2300669 | 5.87 | rs1002675 |
| GFATadj | RP3-323P13.2 | 6 | 133823390 | 134212850 | 0.3 | rs7767007 | 4.75 | rs7767007 |
| GFATadj | CHST8 | 19 | 34112861 | 34264414 | 0.3245 | rs3786920 | 6.91 | rs10415555 |
| GFATadj | EEF1G | 11 | 62327073 | 62342401 | 0.1173 | rs7124057 | −4.83 | rs11231154 |
| GFATadj | ATP1B2 | 17 | 7549945 | 7561086 | 0.1268 | rs2955617 | −5.7 | rs1642800 |
| GFATadj | MUC1 | 1 | 155158300 | 155162707 | 0.2262 | rs6695407 | 4.132 | rs11264341 |
| GFATadj | EML3 | 11 | 62369690 | 62380185 | 0.2193 | rs7124057 | −4.83 | rs11231144 |
| GFATadj | SETD2 | 3 | 47057919 | 47205457 | 0.0882 | rs11130126 | 5.48 | rs11130126 |
| GFATadj | RPS18P9 | 6 | 149915220 | 149915679 | 0.047 | rs7752089 | 4.02 | rs9498368 |
| GFATadj | NMUR1 | 2 | 232387871 | 232395206 | 0.3954 | rs4973442 | 4.587 | rs4973442 |
| GFATadj | CEBPA-AS1 | 19 | 33793763 | 33795941 | 0.0957 | rs3786920 | 6.91 | rs17529595 |
| GFATadj | SENP2 | 3 | 185300284 | 185351339 | 0.099 | rs13095912 | −5.17 | rs13100034 |
| GFATadj | B3GAT3 | 11 | 62382768 | 62389647 | 0.1309 | rs7124057 | −4.83 | rs693698 |
| GFATadj | SNX10 | 7 | 26331541 | 26413949 | 0.5908 | rs10238703 | −4.72 | rs1534696 |
| GFATadj | EP300 | 22 | 41487790 | 41576081 | 0.0531 | rs5996039 | 4.56 | rs2273085 |
| GFATadj | MYEOV | 11 | 69061605 | 69182494 | 0.4279 | rs7102705 | −5.18 | rs12808959 |
| GFATadj | PRDX5 | 11 | 64085560 | 64089283 | 0.1495 | rs35169799 | −7.03 | rs3782101 |
| GFATadj | C4B | 6 | 31982539 | 32003195 | 0.1682 | rs1150753 | 4.13 | rs1150755 |
| GFATadj | RP11-470E16.1 | 1 | 59597608 | 59664293 | 0.36 | rs11207488 | −4.344 | rs12758288 |
| GFATadj | PTH1R | 3 | 46919236 | 46945287 | 0.0411 | rs11130126 | 5.48 | rs9834713 |
| GFATadj | DCAKD | 17 | 43100708 | 43138473 | 0.3235 | rs916661 | −4.91 | rs4128658 |
| GFATadj | MEI1 | 22 | 42095503 | 42195460 | 0.1384 | rs132770 | 4.65 | rs5758405 |
| GFATadj | RP11-309N17.4 | 17 | 72966799 | 72971823 | 0.0731 | rs9907177 | −4.31 | rs11650024 |
| GFATadj | RP11-798G7.5 | 17 | 43580626 | 43612076 | 0.1281 | rs916661 | −4.91 | rs17762769 |
| GFATadj | RP5-1115A15.1 | 1 | 8484705 | 8494898 | 0.1083 | rs301819 | −4.254 | rs301805 |
| GFATadj | RNF157 | 17 | 74138534 | 74236454 | 0.3835 | rs8079062 | −4.86 | rs7225367 |
| GFATadj | CTA-228A9.3 | 22 | 38486134 | 38487566 | 0.3075 | rs2267373 | 6.42 | rs9798787 |
| GFATadj | SLC16A8 | 22 | 38474141 | 38480100 | 0.1419 | rs2267373 | 6.42 | rs139896 |
| GFATadj | FLRT1 | 11 | 63870660 | 63886613 | 0.1561 | rs35169799 | −7.03 | rs693984 |
| GFATadj | TMEM60 | 7 | 77423045 | 77427897 | 0.154 | rs17807185 | 4.06 | rs1544457 |
| GFATadj | CALCRL | 2 | 188207856 | 188313187 | 0.0454 | rs17576323 | 4.021 | rs13417165 |
| GFATadj | RP11-2E11.5 | 7 | 130121332 | 130124233 | 0.0983 | rs2239606 | 4.01 | rs2268382 |
| GFATadj | RP11-196G18.22 | 1 | 149816065 | 149820591 | 0.3245 | rs11205303 | 5.64 | rs7531664 |
| GFATadj | WARS2 | 1 | 119573839 | 119683018 | 0.5978 | rs7543720 | 3.867 | rs2645303 |
| GFATadj | SEPT1 | 16 | 30389531 | 30407312 | 0.1146 | rs4465620 | 4.08 | rs8050812 |
| GFATadj | ACO2 | 22 | 41865129 | 41921352 | 0.0662 | rs132770 | 4.65 | rs8135804 |
| VAT/ASAT | CEBPA-AS1 | 19 | 33793763 | 33795941 | 0.1559 | rs3786897 | 9.36 | rs17529595 |
| VAT/ASAT | CCDC92 | 12 | 124403207 | 124457378 | 0.3169 | rs7133378 | −5.83 | rs4930721 |
| VAT/ASAT | ADCY3 | 2 | 25042038 | 25142708 | 0.1236 | rs713586 | −6.37 | rs1541984 |
| VAT/ASAT | FLOT1 | 6 | 30695486 | 30710510 | 0.0716 | rs3130557 | −4.99 | rs3130557 |
| VAT/ASAT | APOM | 6 | 31623248 | 31625987 | 0.0567 | rs2523578 | −5.67 | rs2855812 |
| VAT/ASAT | HCG23 | 6 | 32358287 | 32361463 | 0.0794 | rs532098 | 5.63 | rs9271055 |
| VAT/ASAT | AC079305.11 | 2 | 177855236 | 178029244 | 0.3692 | rs10183914 | 5.19 | rs2706134 |
| VAT/ASAT | HLA-S | 6 | 31349851 | 31350065 | 0.5473 | rs2523578 | −5.67 | rs2523578 |
| VAT/ASAT | CYP21A1P | 6 | 31973466 | 31976176 | 0.3074 | rs1150755 | −5.33 | rs2269426 |
| VAT/ASAT | HLA-DRB6 | 6 | 32520490 | 32527799 | 0.8525 | rs532098 | 5.63 | rs28366298 |
| VAT/ASAT | CENPO | 2 | 25016252 | 25045245 | 0.1369 | rs713586 | −6.37 | rs7576788 |
| VAT/ASAT | PRRT1 | 6 | 32116136 | 32121621 | 0.1097 | rs532098 | 5.63 | rs521977 |
| VAT/ASAT | HLA-DRB1 | 6 | 32546546 | 32557625 | 0.3931 | rs532098 | 5.63 | rs532098 |
| VAT/ASAT | EFR3B | 2 | 25264999 | 25378243 | 0.1688 | rs713586 | −6.37 | rs2918630 |
| VAT/ASAT | PEMT | 17 | 17408877 | 17495022 | 0.1398 | rs8074272 | 5.52 | rs750546 |
| VAT/ASAT | DNAJC27 | 2 | 25166505 | 25194963 | 0.1047 | rs713586 | −6.37 | rs17046742 |
| VAT/ASAT | RRAS2 | 11 | 14299472 | 14386052 | 0.0676 | rs11023175 | −3.91 | rs11023197 |
| VAT/ASAT | NAA25 | 12 | 112464500 | 112546826 | 0.0709 | rs666951 | −4.48 | rs4767293 |
| VAT/ASAT | C3orf62 | 3 | 49306219 | 49315263 | 0.05 | rs7623023 | −3.9 | rs9874474 |
| VAT/ASAT | MIR4435-1HG | 2 | 111953927 | 112252677 | 0.1112 | rs1345203 | −3.49 | rs36018702 |
| VAT/ASAT | RP11-43F13.3 | 5 | 987295 | 997423 | 0.1335 | rs4975583 | 3.75 | rs6882848 |
| VAT/ASAT | ATG13 | 11 | 46638826 | 46696368 | 0.0726 | rs7109698 | −4.61 | rs12272795 |
| VAT/ASAT | RP11-378A13.1 | 2 | 219120042 | 219122087 | 0.4016 | rs3731861 | 4.68 | rs736731 |
| VAT/ASAT | RPS26 | 12 | 56435637 | 56438116 | 0.7741 | rs877636 | −4.83 | rs10876864 |
| VAT/ASAT | DNAH10OS | 12 | 124410971 | 124419531 | 0.1162 | rs7133378 | −5.83 | rs4765127 |
| VAT/ASAT | DNAH10 | 12 | 124247042 | 124420168 | 0.4157 | rs7133378 | −5.83 | rs12309481 |
| VAT/ASAT | GS1-259H13.2 | 7 | 99195689 | 99208439 | 0.1785 | rs3843540 | −4.47 | rs6947826 |
| VAT/ASAT | RP11-380L11.4 | 12 | 124410008 | 124410630 | 0.0798 | rs7133378 | −5.83 | rs4930726 |
| VAT/ASAT | PNKD | 2 | 219135115 | 219211516 | 0.164 | rs3731861 | 4.68 | rs4672884 |
| VAT/ASAT | HLA-DQA2 | 6 | 32709119 | 32714992 | 0.8413 | rs532098 | 5.63 | rs28366298 |
| VAT/ASAT | RP11-282O18.3 | 12 | 123736577 | 123745527 | 0.0998 | rs4759415 | −3.99 | rs1969354 |
| VAT/ASAT | ARL17B | 17 | 44352150 | 44439130 | 0.6531 | rs17698176 | 3.58 | rs17698176 |
| VAT/ASAT | WDR6 | 3 | 49044588 | 49053236 | 0.2343 | rs6791542 | −3.99 | rs9311433 |
| VAT/ASAT | BTN3A3 | 6 | 26440700 | 26453643 | 0.2595 | rs6921148 | 3.76 | rs1131936 |
| VAT/ASAT | EXOSC10 | 1 | 11126675 | 11158213 | 0.1137 | rs6701524 | −5.09 | rs2791655 |
| VAT/ASAT | TMEM80 | 11 | 695533 | 705028 | 0.6185 | rs1599725 | −4.06 | rs11246262 |
| VAT/ASAT | HLA-DQB1-AS1 | 6 | 32628132 | 32628506 | 0.5356 | rs532098 | 5.63 | rs1063355 |
| VAT/ASAT | PCBD1 | 10 | 72642037 | 72648541 | 0.1287 | rs16928023 | 3.92 | rs16928023 |
| VAT/ASAT | TMBIM1 | 2 | 219138915 | 219157309 | 0.0981 | rs3731861 | 4.68 | rs10932766 |
| VAT/ASAT | TIPARP | 3 | 156391024 | 156424559 | 0.1228 | rs10049090 | 10.51 | rs10049090 |
| VAT/ASAT | CEBPA-AS1 | 19 | 33793763 | 33795941 | 0.0957 | rs3786897 | 9.36 | rs17529595 |
| VAT/ASAT | IRS1 | 2 | 227596033 | 227664475 | 0.1263 | rs908252 | −6.4 | rs1515116 |
| VAT/ASAT | C4B | 6 | 31982539 | 32003195 | 0.1682 | rs1150755 | −5.33 | rs1150755 |
| VAT/ASAT | CENPO | 2 | 25016252 | 25045245 | 0.1447 | rs713586 | −6.37 | rs2033655 |
| VAT/ASAT | DNAH10OS | 12 | 124410971 | 124419531 | 0.131 | rs7133378 | −5.83 | rs4930726 |
| VAT/ASAT | ADCY3 | 2 | 25042038 | 25142708 | 0.2164 | rs713586 | −6.37 | rs1541984 |
| VAT/ASAT | CCDC92 | 12 | 124403207 | 124457378 | 0.3102 | rs7133378 | −5.83 | rs7307053 |
| VAT/ASAT | HLA-DRB6 | 6 | 32520490 | 32527799 | 0.8939 | rs532098 | 5.63 | rs28366298 |
| VAT/ASAT | HLA-DRA | 6 | 32407619 | 32412823 | 0.1423 | rs532098 | 5.63 | rs28366298 |
| VAT/ASAT | PEMT | 17 | 17408877 | 17495022 | 0.3651 | rs8074272 | 5.52 | rs4646385 |
| VAT/ASAT | XXbac-BPG299F13.14 | 6 | 31168262 | 31169695 | 0.0648 | rs2523578 | −5.67 | rs2523578 |
| VAT/ASAT | EXOSC10 | 1 | 11126675 | 11158213 | 0.1386 | rs6701524 | −5.09 | rs2486920 |
| VAT/ASAT | RP11-380L11.4 | 12 | 124410008 | 124410630 | 0.1109 | rs7133378 | −5.83 | rs4930726 |
| VAT/ASAT | RP4-635E18.7 | 1 | 11128528 | 11133154 | 0.1104 | rs6701524 | −5.09 | rs2791653 |
| VAT/ASAT | RP11-524F11.1 | 17 | 17410665 | 17411622 | 0.1149 | rs8074272 | 5.52 | rs750546 |
| VAT/ASAT | CDK2AP1 | 12 | 123746031 | 123756881 | 0.2554 | rs4759415 | −3.99 | rs1879380 |
| VAT/ASAT | MSH5 | 6 | 31707725 | 31730575 | 0.078 | rs2523578 | −5.67 | rs2269426 |
| VAT/ASAT | HLA-S | 6 | 31349851 | 31350065 | 0.5236 | rs2523578 | −5.67 | rs2523578 |
| VAT/ASAT | VEGFB | 11 | 64002010 | 64006259 | 0.1728 | rs35169799 | 4.7 | rs35169799 |
| VAT/ASAT | ADAM1B | 12 | 112364822 | 112366821 | 0.0408 | rs666951 | −4.48 | rs11066118 |
| VAT/ASAT | XXbac-BPG248L24.12 | 6 | 31324424 | 31325414 | 0.2306 | rs2523578 | −5.67 | rs2844623 |
| VAT/ASAT | CYP21A1P | 6 | 31973466 | 31976176 | 0.4095 | rs1150755 | −5.33 | rs2071295 |
| VAT/ASAT | XXbac-BPG154L12.4 | 6 | 32223488 | 32233615 | 0.0977 | rs532098 | 5.63 | rs28366298 |
| VAT/ASAT | HLA-B | 6 | 31321649 | 31324219 | 0.2206 | rs2523578 | −5.67 | rs3130560 |
| VAT/ASAT | PAPPA | 9 | 118916083 | 119164601 | 0.1285 | rs4836749 | −3.62 | rs1998499 |
| VAT/ASAT | C2 | 6 | 31865562 | 31913426 | 0.0897 | rs1150755 | −5.33 | rs3130286 |
| VAT/ASAT | RP11-132M7.3 | 6 | 85399148 | 85419252 | 0.1883 | rs4144149 | 4.79 | rs4320330 |
| VAT/ASAT | AAMP | 2 | 219128850 | 219134980 | 0.0521 | rs3731861 | 4.68 | rs992157 |
| VAT/ASAT | SKIV2L | 6 | 31926888 | 31937532 | 0.4759 | rs1150755 | −5.33 | rs391165 |
| VAT/ASAT | RP11-378A13.1 | 2 | 219120042 | 219122087 | 0.3243 | rs3731861 | 4.68 | rs736730 |
| VAT/ASAT | PNKD | 2 | 219135115 | 219211516 | 0.0782 | rs3731861 | 4.68 | rs4672884 |
| VAT/ASAT | CLIC1 | 6 | 31698395 | 31707540 | 0.0696 | rs2523578 | −5.67 | rs3130484 |
| VAT/ASAT | GSTM1 | 1 | 110230436 | 110236367 | 0.4273 | rs390923 | 3.5 | rs11101992 |
| VAT/ASAT | ARIH2 | 3 | 48958913 | 49023815 | 0.0939 | rs6791542 | −3.99 | rs4974082 |
| VAT/ASAT | PRDX5 | 11 | 64085560 | 64089283 | 0.1495 | rs35169799 | 4.7 | rs3782101 |
| VAT/ASAT | HECTD4 | 12 | 112597992 | 112819896 | 0.0764 | rs2301756 | −4.46 | rs7294902 |
| VAT/ASAT | LINC00910 | 17 | 41447213 | 41466567 | 0.0754 | rs12944458 | 4.16 | rs12944458 |
| VAT/ASAT | HLA-DQA2 | 6 | 32709119 | 32714992 | 0.8335 | rs532098 | 5.63 | rs28366298 |
| VAT/ASAT | DMWD | 19 | 46286205 | 46296060 | 0.1118 | rs123187 | 3.72 | rs725660 |
| VAT/ASAT | NSFP1 | 17 | 44450221 | 44564507 | 0.7903 | rs17698176 | 3.58 | rs17698176 |
| VAT/ASAT | WNT16 | 7 | 120965421 | 120981158 | 0.1369 | rs10276111 | −4.23 | rs10241888 |
| VAT/ASAT | CLTB | 5 | 175819456 | 175843570 | 0.1085 | rs7703742 | −4.07 | rs11959740 |
| VAT/ASAT | WDR6 | 3 | 49044588 | 49053236 | 0.5122 | rs6791542 | −3.99 | rs6446205 |
| VAT/ASAT | RPS26 | 12 | 56435637 | 56438116 | 0.783 | rs877636 | −4.83 | rs10876864 |
| VAT/ASAT | PAN2 | 12 | 56710121 | 56727837 | 0.0699 | rs877636 | −4.83 | rs17118439 |
| VAT/ASAT | HLA-DRB1 | 6 | 32546546 | 32557625 | 0.399 | rs532098 | 5.63 | rs9271170 |
| VAT/ASAT | C11orf49 | 11 | 46958240 | 47185847 | 0.1038 | rs7109698 | −4.61 | rs1352307 |
| VAT/ASAT | C6orf106 | 6 | 34555065 | 34664636 | 0.1107 | rs1150779 | 5.29 | rs16894959 |
| VAT/ASAT | SUOX | 12 | 56390964 | 56400425 | 0.1121 | rs877636 | −4.83 | rs10876864 |
| VAT/GFAT | CCDC92 | 12 | 124403207 | 124457378 | 0.3169 | rs7133378 | −7.72 | rs4930721 |
| VAT/GFAT | CEBPA-AS1 | 19 | 33793763 | 33795941 | 0.1559 | rs17529595 | −6.92 | rs17529595 |
| VAT/GFAT | RP11-380L11.4 | 12 | 124410008 | 124410630 | 0.0798 | rs7133378 | −7.72 | rs4930726 |
| VAT/GFAT | DNAH10OS | 12 | 124410971 | 124419531 | 0.1162 | rs7133378 | −7.72 | rs4765127 |
| VAT/GFAT | HLA-S | 6 | 31349851 | 31350065 | 0.5473 | rs2523578 | −6.39 | rs2523578 |
| VAT/GFAT | DNAH10 | 12 | 124247042 | 124420168 | 0.4157 | rs7133378 | −7.72 | rs12309481 |
| VAT/GFAT | FLOT1 | 6 | 30695486 | 30710510 | 0.0716 | rs3130557 | −5.36 | rs3130557 |
| VAT/GFAT | CYP21A1P | 6 | 31973466 | 31976176 | 0.3074 | rs537160 | −5.99 | rs2269426 |
| VAT/GFAT | PRRT1 | 6 | 32116136 | 32121621 | 0.1097 | rs537160 | −5.99 | rs521977 |
| VAT/GFAT | APOM | 6 | 31623248 | 31625987 | 0.0567 | rs2523578 | −6.39 | rs2855812 |
| VAT/GFAT | HLA-DRB1 | 6 | 32546546 | 32557625 | 0.3931 | rs532098 | 5.81 | rs532098 |
| VAT/GFAT | HLA-DRB6 | 6 | 32520490 | 32527799 | 0.8525 | rs532098 | 5.81 | rs28366298 |
| VAT/GFAT | RP11-378A13.1 | 2 | 219120042 | 219122087 | 0.4016 | rs3731861 | 5.11 | rs736731 |
| VAT/GFAT | C3orf62 | 3 | 49306219 | 49315263 | 0.05 | rs11714957 | 5.57 | rs9874474 |
| VAT/GFAT | HCG23 | 6 | 32358287 | 32361463 | 0.0794 | rs537160 | −5.99 | rs9271055 |
| VAT/GFAT | BTN3A3 | 6 | 26440700 | 26453643 | 0.2595 | rs6456739 | −4.05 | rs1131936 |
| VAT/GFAT | HLA-C | 6 | 31236526 | 31239882 | 0.5466 | rs2523578 | −6.39 | rs1265087 |
| VAT/GFAT | FAM154B | 15 | 82555151 | 82577271 | 0.5902 | rs9972386 | −4.76 | rs9972386 |
| VAT/GFAT | XXbac-BPG248L24.12 | 6 | 31324424 | 31325414 | 0.2052 | rs2523578 | −6.39 | rs2844623 |
| VAT/GFAT | HLA-DQB1-AS1 | 6 | 32628132 | 32628506 | 0.5356 | rs532098 | 5.81 | rs1063355 |
| VAT/GFAT | MAST3 | 19 | 18208603 | 18262502 | 0.0917 | rs12608504 | 5.2 | rs740691 |
| VAT/GFAT | NAA25 | 12 | 112464500 | 112546826 | 0.0709 | rs1980364 | −4.51 | rs4767293 |
| VAT/GFAT | RBM6 | 3 | 49977440 | 50114683 | 0.3976 | rs11714957 | 5.57 | rs4688755 |
| VAT/GFAT | CTC-228N24.3 | 5 | 127276118 | 127418864 | 0.3555 | rs3749748 | −4.36 | rs3749748 |
| VAT/GFAT | SEMA3F | 3 | 50192478 | 50226508 | 0.0448 | rs11714957 | 5.57 | rs3774745 |
| VAT/GFAT | HLA-DQA2 | 6 | 32709119 | 32714992 | 0.8413 | rs532098 | 5.81 | rs28366298 |
| VAT/GFAT | PNKD | 2 | 219135115 | 219211516 | 0.164 | rs3731861 | 5.11 | rs4672884 |
| VAT/GFAT | GS1-259H13.2 | 7 | 99195689 | 99208439 | 0.1785 | rs3843540 | −4.23 | rs6947826 |
| VAT/GFAT | C4A | 6 | 31949801 | 31970458 | 0.276 | rs537160 | −5.99 | rs3101018 |
| VAT/GFAT | TRAPPC10 | 21 | 45432200 | 45526433 | 0.1053 | rs8131020 | −3.53 | rs2838441 |
| VAT/GFAT | RP11-114F10.3 | 12 | 106496941 | 106499943 | 0.0821 | rs12425720 | −4.6 | rs10161316 |
| VAT/GFAT | EXOSC10 | 1 | 11126675 | 11158213 | 0.1137 | rs1057079 | −4.87 | rs2791655 |
| VAT/GFAT | RRAS2 | 11 | 14299472 | 14386052 | 0.0676 | rs11238 | 4.03 | rs11023197 |
| VAT/GFAT | DALRD3 | 3 | 49053387 | 49059726 | 0.052 | rs6795772 | −4.29 | rs7626445 |
| VAT/GFAT | TMBIM1 | 2 | 219138915 | 219157309 | 0.0981 | rs3731861 | 5.11 | rs10932766 |
| VAT/GFAT | TBX15 | 1 | 119425669 | 119532179 | 0.0951 | rs1891222 | −4.4 | rs2645294 |
| VAT/GFAT | WDR6 | 3 | 49044588 | 49053236 | 0.2343 | rs6795772 | −4.29 | rs9311433 |
| VAT/GFAT | MIR4435-1HG | 2 | 111953927 | 112252677 | 0.1112 | rs1345203 | −4.53 | rs36018702 |
| VAT/GFAT | NCKIPSD | 3 | 48701364 | 48723797 | 0.2129 | rs6791542 | −4.28 | rs12493578 |
| VAT/GFAT | CYP21A2 | 6 | 32006042 | 32009447 | 0.1939 | rs537160 | −5.99 | rs3131382 |
| VAT/GFAT | NT5DC2 | 3 | 52558512 | 52569070 | 0.0858 | rs2244461 | 4.83 | rs7614981 |
| VAT/GFAT | ZSCAN12P1 | 6 | 28058932 | 28061442 | 0.1605 | rs2232423 | −4.23 | rs9393902 |
| VAT/GFAT | TMEM116 | 12 | 112369086 | 112450969 | 0.2995 | rs1980364 | −4.51 | rs11066119 |
| VAT/GFAT | DSTYK | 1 | 205111632 | 205180727 | 0.0742 | rs4951182 | 4.23 | rs1572993 |
| VAT/GFAT | SLC12A2 | 5 | 127419458 | 127525380 | 0.1288 | rs3749748 | −4.36 | rs9327455 |
| VAT/GFAT | CCDC92 | 12 | 124403207 | 124457378 | 0.3102 | rs7133378 | −7.72 | rs7307053 |
| VAT/GFAT | DNAH10OS | 12 | 124410971 | 124419531 | 0.131 | rs7133378 | −7.72 | rs4930726 |
| VAT/GFAT | CEBPA-AS1 | 19 | 33793763 | 33795941 | 0.0957 | rs17529595 | −6.92 | rs17529595 |
| VAT/GFAT | RP11-380L11.4 | 12 | 124410008 | 124410630 | 0.1109 | rs7133378 | −7.72 | rs4930726 |
| VAT/GFAT | XXbac-BPG248L24.12 | 6 | 31324424 | 31325414 | 0.2306 | rs2523578 | −6.39 | rs2844623 |
| VAT/GFAT | HLA-S | 6 | 31349851 | 31350065 | 0.5236 | rs2523578 | −6.39 | rs2523578 |
| VAT/GFAT | VEGFE | 11 | 64002010 | 64006259 | 0.1728 | rs35169799 | 5.71 | rs35169799 |
| VAT/GFAT | C4B | 6 | 31982539 | 32003195 | 0.1682 | rs537160 | −5.99 | rs1150755 |
| VAT/GFAT | IRS1 | 2 | 227596033 | 227664475 | 0.1263 | rs908252 | −5.55 | rs1515116 |
| VAT/GFAT | CYP21A1P | 6 | 31973466 | 31976176 | 0.4095 | rs537160 | −5.99 | rs2071295 |
| VAT/GFAT | ZNF664 | 12 | 124457670 | 124499986 | 0.1843 | rs7133378 | −7.72 | rs863750 |
| VAT/GFAT | ATP6V0A2 | 12 | 124196865 | 124246302 | 0.1793 | rs7133378 | −7.72 | rs7975233 |
| VAT/GFAT | EXOSC10 | 1 | 11126675 | 11158213 | 0.1386 | rs1057079 | −4.87 | rs2486920 |
| VAT/GFAT | VARS2 | 6 | 30881982 | 30894236 | 0.2981 | rs2523578 | −6.39 | rs1265048 |
| VAT/GFAT | MSH5 | 6 | 31707725 | 31730575 | 0.078 | rs2523578 | −6.39 | rs2269426 |
| VAT/GFAT | HLA-DRB6 | 6 | 32520490 | 32527799 | 0.8939 | rs532098 | 5.81 | rs28366298 |
| VAT/GFAT | XXbac-BPG299F13.14 | 6 | 31168262 | 31169695 | 0.0648 | rs2523578 | −6.39 | rs2523578 |
| VAT/GFAT | HLA-DRA | 6 | 32407619 | 32412823 | 0.1423 | rs537160 | −5.99 | rs28366298 |
| VAT/GFAT | MST1R | 3 | 49924435 | 49941277 | 0.0658 | rs11714957 | 5.57 | rs2271961 |
| VAT/GFAT | RP4-635E18.7 | 1 | 11128528 | 11133154 | 0.1104 | rs1057079 | −4.87 | rs2791653 |
| VAT/GFAT | AAMP | 2 | 219128850 | 219134980 | 0.0521 | rs3731861 | 5.11 | rs992157 |
| VAT/GFAT | C2 | 6 | 31865562 | 31913426 | 0.0897 | rs537160 | −5.99 | rs3130286 |
| VAT/GFAT | PNKD | 2 | 219135115 | 219211516 | 0.0782 | rs3731861 | 5.11 | rs4672884 |
| VAT/GFAT | FAM154B | 15 | 82555151 | 82577271 | 0.5225 | rs9972386 | −4.76 | rs9972386 |
| VAT/GFAT | CLIC1 | 6 | 31698395 | 31707540 | 0.0696 | rs2523578 | −6.39 | rs3130484 |
| VAT/GFAT | HLA-B | 6 | 31321649 | 31324219 | 0.2206 | rs2523578 | −6.39 | rs3130560 |
| VAT/GFAT | FAM13A | 4 | 89647106 | 90032549 | 0.155 | rs9991328 | 4.57 | rs9991328 |
| VAT/GFAT | DNAH10 | 12 | 124247042 | 124420168 | 0.2465 | rs7133378 | −7.72 | rs12309481 |
| VAT/GFAT | RP11-378A13.1 | 2 | 219120042 | 219122087 | 0.3243 | rs3731861 | 5.11 | rs736730 |
| VAT/GFAT | NEK4 | 3 | 52744800 | 52804965 | 0.067 | rs2581790 | 4.95 | rs2230535 |
| VAT/GFAT | RBM6 | 3 | 49977440 | 50114683 | 0.4539 | rs11714957 | 5.57 | rs4688755 |
| VAT/GFAT | ADAM1B | 12 | 112364822 | 112366821 | 0.0408 | rs1980364 | −4.51 | rs11066118 |
| VAT/GFAT | PAPPA | 9 | 118916083 | 119164601 | 0.1285 | rs1885241 | −3.76 | rs1998499 |
| VAT/GFAT | HLA-DQB1-AS1 | 6 | 32628132 | 32628506 | 0.6081 | rs532098 | 5.81 | rs9271055 |
| VAT/GFAT | ARIH2 | 3 | 48958913 | 49023815 | 0.0939 | rs6795772 | −4.29 | rs4974082 |
| VAT/GFAT | CDK2AP1 | 12 | 123746031 | 123756881 | 0.2554 | rs1790099 | −3.66 | rs1879380 |
| VAT/GFAT | MAP3K13 | 3 | 185000729 | 185206885 | 0.049 | rs4687248 | 4.48 | rs7431357 |
| VAT/GFAT | TMBIM1 | 2 | 219138915 | 219157309 | 0.1315 | rs3731861 | 5.11 | rs1017698 |
| VAT/GFAT | DALRD3 | 3 | 49053387 | 49059726 | 0.0769 | rs6795772 | −4.29 | rs9840050 |
| VAT/GFAT | CTC-228N24.3 | 5 | 127276118 | 127418864 | 0.418 | rs3749748 | −4.36 | rs3749748 |
| VAT/GFAT | XXbac-BPG154L12.4 | 6 | 32223488 | 32233615 | 0.0977 | rs537160 | −5.99 | rs28366298 |
| VAT/GFAT | HLA-DQA2 | 6 | 32709119 | 32714992 | 0.8335 | rs532098 | 5.81 | rs28366298 |
| VAT/GFAT | HLA-DRB1 | 6 | 32546546 | 32557625 | 0.399 | rs532098 | 5.81 | rs9271170 |
| VAT/GFAT | NCKIPSD | 3 | 48701364 | 48723797 | 0.142 | rs6791542 | −4.28 | rs12493578 |
| VAT/GFAT | GSTM1 | 1 | 110230436 | 110236367 | 0.4273 | rs390923 | 3.77 | rs11101992 |
| VAT/GFAT | CELSR3 | 3 | 48673902 | 48700348 | 0.038 | rs6791542 | −4.28 | rs6779394 |
| VAT/GFAT | DMWD | 19 | 46286205 | 46296060 | 0.1118 | rs12972151 | 4.8 | rs725660 |
| VAT/GFAT | SKIV2L | 6 | 31926888 | 31937532 | 0.4759 | rs537160 | −5.99 | rs391165 |
| VAT/GFAT | WDR6 | 3 | 49044588 | 49053236 | 0.5122 | rs6795772 | −4.29 | rs6446205 |
| VAT/GFAT | CLTB | 5 | 175819456 | 175843570 | 0.1085 | rs11959740 | −3.96 | rs11959740 |
| VAT/GFAT | QARS | 3 | 49133365 | 49142553 | 0.0435 | rs6795772 | −4.29 | rs4855864 |
| VAT/GFAT | TMEM116 | 12 | 112369086 | 112450969 | 0.2501 | rs1980364 | −4.51 | rs7295294 |
| VAT/GFAT | HECTD4 | 12 | 112597992 | 112819896 | 0.0764 | rs1980364 | −4.51 | rs7294902 |
| VAT/GFAT | MRAS | 3 | 138066539 | 138124375 | 0.1214 | rs6807945 | 4.47 | rs2293251 |
| ASAT/GFAT | CCDC92 | 12 | 124403207 | 124457378 | 0.3102 | rs7133378 | −5.71 | rs7307053 |
| ASAT/GFAT | TIPARP | 3 | 156391024 | 156424559 | 0.1228 | rs900399 | −8.69 | rs10049090 |
| ASAT/GFAT | DNAH10OS | 12 | 124410971 | 124419531 | 0.131 | rs7133378 | −5.71 | rs4930726 |
| ASAT/GFAT | RP4-712E4.1 | 1 | 119542967 | 119543516 | 0.2441 | rs2645290 | −6.12 | rs1409159 |
| ASAT/GFAT | RP11-380L11.4 | 12 | 124410008 | 124410630 | 0.1109 | rs7133378 | −5.71 | rs4930726 |
| ASAT/GFAT | THBS3 | 1 | 155165379 | 155177708 | 0.0666 | rs11264329 | 4.71 | rs4971079 |
| ASAT/GFAT | PDGFC | 4 | 157681606 | 157892546 | 0.0706 | rs13108763 | −6.22 | rs2113992 |
| ASAT/GFAT | CTC-228N24.3 | 5 | 127276118 | 127418864 | 0.418 | rs3749748 | −4.764 | rs3749748 |
| ASAT/GFAT | CALCRL | 2 | 188207856 | 188313187 | 0.0454 | rs1918901 | −5.019 | rs13417165 |
| ASAT/GFAT | WNT3 | 17 | 44839872 | 44910424 | 0.1306 | rs11079750 | −4.43 | rs12452064 |
| ASAT/GFAT | EYA1 | 8 | 72109668 | 72274467 | 0.1586 | rs10093418 | 5.12 | rs35510588 |
| ASAT/GFAT | MEST | 7 | 130126025 | 130146088 | 0.1716 | rs11556924 | −4.7 | rs17164872 |
| ASAT/GFAT | XXbac-BPG248L24.12 | 6 | 31324424 | 31325414 | 0.2306 | rs2844623 | 4.05 | rs2844623 |
| ASAT/GFAT | ATP6V0A2 | 12 | 124196865 | 124246302 | 0.1793 | rs7133378 | −5.71 | rs7975233 |
| ASAT/GFAT | SETD2 | 3 | 47057919 | 47205457 | 0.0882 | rs6768722 | −4.54 | rs11130126 |
| ASAT/GFAT | RP11-2E11.9 | 7 | 130147501 | 130148123 | 0.1295 | rs11556924 | −4.7 | rs5011386 |
| ASAT/GFAT | RP11-2E11.5 | 7 | 130121332 | 130124233 | 0.0983 | rs11556924 | −4.7 | rs2268382 |
| ASAT/GFAT | PMS2P3 | 7 | 75137069 | 75157478 | 0.2208 | rs17207196 | −4.29 | rs17207196 |
| ASAT/GFAT | POM121C | 7 | 75046069 | 75115548 | 0.1231 | rs17207196 | −4.29 | rs17207196 |
| ASAT/GFAT | GTF2IP1 | 7 | 74602783 | 74653438 | 0.1106 | rs17207196 | −4.29 | rs17207196 |
| ASAT/GFAT | CTD-2380F24.1 | 16 | 19772561 | 19777421 | 0.116 | rs11865578 | −4.6 | rs1858973 |
| ASAT/GFAT | KNOP1 | 16 | 19714902 | 19729016 | 0.4112 | rs11865578 | −4.6 | rs720176 |
| ASAT/GFAT | ZNF664 | 12 | 124457670 | 124499986 | 0.1843 | rs7133378 | −5.71 | rs863750 |
| ASAT/GFAT | PTPN23 | 3 | 47422501 | 47454931 | 0.0271 | rs6768722 | −4.54 | rs11705957 |
| ASAT/GFAT | TBX15 | 1 | 119425669 | 119532179 | 0.0973 | rs2645290 | −6.12 | rs984225 |
| ASAT/GFAT | RP11-708J19.1 | 3 | 47420579 | 47422489 | 0.0347 | rs6768722 | −4.54 | rs11710322 |
| ASAT/GFAT | ARL17B | 17 | 44352150 | 44439130 | 0.5984 | rs11658976 | −3.89 | rs10432043 |
| ASAT/GFAT | RBFOX2 | 22 | 36134783 | 36424473 | 0.138 | rs1894469 | −4.17 | rs10154656 |
| ASAT/GFAT | GNA12 | 7 | 2767746 | 2883958 | 0.1019 | rs7805092 | −4.86 | rs798492 |
| ASAT/GFAT | STAG3L1 | 7 | 74988448 | 75024291 | 0.4615 | rs17207196 | −4.29 | rs17207196 |

| | | | | | | | MODEL | MODEL | | |
|---|---|---|---|---|---|---|---|---|---|---|
| pheno | EQTL.R2 | EQTL.Z | EQTL.GWAS.Z | NSNP | NWGT | MODEL | CV.R2 | CV.PV | TWAS.Z | TWAS.P |
| VATadj | 0.074925 | 5.08 | −7.428 | 469 | 1 | top1 | 0.075 | 5.40E−07 | −7.428 | 1.10E−13 |
| VATadj | 0.219 | 8.35 | −5.58 | 436 | 7 | lasso | 0.24 | 6.70E−21 | −6.6598 | 2.74E−11 |
| VATadj | 0.000918 | 4.09 | −4.856 | 77 | 77 | blup | 0.014 | 0.02 | −6.36471 | 1.96E−10 |
| VATadj | 0.12 | 7.06 | 4.132 | 197 | 8 | lasso | 0.23 | 3.60E−19 | 6.04323 | 1.51E−09 |
| VATadj | 0.503 | 12.53 | 5.968 | 249 | 12 | lasso | 0.66 | 6.80E−75 | 5.98862 | 2.12E−09 |
| VATadj | 0.142 | −7.36 | −6.714 | 239 | 39 | enet | 0.35 | 1.40E−30 | 5.76752 | 8.04E−09 |
| VATadj | 0.063669 | −5.38 | −5.588 | 235 | 1 | top1 | 0.064 | 3.80E−06 | 5.588 | 2.30E−08 |
| VATadj | 0.0146 | −3.63 | −3.264 | 244 | 244 | blup | 0.034 | 0.00064 | 5.57372 | 2.49E−08 |
| VATadj | 0.04 | 3.98 | −5.422 | 358 | 1 | top1 | 0.04 | 0.00022 | −5.422 | 5.89E−08 |
| VATadj | 0.0788 | −5.16 | −5.374 | 159 | 1 | top1 | 0.079 | 2.80E−07 | 5.374 | 7.70E−08 |
| VATadj | 0.001513 | −4.14 | 4.234 | 418 | 418 | blup | 0.024 | 0.0037 | −5.36012 | 8.32E−08 |
| VATadj | 0.00449 | −3.68 | −3.652 | 227 | 19 | enet | 0.037 | 0.00039 | 5.32951 | 9.85E−08 |
| VATadj | 0.141 | 6.77 | −3.09 | 412 | 37 | enet | 0.14 | 3.20E−12 | −5.3025 | 1.14E−07 |
| VATadj | 0.543 | 13.01 | 5.968 | 254 | 11 | lasso | 0.66 | 2.70E−75 | 5.20447 | 1.95E−07 |
| VATadj | 0.174 | −7.47 | 5.876 | 233 | 4 | lasso | 0.21 | 5.10E−18 | −5.17825 | 2.24E−07 |
| VATadj | 0.080672 | −5.12 | 5.356 | 436 | 4 | lasso | 0.082 | 1.50E−07 | −5.12908 | 2.91E−07 |
| VATadj | 0.0169 | 4.12 | −5.806 | 429 | 429 | blup | 0.024 | 0.0037 | −4.9234 | 8.50E−07 |
| VATadj | 0.289171 | −9.55 | 4.473 | 449 | 33 | enet | 0.29 | 4.10E−25 | −4.91928 | 8.69E−07 |
| VATadj | 0.036 | 4.8 | 2.387 | 218 | 25 | enet | 0.08 | 2.40E−07 | 4.78642 | 1.70E−06 |
| VATadj | 0.00846 | 5.07 | 4.152 | 176 | 43 | enet | 0.13 | 2.10E−11 | 4.7196 | 2.36E−06 |
| VATadj | 0.135 | 6.62 | −1.812 | 207 | 207 | blup | 0.26 | 5.70E−22 | −4.71254 | 2.45E−06 |
| VATadj | 0.0666 | −5.2 | −4.708 | 427 | 1 | top1 | 0.067 | 2.30E−06 | 4.708 | 2.50E−06 |
| VATadj | 0.00464 | 3.55 | −3.76 | 203 | 203 | blup | 0.0057 | 0.096 | −4.6831 | 2.83E−06 |
| VATadj | 0.0128 | 3.72 | −3.684 | 189 | 17 | enet | 0.028 | 0.0017 | −4.52794 | 5.96E−06 |
| VATadj | 0.250251 | −8.93 | −4.516 | 265 | 1 | top1 | 0.25 | 2.20E−21 | 4.516 | 6.30E−06 |
| VATadj | 0.031464 | −4.45 | 4.503 | 434 | 1 | top1 | 0.031 | 0.00097 | −4.503 | 6.70E−06 |
| VATadj | 0.05647 | −4.96 | −4.497 | 265 | 1 | top1 | 0.056 | 1.30E−05 | 4.497 | 6.89E−06 |
| VATadj | 0.0648 | 5.81 | −5.7 | 427 | 427 | blup | 0.077 | 3.90E−07 | −4.4758 | 7.61E−06 |
| VATadj | 0.0491 | −5.18 | 4.764 | 559 | 9 | enet | 0.055 | 1.70E−05 | −4.42396 | 9.69E−06 |
| VATadj | 0.45 | −11.89 | −4.301 | 176 | 25 | enet | 0.51 | 7.50E−51 | 4.36803 | 1.25E−05 |
| VATadj | 0.252 | 8.92 | −2.82 | 214 | 23 | enet | 0.36 | 1.30E−31 | −4.2834 | 1.84E−05 |
| VATadj | 0.33443 | 10.54 | −4.438 | 263 | 263 | blup | 0.35 | 4.30E−31 | −4.26917 | 1.96E−05 |
| VATadj | 0.0368 | −4.59 | 4.265 | 596 | 2 | lasso | 0.041 | 0.00018 | −4.259752 | 2.05E−05 |
| VATadj | 0.022153 | −4.53 | −4.442 | 264 | 264 | blup | 0.044 | 0.00011 | 4.1493 | 3.33E−05 |
| VATadj | 0.011284 | 3.95 | 3.441 | 375 | 375 | blup | 0.024 | 0.0032 | 4.1201 | 3.79E−05 |
| VATadj | 0.171798 | −7.43 | −4.119 | 260 | 1 | top1 | 0.17 | 1.30E−14 | 4.119 | 3.81E−05 |
| VATadj | 0.155582 | −7.03 | −4.119 | 272 | 1 | top1 | 0.16 | 2.80E−13 | 4.119 | 3.81E−05 |
| VATadj | 0.000102 | −3.48 | −1.655 | 278 | 278 | blup | 0.023 | 0.0039 | 4.0824 | 4.46E−05 |
| VATadj | 0.0216 | −4.3 | −2.409 | 189 | 37 | enet | 0.039 | 0.00025 | 4.04037 | 5.34E−05 |
| VATadj | 0.264102 | −9.19 | 4.033 | 370 | 1 | top1 | 0.26 | 1.20E−22 | −4.033 | 5.51E−05 |
| VATadj | 0.107289 | −5.96 | 4.013 | 332 | 1 | top1 | 0.11 | 1.90E−09 | −4.013 | 6.00E−05 |
| VATadj | 0.0266 | −5.01 | −4.159 | 314 | 314 | blup | 0.049 | 4.80E−05 | 4.0082 | 6.12E−05 |
| VATadj | 0.093061 | 6.09 | −4.107 | 307 | 27 | enet | 0.12 | 3.90E−10 | −3.9153 | 9.03E−05 |
| VATadj | 0.110123 | 6.05 | −3.9 | 324 | 1 | top1 | 0.11 | 1.10E−09 | −3.9 | 9.62E−05 |
| ASATadj | 0.057137 | −5.04 | −6.258 | 185 | 1 | top1 | 0.057 | 1.30E−06 | 6.258 | 3.90E−10 |
| ASATadj | 0.0458 | 4.8 | −7.794 | 433 | 17 | enet | 0.066 | 1.80E−07 | −6.1224 | 9.22E−10 |
| ASATadj | 0.165 | −8.24 | 5.64 | 246 | 18 | enet | 0.18 | 2.50E−18 | −6.03124 | 1.63E−09 |
| ASATadj | 0.248 | −10.15 | 5.64 | 253 | 1 | top1 | 0.25 | 1.20E−25 | −5.64 | 1.70E−08 |
| ASATadj | 0.077195 | 5.71 | 5.466 | 458 | 1 | top1 | 0.077 | 1.80E−08 | 5.466 | 4.60E−08 |
| ASATadj | 0.0144 | 3.68 | 5.673 | 292 | 292 | blup | 0.021 | 0.0028 | 5.3247 | 1.01E−07 |
| ASATadj | 0.0286 | −4.1 | 5.253 | 487 | 1 | top1 | 0.029 | 0.00051 | −5.253 | 1.50E−07 |
| ASATadj | 0.00126 | 3.62 | −4.948 | 267 | 7 | lasso | 0.025 | 0.0011 | −5.21896 | 1.80E−07 |
| ASATadj | 0.0197 | −3.8 | 5.173 | 438 | 1 | top1 | 0.02 | 0.0034 | −5.173 | 2.30E−07 |
| ASATadj | 0.0588 | 5.17 | 5.173 | 443 | 1 | top1 | 0.059 | 9.00E−07 | 5.173 | 2.30E−07 |
| ASATadj | 0.00438 | −3.61 | 5.06 | 387 | 387 | blup | 0.0053 | 0.082 | −4.9051 | 9.34E−07 |
| ASATadj | 0.194 | −8.79 | −4.873 | 261 | 1 | top1 | 0.19 | 6.90E−20 | 4.873 | 1.10E−06 |
| ASATadj | 0.14 | −7.7 | −4.811 | 269 | 1 | top1 | 0.14 | 1.90E−14 | 4.811 | 1.50E−06 |
| ASATadj | 0.17 | 8.37 | −4.53 | 425 | 11 | lasso | 0.18 | 5.00E−18 | −4.762783 | 1.91E−06 |
| ASATadj | 0.030824 | 4.72 | 1.514 | 291 | 291 | blup | 0.053 | 3.10E−06 | 4.7376 | 2.16E−06 |
| ASATadj | 0.0162 | 4.26 | 4.102 | 338 | 338 | blup | 0.021 | 0.0028 | 4.736622 | 2.17E−06 |
| ASATadj | 0.0517 | 4.58 | 4.734 | 477 | 1 | top1 | 0.052 | 4.00E−06 | 4.734 | 2.20E−06 |
| ASATadj | 0.083342 | 6.2 | 4.678 | 318 | 1 | top1 | 0.083 | 5.00E−09 | 4.678 | 2.90E−06 |
| ASATadj | 0.071545 | 6.24 | 3.911 | 369 | 39 | enet | 0.1 | 8.90E−11 | 4.57085 | 4.86E−06 |
| ASATadj | 0.142 | 7.47 | 4.561 | 547 | 1 | top1 | 0.14 | 1.30E−14 | 4.561 | 5.09E−06 |
| ASATadj | 0.278667 | −10.42 | 4.561 | 332 | 1 | top1 | 0.28 | 3.70E−29 | −4.561 | 5.09E−06 |
| ASATadj | 0.0228 | 5.21 | 6.27 | 436 | 436 | blup | 0.037 | 8.00E−05 | 4.475051 | 7.64E−06 |
| ASATadj | 0.0716 | 5.67 | 5.63 | 289 | 289 | blup | 0.097 | 2.80E−10 | 4.47262 | 7.73E−06 |
| ASATadj | 0.0207 | −4.35 | −4.417 | 427 | 1 | top1 | 0.021 | 0.0027 | 4.417 | 1.00E−05 |
| ASATadj | 0.116665 | 6.83 | 3.911 | 372 | 2 | lasso | 0.12 | 2.80E−12 | 4.34111 | 1.42E−05 |
| ASATadj | 0.106 | 6.89 | 3.615 | 398 | 4 | lasso | 0.15 | 3.10E−15 | 4.250222 | 2.14E−05 |
| ASATadj | 0.0135 | 3.8 | −0.468 | 375 | 375 | blup | 0.022 | 0.0023 | −4.14903 | 3.34E−05 |
| ASATadj | 0.122 | 7.22 | −4.45 | 305 | 23 | enet | 0.13 | 1.90E−13 | −4.116809 | 3.84E−05 |
| ASATadj | 0.000665 | 3.48 | 1.598 | 429 | 429 | blup | 0.014 | 0.012 | 4.07715 | 4.56E−05 |
| ASATadj | 0.188637 | 8.86 | 4.36 | 331 | 3 | lasso | 0.22 | 1.20E−22 | 4.0758 | 4.59E−05 |
| ASATadj | 0.18 | 8.91 | 5.64 | 359 | 36 | enet | 0.23 | 6.40E−24 | 4.06581 | 4.79E−05 |
| ASATadj | 0.213 | −9.24 | 3.96 | 338 | 6 | lasso | 0.22 | 1.80E−22 | −4.04773 | 5.17E−05 |
| ASATadj | 0.0602 | −5.38 | −4.021 | 514 | 1 | top1 | 0.06 | 6.70E−07 | 4.021 | 5.80E−05 |
| ASATadj | 0.0613 | −5.45 | −3.983 | 349 | 1 | top1 | 0.061 | 5.40E−07 | 3.983 | 6.81E−05 |
| ASATadj | 0.0649 | −5.55 | 3.983 | 337 | 1 | top1 | 0.065 | 2.50E−07 | −3.983 | 6.81E−05 |
| ASATadj | 0.304 | −10.84 | 3.983 | 337 | 1 | top1 | 0.3 | 3.50E−32 | −3.983 | 6.81E−05 |
| ASATadj | 0.102837 | 7.29 | 4.463 | 309 | 19 | enet | 0.13 | 4.90E−13 | 3.9801 | 6.89E−05 |
| ASATadj | 0.00789 | 3.88 | −3.846 | 321 | 18 | enet | 0.024 | 0.0013 | −3.9752 | 7.03E−05 |
| ASATadj | 0.056855 | 4.96 | 3.954 | 276 | 1 | top1 | 0.057 | 1.40E−06 | 3.954 | 7.69E−05 |
| ASATadj | 0.419 | 12.7 | −2.366 | 359 | 9 | lasso | 0.49 | 4.10E−58 | −3.94269 | 8.06E−05 |
| ASATadj | 0.338 | 11.45 | −3.924 | 328 | 1 | top1 | 0.34 | 2.40E−36 | −3.924 | 8.71E−05 |
| ASATadj | 0.0617 | 5.14 | −3.903 | 419 | 1 | top1 | 0.062 | 4.90E−07 | −3.903 | 9.50E−05 |
| ASATadj | 0.0446 | 4.71 | 3.9 | 364 | 1 | top1 | 0.045 | 1.80E−05 | 3.9 | 9.62E−05 |
| GFATadj | 0.0287 | 6.21 | 9.851 | 437 | 26 | enet | 0.13 | 1.40E−13 | 12.0222 | 2.72E−33 |
| GFATadj | 0.144 | 7.59 | 10.505 | 428 | 1 | top1 | 0.14 | 8.00E−15 | 10.505 | 8.19E−26 |
| GFATadj | 0.0438 | 5.52 | 10.505 | 430 | 8 | lasso | 0.054 | 2.40E−06 | 9.9709 | 2.04E−23 |
| GFATadj | 0.077195 | 5.71 | 9.141 | 458 | 1 | top1 | 0.077 | 1.80E−08 | 9.141 | 6.19E−20 |
| GFATadj | 0.0745 | 5.67 | 8.79 | 436 | 1 | top1 | 0.075 | 3.30E−08 | 8.79 | 1.50E−18 |
| GFATadj | 0.0286 | −4.1 | 8.488 | 487 | 1 | top1 | 0.029 | 0.00051 | −8.488 | 2.10E−17 |
| GFATadj | 0.0583 | 6.78 | 7.172 | 413 | 5 | lasso | 0.12 | 8.40E−13 | 7.8713 | 3.51E−15 |
| GFATadj | 0.0517 | 4.58 | 7.549 | 477 | 1 | top1 | 0.052 | 4.00E−06 | 7.549 | 4.39E−14 |
| GFATadj | 0.0412 | −6.31 | −7.034 | 345 | 1 | top1 | 0.041 | 3.70E−05 | 7.034 | 2.01E−12 |
| GFATadj | 0.070425 | 5.41 | −6.6 | 463 | 1 | top1 | 0.07 | 7.80E−08 | −6.6 | 4.11E−11 |
| GFATadj | 0.013525 | 3.78 | 6.279 | 367 | 367 | blup | 0.017 | 0.0059 | 6.23236 | 4.59E−10 |
| GFATadj | 0.076088 | −6.02 | 5.495 | 346 | 3 | lasso | 0.08 | 1.10E−08 | −5.953676 | 2.62E−09 |
| GFATadj | 0.045727 | 5.93 | −4.988 | 395 | 15 | enet | 0.079 | 1.20E−08 | −5.89931 | 3.65E−09 |
| GFATadj | 0.021786 | −4.79 | −5.008 | 392 | 392 | blup | 0.052 | 3.40E−06 | 5.83165 | 5.49E−09 |
| GFATadj | 0.062786 | 5.32 | 5.715 | 403 | 1 | top1 | 0.063 | 3.90E−07 | 5.715 | 1.10E−08 |
| GFATadj | 0.06149 | 5.05 | 5.715 | 395 | 1 | top1 | 0.061 | 5.10E−07 | 5.715 | 1.10E−08 |
| GFATadj | 0.289 | 10.6 | 4.811 | 467 | 7 | lasso | 0.33 | 7.50E−35 | 5.6118 | 2.00E−08 |
| GFATadj | 0.0588 | 5.17 | 5.595 | 443 | 1 | top1 | 0.059 | 9.00E−07 | 5.595 | 2.21E−08 |
| GFATadj | 0.103 | 7.18 | −1.65 | 399 | 399 | blup | 0.13 | 7.10E−14 | −5.5738 | 2.49E−08 |
| GFATadj | 0.052989 | −5.68 | 3.583 | 349 | 14 | enet | 0.085 | 3.70E−09 | −5.380997 | 7.41E−08 |
| GFATadj | 0.022614 | −5.02 | 3.732 | 454 | 7 | lasso | 0.058 | 1.10E−06 | −5.378526 | 7.51E−08 |
| GFATadj | 0.178 | −8.49 | −4.664 | 474 | 9 | lasso | 0.2 | 5.20E−20 | 5.36781 | 7.97E−08 |
| GFATadj | 0.048435 | −5.46 | 2.64 | 344 | 6 | lasso | 0.098 | 2.20E−10 | 5.26494 | 1.40E−07 |
| GFATadj | 0.140571 | 7.51 | −3.633 | 219 | 14 | enet | 0.17 | 3.40E−17 | −5.18734 | 2.13E−07 |
| GFATadj | 0.352377 | −11.92 | 5.165 | 379 | 6 | lasso | 0.36 | 6.70E−39 | −5.131345 | 2.88E−07 |
| GFATadj | 0.0228 | −4.58 | 4.902 | 146 | 146 | blup | 0.038 | 7.50E−05 | −5.06472 | 4.09E−07 |
| GFATadj | -0.001449 | −3.57 | 2.484 | 388 | 388 | blup | 0.029 | 0.00044 | −5.0571 | 4.26E−07 |
| GFATadj | 0.20159 | 8.98 | 4.825 | 390 | 8 | enet | 0.2 | 1.10E−20 | 5.021809 | 5.12E−07 |
| GFATadj | 0.0182 | 3.82 | 4.534 | 146 | 146 | blup | 0.02 | 0.0029 | 4.97181 | 6.63E−07 |
| GFATadj | 0.199 | −9.05 | −4.825 | 385 | 1 | top1 | 0.2 | 1.90E−20 | 4.825 | 1.40E−06 |
| GFATadj | 0.0197 | −3.8 | 4.786 | 438 | 1 | top1 | 0.02 | 0.0034 | −4.786 | 1.70E−06 |
| GFATadj | 0.169141 | −8.56 | 4.753 | 437 | 1 | top1 | 0.17 | 2.50E−17 | −4.753 | 2.00E−06 |
| GFATadj | 0.205 | 9.28 | 4.611 | 475 | 21 | enet | 0.26 | 3.50E−27 | 4.7528 | 2.01E−06 |
| GFATadj | 0.0585 | −5.56 | −4.678 | 384 | 1 | top1 | 0.058 | 9.70E−07 | 4.678 | 2.90E−06 |
| GFATadj | 0.073436 | 5.48 | −4.671 | 499 | 1 | top1 | 0.073 | 4.10E−08 | −4.671 | 3.00E−06 |
| GFATadj | 0.0112 | 4.34 | −3.216 | 339 | 52 | enet | 0.086 | 2.70E−09 | −4.6605 | 3.15E−06 |
| GFATadj | 0.122 | 7.75 | −4.753 | 384 | 7 | lasso | 0.13 | 4.40E−13 | −4.62152 | 3.81E−06 |
| GFATadj | 0.0482 | −5.26 | 5.478 | 212 | 212 | blup | 0.057 | 1.20E−06 | −4.61712 | 3.89E−06 |
| GFATadj | -0.000665 | 3.48 | 2.024 | 429 | 429 | blup | 0.014 | 0.012 | 4.59687 | 4.29E−06 |
| GFATadj | 0.289627 | −10.72 | 4.587 | 353 | 8 | lasso | 0.3 | 1.70E−31 | −4.57434 | 4.78E−06 |
| GFATadj | 0.0967 | 6.16 | 4.545 | 469 | 1 | top1 | 0.097 | 2.80E−10 | 4.545 | 5.49E−06 |
| GFATadj | 0.0134 | 4 | −5.026 | 355 | 355 | blup | 0.017 | 0.0067 | −4.48943 | 7.14E−06 |
| GFATadj | 0.0141 | 4.25 | −4.465 | 386 | 1 | top1 | 0.014 | 0.011 | −4.465 | 8.01E−06 |
| GFATadj | 0.47 | −13.43 | −4.344 | 510 | 8 | lasso | 0.47 | 6.30E−55 | 4.37622 | 1.21E−05 |
| GFATadj | 0.056855 | 4.96 | 4.36 | 276 | 1 | top1 | 0.057 | 1.40E−06 | 4.36 | 1.30E−05 |
| GFATadj | 0.292 | −10.68 | −4.664 | 446 | 6 | lasso | 0.31 | 5.10E−33 | 4.35576 | 1.33E−05 |
| GFATadj | 0.0753 | −5.82 | −3.826 | 360 | 4 | lasso | 0.078 | 1.50E−08 | 4.23225 | 2.31E−05 |
| GFATadj | 0.034972 | 5.29 | 3.554 | 190 | 190 | blup | 0.1 | 4.80E−11 | 4.23018 | 2.34E−05 |
| GFATadj | 0.327 | 11.19 | 4.181 | 400 | 1 | top1 | 0.33 | 5.40E−35 | 4.181 | 2.90E−05 |
| GFATadj | 0.00261 | 3.26 | 1.175 | 317 | 317 | blup | 0.013 | 0.016 | 4.16826 | 3.07E−05 |
| GFATadj | 0.114648 | 8.19 | −4.569 | 326 | 25 | enet | 0.24 | 2.50E−25 | −4.1607 | 3.17E−05 |
| GFATadj | 0.102837 | 7.29 | 4.601 | 309 | 19 | enet | 0.13 | 4.90E−13 | 4.128555 | 3.65E−05 |
| GFATadj | 0.016414 | 4.08 | −4.096 | 415 | 1 | top1 | 0.016 | 0.0069 | −4.096 | 4.20E−05 |
| GFATadj | 0.022802 | −3.99 | −3.624 | 157 | 157 | blup | 0.037 | 8.60E−05 | 4.0786 | 4.53E−05 |
| GFATadj | 0.0986 | −6.51 | −4.06 | 306 | 1 | top1 | 0.099 | 1.90E−10 | 4.06 | 4.91E−05 |
| GFATadj | 0.081829 | 7.49 | 3.195 | 424 | 43 | enet | 0.2 | 8.70E−21 | 4.0391 | 5.37E−05 |
| GFATadj | 0.02042 | −4.73 | 1.476 | 355 | 11 | lasso | 0.093 | 6.80E−10 | −4.037479 | 5.40E−05 |
| GFATadj | 0.010368 | 4.06 | −1.757 | 358 | 16 | enet | 0.061 | 6.10E−07 | −4.004164 | 6.22E−05 |
| GFATadj | 0.0617 | −5.38 | −3.987 | 320 | 1 | top1 | 0.062 | 4.90E−07 | 3.987 | 6.69E−05 |
| GFATadj | 0.136 | 7.86 | −3.746 | 523 | 12 | enet | 0.14 | 4.70E−14 | −3.96708 | 7.28E−05 |
| GFATadj | 0.050605 | 4.61 | 3.924 | 249 | 1 | top1 | 0.051 | 5.10E−06 | 3.924 | 8.71E−05 |
| GFATadj | 0.0528 | −4.74 | 3.924 | 399 | 1 | top1 | 0.053 | 3.20E−06 | −3.924 | 8.71E−05 |
| GFATadj | 0.0596 | −5.69 | −2.212 | 120 | 5 | lasso | 0.14 | 3.90E−14 | 3.91361 | 9.09E−05 |
| GFATadj | 0.435 | −12.96 | −3.615 | 416 | 30 | enet | 0.54 | 4.90E−66 | 3.90497 | 9.42E−05 |
| GFATadj | 0.0639 | −5.57 | 3.826 | 204 | 3 | lasso | 0.066 | 2.00E−07 | −3.90136 | 9.57E−05 |
| GFATadj | 0.030824 | 4.72 | 2.086 | 291 | 291 | blup | 0.053 | 3.10E−06 | 3.896287 | 9.77E−05 |
| VAT/ASAT | 0.074925 | 5.08 | −7.263 | 469 | 1 | top1 | 0.075 | 5.40E−07 | −7.263 | 3.79E−13 |
| VAT/ASAT | 0.219 | 8.35 | −5.386 | 436 | 7 | lasso | 0.24 | 6.70E−21 | −6.11718 | 9.52E−10 |
| VAT/ASAT | 0.110123 | 6.05 | −5.784 | 324 | 1 | top1 | 0.11 | 1.10E−09 | −5.784 | 7.29E−09 |
| VAT/ASAT | 0.000918 | 4.09 | −4.988 | 77 | 77 | blup | 0.014 | 0.02 | −5.76811 | 8.02E−09 |
| VAT/ASAT | 0.0146 | −3.63 | −3.216 | 244 | 244 | blup | 0.034 | 0.00064 | 5.52948 | 3.21E−08 |
| VAT/ASAT | 0.00449 | −3.68 | −3.652 | 227 | 19 | enet | 0.037 | 0.00039 | 5.00901 | 5.47E−07 |
| VAT/ASAT | 0.139467 | 6.96 | 4.988 | 495 | 4 | lasso | 0.16 | 2.40E−13 | 4.97844 | 6.41E−07 |
| VAT/ASAT | 0.142 | −7.36 | −5.673 | 239 | 39 | enet | 0.35 | 1.40E−30 | 4.96434 | 6.89E−07 |
| VAT/ASAT | 0.12 | 7.06 | 3.138 | 197 | 8 | lasso | 0.23 | 3.60E−19 | 4.90701 | 9.25E−07 |
| VAT/ASAT | 0.503 | 12.53 | 4.482 | 249 | 12 | lasso | 0.66 | 6.80E−75 | 4.89735 | 9.71E−07 |
| VAT/ASAT | 0.082233 | 5.63 | −4.896 | 316 | 1 | top1 | 0.082 | 1.50E−07 | −4.896 | 9.78E−07 |
| VAT/ASAT | 0.0788 | −5.16 | −4.873 | 159 | 1 | top1 | 0.079 | 2.80E−07 | 4.873 | 1.10E−06 |
| VAT/ASAT | 0.174 | −7.47 | 5.63 | 233 | 4 | lasso | 0.21 | 5.10E−18 | −4.86432 | 1.15E−06 |
| VAT/ASAT | 0.006775 | 4.05 | 4.811 | 336 | 1 | top1 | 0.0068 | 0.078 | 4.811 | 1.50E−06 |
| VAT/ASAT | 0.074723 | 5.03 | 4.725 | 408 | 1 | top1 | 0.075 | 5.60E−07 | 4.725 | 2.30E−06 |
| VAT/ASAT | 0.003078 | 3.42 | −0.842 | 327 | 327 | blup | 0.016 | 0.014 | −4.65349 | 3.26E−06 |
| VAT/ASAT | 0.027774 | −4.61 | 3.382 | 378 | 378 | blup | 0.03 | 0.0012 | −4.617 | 3.89E−06 |
| VAT/ASAT | 0.00464 | 3.55 | −3 | 203 | 203 | blup | 0.0057 | 0.096 | −4.54309 | 5.54E−06 |
| VAT/ASAT | 0.000102 | −3.48 | −2.484 | 278 | 278 | blup | 0.023 | 0.0039 | 4.5164 | 6.29E−06 |
| VAT/ASAT | 0.005796 | −3.38 | −1.927 | 296 | 23 | enet | 0.033 | 0.00078 | 4.49199 | 7.06E−06 |
| VAT/ASAT | 0.036895 | 4.81 | −3.624 | 369 | 4 | lasso | 0.067 | 2.00E−06 | −4.48016 | 7.46E−06 |
| VAT/ASAT | 0.063669 | −5.38 | −4.419 | 235 | 1 | top1 | 0.064 | 3.80E−06 | 4.419 | 9.92E−06 |
| VAT/ASAT | 0.289171 | −9.55 | 3.947 | 449 | 33 | enet | 0.29 | 4.10E−25 | −4.38106 | 1.18E−05 |
| VAT/ASAT | 0.692 | 14.69 | −3.911 | 291 | 44 | enet | 0.74 | 2.50E−93 | −4.29514 | 1.75E−05 |
| VAT/ASAT | 0.0648 | 5.81 | −5.486 | 427 | 427 | blup | 0.077 | 3.90E−07 | −4.27771 | 1.89E−05 |
| VAT/ASAT | 0.141 | 6.77 | −3.195 | 412 | 37 | enet | 0.14 | 3.20E−12 | −4.26057 | 2.04E−05 |
| VAT/ASAT | 0.154 | −7.06 | −3.808 | 311 | 16 | enet | 0.16 | 2.30E−13 | 4.21151 | 2.54E−05 |
| VAT/ASAT | 0.0169 | 4.12 | −5.265 | 429 | 429 | blup | 0.024 | 0.0037 | −4.21028 | 2.55E−05 |
| VAT/ASAT | 0.080672 | −5.12 | 4.288 | 436 | 4 | lasso | 0.082 | 1.50E−07 | −4.19828 | 2.69E−05 |
| VAT/ASAT | 0.543 | 13.01 | 4.482 | 254 | 11 | lasso | 0.66 | 2.70E−75 | 4.18536 | 2.85E−05 |
| VAT/ASAT | 0.0689 | −5.41 | −3.983 | 349 | 349 | blup | 0.1 | 7.10E−09 | 4.08269 | 4.45E−05 |
| VAT/ASAT | 0.098851 | 5.66 | 3.575 | 61 | 25 | enet | 0.19 | 9.90E−16 | 4.03385 | 5.49E−05 |
| VAT/ASAT | 0.33443 | 10.54 | −3.808 | 263 | 263 | blup | 0.35 | 4.30E−31 | 4.0146 | 5.95E−05 |
| VAT/ASAT | 0.00227 | −3.46 | 3.216 | 507 | 507 | blup | 0.021 | 0.0056 | −4.0148 | 5.95E−05 |
| VAT/ASAT | 0.04 | 3.98 | −4.005 | 358 | 1 | top1 | 0.04 | 0.00022 | −4.005 | 6.20E−05 |
| VAT/ASAT | 0.30559 | −10.35 | 2.948 | 476 | 13 | lasso | 0.45 | 1.20E−42 | −3.9963 | 6.44E−05 |
| VAT/ASAT | 0.252 | 8.92 | −2.257 | 214 | 23 | enet | 0.36 | 1.30E−31 | −3.94746 | 7.90E−05 |
| VAT/ASAT | 0.005571 | −4.03 | 3.919 | 685 | 1 | top1 | 0.0056 | 0.099 | −3.919 | 8.89E−05 |
| VAT/ASAT | 0.031464 | −4.45 | 3.895 | 434 | 1 | top1 | 0.031 | 0.00097 | −3.895 | 9.82E−05 |
| VAT/ASAT | 0.0458 | 4.8 | 10.505 | 433 | 17 | enet | 0.066 | 1.80E−07 | 7.93515 | 2.10E−15 |
| VAT/ASAT | 0.0967 | 6.16 | −7.263 | 469 | 1 | top1 | 0.097 | 2.80E−10 | −7.263 | 3.79E−13 |
| VAT/ASAT | 0.077195 | 5.71 | −6.386 | 458 | 1 | top1 | 0.077 | 1.80E−08 | −6.386 | 1.70E−10 |
| VAT/ASAT | 0.034972 | 5.29 | −5.327 | 190 | 190 | blup | 0.1 | 4.80E−11 | −5.6611 | 1.50E−08 |
| VAT/ASAT | 0.116441 | 7.05 | −5.64 | 316 | 8 | lasso | 0.12 | 1.70E−12 | −5.36862 | 7.93E−08 |
| VAT/ASAT | 0.144 | 7.59 | −5.265 | 428 | 1 | top1 | 0.14 | 8.00E−15 | −5.265 | 1.40E−07 |
| VAT/ASAT | 0.130014 | 8.12 | −5.784 | 324 | 19 | enet | 0.17 | 3.70E−17 | −5.22445 | 1.75E−07 |
| VAT/ASAT | 0.0287 | 6.21 | −5.354 | 437 | 26 | enet | 0.13 | 1.40E−13 | −5.1369 | 2.79E−07 |
| VAT/ASAT | 0.523235 | 14.18 | 4.482 | 249 | 15 | lasso | 0.67 | 2.10E−95 | 5.08537 | 3.67E−07 |
| VAT/ASAT | 0.037341 | 5.22 | 4.482 | 217 | 4 | lasso | 0.081 | 8.60E−09 | 5.07064 | 3.96E−07 |
| VAT/ASAT | 0.169712 | 9.12 | 4.329 | 408 | 13 | enet | 0.2 | 3.70E−20 | 5.05418 | 4.32E−07 |
| VAT/ASAT | 0.029149 | −4.2 | −5.673 | 176 | 4 | lasso | 0.03 | 4.00E−04 | 4.9606 | 7.03E−07 |
| VAT/ASAT | 0.00586 | 3.74 | −4.565 | 358 | 358 | blup | 0.021 | 0.0028 | −4.79035 | 1.66E−06 |
| VAT/ASAT | 0.0438 | 5.52 | −5.265 | 430 | 8 | lasso | 0.054 | 2.40E−06 | −4.7698 | 1.84E−06 |
| VAT/ASAT | 0.0796 | −6.24 | −4.692 | 363 | 4 | lasso | 0.082 | 7.30E−09 | 4.73194 | 2.22E−06 |
| VAT/ASAT | 0.039049 | 5.13 | 4.725 | 400 | 1 | top1 | 0.039 | 5.70E−05 | 4.725 | 2.30E−06 |
| VAT/ASAT | 0.242 | −10.15 | −3.867 | 348 | 348 | blup | 0.25 | 2.20E−26 | 4.7132 | 2.44E−06 |
| VAT/ASAT | 0.024513 | 4.22 | 3.138 | 245 | 245 | blup | 0.049 | 7.10E−06 | 4.71102 | 2.46E−06 |
| VAT/ASAT | 0.139733 | −8.38 | −5.673 | 240 | 13 | lasso | 0.32 | 1.50E−33 | 4.70622 | 2.52E−06 |
| VAT/ASAT | 0.0412 | −6.31 | 4.7 | 345 | 1 | top1 | 0.041 | 3.70E−05 | −4.7 | 2.60E−06 |
| VAT/ASAT | 0.0152 | 3.83 | −4.159 | 191 | 191 | blup | 0.023 | 0.0017 | −−4.5971 | 4.28E−06 |
| VAT/ASAT | 0.140571 | 7.51 | 1.701 | 219 | 14 | enet | 0.17 | 3.40E−17 | 4.51781 | 6.25E−06 |
| VAT/ASAT | 0.165899 | 9.08 | 2.366 | 198 | 23 | enet | 0.33 | 1.00E−35 | 4.51439 | 6.35E−06 |
| VAT/ASAT | 0.06275 | 5.06 | 4.482 | 146 | 1 | top1 | 0.063 | 3.90E−07 | 4.482 | 7.39E−06 |
| VAT/ASAT | 0.071386 | −5.37 | −1.927 | 219 | 46 | enet | 0.11 | 1.20E−11 | 4.45197 | 8.51E−06 |
| VAT/ASAT | 0.00193 | 4.11 | −1.175 | 421 | 421 | blup | 0.015 | 0.0089 | −4.3893 | 1.14E−05 |
| VAT/ASAT | 0.001673 | −4.27 | −1.555 | 227 | 227 | blup | 0.029 | 0.00049 | 4.37962 | 1.19E−05 |
| VAT/ASAT | 0.109976 | 6.71 | 4.678 | 494 | 494 | blup | 0.11 | 9.00E−12 | 4.36516 | 1.27E−05 |
| VAT/ASAT | 0.010262 | −4.1 | 3.933 | 436 | 436 | blup | 0.024 | 0.0014 | −4.35944 | 1.30E−05 |
| VAT/ASAT | 0.304199 | 11.34 | 3.41 | 223 | 34 | enet | 0.36 | 6.40E−39 | 4.34483 | 1.39E−05 |
| VAT/ASAT | 0.318708 | −11.61 | 3.963 | 449 | 18 | enet | 0.39 | 4.50E−43 | −4.3128 | 1.61E−05 |
| VAT/ASAT | 0.04019 | −4.84 | 4.288 | 436 | 1 | top1 | 0.04 | 4.50E−05 | −4.288 | 1.80E−05 |
| VAT/ASAT | 0.050414 | −4.71 | −4.265 | 245 | 1 | top1 | 0.05 | 5.30E−06 | 4.265 | 2.00E−05 |
| VAT/ASAT | 0.0507 | 5.44 | −2.989 | 460 | 48 | enet | 0.12 | 6.10E−13 | −4.25933 | 2.05E−05 |
| VAT/ASAT | 0.067 | −6.34 | −3.846 | 267 | 267 | blup | 0.076 | 2.60E−08 | 4.23185 | 2.32E−05 |
| VAT/ASAT | 0.0753 | −5.82 | 4.166 | 360 | 4 | lasso | 0.078 | 1.50E−08 | −4.197395 | 2.70E−05 |
| VAT/ASAT | 0.0289 | −4.29 | −4.197 | 279 | 1 | top1 | 0.029 | 0.00048 | 4.197 | 2.70E−05 |
| VAT/ASAT | 0.052229 | 4.95 | 4.159 | 316 | 1 | top1 | 0.052 | 3.60E−06 | 4.159 | 3.20E−05 |
| VAT/ASAT | 0.514633 | 14.09 | 4.482 | 254 | 12 | lasso | 0.61 | 2.20E−80 | 4.06273 | 4.85E−05 |
| VAT/ASAT | 0.0236 | −4.14 | 2.346 | 400 | 400 | blup | 0.033 | 2.00E−04 | −4.061707 | 4.87E−05 |
| VAT/ASAT | 0.335692 | 11.43 | 3.575 | 65 | 11 | lasso | 0.51 | 4.20E−62 | 4.03227 | 5.52E−05 |
| VAT/ASAT | 0.1 | 6.79 | −4.029 | 364 | 1 | top1 | 0.1 | 1.30E−10 | −4.029 | 5.60E−05 |
| VAT/ASAT | 0.07367 | −6.05 | −4.021 | 333 | 1 | top1 | 0.074 | 3.90E−08 | 4.021 | 5.80E−05 |
| VAT/ASAT | 0.601 | 15.32 | −3.808 | 263 | 11 | lasso | 0.64 | 1.40E−87 | 4.01873 | 5.85E−05 |
| VAT/ASAT | 0.65 | 15.77 | −3.911 | 291 | 35 | enet | 0.68 | 7.80E−98 | −4.0051 | 6.20E−05 |
| VAT/ASAT | 0.00126 | 3.62 | 3.336 | 267 | 7 | lasso | 0.025 | 0.0011 | 3.9787 | 6.93E−05 |
| VAT/ASAT | 0.22286 | 9.4 | −2.326 | 233 | 8 | lasso | 0.27 | 1.30E−27 | −3.97776 | 6.96E−05 |
| VAT/ASAT | 0.0779 | −5.57 | −3.976 | 282 | 1 | top1 | 0.078 | 1.60E−08 | 3.976 | 7.01E−05 |
| VAT/ASAT | 0.07937 | 5.85 | −3.924 | 342 | 1 | top1 | 0.079 | 1.20E−08 | −3.924 | 8.71E−05 |
| VAT/ASAT | 0.042 | −5.2 | −3.911 | 295 | 1 | top1 | 0.042 | 3.10E−05 | 3.911 | 9.19E−05 |
| VAT/GFAT | 0.219 | 8.35 | −6.987 | 436 | 7 | lasso | 0.24 | 6.70E−21 | −8.2202 | 2.03E−16 |
| VAT/GFAT | 0.074925 | 5.08 | −6.924 | 469 | 1 | top1 | 0.075 | 5.40E−07 | −6.924 | 4.39E−12 |
| VAT/GFAT | 0.0169 | 4.12 | −7.187 | 429 | 429 | blup | 0.024 | 0.0037 | −6.5919 | 4.34E−11 |
| VAT/GFAT | 0.0648 | 5.81 | −7.37 | 427 | 427 | blup | 0.077 | 3.90E−07 | −5.9864 | 2.15E−09 |
| VAT/GFAT | 0.142 | −7.36 | −6.386 | 239 | 39 | enet | 0.35 | 1.40E−30 | 5.81825 | 5.95E−09 |
| VAT/GFAT | 0.141 | 6.77 | −4.716 | 412 | 37 | enet | 0.14 | 3.20E−12 | −5.8023 | 6.54E−09 |
| VAT/GFAT | 0.000918 | 4.09 | −5.36 | 77 | 77 | blup | 0.014 | 0.02 | −5.70102 | 1.19E−08 |
| VAT/GFAT | 0.12 | 7.06 | 3.791 | 197 | 8 | lasso | 0.23 | 3.60E−19 | 5.59608 | 2.19E−08 |
| VAT/GFAT | 0.0788 | −5.16 | −5.332 | 159 | 1 | top1 | 0.079 | 2.80E−07 | 5.332 | 9.71E−08 |
| VAT/GFAT | 0.0146 | −3.63 | −3.36 | 244 | 244 | blup | 0.034 | 0.00064 | 5.26671 | 1.39E−07 |
| VAT/GFAT | 0.174 | −7.47 | 5.806 | 233 | 4 | lasso | 0.21 | 5.10E−18 | −4.91709 | 8.78E−07 |
| VAT/GFAT | 0.503 | 12.53 | 4.159 | 249 | 12 | lasso | 0.66 | 6.80E−75 | 4.79794 | 1.60E−06 |
| VAT/GFAT | 0.289171 | −9.55 | 4.159 | 449 | 33 | enet | 0.29 | 4.10E−25 | −4.69058 | 2.72E−06 |
| VAT/GFAT | 0.000102 | −3.48 | −2.432 | 278 | 278 | blup | 0.023 | 0.0039 | 4.68639 | 2.78E−06 |
| VAT/GFAT | 0.00449 | −3.68 | −3.826 | 227 | 19 | enet | 0.037 | 0.00039 | 4.66317 | 3.11E−06 |
| VAT/GFAT | 0.00227 | −3.46 | 3.317 | 507 | 507 | blup | 0.021 | 0.0056 | −4.642 | 3.46E−06 |
| VAT/GFAT | 0.135 | 6.62 | −1.254 | 207 | 207 | blup | 0.26 | 5.70E−22 | −4.63484 | 3.57E−06 |
| VAT/GFAT | 0.234977 | 9.71 | −4.764 | 243 | 17 | enet | 0.3 | 1.30E−25 | −4.61 | 4.10E−06 |
| VAT/GFAT | 0.036 | 4.8 | 3.441 | 218 | 25 | enet | 0.08 | 2.40E−07 | 4.58603 | 4.52E−06 |
| VAT/GFAT | 0.252 | 8.92 | −2.64 | 214 | 23 | enet | 0.36 | 1.30E−31 | −4.51908 | 6.21E−06 |
| VAT/GFAT | 0.001513 | −4.14 | 3.175 | 418 | 418 | blup | 0.024 | 0.0037 | −4.44569 | 8.76E−06 |
| VAT/GFAT | 0.00464 | 3.55 | −2.903 | 203 | 203 | blup | 0.0057 | 0.096 | −4.4372 | 9.11E−06 |
| VAT/GFAT | 0.452569 | 11.91 | −4.397 | 340 | 1 | top1 | 0.45 | 1.10E−42 | −4.397 | 1.10E−05 |
| VAT/GFAT | 0.306693 | −9.89 | −4.36 | 379 | 1 | top1 | 0.31 | 1.10E−26 | 4.36 | 1.30E−05 |
| VAT/GFAT | 0.069249 | 4.83 | −4.344 | 353 | 1 | top1 | 0.069 | 1.50E−06 | −4.344 | 1.40E−05 |
| VAT/GFAT | 0.543 | 13.01 | 4.159 | 254 | 11 | lasso | 0.66 | 2.70E−75 | 4.34362 | 1.40E−05 |
| VAT/GFAT | 0.080672 | −5.12 | 4.7 | 436 | 4 | lasso | 0.082 | 1.50E−07 | −4.2932 | 1.76E−05 |
| VAT/GFAT | 0.154 | −7.06 | −3.719 | 311 | 16 | enet | 0.16 | 2.30E−13 | 4.2836 | 1.84E−05 |
| VAT/GFAT | 0.191 | −7.96 | −4.664 | 222 | 42 | enet | 0.24 | 1.20E−20 | 4.27926 | 1.88E−05 |
| VAT/GFAT | 0.000703 | −3.67 | −1.476 | 374 | 374 | blup | 0.013 | 0.024 | 4.2493 | 2.14E−05 |
| VAT/GFAT | 0.0117 | 4.32 | 2.878 | 600 | 600 | blup | 0.012 | 0.03 | 4.2376 | 2.26E−05 |
| VAT/GFAT | 0.04 | 3.98 | −4.224 | 358 | 1 | top1 | 0.04 | 0.00022 | −4.224 | 2.40E−05 |
| VAT/GFAT | 0.027774 | −4.61 | 3.455 | 378 | 378 | blup | 0.03 | 0.0012 | −4.20895 | 2.57E−05 |
| VAT/GFAT | 0.05647 | −4.96 | −4.197 | 265 | 1 | top1 | 0.056 | 1.30E−05 | 4.197 | 2.70E−05 |
| VAT/GFAT | 0.031464 | −4.45 | 4.132 | 434 | 1 | top1 | 0.031 | 0.00097 | −4.132 | 3.60E−05 |
| VAT/GFAT | 0.0666 | −5.2 | −4.125 | 427 | 1 | top1 | 0.067 | 2.30E−06 | 4.125 | 3.71E−05 |
| VAT/GFAT | 0.33443 | 10.54 | −4.173 | 263 | 263 | blup | 0.35 | 4.30E−31 | −4.10153 | 4.10E−05 |
| VAT/GFAT | 0.005796 | −3.38 | −2.132 | 296 | 23 | enet | 0.033 | 0.00078 | 4.06948 | 4.71E−05 |
| VAT/GFAT | 0.250251 | −8.93 | −4.056 | 265 | 1 | top1 | 0.25 | 2.20E−21 | 4.056 | 4.99E−05 |
| VAT/GFAT | 0.0216 | −4.3 | −3.023 | 189 | 37 | enet | 0.039 | 0.00025 | 4.04007 | 5.34E−05 |
| VAT/GFAT | 0.039915 | 4.71 | 3.998 | 372 | 1 | top1 | 0.04 | 0.00023 | 3.998 | 6.39E−05 |
| VAT/GFAT | 0.0167 | 3.46 | 2.014 | 481 | 481 | blup | 0.025 | 0.0031 | 3.957 | 7.58E−05 |
| VAT/GFAT | 0.168 | 7.98 | −4.36 | 196 | 196 | blup | 0.19 | 3.00E−16 | −3.9137 | 9.09E−05 |
| VAT/GFAT | 0.0368 | −4.59 | 3.846 | 596 | 2 | lasso | 0.041 | 0.00018 | −3.91336 | 9.10E−05 |
| VAT/GFAT | 0.005022 | 3.89 | 1.126 | 380 | 380 | blup | 0.062 | 5.20E−06 | 3.9006 | 9.60E−05 |
| VAT/GFAT | 0.0287 | 6.21 | −6.937 | 437 | 26 | enet | 0.13 | 1.40E−13 | −7.24449 | 4.34E−13 |
| VAT/GFAT | 0.144 | 7.59 | −7.187 | 428 | 1 | top1 | 0.14 | 8.00E−15 | −7.187 | 6.62E−13 |
| VAT/GFAT | 0.0967 | 6.16 | −6.924 | 469 | 1 | top1 | 0.097 | 2.80E−10 | −6.924 | 4.39E−12 |
| VAT/GFAT | 0.0438 | 5.52 | −7.187 | 430 | 8 | lasso | 0.054 | 2.40E−06 | −6.68875 | 2.25E−11 |
| VAT/GFAT | 0.140571 | 7.51 | 3.441 | 219 | 14 | enet | 0.17 | 3.40E−17 | 6.17799 | 6.49E−10 |
| VAT/GFAT | 0.139733 | −8.38 | −6.386 | 240 | 13 | lasso | 0.32 | 1.50E−33 | 5.81626 | 6.02E−09 |
| VAT/GFAT | 0.0412 | −6.31 | 5.715 | 345 | 1 | top1 | 0.041 | 3.70E−05 | −5.715 | 1.10E−08 |
| VAT/GFAT | 0.034972 | 5.29 | −5.384 | 190 | 190 | blup | 0.1 | 4.80E−11 | −5.70654 | 1.15E−08 |
| VAT/GFAT | 0.077195 | 5.71 | −5.53 | 458 | 1 | top1 | 0.077 | 1.80E−08 | −5.53 | 3.20E−08 |
| VAT/GFAT | 0.165899 | 9.08 | 2.903 | 198 | 23 | enet | 0.33 | 1.00E−35 | 5.42329 | 5.85E−08 |
| VAT/GFAT | 0.0745 | 5.67 | −5.376 | 436 | 1 | top1 | 0.075 | 3.30E−08 | −5.376 | 7.62E−08 |
| VAT/GFAT | 0.103 | 7.18 | 2.543 | 399 | 399 | blup | 0.13 | 7.10E−14 | 5.11777 | 3.09E−07 |
| VAT/GFAT | 0.00586 | 3.74 | −4.775 | 358 | 358 | blup | 0.021 | 0.0028 | −5.0852 | 3.67E−07 |
| VAT/GFAT | 0.049509 | 4.61 | 1.015 | 95 | 37 | enet | 0.1 | 7.50E−11 | 5.06232 | 4.14E−07 |
| VAT/GFAT | 0.024513 | 4.22 | 3.791 | 245 | 245 | blup | 0.049 | 7.10E−06 | 4.99746 | 5.81E−07 |
| VAT/GFAT | 0.523235 | 14.18 | 4.159 | 249 | 15 | lasso | 0.67 | 2.10E−95 | 4.93855 | 7.87E−07 |
| VAT/GFAT | 0.029149 | −4.2 | −6.386 | 176 | 4 | lasso | 0.03 | 4.00E−04 | 4.90487 | 9.35E−07 |
| VAT/GFAT | 0.037341 | 5.22 | 4.159 | 217 | 4 | lasso | 0.081 | 8.60E−09 | 4.82216 | 1.42E−06 |
| VAT/GFAT | 0.0368 | 4.7 | 4.775 | 338 | 338 | blup | 0.044 | 2.00E−05 | 4.78673 | 1.70E−06 |
| VAT/GFAT | 0.0796 | −6.24 | −4.753 | 363 | 4 | lasso | 0.082 | 7.30E−09 | 4.74986 | 2.04E−06 |
| VAT/GFAT | 0.010262 | −4.1 | 4.065 | 436 | 436 | blup | 0.024 | 0.0014 | −4.72604 | 2.29E−06 |
| VAT/GFAT | 0.001673 | −4.27 | −2.457 | 227 | 227 | blup | 0.029 | 0.00049 | 4.71409 | 2.43E−06 |
| VAT/GFAT | 0.04019 | −4.84 | 4.7 | 436 | 1 | top1 | 0.04 | 4.50E−05 | −4.7 | 2.60E−06 |
| VAT/GFAT | 0.309 | 10.94 | −4.764 | 243 | 16 | enet | 0.38 | 1.70E−41 | −4.680375 | 2.86E−06 |
| VAT/GFAT | 0.050414 | −4.71 | −4.639 | 245 | 1 | top1 | 0.05 | 5.30E−06 | 4.639 | 3.50E−06 |
| VAT/GFAT | 0.071386 | −5.37 | −1.476 | 219 | 46 | enet | 0.11 | 1.20E−11 | 4.63056 | 3.65E−06 |
| VAT/GFAT | 0.070425 | 5.41 | 4.569 | 463 | 1 | top1 | 0.07 | 7.80E−08 | 4.569 | 4.90E−06 |
| VAT/GFAT | 0.0583 | 6.78 | −4.716 | 413 | 5 | lasso | 0.12 | 8.40E−13 | −4.55255 | 5.30E−06 |
| VAT/GFAT | 0.318708 | −11.61 | 4.145 | 449 | 18 | enet | 0.39 | 4.50E−43 | −4.53831 | 5.67E−06 |
| VAT/GFAT | 0.0525 | −5.41 | −3.36 | 395 | 395 | blup | 0.057 | 1.40E−06 | 4.52674 | 5.99E−06 |
| VAT/GFAT | 0.515 | 14.19 | −4.397 | 340 | 41 | enet | 0.53 | 3.00E−64 | −4.52006 | 6.18E−06 |
| VAT/GFAT | 0.0152 | 3.83 | −4.344 | 191 | 191 | blup | 0.023 | 0.0017 | −4.47403 | 7.68E−06 |
| VAT/GFAT | 0.00193 | 4.11 | −2.308 | 421 | 421 | blup | 0.015 | 0.0089 | −4.3956 | 1.10E−05 |
| VAT/GFAT | 0.124191 | 8.35 | −3.826 | 214 | 7 | lasso | 0.33 | 5.70E−35 | −4.38274 | 1.17E−05 |
| VAT/GFAT | 0.067 | −6.34 | −4.189 | 267 | 267 | blup | 0.076 | 2.60E−08 | 4.33369 | 1.47E−05 |
| VAT/GFAT | 0.242 | −10.15 | −3.441 | 348 | 348 | blup | 0.25 | 2.20E−26 | 4.3031 | 1.68E−05 |
| VAT/GFAT | 0.0174 | −4.56 | 4.244 | 341 | 1 | top1 | 0.017 | 0.0055 | −4.244 | 2.20E−05 |
| VAT/GFAT | 0.155523 | −8.12 | 4.344 | 434 | 5 | lasso | 0.18 | 1.80E−18 | −4.2151 | 2.50E−05 |
| VAT/GFAT | 0.0743 | −6.3 | −4.197 | 265 | 1 | top1 | 0.074 | 3.40E−08 | 4.197 | 2.70E−05 |
| VAT/GFAT | 0.352377 | −11.92 | −4.36 | 379 | 6 | lasso | 0.36 | 6.70E−39 | 4.16434 | 3.12E−05 |
| VAT/GFAT | 0.06275 | 5.06 | 4.159 | 146 | 1 | top1 | 0.063 | 3.90E−07 | 4.159 | 3.20E−05 |
| VAT/GFAT | 0.514633 | 14.09 | 4.159 | 254 | 12 | lasso | 0.61 | 2.20E−80 | 4.15084 | 3.31E−05 |
| VAT/GFAT | 0.22286 | 9.4 | −2.273 | 233 | 8 | lasso | 0.27 | 1.30E−27 | −4.14811 | 3.35E−05 |
| VAT/GFAT | 0.224 | −9.32 | −4.056 | 265 | 1 | top1 | 0.22 | 5.30E−23 | 4.056 | 4.99E−05 |
| VAT/GFAT | 0.0507 | 5.44 | −2.484 | 460 | 48 | enet | 0.12 | 6.10E−13 | −4.04713 | 5.18E−05 |
| VAT/GFAT | 0.0143 | −4.03 | −4.042 | 257 | 1 | top1 | 0.014 | 0.011 | 4.042 | 5.30E−05 |
| VAT/GFAT | 0.0236 | −4.14 | 2.346 | 400 | 400 | blup | 0.033 | 2.00E−04 | −4.03388 | 5.49E−05 |
| VAT/GFAT | 0.304199 | 11.34 | 2.87 | 223 | 34 | enet | 0.36 | 6.40E−39 | 4.00335 | 6.25E−05 |
| VAT/GFAT | 0.601 | 15.32 | −4.132 | 263 | 11 | lasso | 0.64 | 1.40E−87 | −3.991 | 6.58E−05 |
| VAT/GFAT | 0.07367 | −6.05 | −3.957 | 333 | 1 | top1 | 0.074 | 3.90E−08 | 3.957 | 7.59E−05 |
| VAT/GFAT | 0.0304 | 4.02 | 3.921 | 257 | 1 | top1 | 0.03 | 0.00035 | 3.921 | 8.82E−05 |
| VAT/GFAT | 0.172 | 8.4 | −4.397 | 196 | 196 | blup | 0.18 | 6.80E−19 | −3.91608 | 9.00E−05 |
| VAT/GFAT | 0.0289 | −4.29 | −3.913 | 279 | 1 | top1 | 0.029 | 0.00048 | 3.913 | 9.12E−05 |
| VAT/GFAT | 0.0432 | −4.77 | 3.903 | 308 | 1 | top1 | 0.043 | 2.40E−05 | −3.903 | 9.50E−05 |
| ASAT/GFAT | 0.0287 | 6.21 | −4.953 | 437 | 26 | enet | 0.13 | 1.40E−13 | −6.138826 | 8.31E−10 |
| ASAT/GFAT | 0.0458 | 4.8 | −8.574 | 433 | 17 | enet | 0.066 | 1.80E−07 | −5.86037 | 4.62E−09 |
| ASAT/GFAT | 0.144 | 7.59 | −5.541 | 428 | 1 | top1 | 0.14 | 8.00E−15 | −5.541 | 3.01E−08 |
| ASAT/GFAT | 0.17 | 8.37 | −5.554 | 425 | 11 | lasso | 0.18 | 5.00E−18 | −5.43884 | 5.36E−08 |
| ASAT/GFAT | 0.0438 | 5.52 | −5.541 | 430 | 8 | lasso | 0.054 | 2.40E−06 | −5.419753 | 5.97E−08 |
| ASAT/GFAT | 0.0162 | 4.26 | 4.314 | 338 | 338 | blup | 0.021 | 0.0028 | 4.94514 | 7.61E−07 |
| ASAT/GFAT | 0.013525 | 3.78 | −5.199 | 367 | 367 | blup | 0.017 | 0.0059 | −4.77453 | 1.80E−06 |
| ASAT/GFAT | 0.352377 | −11.92 | −4.764 | 379 | 6 | lasso | 0.36 | 6.70E−39 | 4.750221 | 2.03E−06 |
| ASAT/GFAT | 0.050605 | 4.61 | −4.708 | 249 | 1 | top1 | 0.051 | 5.10E−06 | −4.708 | 2.50E−06 |
| ASAT/GFAT | 0.051297 | −5.76 | −3.195 | 258 | 30 | enet | 0.13 | 9.60E−14 | 4.68756 | 2.76E−06 |
| ASAT/GFAT | 0.142 | 7.47 | 4.596 | 547 | 1 | top1 | 0.14 | 1.30E−14 | 4.596 | 4.31E−06 |
| ASAT/GFAT | 0.106 | 6.89 | 3.39 | 398 | 4 | lasso | 0.15 | 3.10E−15 | 4.59509 | 4.33E−06 |
| ASAT/GFAT | 0.140571 | 7.51 | 4.046 | 219 | 14 | enet | 0.17 | 3.40E−17 | 4.5725 | 4.82E−06 |
| ASAT/GFAT | 0.103 | 7.18 | 2.226 | 399 | 399 | blup | 0.13 | 7.10E−14 | 4.56116 | 5.09E−06 |
| ASAT/GFAT | 0.0482 | −5.26 | −4.36 | 212 | 212 | blup | 0.057 | 1.20E−06 | 4.3274 | 1.51E−05 |
| ASAT/GFAT | 0.023 | 4.8 | 3.138 | 399 | 399 | blup | 0.039 | 5.20E−05 | 4.32302 | 1.54E−05 |
| ASAT/GFAT | 0.0528 | −4.74 | −4.314 | 399 | 1 | top1 | 0.053 | 3.20E−06 | 4.314 | 1.60E−05 |
| ASAT/GFAT | 0.13 | 7.14 | −4.288 | 218 | 1 | top1 | 0.13 | 1.80E−13 | −4.288 | 1.80E−05 |
| ASAT/GFAT | 0.073 | 5.47 | −4.288 | 199 | 1 | top1 | 0.073 | 4.50E−08 | −4.288 | 1.80E−05 |
| ASAT/GFAT | 0.064 | 5.27 | −4.288 | 11 | 1 | top1 | 0.064 | 3.00E−07 | −4.288 | 1.80E−05 |
| ASAT/GFAT | 0.0267 | −5.02 | −4.113 | 453 | 6 | lasso | 0.032 | 0.00025 | 4.13061 | 3.62E−05 |
| ASAT/GFAT | 0.429 | 13.07 | −3.919 | 450 | 5 | lasso | 0.44 | 1.20E−50 | −4.1254 | 3.70E−05 |
| ASAT/GFAT | 0.0745 | 5.67 | −4.125 | 436 | 1 | top1 | 0.075 | 3.30E−08 | −4.125 | 3.71E−05 |
| ASAT/GFAT | 0.0182 | 3.82 | −3.891 | 146 | 146 | blup | 0.02 | 0.0029 | −4.12206 | 3.75E−05 |
| ASAT/GFAT | 0.0207 | −4.35 | −4.102 | 427 | 1 | top1 | 0.021 | 0.0027 | 4.102 | 4.10E−05 |
| ASAT/GFAT | 0.0228 | −4.58 | −3.808 | 146 | 146 | blup | 0.038 | 7.50E−05 | 4.09951 | 4.14E−05 |
| ASAT/GFAT | 0.07085 | 5.49 | −3.39 | 61 | 20 | enet | 0.19 | 1.80E−19 | −4.09096 | 4.30E−05 |
| ASAT/GFAT | 0.051922 | −5.54 | 3.652 | 486 | 3 | lasso | 0.053 | 3.30E−06 | −4.05445 | 5.03E−05 |
| ASAT/GFAT | 0.0606 | 5.59 | 4.234 | 566 | 3 | lasso | 0.064 | 2.90E−07 | 4.00696 | 6.15E−05 |
| ASAT/GFAT | 0.279 | 10.38 | −4.288 | 164 | 31 | enet | 0.35 | 8.50E−38 | −3.9866 | 6.70E−05 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating a metabolic disorder comprising:
detecting one or more indicators of metabolic disease in a subject having a variant that increases risk for the metabolic disorder or a variant that decreases risk for the metabolic disorder; and
treating the subject with one or more agents capable of treating the metabolic disorder if the one or more indicators of metabolic disease are detected in the subject having a variant that increases risk for the metabolic disorder, optionally,
wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657; or
detecting one or more indicators of metabolic disease in a subject having a polygenic risk score (PRS) for an adiposity trait adjusted for BMI and height selected from the group consisting of GFAT, VAT, and ASAT; and
treating the subject with one or more agents capable of treating the metabolic disorder if the one or more indicators of metabolic disease are detected in the subject having a low PRS for BMI and height adjusted GFAT, a high PRS for BMI and height adjusted VAT, and/or a high PRS for BMI and height adjusted ASAT; or
treating the subject with a healthy lifestyle regimen if the one or more indicators of metabolic disease are detected in the subject having a high PRS for BMI and height adjusted GFAT, a low PRS for BMI and height adjusted VAT, and/or a low PRS for BMI and height adjusted ASAT.

2. The method of claim 1, wherein the one or more indicators of metabolic disease is selected from the group consisting of: increased visceral adipose tissue (VAT), increased abdominal subcutaneous adipose tissue (ASAT), decreased gluteofemoral adipose tissue (GFAT), increased serum triglycerides, decreased HDL-c (HDL-cholesterol), increased LDL-c (LDL-cholesterol), increased liver enzymes, optionally, alanine aminotransferase (ALT), and increased HbA1C (hemoglobin A1C).

3. (canceled)

4. The method of claim 1, wherein the one or more indicators of metabolic disease are detected by a blood test, a CT-scan, a DEXA-scan, or an MRI.

5. (canceled)

6. The method of claim 1, wherein the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance.

7. (canceled)

8. The method of claim 1, wherein the variant activity of the PRS is enriched in adipose tissue; or
wherein the PRS includes up to 1,125,301 variants.

9-14. (canceled)

15. The method of claim 1, wherein the one or more agents comprise a PPAR-alpha agonist, a PPAR-gamma agonist, optionally, wherein the PPAR-gamma agonist is a thiazolidinedione selected from the group consisting of Pioglitazone, Rosiglitazone, Lobeglitazone, Ciglitazone, Darglitazone, Englitazone, Netoglitazone, Rivoglitazone, Troglitazone, Balaglitazone, and AS-605240, a PPAR-delta agonist, a dual or pan PPAR agonist, a growth hormone-releasing hormone (GHRH), optionally, wherein the GHRH is selected from the group consisting of Tesamorelin, Somatocrinin, CJC-1295, Modified GRF (1-29), Dumorelin, Rismorelin, Sermorelin, and Somatorelin, a sodium-glucose transporter 2 (SGLT2) inhibitor, optionally, wherein the SGLT2 inhibitor is selected from the group consisting of Canagliflozin, Dapagliflozin, Empagliflozin, Ertugliflozin, Ipragliflozin, Luseogliflozin, Remogliflozin, Sotagliflozin, and Tofogliflozin, metformin, an alpha-glucosidase inhibitor, an incretin based therapy, a sulfonylurea, metreleptin, an antisense oligonucleotide (ASO), or a gene modifying agent, optionally, wherein the gene modifying agent is a CRISPR-Cas gene editing agent.

16-31. (canceled)

32. A method of treating a metabolic disorder in a subject in need thereof comprising: administering one or more agents targeting a gene associated with a variant selected from 3:49799046_CA_C, 5:55802127_TCAAGGATTCCTTGACTTAAG_T, rs73221948, rs56094641, rs62120394, 19:33785832_CA_C, rs3786897, rs34670319, rs147603433, rs4801774, rs62106258, rs1325033, rs7461961, rs56094641, rs62120394, rs79818747, rs56094641, rs11642015, rs2820468, rs200472737, rs355906, rs78058190, rs2972147, rs16885714, rs9379833, rs9265830, rs115250958, rs35381162, rs529311472, rs141958096, rs4711750, rs1325033, 6:105373111_CT_C, rs72959041, rs487060, rs1074742, rs147730268, rs138756410, rs7133378, rs825453, rs4765159, rs56094641, 19:34019403_GAC_G, rs79818747, rs6001008, rs2943653, rs56094641, rs13389219, rs146623665, rs4711750, 6:105373111_CT_C, rs7133378, rs825453, rs12089366, rs56006999, rs35932591, rs3731861, rs56082403, rs30351, rs72810972, rs9266218, rs76072243, rs115250958, rs2858856, rs185139895, rs998584, rs2800736, rs577721086, rs5880430, rs149643430, rs11992444, rs4872393, rs1329254, rs11031796, 11:46610325_CA_C, rs7933253, rs7133378, 12:124503803_CAA_C, 19:33785832_CA_C, rs7250362, rs55865721, rs10406327, rs28451064, 1:11099387_GTGGATGGATGGA_G, rs35932591, rs30351, rs10054063, rs113602321, rs998584, rs11992444, rs35641603, rs73026242, rs10406327, rs28451064, rs56006999, rs1500714, rs13322435, rs9266627, 6:32621590_T_C, rs577721086, rs4052908, rs73221948, rs1962883, 12:122820960_TAA_T, rs7133378, 12:124503803_CAA_C, 19:33785832_CA_C, rs10406327, rs73041147, rs33845, rs1779445, rs3850625, rs6685593, rs7538503, rs2943647, rs527620413, rs7649153, rs13322435, rs55744247, rs3936510, rs1159619, rs553015785, rs73221948, rs2048235, rs6474550, rs17205757, rs768397327, 15:85091836_CA_C, rs8077609, rs4444401, rs2302209, rs6704389, rs7538503, rs2943646, rs527620413, rs6807940, rs9854955, rs768397327, rs112489358, rs749166380, rs6691427, 5:55860907_GC_G, rs998584, rs1558919, rs553015785, rs776481989, 15:84570588_TGA_T, rs72641832, rs11205303, rs559230165, rs7588285, rs13389219, rs3820981, rs34224594, rs78058190, 2:226768344_CA_C, rs2943634, rs35414396, rs71304101, rs9855622, rs2300669, rs199874557, rs62271373, rs13099700, rs4450871, rs874040, rs13142096, rs3822072, rs546560809, rs6822892, rs142369482, rs11429307, rs10044492, rs1294437, 6:32936748_TG_T, rs199679345, rs998584, rs5875852, rs72959041, 6:127457071_CA_C, rs2982521, rs11390479, rs1962883, rs111874795, rs1907218, rs10501153, rs71468663, rs71455776, rs748889, rs12814794, rs4759309, rs147730268, rs150792771, rs7133378, rs11057402, rs825453, rs2955617, rs8075019, rs3786920, rs1883711, rs55951234, rs4846303, rs6704389, rs78058190, rs2943648, rs71304101, rs528845403, rs6822892, rs199679345, rs11967262, rs364663, rs72959041, 6:127457071_CA_C, rs7550430, rs559230165, rs17326656, rs13389219, rs386652275, rs13410987, rs34224594, rs2943634, rs55664914, rs1872113, rs62271373, rs11429307, rs115177000, rs998584, rs140626545, rs191578827, rs4273712, rs72959041, 6:127457071_CA_C, rs4052908, rs1561105, rs6994124, rs1962883, rs56271783, rs12814794, rs894739, rs147730268, rs7133378, rs825453, rs139254114, rs2925979, rs13303359, rs2384054, rs13028464, rs2396316, rs17036328, rs56082403, 5:55860907_GC_G, rs112299234, rs6903044, rs70987287, rs2853951, rs17193640, rs76072243, 6:32900378_CCT_C, rs185139895, rs1936789, rs577721086, rs2982521, rs9484299, rs3890765, rs73221948, rs6997996, rs6474552, rs55767272, rs11199845, rs11031796, rs7133378, rs4925049, rs269967, 19:33785832_CA_C, rs55865721, rs10406327, rs12321, rs13390751, 2:227100579_TC_T, rs527620413, rs56082403, rs10054063, 6:19949170_GT_G, rs2524137, rs375009120, rs11967262, rs73221948, rs11199844, rs11031796, 19:33785832_CA_C, rs73026242, rs10406327, rs28451064, rs916485, rs13322435, rs70987287, rs185139895, rs577721086, rs2982521, 7:130451984_CTTTA_C, rs73221948, rs3809060, rs59757908, rs7133378, 19:33785832_CA_C, rs889138, rs55920843, rs2396316, rs17036328, 3:49799046_CA_C, rs490701, rs455660, rs72812818, rs2853951, rs3117109, 6:32621590_T_C, rs185139895, rs998584, rs9472136, 6:127333964_AG_A, rs1936789, rs577721086, rs2982521, rs11992444, rs10086575, rs568011588, rs35169799, rs718314, rs7133378, 12:124503803_CAA_C, rs28929474, 19:33785832_CA_C, rs10406327, rs73041147, rs28451064, rs12321, rs30351, rs55646464, rs9266247, rs2647006, rs11967262, rs6916318, rs72959041, rs73221948, rs5418, rs9660318, rs11399916, rs10221833, rs9276981, rs185139895, rs1936789, rs577721086, rs151288714, rs11992444, 12:122820960_TAA_T, rs7133378, 19:33785832_CA_C, rs3786901, rs1779445, rs564667, 3:49803078_TA_T, rs9854955, rs28730491, rs39837, rs3843467, rs998584, rs744103, rs9375487, rs7843475, rs7133378, rs8006225, rs1421085, rs1552657, rs2302209, rs1423062, rs4680338, rs56094641, rs2645290, rs39837, rs3936510, rs998584, rs744103, rs10246191, rs553015785, rs71468663, and rs7133378, or
administering one or more agents targeting one or more genes associated with an adiposity trait adjusted for BMI and height selected from the group consisting of GFAT, VAT and ASAT, wherein the one or more genes are selected from CEBPA-AS1, CCDC92, FLOT1, CYP21A1P, HLA-DRB6, HLA-S, ATG13, APOM, EXOSC10, PRRT1, MAST3, HCG23, DNAH10, HLA-DQA2, HLA-DRB1, PNKD, RP11-380L11.4, RP11-378A13.1, XXbac-BPG248L24.12, HCG27, HLA-C, TBX15, NAA25, C4B, NCKIPSD, TMBIM1, DALRD3, DNAH100S, JAZF1, PSORS1C1, HLA-DQB1-AS1, WDR6, DSTYK, P4HTM, IFT80, CCDC36, RP11-3B7.1, C3orf62, CYP21A2, RP5-935K16.1, CD79B, LMBR1L, ALKBH5, ADCY3, CENPW, TIPARP, AC103965.1, CSPG4P11, IRS1, RP11-671M22.4, RIMKLBP2, PAN2, XYLB, EXOG, CTD-2007L18.5, RP11-977G19.11, STAT2, RP4-712E4.1, ACO2, THBS3, RP11-392O17.1, RFTN2, RP11-43F13.3, EYA1, CD79B, KLF14, RN7SL417P, TBX15, NKD2, MEST, SCAND2P, ARNT, RPS18P9, NMT1, LINC00933, RP11-347119.8, RAF1, RP11-419C23.1, RHOF, AC084018.1, MEI1, RP11-182J1.13, EP300, GOLGA6L5, GBAP1, RP11-328C8.2, RP11-182J1.5, CCDC92, DNAH100S, RP11-380L11.4, IRS1, ZNF664, RIMKLBP2, DNAH10, RP11-392O17.1, VEGFB, FAM13A, PDGFC, MAFF, TMEM165, RP11-177J6.1, CLOCK, SRD5A3-AS1, PEPD, EXOG, ATP6V0A2, BAIAP2L2, RP11-32D16.1, RP11-211G23.2, GRB14, XXbac-BPG248L24.12, CTC-228N24.3, RP11-708J19.1, SUMO2, KREMEN1, PTPN23, ROM1, XYLB, RP3-323P13.2, CHST8, EEF1G, ATP1B2, MUC1, EML3, SETD2, RPS18P9, NMUR1, CEBPA-AS1, SENP2, B3GAT3, SNX10, EP300, MYEOV, PRDX5, C4B, RP11-470E16.1, PTH1R, DCAKD, MEI1, RP11-309N17.4, RP11-798G7.5, RP5-1115A15.1, RNF157, CTA-228A9.3, SLC16A8, FLRT1, TMEM60, CALCRL, RP11-2E11.5, RP11-196G18.22, WARS2, SEPT1, ACO2, CEBPA-AS1, CCDC92, ADCY3, FLOT1, APOM, HCG23, AC079305.11, HLA-S, CYP21A1P, HLA-DRB6, CENPO, PRRT1, HLA-DRB1, EFR3B, PEMT, DNAJC27, RRAS2, NAA25, C3orf62, MIR4435-1HG, RP11-43F13.3, ATG13, RP11-378A13.1, RPS26, DNAH100S, DNAH10, GS1-259H13.2, RP11-380L11.4, PNKD, HLA-DQA2, RP11-282018.3, ARL17B, WDR6, BTN3A3, EXOSC10, TMEM80, HLA-DQB1-AS1, PCBD1, TMBIM1, TIPARP, CEBPA-AS1, IRS1, C4B, CENPO, DNAH100S, ADCY3, CCDC92, HLA-DRB6, HLA-DRA, PEMT, XXbac-BPG299F13.14, EXOSC10, RP11-380L11.4, RP4-635E18.7, RP11-524F11.1, CDK2AP1, MSH5, HLA-S, VEGFB, ADAM1B, XXbac-BPG248L24.12, CYP21A1P, XXbac-BPG154L12.4, HLA-B, PAPPA, C2, RP11-132M7.3, AAMP, SKIV2L, RP11-378A13.1, PNKD, CLIC1, GSTM1, ARIH2, PRDX5, HECTD4, LINC00910, HLA-DQA2, DMWD, NSFP1, WNT16, CLTB, WDR6, RPS26, PAN2, HLA-DRB1, C11orf49, C6orf106, SUOX, CCDC92, CEBPA-AS1, RP11-380L11.4, DNAH100S, HLA-S, DNAH10, FLOT1, CYP21A1P, PRRT1, APOM, HLA-DRB1, HLA-DRB6, RP11-378A13.1, C3orf62, HCG23, BTN3A3, HLA-C, FAM154B, XXbac-BPG248L24.12, HLA-DQB1-AS1, MAST3, NAA25, RBM6, CTC-228N24.3, SEMA3F, HLA-DQA2, PNKD, GS1-259H13.2, C4A, TRAPPC10, RP11-114F10.3, EXOSC10, RRAS2, DALRD3, TMBIM1, TBX15, WDR6, MIR4435-1HG, NCKIPSD, CYP21A2, NT5DC2, ZSCAN12P1, TMEM116, DSTYK, SLC12A2, CCDC92, DNAH100S, CEBPA-AS1, RP11-380L11.4, XXbac-BPG248L24.12, HLA-S, VEGFB, C4B, IRS1, CYP21A1P, ZNF664, ATP6V0A2, EXOSC10, VARS2, MSH5, HLA-DRB6, XXbac-BPG299F13.14, HLA-DRA, MST1R, RP4-635E18.7, AAMP, C2, PNKD, FAM154B, CLIC1, HLA-B, FAM13A, DNAH10, RP11-378A13.1, NEK4, RBM6, ADAM1B, PAPPA, HLA-DQB1-AS1, ARIH2, CDK2AP1, MAP3K13, TMBIM1, DALRD3, CTC-228N24.3, XXbac-BPG154L12.4, HLA-DQA2, HLA-DRB1, NCKIPSD, GSTM1, CELSR3, DMWD, SKIV2L, WDR6, CLTB, QARS, TMEM116, HECTD4, MRAS, CCDC92, TIPARP, DNAH100S, RP4-712E4.1, RP11-380L11.4, THB S3, PDGFC, CTC-228N24.3, CALCRL, WNT3, EYA1, MEST, XXbac-BPG248L24.12, ATP6V0A2, SETD2, RP11-2E11.9, RP11-2E11.5, PMS2P3, POM121C, GTF2IP1, CTD-2380F24.1, KNOP1, ZNF664, PTPN23, TBX15, RP11-708J19.1, ARL17B, RBFOX2, GNA12, and STAG3L1.

33. The method of claim 32, wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657.

34. The method of claim 32 or 33, wherein the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), non-alcoholic fatty liver disease (NAFLD), and impaired glucose tolerance.

35. The method of claim 32, wherein the expression of the gene associated with a variant is regulated by the variant; or
wherein the gene associated with a variant is in contact with a genomic loci comprising the variant.

36-37. (canceled)

38. The method of claim 32, wherein the one or more genes associated with an adiposity trait adjusted for BMI and height are selected from the group consisting of:
a) CEBPA-AS1, CCDC92, FLOT1, CYP21A1P, HLA-DRB6, and HLA-S; or
b) CENPW, TIPARP, and AC103965.1; or
c) CCDC92, DNAH100S, RP11-380L11.4, IRS1, ZNF664, RIMKLBP2, DNAH10, RP11-392O17.1, VEGFB, FAM13A, PDGFC, MAFF, TMEM165, RP11-177J6.1, CLOCK, and SRD5A3-AS1; or
d) CEBPA-AS1, CCDC92, ADCY3, FLOT1, TIPARP, CEBPA-AS1, and IRS1; or
e) CCDC92, CEBPA-AS1, RP11-380L11.4, DNAH100S, HLA-S, DNAH10, CCDC92, DNAH100S, CEBPA-AS1, RP11-380L11.4, XXbac-BPG248L24.12, HLA-S, and VEGFB; or
f) CCDC92, and TIPARP.

39. (canceled)

40. The method of claim 32, wherein the one or more agents is an agonist of the gene, an antagonist of the gene, a small molecule, an antisense oligonucleotide (ASO), or a gene modifying agent, optionally, wherein the gene modifying agent is a CRISPR-Cas gene editing agent; or
wherein the one or more agents increase or decrease expression of the gene.

41-47. (canceled)

48. The method of claim 32, further comprising monitoring treatment efficacy by detecting one or more indicators of the metabolic disorder in the subject.

49. A method of detecting one or more risk variants or a risk for a metabolic disorder comprising detecting in a subject one or more risk variants associated with an adiposity trait adjusted for BMI and height selected from the group consisting of GFAT, VAT and ASAT.

50. The method of claim 49, wherein the variant is selected from the group consisting of: rs1074742, rs138756410, rs4765159, rs35932591, rs1329254, rs7933253, rs1500714, rs3850625, rs2048235, rs6474550, rs17205757, rs4444401, rs749166380, rs776481989, rs7588285, 2:226768344_CA_C, rs13099700, rs142369482, rs1907218, rs528845403, rs7550430, rs386652275, rs13028464, rs70987287, rs3890765, rs6474552, rs55767272, rs11199845, rs13390751, 6:19949170_GT_G, rs11199844, rs59757908, rs28929474, rs9660318, rs11399916, rs9276981, rs39837, rs8006225, and rs1552657.

51. The method of claim 49, wherein the metabolic disorder is selected from the group consisting of coronary artery disease (CAD), hypertension, type 2 diabetes (T2D), lipodystrophy, familial partial lipodystrophy (FPLD), insulin resistance, dyslipidemia, metabolic syndrome, non-alcoholic steatohepatitis (NASH), Nonalcoholic fatty liver disease (NAFLD), and impaired glucose tolerance.

52. The method of claim 49, wherein the one or more variants are polygenic risk variants.

53. The method of claim 1, wherein the subject is female.

54-55. (canceled)

56. The method of claim 50, wherein 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 of the risk variants are detected in a sample from the subject.

57. The method of claim 50, wherein the one or more risk variants are detected by hybridization, nucleic acid amplification, or sequencing.
